SEQUENCE LISTING (67 sequences)

SEQ ID NO: 1 - DNA, 46 bp, Artificial Sequence
  Promoter consensus sequence
  misc_feature 29: /note=“Wherein n represents a gap in the consensus sequence or is absent”
gtgcggctcggaaattcacawggkccgtntygtgcggctcggaaat

SEQ ID NO: 2 - DNA, 1000 bp, Yarrowia lipolytica
  1.0 Kb promoter region YALI0A12353g
ttatctgaaatttatccttgtcaaggttacaactgcctaatcatcgtccccagactccaagtccgatttgcaggaatgacattgtttataggtctaagtaactgaaaatgtattcctctagaacgaatagaatatcccccgattaatcttggattttttagacgtttttaacatacatacattcatcaacattattcagtaaatattcagtatttttagcctaagacgattacaaccgtctcaccaaatgtgtgatacatacgtctatcaaatgtgtatgattttactggaaacgtcgcaaaactgtcgtgtggttgtccttgaccgattcaactaatagtgctctacatagaccaaatttttgagaggaagaccaaggaggtttcgtatactaaggaaaaccaaggtggtttcgtatactaaggaaaaccaaggtggtttcgtatactaaaaaatcaaggcggtttcgtatacaaaagaagaccaagaagtcttcgtatcagtaaaaaaaaccaaccagagttttagtatacttgcggcctacgattgatataatcggactagtcttccaccccggtcataactgtgcggctcgaaaatgcaatatagccgagagtgcggctcggaaatgtcgatccaggtgttcggaaggtgttcatatctgcgaatattgctagcgaatgtggaataaaaacgacagaatcagttagcaaatgattaatatatcgatttattgttttatctgcccaggtggcagtacaatatcttccactaatggtacatgttctgatcccgggctgttaaacgggttccaaattataattaagcgagccgcacttgacatccgtctaatcaccgtgtctggacatgattcgtgcaagtcgcacagatcgtcagtctcaaggcaccacgatgtttggggtataaaaggaggccgggggacgtctcaattccccatccacataccacctccaccactaccaccaccactacaaccaat

SEQ ID NO: 3 - DNA, 1000 bp, Yarrowia lipolytica
  1.0 Kb promoter region YALI0F15983g
gaagtttgaggccacgaaggtcaagggcgaggaagagttagtacaggacacggacaaggtgtttggagctcccgatctcgccatttacaagaagcggtttgcctacggcgagaaacttgtgctggccgaactcagagctaggaagaagaggttgaagcaacttattgagcgcatctcggcaaccaaatccaagcgggagagagaaagaaccaatatccagctttccctgtcgcctttttatgcctgggccgacgaagatgagttcgatgaggagcttgcccgctgggatggaagttatgatgatattaacgctagtagtggtataagaagaggtatttaatattgcaattgttgttattccactgattaaaaaaatagcttgctacgagtacagtacccgtactttcacttgcactcctactgtatataccgcatttgcttgtagagttagactcctacggacagtccaactctctcaagtcaccaagtacaagtacttgtaaatgggagccatatactgctacggaactcgtctctaaagtctcggctgtcttcacatgtggcctgcgggccgttgtgctgcgctcgtgcttccgttgtgcggcgttgtaagaatgtggagacgatgccgtggtcgagagaaggcagcgttcttcatgagcagacacgaaatgatactcgtcgtcagtgatgcacaaacacattgaacgagttgtttacattgataccaaggcattgataccccctactgccaaactcgacatcccttactgaaacccgtccgtcgagcacaacatcacgcacatcatgcacattatgcggagagccgctcagatcggagtatactcgatcctatccacggatagtgctggtgttcttgtgcggcgggagcttctggattatgtaagaccattggactttggtactttcgtaccatatataagacgatcaatcaccctgttcagtctccatcaaacaacacccatttcaacattacatc

SEQ ID NO: 4 - DNA, 1000 bp, Yarrowia lipolytica
  1.0 Kb promoter region YALI0B22506g
tgtgtaagaatgcatcgttttattcaccacgcgtttaatttcacagttaggttccggggtacagcatttgaaagaatcttgatcacgttatcattatcatcacatacgttcgtcctccgttaatacttcatacaatcgtgccaactggcggtacggagcatgtcggtgttcggtctcataagctcctaagcccagccggaacccgtctgcccgagagccattatctgcagctcttaacccctcaaagtccacacctacgactgtacacaccaaggatgcatgatctctttacacactgcggccaaaatatttgagacgacgttctacacgagaccaagaaggttaaaagttgcgatttgggatcctcgaagtcgcagcagaatgaggtcttgtacaaggtcaccagggtatcactgtctgtatacgagtaaaggaggacctgcttgtagcagacaatgatgggtatcgtgtgtgatagatgtccagacatagctgattgctctgcattagtctcctcagctgcagactagtgtaagcgggcctggatacaattccaagccggttcgtgcggctcggaaatacgcaatagtagtatggtttgtatggggaaacactagtgcggggagggactgatctcattgctttcaggagaatgaggttgaaatcagaaggtgaatatggaacgattctcggtcaaaaaacatcgagttagctatgtctttgcaattgtcctgtatccacgcatcacgtgagccattgctctgggcttgagagtgctcgctacttgtctccagcatcgcgataatgtgccatatgcatgcattgatgcaagagaatggctagtggcctcaacaagcactaaggactggggcttgtgggggagactgacggaatagtcgaaacccatggttattttaacagctccgatgtcaatataaataccactggataccccctccagttacaatcatcacacacaaacacaaacacaaccactaca

SEQ ID NO: 5 - DNA, 1000 bp, Yarrowia lipolytica
  1.0 Kb promoter region YALI0C08074g
gcctgctccccctagaattctgcatggaacaggcaaaggaccaaggacgtttgaagagatgggcattaatccggtgaccaaggaggccgacgattgtgttattatgtaatgtatgatatcagtatttaatcatagtgcggcgagtaggagctacttgcaggtgcgtagagggctgtttgtagtcatcgcactgagccagaagatgagatgaagtgtctggacgaagagccagctcctacgaatccagatgttcagatggacccagcttcttttctacttacttgctgaactcaggtatcacttccggctgctttcatacgtgttacggtattcactgttgacatcatttgacgcatgttaccattacttgaagagtacttgaagataagtcagctgggggtgacttcgtcattcctgaaccattaaaaaggagtggggcagaccaagtgatggagagatagctatttattaaaaatgaagtaaaatcagcaacacagcatctacaaatgagccgggttaagggttctgtcgttccaattgagttgctgcattagttaggacttgcgttccgaagacagtgcggctcggaaatttccaaggaccgtctgtgcggctcagatattgtttgccaggctttcgggagtgtcaataaatggcatatttgctagaaaaaatgtcgactttccaggttcccaagttgattgtaggggagttattgtatcatgaccagcttttttagtgctattttcttctttgagatgctctttttagcatcccgaatttccacactaaccgatgttcggtaccttagcgagcacgaataaataagccgcacagtagatccgactagacaccgcgacttgacatgattgacgcaaaccatgtggagactcaacctcaaggcatcatggtgtttggggtataaaagggggcgcgaagacctctctcaagtccctatcaacactcaactcaacccactacaaccacaactactacaact

SEQ ID NO: 6 - DNA, 1000 bp, Yarrowia lipolytica
  1.0 Kb promoter region YALI0F13937g
atttcaaaaataaaggacctaaataccactgcacctgtttggagaaataacgactgtgtatcccgcgtaataggtcaggtgcagtaagataagtctagggtgttttctgttgatatggaaacagggaaccatgagttagataaccgactccgcgaaatctctccgaactcaccaattagagccagttccgtgctattggtatatctgggctgagaggtgcgctacccctccccgtgtatggtggtaatacgggagagaaaagtgcaagtacaggaagatacagagagcgtaaatcttaatctatttttgagagacaggatatgaataaattgtactttagaggagttttgtggtgacttcagcttggctgaggaaggattgtatacgatgtactatgattatcgagaaaagcaatggttttctgattcattgttttatgtttccatcataccgattccgcaatataattgtaattgcacaaatactaaccatttacttttgcggccattttctggaggtttcgtgtctatgtacatcattaacagagacggtactgtggcggatgaatcatgtgcggctcgaaaattcagtcggtgcggctcgaaaattcaggcggtccgtctgtgcggctcagaaattgtcagacgggatgcttggaataatggcgggatccgttaccaaattaaaatgtgtgattaatgttacattagattgtaattgttgcaatctatcggaatcacctgtttgaagtcatattttttcagcaaaaatggcaatttttcagacgtgtttagttaaatacaaaattgcttcaagcggcgacaaggaattaatgagccgcacgcttacccgttgaaacaccgcgtctcgacatgatacatgcaagttggtcagatcaaggcgggggcaagatggcggtatttggaatataaaagggctcaaaactccagtcacttcatcatcaacacccacacaatcccccacaacaactactacagat

SEQ ID NO: 7 - DNA, 1000 bp, Yarrowia lipolytica
  1.0 Kb promoter region _YALI0C14344g
taaaacgcgagatatacaggaaaggcacacactaaaggcaagaataacaataataagtgtaacaagagttatcgtgttgctctggaattcaaccaaaccccaagccaccatgtgtcaatggtttcaaaaacaagagctccaaatttgaagttttcaataacgtagaaatcattcaaaataacttgaaactctacaaaaaaggagcagaggaaattaatgacccatagaagactttatcttttattgaaattatcacatctagtaaaacggccgatctggctcattattggatagttatagaaacttcaatttttgacatcaagaaaaaaagcgccgatggtttagtggtaaaatccatcgttgccatcgatgggcccccggttcgattccgggtcggcgcaatccgttcagagatctaactagtaaatactttttgtagtgcgagattcgtggttggtgtaatctttttgatattgaattattgtactttttattccttccccctaggcttatacacctctgcgagctggtgcttttcagatgtgtgcattgtttgtttgcataatccggcccaagttggtgcggctcggaaattcacatggaccgttgatgcggctcggaaatagtttagcggatgtttcgagacggtcaaaaaagcggtatatttgtagaaaaacatggcttcaatagtaccgggtgaattaagatgacctaatgatttgatttccttactttcactaccaggtaacaatctaattcgtcgaaaatggagctgcaaagttcttcgcttaattagcaagatattgaaataaatgagccgcacagtccatccgtctaaacgccgcgattcagcaggattaatgcaacctatacaaaccaggcggggacgaaatggcgaaaaaacgatataaaaggggctccaagtcctctctaatcaccaccagcacaaaaacgcacaccaccaccaccacagcaccaccataccactacaaccaccaag

SEQ ID NO: 8 - DNA, 1000 bp, Yarrowia lipolytica
  1.0 Kb promoter region YALI0E14256g
acttgtttgacaagagccaaacgatacaccaatgtgtatgaaacctacgcactcttggcatatctactggtactgtagcgacagattcacttgttgagagctgtgcttccgagcatcggatgtacctctttctcatataattatcgtcaataataccgcgcataacccaggcactcatcagggctgtacaccctcctctccaatggcaggcgctcgtagcagcaactaaaccttggggagggggcgtgatcgaggaaagggcttccagtgcgtaccacacacgtatatcgacgtaatcgtgccatgcagacggcgtgagatagtgtagtttgagctgtattctgaagccggtctgccaccgtatgtataggatccacgtccaagaagccgcctcgctggagccaccggatcataccccatgttccaataccccgctgaaaggacaaacagaagccggaccgtgcggtgcggcgagatattcggatttggctccattatctttgtgtatccggtgcaagtcggcttttgcggctcggaaatggctacttgtagctctgggtttgtgtttgaggggagagttggatatggaaaaacgtggatggtgaagccttcggggaattggtgtggttcccaatcaactactaggtcaattgatgccgtctttttggagatttctggacgccattgaattgctgtccatgagacaccccatattcgcttaagcagcttccttaccttagcgaggcacagaacattccgcctgtcagccccaacccaatctctgagggccacaactctcccccaatagccagctgccccagttgctcgatcagccaccgaagcttcagacaaggcagttacacactgagcctcaaggttgtgcgggcggatggggtataagggttgaggtggtaaccgtgtgagctcagaagatatataaaggggtggccatgtccccctatcgctccttaccaaacaacaaacaacaaacaactacaat

SEQ ID NO: 9 - DNA, 1000 bp, Yarrowia lipolytica
  1.0 Kb promoter region YALI0B19976g
agagagccagaatggagtatctactgtatgggattatactgtagactttttaagagagaacatctcatcattgaaagaatttatgtaataacaccatcaacgtatttcatgtaagcttcacctgttggtgagccacaatacattgctattcgactgaagtatacaagtaactcttgccaatgaaaatcgagaatattatggacgtttgggatgaatggtgaaccagtcaaccaatttagttgaaaagtcatccagctttaccgaagcaacgccgtttaaatatactggggatccacagttttagtactttctgagtgccacatctgtacaccactacagattatagggtaccaaaagacaaaaaaatgtcggtgaagatgatgagatggatttatgagactcagatggtgactccaagtgacccaggaagtacatagacaggggaggtagcggcgcaaataattctgttgagttggatggcgtcacgatatttttctattcgcttaaatctccacattgaggcgtcttttcaaggcatcatcattattgacagcctttgatccgtcatgtcatgcggcttggaaatgaaattcggctccgtcgtgcggctcgaaaatgattgtctcggataaagcgggggagtaaagaggtgtttgaagttgtggtagtttggatggggaggaatgaagtaaaatataaacattttcgtgcggaaaaatgaagggtttttatataaaaaccctacttatcttgtcatttaaagccgctaaatgcgctgttactgtagcatacagatagttagtgagaacatgttgacaaactcaatcggatcctccaaccttgtatttatgcgccgcacaatgagcaaaccgcggatcaatgcccaggacatggtgtggagactgtgaaaagatataaaaagaggtcctcaccatccattatttcttcatcgactcacagtctttagcaaccactacatctaactacaacaacaactaaa

SEQ ID NO: 10 - DNA, 1000 bp, Yarrowia lipolytica
  1.0 Kb promoter region YALI0E15840g
ctttgctccaattgttgtttttggaacgtgtgttgctggaggtgtggatcttttggcagatacaggtatggacgttcagttgatagacaccaagtaaactacttgtagttggtcatatgcatcgtccatcactcgtgttaatttcgaaggaagctgtttatttgatgagtagctcattttccaacttactcggtgaacgttgccagcggctggccggtattggacaagatcttactgttggacaagccattgtgagtcctcttgtgggtgtgtttggatgaaaaacagaaccaatcgcgagtctcggcccgactccactcagaccttttgtatctggataaatggagtgtcgcttggctcaggggtacgagacttcgcccaagttggctcaattgactaaaagtccattttcaacccgtgtcatggtcgttaaacacaccaatttcactctgatacaccaatgagtctcattggaccttccaaagcccccatttagtacttttttagccctctcgggacactggctactaacattgaacaccggccccgcatcgtgcggcttgaaaattcaggtggtccgttcgtgcggctcgaaaattgtagagcgcgggggcagttcaggggaggagcccgaataactcatattttggtagccacaccgacctcaaagtaatttcagagggccattaattgattcttaattgattattaattgatctttgttgtttcaacgtgccggaatggtctttttataggtccattgtctagttcactcagaccacatcaacaagagttctatgcagcacgagtgctttagcgacatgcaggaaataataatgagccgcatagctctccggtctgaaaaccgtcgaacaacaccattcctgcaagtcccacagatcgaggcggacatgatatggtgtaggaaacgatatataagggacccaaaagtcctgaatttggtcttcatcaacacaccggtctaccacccactactacc

SEQ ID NO: 11 - DNA, 1000 bp, Yarrowia lipolytica
  1.0 Kb promoter region YALI0F28765g
aaatgcttggaagttacgctactgtatatctggccaaccatcgatttacttgtatagtgttatgttgaagaacaagacgctatagacacacagtatatctacaggcacttgtacgcaatgaatggacgccgtatactgttcaaatgtttgatattcagtatcaccagctcacatctacaaatactccactcccacatgccggagccgcacgaacttatgagatcaacttgtgctccttgtacaagtaccggtacaagtacctgttcctatagatctgcgtgatcaaatgcaggttgcacacagaacaacaacaagagagtacaacaccatcaagtgaaatctaatatgctgccacacactgatgagagacaccgcttctatgacatgattgaaatgagttacagtaggatggaaagactggtcgtagctaggtacaaggatgtggggaggtatgcgtttattagttcatagaccacatggtctttccatgggttaatatctcgtataaatgtccaataccagccactctcattgtataccactgctgcctgcctcacgtcccgtttcacgggtcatacgtgcggctgaactctacaacttccgtgctgtgcggctcatcatcaagtagcctttcattctgttgagcatcccgttgtgatacagaatatgatccacatgtccatctcttcaccacgacatcaagtgacacgaaaacgacacatagcagcgcaagatacgacgaaaaatacccagtgatttcagcaagaaatgtacactcaatcatgataccataacgaggtcactagcctttcaaaccaccacctctagatgacatcaagaacgcatcacagtgcatgtcatgcacctctcgtcacccacaaccgggtcaaggactacggaaatccacttgaacgatacccgaagcgtctcggggtatctatataaggcacccatggaaccctgttgattcctcattcaaacacgcacacgaacaaacact

SEQ ID NO: 12 - DNA, 1000 bp, Yarrowia lipolytica
  1.0 Kb promoter region YALI0E19657g
ctgtgcatgggaaaaatagaaaaatgaagaagaaaaaaatgaagaaaaaaaacataccaacaatcaactcctccttctttcgtcgactcttacaaatcatcacgtgaccacacttctccacaacatactcccccaaaatagaatgcactatactgtagtatatcttataccataccacaaccgaacacaccgactaagagaaaaaatcgggcatttccacacctggagacacaaaatctcctccctccaaaacaagacatatataaacactcaaaaatcgcccttatcatatccaaaaagtcgaaaaaaatggcacttttgccaaccctcattttcttcaaccccgggtcattaaacttccgtcagacgcacgtcgcatcaacaatagtctttaatataattactggagaagcggagatcacggggtttggaggagagacaggacacggcgaggacggtggtcacgtgaccagaacaaaccccacatgaccggggccagtgtattcacgtgacccaccctctctcaaccgaaatggcgactgttgctgctgttgtattttggtctgttatcccagtctaacttcaatgttggagttgtcggaaaaaaaatgtaaaaatggatgataaaatgtaaaaatggaattaaaaaaaatgggttaaatgttaaaatggattaaatgtaaaaaaggaattaatgtataaagtgaaaataagtcgggtttatccggggtccaccagggagctgaaaagttgtcagatttgaaaaagcagagaaccaatggattctccgagttcctgcgattccagttctcttttctccacccagatcagacctccgcaacgtccaatatttgcattccaccccggatccacacaaggttatattctgcagaaatagtacaaccaagggtgtccacctcaacacatttatccaaccacccacccactcctactgttacgtgtaccgacacagaacgtcactcttttaacaccctcattg

SEQ ID NO: 13 - DNA, 1000 bp, Yarrowia lipolytica
  1.0 Kb promoter region YALI0B21670g
ccacaatgatcaccgtggggctatcgaacttcttagcaaacatgtaggcggtctcttttccgattccggacgttccgccgaagaagacggcaacggcatccttaacggtgggcttataggccgcattgacctgtttgatatcggctagagagggcatggtgtgtagttgcgaaaggtggcgaaatccggggcttaagtattagagtcctgctgtgcagggctgatggctttaggtatacaacgctatttggacgcaaggtacatttgagacagggcagcaggagggaggggaggagctagagggacggagaataggcatttccagccggagttttcggaaatcaattgaataaataattttaaacagatatatatctttagatttttacagttctcatgggagctctccagataagtccgccttggaacacatctcatctgcatgaattactaaggtggcggaaaaaaaacaacgtctgaggcaagttcaagcaggctggcggtgggatctaattattcagcgccaaacagatatgacccttatatgatgaagggctattatagcagctacagcagatgcaaaggaggctagatgatagcctggcattggtacaagtagctcacctcgtggcgaacctttgctcccatacatgtctctttcagactgcactattgcgcatttatacgtggttatacgtcttctttcttcgagttacatgccctgtgtgtaactgtaaggagtggtaccccgtctcgtacaaaacctgcaactgtcaattgtaaccgcacaagtgcgtgtgtgggcgtgctattgagtcagactttgactcatgtgggccaccaataaaagcctgaggatagagccctcaaaagtcccaatgaccttgtgggggtgctccgcttagcgtgggggtcaacctaaaggaggaagtacccaaggagctaaccccagactaatagtacagtacgcatccaacaacacactcaattgcaacacaca

SEQ ID NO: 14 - DNA, 1000 bp, Yarrowia lipolytica
  1.0 Kb promoter region YALI0F29315g
tgtcctcaggtgcagaataactgtcgctatggctgttaatttggggccatatacaagtagctcaaatccacgctccaccgctgctggggttgagacaaaacctgtgagcctccacattgcacaaaagagggaaataatcggcaccaatcggtatcaatcagaaccaaccgatttatctgtttaacaccaaaacccctttctacaagtagccacatctaaccactcaccaggcccattaccccgcctgttgatcaacggctatttctacctcatccatattgggggtgcataacccatcagactgggcgtctaataggaagagcaggaggaggatattagacgacgtcctcacccagctgggtaagagcagagtgacaaccagctctctttccgaaagccgtatatactctcaaaacactccaccatctacgataagcatatcggcgcgagtcttatttcatatatctggtgttttaccgtaagatccaccttccatccgaagtatgaggcttcgggttcaacccccgccctcatacactacttgtagaaggcggaggtgtccacacacttttttttctttctgcaaacaatactctccgttgcactgcggctcaataatgctacatacgacttatccggaattaggttgggaccgttgagtacgaccgctgatcgacgactctgaagatggtgatatcgctgaaagagataccattacactacatttgaaatacagaacattatttccaggagtaatgtaccacttgaagtctgtgattttaaatctctcatcaactgtgtatacctggctcttgcaaacctccacatgcatgcagcactacctccatgaacggatctcggatcccctactgcggcgattcagtgggtatatacctcaattggacctataaactaattattcttctcctcgacttttctatttcatcacacacacttacacgcacacgaacaaacactacgatggtccttcttattctcc

SEQ ID NO: 15 - DNA, 1000 bp, Yarrowia lipolytica
  1.0 Kb promoter region YALI0D25256g
tccccaacttcaactagtgcaaatctcggtagtattttgtcattgttttataaaagaaagctcatcattattatgaaagattgcagcatttcaccgagaggtagagcggcttttcaaagagttcaaatcggctcaaatctgttcaaatcagctcaatctgttcaaatcagctcaatctgttcaaatcagctcaaatcaactcaattcagttcaaattcgacaagacactctcatcgctcattcgatgaatcccttttataatccagcagtattcatatgctctcccgtccacgtgggcagtttccttcagctacaatacatctgcaaagacactctgttctatacctcccgggtatccgctatgttcaattgccttgctaaaacctcacaagccttgctatgcagtccagtcgaattatccgggatttggtacgcttcaacacgaaagaggatgtacagtacaagtagaccgacagacctgcaccccgcctactacaagtagccgctttgaccccccactagacaacacacacacagtgagatgtaaacggccagtttctagcagccacgtacgtacttgagcagtatagaaccatgcaactgcatctctccctctgtgacccccaattgcaaaccgtgtttgaaaatacctttgccaatcagcgcaggttgaatgtgggtgaacaaggtcaaaacgggtgaacaaaggtcaaaacgggtgaacaaggtcaaaacgggtgaacaaatgtcaagcgcaagacggagacagaagtttcggggtcacgtgactatcacgagttgcgattctacgacgctcatcagcgacgcccttggatttgctccattctcgcttaatttcgcttatcgccaattgcgtggggggtgtggggggtcccaactttaaaacacttacccaacatgtcccccacgctctagatatataccacctcgaaaatcccactcccccacacatcgctcaattcctccacgcgtgcgccaa

SEQ ID NO: 16 - DNA, 1000 bp, Yarrowia lipolytica
  1.0 Kb promoter region YALI0C11099g
atcctgggcaaggcgctggctgaaaattatcaggccacctgtagagcattctttccgtccacgtggggacaccttgtactcggtgtggttgacaaaagcagaccatctcggatcccttgagcatcttagcaagtggcgtcacctactcgagcttgtctcccaccagtgtccttgagcttcaaatccagagacggccccgatacattctcttatgattgttgaataccgtttccgctccttgaagagcagggaatcttgacggactggttgtggtagaagagccagccggcagaaacacggccactgaatcaaggagagattgtagtagagcgtctttttgtcgcccttgcctccgattccatactttttcgtcagggtgtgtttgatatccatcagtttgagctgctttacaggggcatgggagccccttctccctctttacccgtcagactccatccttcttcttcgttctcttctccgtcttctctcatcaccatagtggtagaggtgtgcagaccttgatatcgtcggagcagaagttgtcgtcggaactagccaggctcattttgtcaatgtattgagctgcattgcgtatctcgcgtcttattctaagacaccgtctcagcacattagtgcggcttgaaatttcattcacgacttggaaattatgccgcccgtaccgtcgaaagatgttagtaagcaatagctgaaaataccgggaatcgattggtgcagtctcaatgtgtctatttaaaccaacttggtctgtttttgctatattggagccccgctagaactcacgatcgagcctctggttcttagcgagaaaataagcgagtcgcaccacagccgtcgaaacacccgctggcttgcaagttgaacacctcaaggagggctccagatggtgatcatgacaataaaaaggaccccaaatcctctctattcctatctactcacaaacaccaccaatatgaaagtccttgtcctcccacgccagctc

SEQ ID NO: 17 - DNA, 1000 bp, Yarrowia lipolytica
  1.0 Kb promoter region YALI0F09966g
ctccccttccgtacctctctgccccttctggacaggtcaatgatagactcagagcgacacacatgtctgacgtaccatgttagaccttgtattgacctggacgaatgtgtgtgaggagtgaggcaggccaagacgaaccacggtctttatatatgcccacggagtgacacggtctgtgtcgtcaccgcagctccactcaccacccgcatcatgatcgtccaaccagaacccactccccagtttcgacccaacaccattctcaactgtaagtatgagtaccacagtgatactcgcccagtgccgcactcgtactgtagccactccactgcaacatccgtatcgtattgcaccgccccgattcacctgcttccttcaagccttcaaccacgtactgctccacctcctaccgttgagcccactcggatcggccagagtcatgtcttagggtttggctgcagttgtggcgtaaactatggagaaggcgacggaaacgagagcgctaccggtagcgacttggcgacacgtctggctcgggaagggggccgttgcagagaccaagacttccgtcacgtgaccgctgtttggtcaattctaacgcagttattttccgtctgattcgctgatacgagtactcgcttgctgtagatgactcagaccaagacaagagaaggggaaataaaaaaaacttccaaaaaaaacttccaaaaaaaaaaaatcaaaatttgacaaaccttttctgcctgggaccaggaactttgtgagtccattgagggagttagccacccatcagccacagccacagtttggacaagaagtaaaagtggatatatttatgttatggagaccatgtagtgttgtgggagggaggggtttttttgtttgttttggctgagtaatcaacagcaagtggcgtatatcgtatatctatcgtgactcagactattcaccgcttgtatggtgctatctcgacttgtgcttagtctcaggtacacgtgattgc

SEQ ID NO: 18 - DNA, 1500 bp, Yarrowia lipolytica
  1.5Kb promoter region YALI0A12353g
gtcggatgcttcttccactgacgccgagctggacgacgttcccgatcagggtgctctgggtgccattcacgagtcccggtccggcactggaagtcctcatcttaacatggaggagtttctgaagcagacatagatactaattattaatacatcgcatgattgaccaactgcaagtacatactgtatgtactcacttgtagagattctcgttgcgacaaaattgtggacaagacacgaaacgaccatgtctgtaaagtcacgtgaccaaaatcatcacagcagaaagtcgagttcttagatatcttccacaagagttgcatgttttatattggacagagaaaaagggatgtgtatatgaaaaactttaatttcgggcagattccaaaaaataatgacatccgagagattcgaactctcgcctccggagagaccaggatattgattcagtcacctgaacctggcgccttagaccactcggccagaatgccaattgttgaaaattatctgaaatttatccttgtcaaggttacaactgcctaatcatcgtccccagactccaagtccgatttgcaggaatgacattgtttataggtctaagtaactgaaaatgtattcctctagaacgaatagaatatcccccgattaatcttggattttttagacgtttttaacatacatacattcatcaacattattcagtaaatattcagtatttttagcctaagacgattacaaccgtctcaccaaatgtgtgatacatacgtctatcaaatgtgtatgattttactggaaacgtcgcaaaactgtcgtgtggttgtccttgaccgattcaactaatagtgctctacatagaccaaatttttgagaggaagaccaaggaggtttcgtatactaaggaaaaccaaggtggtttcgtatactaaggaaaaccaaggtggtttcgtatactaaaaaatcaaggcggtttcgtatacaaaagaagaccaagaagtcttcgtatcagtaaaaaaaaccaaccagagttttagtatacttgcggcctacgattgatataatcggactagtcttccaccccggtcataactgtgcggctcgaaaatgcaatatagccgagagtgcggctcggaaatgtcgatccaggtgttcggaaggtgttcatatctgcgaatattgctagcgaatgtggaataaaaacgacagaatcagttagcaaatgattaatatatcgatttattgttttatctgcccaggtggcagtacaatatcttccactaatggtacatgttctgatcccgggctgttaaacgggttccaaattataattaagcgagccgcacttgacatccgtctaatcaccgtgtctggacatgattcgtgcaagtcgcacagatcgtcagtctcaaggcaccacgatgtttggggtataaaaggaggccgggggacgtctcaattccccatccacataccacctccaccactaccaccaccactacaaccaat

SEQ ID NO: 19 - DNA, 1500 bp, Yarrowia lipolytica
  1.5Kb promoter region YALI0F15983g
gtaccgtcgtgagatactgctattgttagctactcttctttctctatataatgcgttgtgagatcggagtgtccctgccacgactcaaccatgatcgacaaactattagaccagtacattggagatgtgagtttttcgaacgtttattccgccgaggagccgtggagtatctacgacgagtatcctgagattccgccgcgtgttcaaccagtggagaattctctgattggagacttggacaatttcagtctcagtgtaccttctgagatttctttggacgcgggcggtttggaaatcacgtcatcggtcaccgttgtcaccaagagagcggacagagttgcaggcggtcagactctctatggagacattctggggagtttgagtggtttggatgggtatgggcaggctggttcagagagaactcatcaagtgacactggagaagtcgactgaagcagatgggctgggtaagagcgtgttggaggagctgtctgaggaagagaagtttgaggccacgaaggtcaagggcgaggaagagttagtacaggacacggacaaggtgtttggagctcccgatctcgccatttacaagaagcggtttgcctacggcgagaaacttgtgctggccgaactcagagctaggaagaagaggttgaagcaacttattgagcgcatctcggcaaccaaatccaagcgggagagagaaagaaccaatatccagctttccctgtcgcctttttatgcctgggccgacgaagatgagttcgatgaggagcttgcccgctgggatggaagttatgatgatattaacgctagtagtggtataagaagaggtatttaatattgcaattgttgttattccactgattaaaaaaatagcttgctacgagtacagtacccgtactttcacttgcactcctactgtatataccgcatttgcttgtagagttagactcctacggacagtccaactctctcaagtcaccaagtacaagtacttgtaaatgggagccatatactgctacggaactcgtctctaaagtctcggctgtcttcacatgtggcctgcgggccgttgtgctgcgctcgtgcttccgttgtgcggcgttgtaagaatgtggagacgatgccgtggtcgagagaaggcagcgttcttcatgagcagacacgaaatgatactcgtcgtcagtgatgcacaaacacattgaacgagttgtttacattgataccaaggcattgataccccctactgccaaactcgacatcccttactgaaacccgtccgtcgagcacaacatcacgcacatcatgcacattatgcggagagccgctcagatcggagtatactcgatcctatccacggatagtgctggtgttcttgtgcggcgggagcttctggattatgtaagaccattggactttggtactttcgtaccatatataagacgatcaatcaccctgttcagtctccatcaaacaacacccatttcaacattacatc

SEQ ID NO: 20 - DNA, 1500 bp, Yarrowia lipolytica
  1.5Kb promoter region YALI0B22506g
tcctgaatcaagagcaacccggtcttgaatcatcttttcacaccttcaaacaccaacagatgtaacagtatcccctcttcgataaagttggtgtgggggtcaagtgtctctgaacctgtatgaggatgtatcacattgtatcgtacgcacgtaccggtggctatacgggttcgctcattattgtgtattattagacatgggaaaatgaaccggcttgatgaacaggaaattaacacgtcatttgcgtcagccatgaacttccacttcgccggtgataaactaacacaccagtatgtagttgccgtaatctgaaagcccacgtgacgcatctctttttagcagcaaagtggtcaccaagactacttatgatgcagaagtatccaaccatgcacgtactgtacgcacatgaattgccgagcagttcctggagctattgtatttgtagtcgtactatattcggaggaggggttcccggctattgtctttccatagctatgatatgtgtaagaatgcatcgttttattcaccacgcgtttaatttcacagttaggttccggggtacagcatttgaaagaatcttgatcacgttatcattatcatcacatacgttcgtcctccgttaatacttcatacaatcgtgccaactggcggtacggagcatgtcggtgttcggtctcataagctcctaagcccagccggaacccgtctgcccgagagccattatctgcagctcttaacccctcaaagtccacacctacgactgtacacaccaaggatgcatgatctctttacacactgcggccaaaatatttgagacgacgttctacacgagaccaagaaggttaaaagttgcgatttgggatcctcgaagtcgcagcagaatgaggtcttgtacaaggtcaccagggtatcactgtctgtatacgagtaaaggaggacctgcttgtagcagacaatgatgggtatcgtgtgtgatagatgtccagacatagctgattgctctgcattagtctcctcagctgcagactagtgtaagcgggcctggatacaattccaagccggttcgtgcggctcggaaatacgcaatagtagtatggtttgtatggggaaacactagtgcggggagggactgatctcattgctttcaggagaatgaggttgaaatcagaaggtgaatatggaacgattctcggtcaaaaaacatcgagttagctatgtctttgcaattgtcctgtatccacgcatcacgtgagccattgctctgggcttgagagtgctcgctacttgtctccagcatcgcgataatgtgccatatgcatgcattgatgcaagagaatggctagtggcctcaacaagcactaaggactggggcttgtgggggagactgacggaatagtcgaaacccatggttattttaacagctccgatgtcaatataaataccactggataccccctccagttacaatcatcacacacaaacacaaacacaaccactaca

SEQ ID NO: 21 - DNA, 1500 bp, Yarrowia lipolytica
  1.5Kb promoter region YALI0C08074g
gtgcctcccaaggactctccctttgacagatcgacctatttcgaccggtccatctcgcccactaagaacagagcaggctccccctctggccgcgtcagcagtctgtctgctgtcgagatttcaaacaaactgaagaagtttgccggcaagattgtaccttcctcacctcccaaacgaggtgccgagtttgaaagcccgtccagaagcatgtcaccagtcaaggaccgagtggcgtcctcacctaaccctggacggcccatgtcggctctatctggccattcggacacctcccgatcgtcttctcctgtcgattccatcatgtcacattctcgttcgtccactatgatgacctcttcctcgggatcttcaggtaacagtatgctctcgggcttcattcccaacatgagcatgcccagtatgccaaacgtgaaactgcccagggccagcagccccctcaaaaacccgctgtcggctaaagatttgcaacacactccggtcgagcctgctccccctagaattctgcatggaacaggcaaaggaccaaggacgtttgaagagatgggcattaatccggtgaccaaggaggccgacgattgtgttattatgtaatgtatgatatcagtatttaatcatagtgcggcgagtaggagctacttgcaggtgcgtagagggctgtttgtagtcatcgcactgagccagaagatgagatgaagtgtctggacgaagagccagctcctacgaatccagatgttcagatggacccagcttcttttctacttacttgctgaactcaggtatcacttccggctgctttcatacgtgttacggtattcactgttgacatcatttgacgcatgttaccattacttgaagagtacttgaagataagtcagctgggggtgacttcgtcattcctgaaccattaaaaaggagtggggcagaccaagtgatggagagatagctatttattaaaaatgaagtaaaatcagcaacacagcatctacaaatgagccgggttaagggttctgtcgttccaattgagttgctgcattagttaggacttgcgttccgaagacagtgcggctcggaaatttccaaggaccgtctgtgcggctcagatattgtttgccaggctttcgggagtgtcaataaatggcatatttgctagaaaaaatgtcgactttccaggttcccaagttgattgtaggggagttattgtatcatgaccagcttttttagtgctattttcttctttgagatgctctttttagcatcccgaatttccacactaaccgatgttcggtaccttagcgagcacgaataaataagccgcacagtagatccgactagacaccgcgacttgacatgattgacgcaaaccatgtggagactcaacctcaaggcatcatggtgtttggggtataaaagggggcgcgaagacctctctcaagtccctatcaacactcaactcaacccactacaaccacaactactacaact

SEQ ID NO: 22 - DNA, 1500 bp, Yarrowia lipolytica
  1.5Kb promoter region YALI0F13937g
atacaacccatatttatctgtggtatacagttatacagtggggtctatcccttatcgctgttccagtgcaaggtagcttcattgtaatctttgaagcgctcgtattattccaatgtcgagtcgcgttttctacgattgcgggaggtcgtgggggtcaactacgccacggttcaggcggaacacatttctccgcagttattttgacagaatgaggtaacgatatcaatccaaatacattttccgcgtggatattcattgatatctccgtttattttacctaaagccctgatttctagagataagcgaaagtgccagccctgtaattttggatatataattagtagctttttaaacaacgagctttacaaagcttaccacacaagtatgagccagggactcgcccaggctgcttgtcttgttagcccgaactgtctttacctggactactgacgtctccatactatcgaccttgtcggccccaggcaaatgagaaacattgaatttcaaaaataaaggacctaaataccactgcacctgtttggagaaataacgactgtgtatcccgcgtaataggtcaggtgcagtaagataagtctagggtgttttctgttgatatggaaacagggaaccatgagttagataaccgactccgcgaaatctctccgaactcaccaattagagccagttccgtgctattggtatatctgggctgagaggtgcgctacccctccccgtgtatggtggtaatacgggagagaaaagtgcaagtacaggaagatacagagagcgtaaatcttaatctatttttgagagacaggatatgaataaattgtactttagaggagttttgtggtgacttcagcttggctgaggaaggattgtatacgatgtactatgattatcgagaaaagcaatggttttctgattcattgttttatgtttccatcataccgattccgcaatataattgtaattgcacaaatactaaccatttacttttgcggccattttctggaggtttcgtgtctatgtacatcattaacagagacggtactgtggcggatgaatcatgtgcggctcgaaaattcagtcggtgcggctcgaaaattcaggcggtccgtctgtgcggctcagaaattgtcagacgggatgcttggaataatggcgggatccgttaccaaattaaaatgtgtgattaatgttacattagattgtaattgttgcaatctatcggaatcacctgtttgaagtcatattttttcagcaaaaatggcaatttttcagacgtgtttagttaaatacaaaattgcttcaagcggcgacaaggaattaatgagccgcacgcttacccgttgaaacaccgcgtctcgacatgatacatgcaagttggtcagatcaaggcgggggcaagatggcggtatttggaatataaaagggctcaaaactccagtcacttcatcatcaacacccacacaatcccccacaacaactactacagat

SEQ ID NO: 23 - DNA, 1500 bp, Yarrowia lipolytica
  1.5Kb promoter region _YALI0C14344g
gtccaaacatggtctgtccagaagggagacaatcaaacgaggtcgggggatgtgaacggctccccgaaagatttccatcagtagaaactggtttcttgcctttgaactgttttgctacaagtttcgcgatgaagggtccagttgcaccagtctgaaaaaagtgttccgattaattttgtatcgcaggaatgttgaacttgggaaatacaaactgaatcagacagcttgagattgctttgattgcagttgaaaacccatctcattacctcgcatcatgtccacaatcggaaatctgttcgtcccgcaggtctcactagcctccatccgtagacttctcaccctagtcagtgatgcaagaaaacaagtacagtagtgaatagatagagaaattgaatgaattgtaactaggaaatcaactccaccagcttctcttcgagtctcagtttctgttacgccatgttttggatcgtgccacatccagggtcgatgttaatcctgtataaaacgcgagatatacaggaaaggcacacactaaaggcaagaataacaataataagtgtaacaagagttatcgtgttgctctggaattcaaccaaaccccaagccaccatgtgtcaatggtttcaaaaacaagagctccaaatttgaagttttcaataacgtagaaatcattcaaaataacttgaaactctacaaaaaaggagcagaggaaattaatgacccatagaagactttatcttttattgaaattatcacatctagtaaaacggccgatctggctcattattggatagttatagaaacttcaatttttgacatcaagaaaaaaagcgccgatggtttagtggtaaaatccatcgttgccatcgatgggcccccggttcgattccgggtcggcgcaatccgttcagagatctaactagtaaatactttttgtagtgcgagattcgtggttggtgtaatctttttgatattgaattattgtactttttattccttccccctaggcttatacacctctgcgagctggtgcttttcagatgtgtgcattgtttgtttgcataatccggcccaagttggtgcggctcggaaattcacatggaccgttgatgcggctcggaaatagtttagcggatgtttcgagacggtcaaaaaagcggtatatttgtagaaaaacatggcttcaatagtaccgggtgaattaagatgacctaatgatttgatttccttactttcactaccaggtaacaatctaattcgtcgaaaatggagctgcaaagttcttcgcttaattagcaagatattgaaataaatgagccgcacagtccatccgtctaaacgccgcgattcagcaggattaatgcaacctatacaaaccaggcggggacgaaatggcgaaaaaacgatataaaaggggctccaagtcctctctaatcaccaccagcacaaaaacgcacaccaccaccaccacagcaccaccataccactacaaccaccaag

SEQ ID NO: 24 - DNA, 1500 bp, Yarrowia lipolytica
  1.5Kb promoter region YALI0E14256g
ttccagcagaggcaacgccgttgatcaaactctgcaggttgtgggaatccaatgagacatccatggccttccataaccaggctttggagtcaaggcaccacctatgagttggccgagacgataaccttctaacttcctcacagaagatggaactttgggggggatagagtagctgccttggaggttggtgattgttggtaacacagatgaaatagctataagcgcctgattagggcgaggaaagttgcaggagccgaatccgaagggtcaattgtacatccgaagaagtcgatattcgctcaaacttccggcttgtgctcttttgcgtccccgaggatgtgtgtgcaaggtttgccaagtgcagcagcggttacattgctggaagatgtacactatgggtgttgattggggggtgctatggaagggtgtatggagcaggggtgctaggtggtccacggacaaatcataaataacacctattgctggtagttggatacgagacttgtttgacaagagccaaacgatacaccaatgtgtatgaaacctacgcactcttggcatatctactggtactgtagcgacagattcacttgttgagagctgtgcttccgagcatcggatgtacctctttctcatataattatcgtcaataataccgcgcataacccaggcactcatcagggctgtacaccctcctctccaatggcaggcgctcgtagcagcaactaaaccttggggagggggcgtgatcgaggaaagggcttccagtgcgtaccacacacgtatatcgacgtaatcgtgccatgcagacggcgtgagatagtgtagtttgagctgtattctgaagccggtctgccaccgtatgtataggatccacgtccaagaagccgcctcgctggagccaccggatcataccccatgttccaataccccgctgaaaggacaaacagaagccggaccgtgcggtgcggcgagatattcggatttggctccattatctttgtgtatccggtgcaagtcggcttttgcggctcggaaatggctacttgtagctctgggtttgtgtttgaggggagagttggatatggaaaaacgtggatggtgaagccttcggggaattggtgtggttcccaatcaactactaggtcaattgatgccgtctttttggagatttctggacgccattgaattgctgtccatgagacaccccatattcgcttaagcagcttccttaccttagcgaggcacagaacattccgcctgtcagccccaacccaatctctgagggccacaactctcccccaatagccagctgccccagttgctcgatcagccaccgaagcttcagacaaggcagttacacactgagcctcaaggttgtgcgggcggatggggtataagggttgaggtggtaaccgtgtgagctcagaagatatataaaggggtggccatgtccccctatcgctccttaccaaacaacaaacaacaaacaactacaat

SEQ ID NO: 25 - DNA, 1500 bp, Yarrowia lipolytica
  1.5Kb promoter region YALI0B19976g
tctcaagctcaaatagatggatcgtgaggaggatgtgaaggattttgtttattattattaattgattgtatcttattatgttattgaggttgtacagtttgaactaagtactgtaactggatatatacgatactgtaaactgtgctgtagttgtgtttgttactgtagtcagattcgtacgagagggtcatgacgtcttctaggaaaatcacgtgaccaatatgtggtgtttctttaattcaatgggttactaagcacacaataccttatgagtcaatttgggatcatacattatagttgcggccatatcctggtgaaaatacggcttcccgtccgatcagccatagtcaagcaccagagagcctagttagtattgtagtgggagaccatacgagaatcctaggtgctgcaattcttttgccacagcggcgggcttttctttttttctctgttaagaggcgacctcatatctgtgagagttgctgatgtcttaccacttgagagagccagaatggagtatctactgtatgggattatactgtagactttttaagagagaacatctcatcattgaaagaatttatgtaataacaccatcaacgtatttcatgtaagcttcacctgttggtgagccacaatacattgctattcgactgaagtatacaagtaactcttgccaatgaaaatcgagaatattatggacgtttgggatgaatggtgaaccagtcaaccaatttagttgaaaagtcatccagctttaccgaagcaacgccgtttaaatatactggggatccacagttttagtactttctgagtgccacatctgtacaccactacagattatagggtaccaaaagacaaaaaaatgtcggtgaagatgatgagatggatttatgagactcagatggtgactccaagtgacccaggaagtacatagacaggggaggtagcggcgcaaataattctgttgagttggatggcgtcacgatatttttctattcgcttaaatctccacattgaggcgtcttttcaaggcatcatcattattgacagcctttgatccgtcatgtcatgcggcttggaaatgaaattcggctccgtcgtgcggctcgaaaatgattgtctcggataaagcgggggagtaaagaggtgtttgaagttgtggtagtttggatggggaggaatgaagtaaaatataaacattttcgtgcggaaaaatgaagggtttttatataaaaaccctacttatcttgtcatttaaagccgctaaatgcgctgttactgtagcatacagatagttagtgagaacatgttgacaaactcaatcggatcctccaaccttgtatttatgcgccgcacaatgagcaaaccgcggatcaatgcccaggacatggtgtggagactgtgaaaagatataaaaagaggtcctcaccatccattatttcttcatcgactcacagtctttagcaaccactacatctaactacaacaacaactaaa

SEQ ID NO: 26 - DNA, 1500 bp, Yarrowia lipolytica
  1.5Kb promoter region YALI0E15840g
catttgttggtggcaatgtgaactggatgatttctgggtccaacagtgcaattatcattacagcagtagatagtccggtaccacccatcacgaggtaaacgccgacgcgggggtgagtgagctctttttgacttttgttaactccgacgaaatttgggggggggagagggatttcgtgcgtccttgcatggtctagtccgaaactaacaacagctcaactggcggatcaggacactttcagacaagtttcaaacagcaaattcggagtgaagaaccctcttgttgctctgcttctcttctgtgggtcgcttctttgtcttctccgtgtttttgccgaactgtacatcagcccacattacaaaactgcacggtgcaggacacgtttagccgcccaactatggcgaggtgggggtcgtcggaacgaggttcagaaaagaaaaagaccggctcgagggcttccggaggataaaaggaccgttcaaatgagtatctccacccgcctttgctccaattgttgtttttggaacgtgtgttgctggaggtgtggatcttttggcagatacaggtatggacgttcagttgatagacaccaagtaaactacttgtagttggtcatatgcatcgtccatcactcgtgttaatttcgaaggaagctgtttatttgatgagtagctcattttccaacttactcggtgaacgttgccagcggctggccggtattggacaagatcttactgttggacaagccattgtgagtcctcttgtgggtgtgtttggatgaaaaacagaaccaatcgcgagtctcggcccgactccactcagaccttttgtatctggataaatggagtgtcgcttggctcaggggtacgagacttcgcccaagttggctcaattgactaaaagtccattttcaacccgtgtcatggtcgttaaacacaccaatttcactctgatacaccaatgagtctcattggaccttccaaagcccccatttagtacttttttagccctctcgggacactggctactaacattgaacaccggccccgcatcgtgcggcttgaaaattcaggtggtccgttcgtgcggctcgaaaattgtagagcgcgggggcagttcaggggaggagcccgaataactcatattttggtagccacaccgacctcaaagtaatttcagagggccattaattgattcttaattgattattaattgatctttgttgtttcaacgtgccggaatggtctttttataggtccattgtctagttcactcagaccacatcaacaagagttctatgcagcacgagtgctttagcgacatgcaggaaataataatgagccgcatagctctccggtctgaaaaccgtcgaacaacaccattcctgcaagtcccacagatcgaggcggacatgatatggtgtaggaaacgatatataagggacccaaaagtcctgaatttggtcttcatcaacacaccggtctaccacccactactacc

SEQ ID NO: 27 - DNA, 1500 bp, Yarrowia lipolytica
  1.5Kb promoter region YALI0F28765g
taaccgtgttaaatccacatatctgacccggattcgggggttcagaagagcttctgataattgtgtttgttgattcccttcacttcctcctctggaccagataaatacaaaatatcaaccgtcaaacattgcaaattgtagggaattataaaaaaaattataaaatattttttttattctaatgattcattacagcacagcaaaaaaaattgggaaaaattgttttaagctgctgtagacaaaatcattgtaaatctgcatctctaggcaacctggccgagcggtctaaggcgccaggttaaggctaacaccttaacgaatatcctggtctcttcggaggcgagagttcgaatctctcggttgtcaacttttttggctgactcttctattgacgtcggcttttatcatgcatttgtcttaagtacttctttttcattgatttgaggtgtgttctgtcgatgtcatcatatagtacaagtagtgtctcactaccgagcttcaaatgcttggaagttacgctactgtatatctggccaaccatcgatttacttgtatagtgttatgttgaagaacaagacgctatagacacacagtatatctacaggcacttgtacgcaatgaatggacgccgtatactgttcaaatgtttgatattcagtatcaccagctcacatctacaaatactccactcccacatgccggagccgcacgaacttatgagatcaacttgtgctccttgtacaagtaccggtacaagtacctgttcctatagatctgcgtgatcaaatgcaggttgcacacagaacaacaacaagagagtacaacaccatcaagtgaaatctaatatgctgccacacactgatgagagacaccgcttctatgacatgattgaaatgagttacagtaggatggaaagactggtcgtagctaggtacaaggatgtggggaggtatgcgtttattagttcatagaccacatggtctttccatgggttaatatctcgtataaatgtccaataccagccactctcattgtataccactgctgcctgcctcacgtcccgtttcacgggtcatacgtgcggctgaactctacaacttccgtgctgtgcggctcatcatcaagtagcctttcattctgttgagcatcccgttgtgatacagaatatgatccacatgtccatctcttcaccacgacatcaagtgacacgaaaacgacacatagcagcgcaagatacgacgaaaaatacccagtgatttcagcaagaaatgtacactcaatcatgataccataacgaggtcactagcctttcaaaccaccacctctagatgacatcaagaacgcatcacagtgcatgtcatgcacctctcgtcacccacaaccgggtcaaggactacggaaatccacttgaacgatacccgaagcgtctcggggtatctatataaggcacccatggaaccctgttgattcctcattcaaacacgcacacgaacaaacact

SEQ ID NO: 28 - DNA, 1500 bp, Yarrowia lipolytica
  1.5Kb promoter region YALI0E19657g
ggagggaagaggggtatttaaggctgcgggagtggttttgtgtcgttttgcggtggaacgatgtttctgtgtcactcagcaatgtctggtgtcatatggctcggagtccgcatgtagtgtatcctcttcaatatgatggtgacattaaagatgtttagaggcggattgagacggttgtggagaatgctgtggtttgttatgggctgtatttatatctttggggaccttgtgtagtttttttttcaggatattcgtcgtttgaagtgactttttttttctgtattattcgactactgtacttgatccaaacgtttttacttggattttgttaaccatgcatttgataaaataaaaaaattaacccagttttattataaaaaaataacttcaataattacttgggatatatttttattacttggggaagtagttgggaggaagaggagcaaaaataaataaaaggaacatgtgggaaaatggaagttacaaccaagtatgtactgtgcatgggaaaaatagaaaaatgaagaagaaaaaaatgaagaaaaaaaacataccaacaatcaactcctccttctttcgtcgactcttacaaatcatcacgtgaccacacttctccacaacatactcccccaaaatagaatgcactatactgtagtatatcttataccataccacaaccgaacacaccgactaagagaaaaaatcgggcatttccacacctggagacacaaaatctcctccctccaaaacaagacatatataaacactcaaaaatcgcccttatcatatccaaaaagtcgaaaaaaatggcacttttgccaaccctcattttcttcaaccccgggtcattaaacttccgtcagacgcacgtcgcatcaacaatagtctttaatataattactggagaagcggagatcacggggtttggaggagagacaggacacggcgaggacggtggtcacgtgaccagaacaaaccccacatgaccggggccagtgtattcacgtgacccaccctctctcaaccgaaatggcgactgttgctgctgttgtattttggtctgttatcccagtctaacttcaatgttggagttgtcggaaaaaaaatgtaaaaatggatgataaaatgtaaaaatggaattaaaaaaaatgggttaaatgttaaaatggattaaatgtaaaaaaggaattaatgtataaagtgaaaataagtcgggtttatccggggtccaccagggagctgaaaagttgtcagatttgaaaaagcagagaaccaatggattctccgagttcctgcgattccagttctcttttctccacccagatcagacctccgcaacgtccaatatttgcattccaccccggatccacacaaggttatattctgcagaaatagtacaaccaagggtgtccacctcaacacatttatccaaccacccacccactcctactgttacgtgtaccgacacagaacgtcactcttttaacaccctcattg

SEQ ID NO: 29 - DNA, 1500 bp, Yarrowia lipolytica
  1.5Kb promoter region YALI0B21670g
ggccacgagaccggggtgagcgtgaatgaaggacagatcaggatgctctcgagcaaacttcatgactgctgcagagctgaactgcacaatatgacggttctggttcatcatggagaacttagttcgcagctgcatgtcgtcctccacgtagggaccctcgtttccgggtgccagcacagtcatgacacgtgcattgtccttgtctccttcgtcgtaggccgtctgaaccagaggcagcagttccttgacggcgagccatcgaccgtagtagttgagcgacattcgcagctcgattccctccgaggtctcagttcttccagcggtgctcagaatgccctgggacagaaacagcaggttgatcttggtcacgtcccgcttgacaatgtccgacactcgcaccacctccttcatctgcgacagatcgctcttaatgaaggttgcctttgcattgagctccttgagatcattgacaatcttgttgcctgcctcttcacttcgtcccacaatgatcaccgtggggctatcgaacttcttagcaaacatgtaggcggtctcttttccgattccggacgttccgccgaagaagacggcaacggcatccttaacggtgggcttataggccgcattgacctgtttgatatcggctagagagggcatggtgtgtagttgcgaaaggtggcgaaatccggggcttaagtattagagtcctgctgtgcagggctgatggctttaggtatacaacgctatttggacgcaaggtacatttgagacagggcagcaggagggaggggaggagctagagggacggagaataggcatttccagccggagttttcggaaatcaattgaataaataattttaaacagatatatatctttagatttttacagttctcatgggagctctccagataagtccgccttggaacacatctcatctgcatgaattactaaggtggcggaaaaaaaacaacgtctgaggcaagttcaagcaggctggcggtgggatctaattattcagcgccaaacagatatgacccttatatgatgaagggctattatagcagctacagcagatgcaaaggaggctagatgatagcctggcattggtacaagtagctcacctcgtggcgaacctttgctcccatacatgtctctttcagactgcactattgcgcatttatacgtggttatacgtcttctttcttcgagttacatgccctgtgtgtaactgtaaggagtggtaccccgtctcgtacaaaacctgcaactgtcaattgtaaccgcacaagtgcgtgtgtgggcgtgctattgagtcagactttgactcatgtgggccaccaataaaagcctgaggatagagccctcaaaagtcccaatgaccttgtgggggtgctccgcttagcgtgggggtcaacctaaaggaggaagtacccaaggagctaaccccagactaatagtacagtacgcatccaacaacacactcaattgcaacacaca

SEQ ID NO: 30 - DNA, 1500 bp, Yarrowia lipolytica
  1.5Kb promoter region YALI0F29315g
aagggtgtactacaaatacgtagcaactgtgggtgtgactgacatgtgtatgtatgtatgtatagtatgtacttgtacgggatgtactggtacgatacatacacgtgttgctaccagtgcatgcccataccctcgttccaggtgacgtcacagattgctcatcacggcttaaactggtcaggcgacatggttcaagttgtaaccatacactctgtgagtcttcaactgcaataggagatgagctcgacggagctgccagtcacgataattattcaacaacccaccattgaacagtacaagtaatattaatttaaccgaagaagtcttggcaacgtgctgtgcacagagacttacgtggggggtcatgggggggggtgataatgggccataatggcggcgcttgtacaatgtagattacttggtgggatataaacatgatgtacaagaacaagtatttttactgagctgctgaatggctgtcactcatttgcgcttaaaaatgtcctcaggtgcagaataactgtcgctatggctgttaatttggggccatatacaagtagctcaaatccacgctccaccgctgctggggttgagacaaaacctgtgagcctccacattgcacaaaagagggaaataatcggcaccaatcggtatcaatcagaaccaaccgatttatctgtttaacaccaaaacccctttctacaagtagccacatctaaccactcaccaggcccattaccccgcctgttgatcaacggctatttctacctcatccatattgggggtgcataacccatcagactgggcgtctaataggaagagcaggaggaggatattagacgacgtcctcacccagctgggtaagagcagagtgacaaccagctctctttccgaaagccgtatatactctcaaaacactccaccatctacgataagcatatcggcgcgagtcttatttcatatatctggtgttttaccgtaagatccaccttccatccgaagtatgaggcttcgggttcaacccccgccctcatacactacttgtagaaggcggaggtgtccacacacttttttttctttctgcaaacaatactctccgttgcactgcggctcaataatgctacatacgacttatccggaattaggttgggaccgttgagtacgaccgctgatcgacgactctgaagatggtgatatcgctgaaagagataccattacactacatttgaaatacagaacattatttccaggagtaatgtaccacttgaagtctgtgattttaaatctctcatcaactgtgtatacctggctcttgcaaacctccacatgcatgcagcactacctccatgaacggatctcggatcccctactgcggcgattcagtgggtatatacctcaattggacctataaactaattattcttctcctcgacttttctatttcatcacacacacttacacgcacacgaacaaacactacgatggtccttcttattctcc

SEQ ID NO: 31 - DNA, 1500 bp, Yarrowia lipolytica
  1.5Kb promoter region YALI0D25256g
aaacgagtctcagaagtaactgaaactagacacagtcggaatcggtccacctttgtcattcccaactgtactgtacttgtagatgaacacctgtcttcagagctccatgtactatcccccaaccgagcaatcttggtgtttggtacccaacgcctcgcaaaagctcaagaggggaatggcccgcggcgttggtaaaccgacaagactgagtcgattggtagttgcatgttgggaatttggagggtcaatcacgagagatggtactgttgcagaagtcatggtgtattcataattcaaggtgatcagcaggaactggagtccttcaatcatccgaaagaatgatcatggtatatgtgagactcagtcggttcattattcctgtttgtctaattccctttctctacaatgattcgatatccgtcaatattgggcgagtaagagaggtttgtagttcactagaaagttccccagaaagtttcccagaaagttccccagcaagttccccaacttcaactagtgcaaatctcggtagtattttgtcattgttttataaaagaaagctcatcattattatgaaagattgcagcatttcaccgagaggtagagcggcttttcaaagagttcaaatcggctcaaatctgttcaaatcagctcaatctgttcaaatcagctcaatctgttcaaatcagctcaaatcaactcaattcagttcaaattcgacaagacactctcatcgctcattcgatgaatcccttttataatccagcagtattcatatgctctcccgtccacgtgggcagtttccttcagctacaatacatctgcaaagacactctgttctatacctcccgggtatccgctatgttcaattgccttgctaaaacctcacaagccttgctatgcagtccagtcgaattatccgggatttggtacgcttcaacacgaaagaggatgtacagtacaagtagaccgacagacctgcaccccgcctactacaagtagccgctttgaccccccactagacaacacacacacagtgagatgtaaacggccagtttctagcagccacgtacgtacttgagcagtatagaaccatgcaactgcatctctccctctgtgacccccaattgcaaaccgtgtttgaaaatacctttgccaatcagcgcaggttgaatgtgggtgaacaaggtcaaaacgggtgaacaaaggtcaaaacgggtgaacaaggtcaaaacgggtgaacaaatgtcaagcgcaagacggagacagaagtttcggggtcacgtgactatcacgagttgcgattctacgacgctcatcagcgacgcccttggatttgctccattctcgcttaatttcgcttatcgccaattgcgtggggggtgtggggggtcccaactttaaaacacttacccaacatgtcccccacgctctagatatataccacctcgaaaatcccactcccccacacatcgctcaattcctccacgcgtgcgccaa

SEQ ID NO: 32 - DNA, 1500 bp, Yarrowia lipolytica
  1.5Kb promoter region YALI0C11099g
ggtaccatcactcaactggttgcggcacctctcgacatgtgtcaatgggacacttcctgctgtccgctttgcggttggggagttttcggtcagtaagggacgaagcgaaacgaccgaggttggttcgtaccctgaacatggtggtatcgaaaggtagctctggagtgaagaccatgacttcattctcatcttcaatcacaccctcctcgtagagatcctggttaccaccagaatcaacatctacaaaatagtctgactcgtctgagtcgtaacaagagacattatcctccttattcctttgcttaggtcccatcgtctgcttttcgcagtggcagcagtccgttgacgagctctcctcttcttccaccacgccaggcttttgagagtcaacttgcgcagaacagaaaactccagttcgttgttgaagacgtcctcgtgttctctcgatccaacaacctgggagatctatgctaggtgccacctccttgatagattcagcaatcctgggcaaggcgctggctgaaaattatcaggccacctgtagagcattctttccgtccacgtggggacaccttgtactcggtgtggttgacaaaagcagaccatctcggatcccttgagcatcttagcaagtggcgtcacctactcgagcttgtctcccaccagtgtccttgagcttcaaatccagagacggccccgatacattctcttatgattgttgaataccgtttccgctccttgaagagcagggaatcttgacggactggttgtggtagaagagccagccggcagaaacacggccactgaatcaaggagagattgtagtagagcgtctttttgtcgcccttgcctccgattccatactttttcgtcagggtgtgtttgatatccatcagtttgagctgctttacaggggcatgggagccccttctccctctttacccgtcagactccatccttcttcttcgttctcttctccgtcttctctcatcaccatagtggtagaggtgtgcagaccttgatatcgtcggagcagaagttgtcgtcggaactagccaggctcattttgtcaatgtattgagctgcattgcgtatctcgcgtcttattctaagacaccgtctcagcacattagtgcggcttgaaatttcattcacgacttggaaattatgccgcccgtaccgtcgaaagatgttagtaagcaatagctgaaaataccgggaatcgattggtgcagtctcaatgtgtctatttaaaccaacttggtctgtttttgctatattggagccccgctagaactcacgatcgagcctctggttcttagcgagaaaataagcgagtcgcaccacagccgtcgaaacacccgctggcttgcaagttgaacacctcaaggagggctccagatggtgatcatgacaataaaaaggaccccaaatcctctctattcctatctactcacaaacaccaccaatatgaaagtccttgtcctcccacgccagctc

SEQ ID NO: 33 - DNA, 1500 bp, Yarrowia lipolytica
  1.5Kb promoter region YALI0F09966g
tcggacgcctttatcaatccccatgtcttcttcagcatttagtagcagcgacaatggtgccttggagcaatgttttgttgcgagatttgtggcagggtccagaggtaaatcacttggtctcgacaaagtgcgacgaggacccacttgggccgccttccaagaggagtgtggagacaagaccaagaccagtggattcggagccgtctcgatgattgattcgccgactcttccctcccgtaaccgtgtcattctccttccacagccgggttccgccccttctggcgaaagttgcactctttttcagaccccgaaaaaaagactttggctttaactgtcgtacatgatgtcgatgtcgatagtggttgaagagagttgccagaaaggtcagagcgatgcgattgatgcggtgaaaaaaaaaaaaaaagccatgttgatatgcattttgcacttactctagagagtgccctagatctttccattgcccggacctccctccggacctccccttccgtacctctctgccccttctggacaggtcaatgatagactcagagcgacacacatgtctgacgtaccatgttagaccttgtattgacctggacgaatgtgtgtgaggagtgaggcaggccaagacgaaccacggtctttatatatgcccacggagtgacacggtctgtgtcgtcaccgcagctccactcaccacccgcatcatgatcgtccaaccagaacccactccccagtttcgacccaacaccattctcaactgtaagtatgagtaccacagtgatactcgcccagtgccgcactcgtactgtagccactccactgcaacatccgtatcgtattgcaccgccccgattcacctgcttccttcaagccttcaaccacgtactgctccacctcctaccgttgagcccactcggatcggccagagtcatgtcttagggtttggctgcagttgtggcgtaaactatggagaaggcgacggaaacgagagcgctaccggtagcgacttggcgacacgtctggctcgggaagggggccgttgcagagaccaagacttccgtcacgtgaccgctgtttggtcaattctaacgcagttattttccgtctgattcgctgatacgagtactcgcttgctgtagatgactcagaccaagacaagagaaggggaaataaaaaaaacttccaaaaaaaacttccaaaaaaaaaaaatcaaaatttgacaaaccttttctgcctgggaccaggaactttgtgagtccattgagggagttagccacccatcagccacagccacagtttggacaagaagtaaaagtggatatatttatgttatggagaccatgtagtgttgtgggagggaggggtttttttgtttgttttggctgagtaatcaacagcaagtggcgtatatcgtatatctatcgtgactcagactattcaccgcttgtatggtgctatctcgacttgtgcttagtctcaggtacacgtgattgc

SEQ ID NO: 34 - DNA, 1148 bp, Yarrowia lipolytica
  YALI0A12353g
atgaaggtccttcttattctctacgatgccggctcccacgccaaggacgagcccaagctgcttggatgcaccgagaacgagcttggtattcgagactggctcgagtcgcagggccacactctcgtcaccacctcttccaaggacggtgccgactctgtgcttgacaaggagattgttgacgctgatgtagtcatcaccaccccgttccaccccggctacatcaacaaggagcgaattgacaaggccaagaagctcaagatctgcatcaccgccggtgtcggctccgaccacgtggacctcgatgctgccaacgcccgaaacattgccgttctcgaggtcaccggctcgaacgtccagtccgtcgctgagcatgttgtcatgaccatgctggtgctggtccgaaactttgtccctgcccacgagcagattatttctggcggctgggacgttgctgcggttgccaaggactcttacgatctcgagggcaaggtcatcggtaccgttggaggcggtcgaatcggccagcgagtgctcaagcgatgcaagccctttgaccccatggagatgctctactacgactaccaggctatgccagctgatgtggagaatgagattggatgccgacgagtcgagtctctggaggagatgctctctctctgtgacgttgtaaccatcaactgtcctctccatgcctccaccaagggtctcttcaacaaggagctcatttcccacatgaaggacggcgcttggctcgtcaacactgctcgaggagccatctgcgtcaccgaggacattgtcgacgctctcgagtccggcaagatccgaggctacggtggtgacgtctggttcccccagcctgcccccaaggaccacccctggagaaccatgcgaaacaagtacggcggtggaaacgccatgactccccatatctccggtacttccatcgacgcccagggccgatacgctgagggtaccaagaagattcttgaggtctttttctctggcaagcaggattaccgcccccaggacattatctgcatcaacggtcactacggtaccaaggcttacggtgacgacaaggagcacaaggcccacgtttccaagtagctgtttaacttatataagcaattgactggatttgataacga

SEQ ID NO: 35 - DNA, 1170 bp, Yarrowia lipolytica
  YALI0F15983g
atgaaggtccttcttattctctactctgccggttctcacgccgttgacgagcccaagctactcggttgcactgagaacgagctcggtctccgaaagtggctcgagtcccggggccacacgcttgtgaccacctcttcaaaggagggagccgactctgtgctcgacaaggagattgtcgatgccgacattgtcatcactactcccttccaccccggttacatcacccgagagcgaatcgccaaggccaagaacctcaaaatttgcatcactgccggtgttggttcggaccacgtcgatcttgatgctgccaacgagcgagatattgccgttctcgaggtcactggctccaacgtgcagtctgtggctgagcacgttgtcatgaccatgctggtcctggtccgaaactttgtgcctgcccacgagcaggtcatggctggtggctgggacgttgctgctgtcgccaaggactcctacgacattgagggaaaggtcattggtactgtgggaggtggccgaattggtcagcgagtcctaaagcgagttgctcccttcaaccctaaggagatgctctactacgactaccagggtctgtctgctgagaccgagaaggagctgaactgccgacgagtcgagaagctcgaggatatgcttgcccagtgtgatatcgtcaccatcaactgtcccctccacgagtccaccaagggtctcttcaacaaggagatgctctctcacatgaagaaaggtgcctggcttgtcaacaccgctcgaggagccatctgtgtcaaggaagacgttgctgaggctctcaagaacggccagctgcgaggctacggaggtgacgtctggttcccccagcctgctcccgctgaccacccctggagatccatgcgaaacaagtacggagctggaaacgccatgaccccccatatctccggtacttgcatcgatgcccaggttcggtacgcccagggaaccaagaacattctcgacatgttcttctccggtaagcaggactaccgaccccaggacatcatctgcatcaacggtcactacggaaccaaggcctacggtgatgacaaggagcatgctaaaaagtaggctggacgtcgaaataaacacattgttcatctctgggtagcccaatagtaaataggtaagctgtgatcatta

SEQ ID NO: 36 - DNA, 1199 bp, Yarrowia lipolytica
  YALI0B22506g
atgaagattctccttatcctttacgacgccggctcccacgctgctgacgaacccaagctgctaggttgcaccgagaacgagctcggcatccgatcctggctcgagtcccagggccataccctggtcaccacctcttccaaggagggtgctgactctgttctcgacaaggagattgttgatgccgatgttgtcatcaccactcccttccaccccggctacatcacccgagagcgaattgccaaggccaagaacctcaagatctgtgtcactgccggtgttggctctgaccacgttgatcttgctgccgccaacgagagaaacattgccgtgctcgaggtcactggttccaacgtgacttctgttgctgagcacgttgtcatgaccatgcttgttcttgtccgaaacttcgtgcctgccaacgagcaggttcgaggcggcggctgggacgttgccggtgttgccaaggattcctacgatattgagggtaaggtcattggtaccgtcggagtcggccgaatcggcaagcgagtgctccagcgactcaagcccttcgaccccaaggagctgctctactacgactatcagcctctgtctgctgccgacgagaaggagattggcgcccgacgagttgagaagctcgaggacatgcttgcccagtgtgatgttgtcaccatcaactgccccctgcacgaatccaccaagggtctctttaacaaggagctgctgtcccacatgaagaagggcgcctggctcgtcaacaccgctcgaggagccatctgtgtaaaggaggacgttgccgccgctctcaagtctggtcagctccgaggctacggcggagatgtctggttcccccagcctgcccctgctgaccacccctggagaaagatggtcaacaagtacggtgccggtaacgccatgaccccccacatgtctggaacctctctggacgcccaggcccgatacgctgctggtgtcaagcagattctcgacgagttcttctccggccgagaacagtaccgaccccaggacattatctgctacggtggtaactacggcaccaaggcctacggagacgacaagaaggtcgtcgacaagaagtaatccgtccaacgttcttctcagcgcacaggtacctatactactggtagtatcaggttaaacctcttttagtttaatgataagataagataacataccgt

SEQ ID NO: 37 - DNA, 1142 bp, Yarrowia lipolytica
  YALI0C08074g
atgaaggtccttcttgttctttacgatgctggctcccatgccaaggatgagcccaagctgcttggatgcaccgagaacgagctcggtatcagagactggctcgagtctcagggacatacccttgtaactacgtcttccaaggacggtgccgactctgtcctcgacaaggagattgtcgatgccgatgtagtcatcaccaccccctttcatcctggatacatcaacaaggagcgaatcgacaaagccaagaagctcaagatttgcatcactgctggtgtcggctctgaccacgtcgatctcgacgctgccaacgcccgagatattgctgttctcgaagtcaccggctccaatgtccagtctgttgccgagcacgttatcatgaccatgttggtcctggtgcgaaactttgtccctgctcacgagcagatcatctctggcggttgggacgttgctgctgttgccaaggactcatacgatctcgagggtaaggtcatcggcaccgttggaggaggccgaattggccagcgagtgctcaagcgatgcaagcccttcgaccccatggagatgctctactacgactatcaggccatgccagctgacgttgagaaggagattggatgtcgtcgagtcgagtctctcgaggagatgctctctctttgtgacgtggtcaccatcaactgccctctgcacgcttccaccaagggcctgttcaacaaagagctcatctcgcacatgaaggatggtgcctggctcgttaacaccgctcgaggagccatttgtgtcaccgaggacattgtcgaggctctcgagtccggtaagatccgaggttacggcggagacgtttggttcccccagcctgcccctaaggaccacccctggagaaccatgcgaaacaagtacgggggaggaaacgccatgactcctcatatctccggaacttccatcgacgcccagggccgatatgccgagggcaccaagaagatccttgaggttttcttttctggaaagcaggattatcgaccccaggacattatctgcatcaatggtcactacggcacaaaagcttacggagatgacaaggagcacaaggagcaccagctcaagtaaatatatatagctagaaacatatctcgttgttgctt

SEQ ID NO: 38 - DNA, 1135 bp, Yarrowia lipolytica
  YALI0F13937g
atgaaggtccttcttattctctatgatgccggctctcacgctaaggatgagcccaagctccttggatgcaccgagaacgagctcggtatccgagactggctcgagtctcagggccatactctcgttaccacctcttccaaggatggcgccgactctgtgcttgacaaggagattgttgacgccgatgtagtcatcactacccccttccaccctggctacatcaacaaggagcgaatcgacaaggccaagaagctcaagatctgcatcactgccggtgtgggctcggaccacgttgatctcgatgctgccaacgctcgagatatcgctgttctggaagtcactggttctaatgtccagtccgtcgctgagcatgttgtcatgactatgctcgttctggtccgaaactttgtccccgcccacgagcaaatcatctctggcggctgggatgttgctgctgtcgccaaggactcctacgatctcgagggtaaggtcatcggtaccgttggtggcggccgaatcggccagcgtgtcctcaagcgatgcaagcccttcgaccccatggaaatgctctactacgactaccagcccatgcctgccgacgtcgaaaaggagattggttgccgacgagttgagtctctggaggagatgctctctctgtgtgatgtcgtgaccatcaattgccctctgcacgcctccaccaagggcctcttcaacaaggagctcatctcgcacatgaagaatggtgcttggctcgtcaacactgctcgaggagccatctgcgttaccgaggacattgtcgaggcactcgagtccggcaagatgcgaggatacggtggtgatgtctggttcccccagcctgcccccaaggaccatccctggagaaccatgcgaaacaagtacggtggtggaaacgccatgactcctcatatctctggcacttccatcgatgcccagggccgatacgccgagggaacaaagaagatcctcgaggtcttcttctctggcaagcaggattaccgaccccaggacattatctgcatcaacggtcactacggtaccaaggcttacggtgacgacaaggagcacaaggagcaccagcttaagtaaacatttttatagtaacatctcattttgt

SEQ ID NO: 39 - DNA, 1107 bp, Yarrowia lipolytica
  YALI0C14344g
atgaaggtccttcttgttctatacgatgccggctcccacgccaaggatgagccaaagctacttggatgcaccgagaacgagctcggaatcagagactggctcgagtcacagggccacactcttgtcaccacctcttccaaggacggtgcccactctgtgcttgacaaggagattgtcgatgccgatgtagtcatcaccacccctttccaccccggctacatcaacaaggagcgaattgacaaggccaaaaagctcaagatctgcatcaccgctggtgttggctccgaccacgtcgatctcgacgcagccaacgcccgagatatctctgttcttgaggttactggttctaatgtccagtctgtcgcagagcacgtcgttatgaccatgctggtgctggtccgaaactttgtgcccgcccacgagcagatcatcgagggcggctggaatgtggctgctgtcgccaaggactcgtacgatctcgagggaaaggtcattggcaccgttggaggcggtcgaatcggccagcgagtgctcaagcgatgcaagccctttgaccccatggagatgctctactacgactaccaggctatgccagctgatgtggagaaggagattggatgccgacgagtcgagtctctggaggagaagctctctctctgtgacgttgtcaccatcaactgtccccttcacgcctccaccaagggtctcttcaacaaggagctcatttcccacatgaaggacggcgcttggctcgtcaacactgctcgaggagccatctgcgtcaccgaggacattgtcgacgctctcgagttgggcaagatccgaggatatggcggtgacgtctggttcccccagcctgcctccaaggaccacccctggagaaccatgcgaaataagtacggcggtggaaacgccatgactccccacatctccggcacttccatcgacgcccagggccgatacgctgagggtaccaagaagatcctcgaggtctttttctcgggaaagcagaattacagaccccaggatatcatctgtatcaacggccattatggtacaaaggcctacggtgacgacaaggagcacaaggggcaccagcagaagtga

SEQ ID NO: 40 - DNA, 1153 bp, Yarrowia lipolytica
  YALI0E14256g
atgaagatccttcttgttctctacgacgccggttcccacgccaaggatgagcctcgacttctcggatgtaccgagaacgagcttggtctccgtgactggatcgagtcccagggacacaccttggtgaccacctccgacaaggacggtgagaactcaaccgtcgacaaggagattgtggatgctgagattgttatcaccacccccttccaccccgcttacatcaccaaggagcgaattgacaaggccaagaagctcaagatctgtatcactgccggtgttggttccgaccatgtcgaccttgatgccgccaacgcccgagacattgccgtcctcgaggttaccggatctaacgtccagtccgtcgctgagcacgttgtcatgaccatgctggttctggtccgaaacttcgtccccgcccacgagcagatcatcgagggtggctggaatgtcgctgctgtcgccaaggactcttacgacattgagggtaaggtcattggtactgtcggcggtggccgaattggtcagcgagtcctgaagcgacttgcacccttcaaccccatggagctcctctactacgactaccagcccatgcccaaggacgtggagaaggagattggctgccgacacgtccctgatcttaaggagatgctctctgtctgtgacattgttaccatcaactgtcctctccacgactctaccaagggtatgttcaacaaggaactcatctcccacatgaaggatggtgcttggctcgtcaacaccgcccgaggtgccatctgtgtcactgacgacattgttgaggccctcaagtccggtaagatccgaggctacggtggtgatgtctggaacccccagcctgcccccaaggaccacccctggagatacatgcgaaacaagtggggcggtggaaacgccatgaccccccatatctccggtacctccatcgatgcccagggccgatactccgagggtaccaagaacattctcgaggtctacttctccggaaagcagaactaccgacctcaggatgtcatctgtatcaacggccactacggcactaaggcttacggtgacgacaaggagcacaaggcccacgtttccaaataagcactttagctgtttaacttataagcaattgactactagaatttgc

SEQ ID NO: 41 - DNA, 1171 bp, Yarrowia lipolytica
  YALI0B19976g
atgaaggtccttcttgttctctacgacgccggctcccacgccaaggatgagcccaagctacttggatgcactgaaaacgagctcggtatccgagactggctcgagtcccagggccataccctggtaactacctcttctaaggatggcgccgactccgttctcgacaaggagattgttgatgccgatgttgtcatcactacccccttccatcccggttacatcaacaaggagagaattgacaaggccaagaagctcaagatctgtatcactgccggtgttggttctgaccatgtcgatctagatgccgccaacgcccgagacattgctgtccttgaagtcactggctccaacgttcagtcggtcgctgagcatgttgtcatgaccatgcttgttctggtcagaaacttcgtccctgctcatgagcagatcatttctggtggctgggacgttgccgctgtcgccaaggactcttacgacctagagggtaaggtcattggtacagttggaggtgggcgaatcggtcagcgagtcctcaagcgatgcaagcccttcgaccccatggagatgctctactacgactaccagcccatgcccgctgatgtcgagaaggagattggctgtcgacgagtggagtctctcgaggagatgctctctctctgtgacgttgtcaccatcaactgtcctctgcacgcctccaccaagggcctcttcaacaagaagctcatctcccacatgaaggatggcgcctggctcgtcaacaccgctcgaggagctatttgtgtcactgaggacattgttgaggctctcgagtccggaaagattcgaggctacggtggtgatgtctggttccctcagcccgccccaaaggaccacccctggagaacaatgcgaaacaagtacggtggaggaaacgccatgacccctcatatctctggtacttctattgatgcccaaggccgatacgctgagggcaccaagaaaatccttgaggtcttcttctcaggaaagcaagactaccgaccccaggatatcatctgtatcaacggtcactacggtaccaaggcttacggtgacgacaaggagcacaaggagcacaaggagcacgaggtcaaataagctatcagctctatagtactgtacagtaggtacaataggatcttgatattacagt

SEQ ID NO: 42 - DNA, 1107 bp, Yarrowia lipolytica
  YALI0E15840g
atgaaggtccttcttgttctttacgatgctggctctcacgctgctgacgagcccaagctccttggatgcaccgaaaacgagctcggtatccgagactggctcgagtcccagggccacacgcttgtcaccacctcttccaaggatggcgccgactctgtgctcgacaaggaaattgttgacgccgacgttgtcatcaccacccccttccaccccggctacatcaacaaggagcgaatcgacaaggccaagaagctcaagatctgcatcaccgccggagtcggctcggaccacgttgatctcgatgctgccaacgctcgagatatcgctgttctggaggtcactggttctaatgtccaatccgtcgctgagcatgttgtcatgactatgctcgttctggtccgaaactttgtccccgcccacgagcagatcatttccggcgggtgggacgttgcggcggtcgccaaggactcctacgatctagagggtaaggtcatcggtaccgtgggaggcggccgaatcggtcagcgtgtcctcaagcgatgcaagcctttcgaccccatggaaatgctctactacgattaccagcccatgcctgccgacgtcgaagaggagattggctgccgacgagtcgagtcgctcgagcaaatgctttctctgtgtgatgttgtcactatcaactgccctctgcacgcctccaccaagggtctcttcaacaaggagctcatctcccacatgaaggatggagcctggctcgtcaacactgctcgaggagccatctgcgtcaccgaggacattgtcgaggccctagagtccggcaaaatccgaggatacggaggagacgtctggttcccccagcctgcccccaaggaccacccctggagaaccatgcgaaacaattacggtggtggaaacgccatgactccccacatctccggaacctccattgatgcccagggccgatacgctgagggtaccaagaagatcctcgaggtctttttctcgggcaagcaggattatcgaccccaggatatcatctgtatcaacggccattatggtaccaaggcctacggtgacgacaaagagcataaggagcatcagctcaagtaa

SEQ ID NO: 43 - DNA, 1158 bp, Yarrowia lipolytica
  YALI0F28765g
atgaaggtccttctcattctctacgacgccggctcccacgctgttgatgagcccaaactactcggatgcaccgagaacgagctcggtatccgatcatggctcgagtcccagggccatactctggtaaccacctcttccaaggatggcgatgactctgtcctcgacaaggaaattgtcgacgccgatgtcgtcatcaccactcccttccatcccggttacattacccgagagcgaattgccaaggccaagaacctcaaaatatgtgttactgcaggtgttggttccgaccatgtcgaccttgatgccgccaacgagcgagacattgccgttctcgaggtcactggttccaacgtccaatccgtcgctgagcacgtcgtcatgaccatgctcgtcctcgtccgaaactttgtccccgctcacgagcaggtcatggctggtggttgggatgttgctgctgtcgccaaggactcttatgacatcgagggtaaggtcattggtaccgttggtggtggccgaattggtcagcgagtcctcaagcgagtggctcccttcaaccctaaggagatgctctactacgactaccaaggactttctgctgagaccgagcaggagctcaactgtcgacgagtcgagaagcttgaagatatgcttgcccaatgtgacattgtcaccattaactgtcctctccacgagtctaccaagggcctcttcaacaaggagatgctctctcacatgaagaagggtgcttggcttgtcaacaccgctcgaggagctatctgtgtcaaggaggatgtcgccgaggctcttgccaacggccagcttcgaggctacggtggtgatgtgtggttccctcagccagcccctgccgaccacccttggagatcgatgcgaaacaagtacggcgccggtaacgctatgaccccacacatatctggtacttccattgacgcccaggcccgatacgccgagggcaccaagaacattctcgaggtcttcttctccggaaagcaggactaccgaccccaggatatcatctgtatcaacggtcactacggtaccaaggcttacggcgacgacaaggagcacgccaagaaatagataatattcattaacatacaaatacagtctacagtcctatatcgattcggtcgcttcgag

SEQ ID NO: 44 - DNA, 1399 bp, Yarrowia lipolytica
  YALI0E19657g
atgtctacgaaaccaaccattgcgctagcgggagggacgtcgcggttcgaccaggttctgagccggctggaacacttcaacaccatcttctaccccgtgcctgccaccaaggaggagctgttcaaagactgtctacccggcggtcccctggccaacgtggagggtctgttttgctcctggccggcattctacgccatgggaggcctcaagaccgaggaggagattgcccagttgccggcgagtctcaaggtggtggctctctgtgccacggggtacgaccagttcaacgttgctgcgttccggaaacgtggcatcattgtgaccaacactccgtcgatggagccctctcacagcggcgagcaggtcgcggacattgcgctctatctgtcgattggatgcatgcgcaaaatccccctgtttgagggctcgttccagagagagaagaactcgatcgacgcgcggcgtgtggtggcgcagggcgagtttgaccccgcttcgggccaggttctcggtggtccgccctcaggattcgcctttggagatctcaccgcgaaaggacccgccagaatgtgccgaaaccgggcggtgggcgtggcgggactcgggaacattggaaaggccattgtgcgacgcttggcgcctctgggggtcgaaattcactactacaagcgatccgagctgagcgctgaggagctggcggcctcaaatctcccctcagacctcatcttccactcctcctttgacgatctgtgcgccgcttcggaccagctcattctggcgctccccggaggagcagacaccctcaacattgtcaacaaacggaccctgaacctcatgccccgaggagcgtccatcgtcaacattggtcgaggcactctcatcaacgaagacgatctgctcgaggcgctgggctccaaacagattgccaccgcaggcctcgacgtccaggtgggtgagccgtttgtcaatcccaagctatttgggcggtgggatatccaactgctgccccatctgggcagtggaaccgaggacaatgccctggcggccgagctcaatgtgattgacaacattgaaaatgtcctgaacggcggtcctggtcttcatcctgtcaattgagctttgacaatgacaaccatgacaaccatgacaactatggcaaccatgacgaacagcgaccaacaacgaccaacaatgacaaacagcgacgaacaacgacgacagcgacgaacagcgacgacagcgacgaaacatgttcatggatagtacatgccctacaaaagggacattcgttccctagactagctaatacgagcccctttctcgtcgccaaagacatctataagctatattgaactaaactgtacgaaacttgtggtaacatgaaccactagagtcactg

SEQ ID NO: 45 - DNA, 1160 bp, Yarrowia lipolytica
  YALI0B21670g
atgctccgtatccgaccacagtttgcgcgacagtcgctcgcccgattgtactcgacccaaagacagaagatcctgttccttgacaacgtggtggacgccaaagacgagtttgaaaagctcaagaagaaggccgacgtggtgactctcaaggatggaactgaccgagaatcgtttctcaaggacctcaaaaacacctacaaggacatcaatgccatcttccgaaccttcatcagtgtcaaaaagaccggccggttcgacgaggagctggccaaggctcttcctgagtcctgcaaagctgtctgccattacggagcaggctatgaccagatcgatgtgcccttcttctccgaaagaggcattcaggtcagcaacgtgcagtccatggccgacgagtcgactgctctcacgaacctgtatctaatgatcggcaccctgcgaaacttcggagacggagctctcaacttgcaaaagggccaatggctcaagggagtggccctgggcaacgacatctccggcaagactctcggaattctcggtatgggcggaatcggccgagaaatcagagactacgtggctcctctgggtttcagcaaggtgctgtactacaaccgaaatcgcctggcccccgagctggaaaaggactctgtctactgccagtcgcctgaagatctattttccaaggccgatgtcatttccatcaacgtgcctcttaatgctgctaccaagcacctcgtcaacgcagagtccatctccaaaatgaaggacggagtcatcattgtcaacaccgctcgaggccctgtttgcgacgagaaggccctagtggacggtctaaactctggaaagattggaggagtcggtctcgatgtattcgagcgagagcctgccattgaagaaggtcttttgaaacaccctagaaccctgcttctgcctcatatgggaacctggtcccacgagacccatttcaagatggagaaggcggttctggacaacctcgagagctttgttgatactggaaaggttatctccattgttcctgagcagaagggcaagttttaaaatggtaacaactaacatagatagggggagggggagctacagtttccagtagctaagtaagtacaagtagagctaaacgcataaacatgcattgtgtagaaagt

SEQ ID NO: 46 - DNA, 1069 bp, Yarrowia lipolytica
  YALI0F29315g
cacgcggctgatgagcccaaactcctcggatgcaccgagaacgagctcggtatccgatcattgctcgagtcccagggccatactctggtaaccacctcttccaaggatggcgatgattctgtcctcgacatggaaattgtcgacgccgatgtcgtcatcaccactcccttccaccccggttacattactcgagagcgaattgacaaggccaagaagctcaagatctgtatcactgccggtgttggttccgaccatgtcgatctagatgccgccaacgcccgagacattgctgtccttgaagtcactggctccaacgttcagtcggtcgctgagcatgttgtcatgaccatgcttgttctggtcagaaacttcgtccctgctcatgagcagatcatttctggtggctgggacgttgccgctgtcgccaaggactcttacgaccttgagggtaaggtcattggtacagttggagggacgaattggtcagcgagtactcaagcgatgcaagcccttcgaccccatggagatgctctactacgactaccagcccatgcccgctgatgtcgagaaggagattggctgtcgacgagtggagtctctcgagaagatgctctctccctgtgacgttgtcaccatcaactgtcctctgcacgcctccaccaagggcctctccaacaagaagctcatttcccacatgaaggatggtgcctggctcgtcaacaccgctcgaggagctatctgtgtcaccgaggacattgttgaggctctcgagtccggaaagattcgaggctacgtgggcgatgtctggttccctcagcccgagaataatgcgaaacaagtacggtggaggaaacgccatgacccctcatatctctggtacttctattgatgcccaggcccgatacgccaaaggtaccaagaacattttcgaggtcttcttctccggaaagcagggctatcgaccccaggacatcatctgtatcaacggtaactacggtaccaaggcttacggtgatgacaaggaacattctaagaaataagttagttacgtgaatccactagattattt

SEQ ID NO: 47 - DNA, 1199 bp, Yarrowia lipolytica
  YALI0D25256g
atgacacaaaaagtacttttcctggacgagatccacgacgccaccaaggactacgcggctctggcgcagaaatgcgatattcagcatgtcggaacaacgtcgcgagagcagtttctcaaggagtgcaaggagggcaagtacgacgggtttgtggccatctaccgaacgttcaccacgctcggcaaggtcggaaggttcgacaaggaactatgtgacgctctgccggcctccatcaaggctgtgtgccactacggagccggctacgaccaggtggatgtggcgccgttcaccgagcggggaatccaggtcagcaacgtccaaggagccgctgacgccgccacggcgctgaccaacgtatatttgatgctgggctgtctgcgaaactttggtcatgcggccatttcgctccgacagggcaattggattggagacgtgccgttgggccatgatcccgacggcaaagtgctcggaatcatgggcatgggcggcattggccgtcaggtgagagattacgtcaagccgttcgggttcgagaaaatcatctactacaatcgaagccgactgtctccggagctcgagggcggatgccattacgtgactctggacgagctgtatgcccaggccgacgtcatctccgtcaacgtccccctcaacgccgccacccgtcacatgatcaactccgagtccatttccaagatgaaggacggtgttatcatcgtcaacaccgcccgaggcccggttattgacgagcaggctcttgttgatggtctcaactccggcaagatcagctccgccggtctggacgtgtatgagcacgagcccaagattaaccccggtctgctgaagaacccccaggccctgctgctgccccactttggcacttttaccattgagacccatcgaaaaatggaggaggcggtactcaacaacattgagacgttcctgaagaccggtaaggtggccacgattgtacccgagcagaacggcaagttctagctcggttgcgcggcgagattgtaatggaagtgcccggccaaaggtcaggcacggacccgataaccagttgtgggagaaggacagtttgattgttcaaccaaattcggtcaatctgctaaccctttgatcctggtgagactacaagtagccagaaatagagaatgaaatgttgacatagagccactaggtggctgcttggatga

SEQ ID NO: 48 - DNA, 968 bp, Yarrowia lipolytica
  YALI0C11099g
aagctccttggatgcaccgagaacgagctcggtatttgagactggctagagtaacaggaccacactctcgttaccaactctttgaaggacagccgactccgttttcgacaaggagattgtcgacgccgacgtagttatcaccacccccttccaccccggctacatcaacaaggagtaaattaacaaggccaagaggctcaagatctacatcaccgccggtgtcggctcggaccacgccaatattgacgcagccaacgcccgagacattgccgtactcgaggtcaccggcttcaacgtccagtccgttgctgagaacgttgttatgaccatgctggtgctggtccgaaacttgtcgctgcccacgagcagatcatttctggtggcagcgagtcctgaagagacttgcacccttcaaccccttggagctcctctactacgactaccagcccatgcctgccaacgtcgagaaggaaattggatgccgacaagtcgagtctctggagaagatgctttctctctgtgacgctgttaccatcaattgtccccttcacacctccaccaagggtctcttcaacaaagtgctcttctcccacatgaaggacggagtctggctcgtcaacaccgcccgaggagccatctgcgtcaccgaggacattgccaaagccctcaagtctggcaagatccgagcatacggcggagacgtctggtttctccagcctgcccccaaataccacccctggagaaccatgcgaaacaagtacagcgggggaaacgccatgactccccgtatctccggcacttccattggtgcccagggccggtccgccgagggtaacaaaaagatcctcaaggtctttttgtcgggcaagcaggaataccgacctcaggatatcatctgtatcaacggccattatggtaccaaggcttacggtcacgacaaagagcacaaggagcaccagaccaagtag

SEQ ID NO: 49 - DNA, 1634 bp, Yarrowia lipolytica
  YALI0F09966g
atgaaatggatgcgtcatagttcagacaaaatttcgaacctaaaacggcccacagataaaacggtgcattgtcgcacatacgaacagaccagtcgtcgttacatcgccaacaacccccgccacaccatgtccgccgtcaacattcccgtgaacgaaaacgaggtttcggtctcgtcttcgcccatcacgtcgtacggatcgcccgtctcgggctcgttccagggcaagccccgacagcgacgatactcttacaccgctgcgcgaaccaacaagctgaagccgttttccaccggcgacatcaagatcctgctgctggaaaacgtcaaccagacagccattgacattctcgagggccagggctaccaggtggagacccacaagtcgtcgctggacgaggaggagctgattgagaagatccgagatgtgcaggtggttggtatccgatccaagaccaagctcaactcacgggtgctcaaggaggccaagaacctcattgccattggttgtttctgcatcggtaccaaccaggtcgatctcgagtacgccgccaacaacggtattgccgtgttcaactcgcctttctccaactcgcggtctgtggccgagctggtcatctgcgagatcatcatgctgtcgcgacagctgggcgaccgaaacatcgagatgcacgccggcacctggaacaaggtctccgccaagtgctgggagatccgaggcaagactctcggaattgtgggctacggccacattggctcccagctgtccgttctcgccgagtccatgggcatgaacgtcatttactacgacgtgatcatgatcatgggtctcggtaccgccaagcaggtgcccacccttgccgagctgctggcccaggccgactacgtctctctgcacgtgcccgagctgcccgagaccatgaacctcatgtccaaggcccagttcgacggcatgaagaacggctcgtacctcatcaacaacgcccgaggcaaggtcattgacattcccgagctcatctctgccatgaagtccggaaagcttgctggagccgccgtcgacgtcttccccaaggagcctgccaagaacggctcaaacgagtttggctcccacctcaacgagtggaccaatgagctcctcaccctgcccaacctcatcatgtctccccatattggaggctccaccgaggaggcccagtccgccattggtatcgaggtcggcaccgctctcaccaagtacatcaacgagggttcctctgttggtgccgtcaacttccccgaggtcaacctcaacgtggtcaaccacgccgaggagcacgaccacgtgcgaatcctgtacgtgcataagaacgtgcctggtgtgcttctgtccgtcaacgagattttcgcctcccataacattgagaagcagtactctgagtcgcgtggtgatattgcctacctgatggccgacattgctgacgtcgatcaggccgacatcaagactctgtacgagaagctcgagcagacccccttcaagatctccacccgactgttgtattaagtggtgaggagagaccaatatgctaagacaagacaaacaagagattcaactaattttatagatgtaaatacattcaaccgtggttaacgacaatgctg

SEQ ID NO: 50 - DNA, 28 bp, Artificial Sequence
  Yarrowia lipolytica consensus
gtgcggctcggaaattcacawggkccgt

SEQ ID NO: 51 - DNA, 17 bp, Artificial Sequence
  Yarrowia lipolytica consensus
tygtgcggctcggaaat

SEQ ID NO: 52 - DNA, 46 bp, Yarrowia lipolytica
  YALI0B22506 consensus alignment
agcgggcctggatacaattccaagccggttcgtgcggctcggaaat

SEQ ID NO: 53 - DNA, 21 bp, Yarrowia lipolytica
  YALI0E14256 consensus alignment
cggcttttgcggctcggaaat

SEQ ID NO: 54 - DNA, 16 bp, Yarrowia lipolytica
  YALI0F15983 consensus alignment
atactgctacggaact

SEQ ID NO: 55 - DNA, 21 bp, Yarrowia lipolytica
  YALI0F15983 consensus alignment
cgtctctaaagtctcggctgt

SEQ ID NO: 56 - DNA, 30 bp, Yarrowia lipolytica
  YALI0F28765 consensus alignment
gaactctacaacttccgtgctgtgcggctc

SEQ ID NO: 57 - DNA, 46 bp, Yarrowia lipolytica
  YALI0B19976 consensus alignment
atgcggcttggaaatgaaattcggctccgtcgtgcggctcgaaaat

SEQ ID NO: 58 - DNA, 28 bp, Yarrowia lipolytica
  YALI0C08074 consensus alignment
gtgcggctcggaaatttccaaggaccgt

SEQ ID NO: 59 - DNA, 17 bp, Yarrowia lipolytica
  YALI0C08074 consensus alignment
ctgtgcggctcagatat

SEQ ID NO: 60 - DNA, 28 bp, Yarrowia lipolytica
  YALI0E15840 consensus alignment
gtgcggcttgaaaattcaggtggtccgt

SEQ ID NO: 61 - DNA, 17 bp, Yarrowia lipolytica
  YALI0E15840 consensus alignment
tcgtgcggctcgaaaat

SEQ ID NO: 62 - DNA, 28 bp, Yarrowia lipolytica
  YALI0F13937 consensus alignment
gtgcggctcgaaaattcaggcggtccgt

SEQ ID NO: 63 - DNA, 17 bp, Yarrowia lipolytica
  YALI0F13937 consensus alignment
ctgtgcggctcagaaat

SEQ ID NO: 64 - DNA, 28 bp, Yarrowia lipolytica
  YALI0A12353 consensus alignment
gtgcggctcgaaaatgcaatatagccga

SEQ ID NO: 65 - DNA, 17 bp, Yarrowia lipolytica
  YALI0A12353 consensus alignment
gagtgcggctcggaaat

SEQ ID NO: 66 - DNA, 28 bp, Yarrowia lipolytica
  YALI0C14344 consensus alignment
gtgcggctcggaaattcacatggaccgt

SEQ ID NO: 67 - DNA, 17 bp, Yarrowia lipolytica
  YALI0C14344 consensus alignment
tgatgcggctcggaaat

(19) United States
(12) Patent Application Publication
(10) Pub. No.: US 2023/0332166 A1
(43) Pub. Date: Oct. 19, 2023

(54) FORMATE-INDUCIBLE PROMOTERS AND METHODS OF USE THEREOF

(71) Applicant: Imperial College Innovations Ltd, London (GB)

(72) Inventors: Rodrigo Ledesma Amaro, London (GB); Johannes Kabisch, Darmstadt (DE); Stefan Bruder, Darmstadt (DE); Eva Moldenhauer, Darmstadt (DE)

(21) Appl. No.: 18/005,016

(22) PCT Filed: Jul. 9, 2021
(86) PCT No.: PCT/GB2021/051765

§ 371 Date: Jan. 10, 2023

(30) Foreign Application Priority Data
Jul. 10, 2020 (GB) 2010630.8

(51) Int. Cl. C12N 15/81; A23K 20/153; A23K 10/16

(52) U.S. Cl. CPC C12N 15/81; A23K 20/153; A23K 10/16; C12N 2830/002

(57) ABSTRACT

The invention provides nucleic acids capable of acting as inducible promoters in yeast species, in particular Yarrowia. The invention vectors, cells and associated methods of producing expression products from cells using the inducible promoters.

### FIELD OF INVENTION

The present invention relates to the field of engineering biology, and in particular to the use of microbes in bio-manufacture.

### BACKGROUND

The use of conventional yeast such as Candida, Saccharomyces, and Schizosaccharomyces in industry and biotechnology is well-known to the skilled person. In addition, the use of non-conventional yeast from genera including but not limited to Ashbya, Blastobotrys, Debaromyces, Dekkera, Hansenula, Kluveromyces, Lipomyces, Pichia, Rhodosporidium, and Yarrowia are increasingly significant organisms in industry, biotechnology, and synthetic biology. In particular, the non-conventional, non-methylotrophic oleaginous yeast, Yarrowia lipolytica, is an important organism for use in industry and biotechnology. Y. lipolytica is useful in the generation of products including but not limited to lipids, lipid by-products and fatty acids; oils and biofuels; proteins; and secondary metabolites such as citric acid and carotenoids.

Despite the significance of Y. lipolytica in industry and biotechnology, no widely-applicable, robust gene expression system is available. For example, systems utilising the POX2 promoter require oleic acid to induce expression (Müller et al, 1998), and systems utilising the EYK1 promoter are induced by erythritol (Blazeck et al, 2011). Oleic acid and erythritol are themselves complex chemicals which are produced by fermentation. Such inducing agents are expensive and labour-intensive to produce and are hence unsuitable for use in large-scale manufacturing and biotechnological applications.

The present invention solves these and other issues associated with currently available inducible promoter systems.

### BRIEF DESCRIPTION OF THE INVENTION

Formate dehydrogenase (FDH) is required for the metabolism of methanol and is typically only found in methylotrophic organisms. However, the inventors of the present invention have unexpectedly found that Yarrowia, a non-methylotrophic yeast, comprises a number of FDH genes that are regulated by promoters that are inducible by formate and that have been shown to be suitable for use in inducible expression systems, for example at least some of the newly identified promoters have a very low or absent level of basal transcription, i.e. in a very low or absent level of expression in the absence of the inducing agent.

The identification of the presence of formate inducible promoters in yeast that would not be expected to comprise genes such as FDH necessary for the metabolism of methanol represents a significant expansion in the tool-kit available for engineering not only Yarrowia but also other organisms that comprise FDH genes and the corresponding inducible promoters, and can be used in, for example, the bio-production of various compounds, as described herein, since the various promoters are induced to different degrees (i.e., provides a range of available induction levels), and are induced by a cheap, easy-to-produce inducing agent, formate.

### DETAILED DESCRIPTION OF THE INVENTION

As described above, the inventors have identified a number of formate-inducible nucleic acid promoters in Yarrowia species. Promoters that have previously been identified in non-methylotrophic yeast species have a significant basal level of expression meaning that they are less suitable for use in engineered expression systems. It was therefore unexpected that such non-methylotophic yeast would comprise such promoters that are suitable for us in engineered expression systems.

Accordingly, in one aspect the invention provides an isolated nucleic acid capable of acting as an inducible promoter in a non-methylotrophic yeast species, wherein expression from the promoter is induced by any one or more of a compound selected from the group consisting or comprising of: formate, formic acid, formaldehyde, methanol, ethanol, propanol, butanol and glycerol.

In preferred embodiments, expression from the promoter in the absence of the inducing agent is low or absent. It will be clear to the skilled person that in some situations it is preferable to use an inducible promoter that in the absence of the inducer results in a very low, or undetectable level of expression from the promoter. For example in some instances the inducible promoter may be used to express a product that is toxic to the cell. In these cases, it is important to maintain a low or at least non-toxic level of expression of the product in the absence of the inducer. That it was possible to identify formate inducible promoters in Y.lipolytica that show an appropriately low level of expression in the absence of the inducer was unexpected, since other fdh genes in non-methylotrophic yeast have been shown to drive a significant level of expression in the absence of the inducer.

In some instances, as well as, or instead of the basal level of expression in the absence of the inducer being a key determinant in selecting a promoter for use in a particular situation, the fold-induction of expression in the presence of the inducer is considered to be important. For example in some instances a relatively high level of background expression from the promoter in the absence of the inducer may be tolerable if the fold induced expression in the presence of the inducer is sufficiently high. Table 1 shows the fold induction of expression from a range of promoters of the invention when present in Y. lipolytica and when grown in YNB. It can be seen that all of the promoters are capable of being induced by formate—and some of these to very high levels of over 30 fold induction. Accordingly, the range of promoters presented in the present invention provide a suite of tools from which the skilled person can select the most appropriate promoter—for example based on basal expression level or fold induction in the presence of formate.

Accordingly in some embodiments the isolated nucleic acid is such that expression from the promoter is increased by at least 2-fold or at least 3, 4, 5, 6, 7, 8, 9, 10, 11, 12, 14, 15, 20, 22, 24, 26, 28, 30, 32, 34, 36, 38, 40, 45 or at least 50-fold when the non-methylotrophic yeast species is cultured in YNB with 0.5% sodium formate.

In some embodiments the nucleic acid is such that:
a) expression from the promoter in the absence of the inducing agent is low, absent or undetectable; and/or
b) expression from the promoter is increased by at least 2-fold or at least 3, 4, 5, 6, 7, 8, 9, 10, 11, 12, 14, 15, 20, 22, 24, 26, 28, 30, 32, 34, 36, 38, 40, 45 or at least 50-fold when the non-methylotrophic yeast species is cultured in YNB with 0.5% sodium formate.

The skilled person will understand that typically, the sequences necessary to provide a functional inducible promoter are located in a region up to 1 kb or up to 1.5Kb directly upstream of the translation start codon (typically the ATG). Accordingly, in one embodiment, the isolated nucleic acid of the invention comprises or consists of a region of up to 1Kb or up to 1.5Kb directly upstream of the translation start codon of a FDH gene, or of a putative FDH gene identified in a non-methylotrophic organism.

The skilled person will recognise however that it is likely that all of the 1Kb or up to 1.5Kb sequence is not necessary for promoter activity, nor that the exact sequence within this region has to have 100% identity to the native sequence. Once the skilled person has the knowledge that a particular 1Kb or up to 1.5Kb region is able to or is likely to act as an inducible prompter, the identification of, for example, minimal promoter requirements within this upstream region is largely routine. For example the skilled person is readily able to produce truncated or mutated versions of the promoter regions and assay the ability of the region to a) function as a promoter; and b) function as an inducible promoter. This can typically be performed by cloning the nucleic acid into a reporter vector and assaying the level of transcription or protein production in the presence and absence of the inducing agent. Such an example is given in the Examples. Trassaert et al (Microb. Cell Fact., 16:141 (2017)) demonstrates a 300 bp inducible promoter fragment, and Hussain et al. (ACS Synth. Biol., 5:213-223 (2016)) demonstrates a 55 bp inducible promoter fragment.

Accordingly, the invention also provides a nucleic acid that comprises or consists of a mutated or truncated version of the region that is 1Kb or up to 1.5Kb upstream of an FDH or a putative FDH gene identified in a non-methylotrophic yeast wherein the mutated or truncated version of the region is capable of functioning as a formate inducible promoter in a non-methylotrophic yeast, for example capable of functioning as a formate inducible promoter in the native non-methylotrophic yeast species.

The invention also provides a nucleic acid that comprises or consists of a sequence of a portion of a region that is 1Kb or up to 1.5Kb upstream of an FDH or a putative FDH gene identified in a non-methylotrophic yeast wherein the nucleic acid is capable of functioning as a formate inducible promoter in a non-methylotrophic yeast, for example capable of functioning as a formate inducible promoter in the native non-methylotrophic yeast species, for example in Yarrowia sp, for example Yarrowia lipolytica.

It will be understood that a portion of a region that is 1Kb or up to 1.5Kb upstream of an FDH or a putative FDH gene identified in a non-methylotrophic yeast may consist or comprise a portion of any length. Accordingly, in some embodiments, the nucleic acid of the invention comprises a portion of a region that is 1Kb or up to 1.5Kb upstream of an FDH or a putative FDH gene identified in a non-methylotrophic yeast where the portion is between 46 and 1500 bp in length, for example between 50 and 1500 bp in length, for example between 75 and 1500 bp in length, for example between 100 and 1500 bp in length, for example between 150 and 1400, 200 and 1300, 200 and 1200, 250 and 1100, 250 and 1000, 300 and 950, 350 and 900, 400 and 850, 450 and 800, 500 and 750, 550 and 700, 600 and 650 bp in length.

In the same or different embodiments the nucleic acid of the invention comprises a sequence of a portion of a region that is 1Kb or up to 1.5Kb upstream of an FDH or a putative FDH gene identified in a non-methylotrophic yeast where the portion is about 46, 50, 75, 100, 150, 200, 250, 300, 350, 400, 450, 500, 550, 600, 650, 700, 750, 800, 850, 900, 950, 1000, 1100, 1200, 1300, 1400 or about 1500 bp in length. In the same or different embodiments the nucleic acid of the invention comprises a sequence of a portion of a region that is 1Kb or up to 1.5Kb upstream of an FDH or a putative FDH gene identified in a non-methylotrophic yeast where the portion is at least 46, 50, 75, 100, 150, 200, 250, 300, 350, 400, 450, 500, 550, 600, 650, 700, 750, 800, 850, 900, 950, 1000, 1100, 1200, 1300, 1400 or at least 1500 bp in length.

It will be understood that a portion of a region that is 1Kb or up to 1.5Kb upstream of an FDH or a putative FDH gene identified in a non-methylotrophic yeast consist or comprise a portion spanning any range within the region. Accordingly, in some embodiments, the portion of a region that is 1Kb or up to 1.5Kb upstream of an FDH or a putative FDH gene identified in a non-methylotrophic yeast spans between about position 1 and 1500 bp, or between about position 46 and 1500 bp, 50 and 1500 bp, 100 and 1400 bp, 200 and 1300, 200 and 1200, 250 and 1100, 250 and 1000, 300 and 950, 350 and 900, 400 and 850, 450 and 800, 500 and 750, 550 and 700, 600 and 650 bp.

The skilled person will appreciate that the portion could span any region of the 1Kb or up to 1.5Kb upstream of an FDH or a putative FDH gene identified in a non-methylotrophic yeast sequences of the invention, for instance may span from position 25 to position 254; or from position 500 to position 725. Naming convention is that the sequence is orientated 5′ to 3′.

In some embodiments, the portion of a region that is 1Kb or up to 1.5Kb upstream of an FDH or a putative FDH gene identified in a non-methylotrophic yeast comprises or consists of a portion that is directly upstream of the translational start codon of the corresponding FDH gene. For example in one embodiment the invention provides an isolated nucleic acid capable of acting as an inducible promoter in a non-methylotrophic yeast species, wherein expression from the promoter is induced by any one or more of a compound selected from the group consisting or comprising of: formate, formic acid, formaldehyde, methanol, ethanol, propanol, butanol and glycerol, wherein the isolated nucleic acids comprises or consists of a portion of a region that immediately upstream of the translational start codon of an FDH or a putative FDH gene identified in a non-methylotrophic yeast, wherein the nucleic acid is capable of functioning as a formate inducible promoter in a non-methylotrophic yeast, for example capable of functioning as a formate inducible promoter in the native non-methylotrophic yeast species, and wherein said portion is:
about 46, 50, 75, 100, 150, 200, 250, 300, 350, 400, 450, 500, 550, 600, 650, 700, 750, 800, 850, 900, 950, 1000, 1100, 1200, 1300, 1400 or about 1500 bp in length; and/or is
between about 46, 50 and 1500 bp in length, for example between about 75 and 1500, 100 and 1500, 150 and 1400, 200 and 1300, 200 and 1200, 250 and 1100, 250 and 1000, 300 and 950, 350 and 900, 400 and 850, 450 and 800, 500 and 750, 550 and 700, 600 and 650 bp in length.

Accordingly, a nucleic acid of the invention may comprise a 150 bp region that spans the position 200 to 350 in a sequence that is 1.5 kb directly upstream from the start codon on an FDH gene identified in a non-methylotrophic yeast—provided that the nucleic acid of the invention is capable of acting as a formate inducible promoter in a non-methylotrophic yeast. In this instance, position 200 and 350 will correspond to a portion that is 1.3 kb to 1.15 kb upstream of the ATG start codon. The nucleic acid of the invention may also comprise a 300 bp region that is found directly upstream of the start codon of an FDH gene or putative gene identified in a non-methylotrophic yeast.

The inventors have identified a number of specific promoter sequences that are inducible by formate in Yarrowia lipolytica. Bioinformatics and alignment has identified the following consensus sequences depicted in SEQ ID NO: 1 (see FIG. 1) OR

SEQ ID NO: 1 GTGCGGCTCGGAAATTCACAWGGKCCGT-TYGTGCGGCTCGGAAAT

Since this consensus sequence shows the regions that are common to all 10 identified and validated inducible sequences it is reasonable to expect that further sequences that fall within the scope of the consensus are also formate inducible promoter sequences. Again, as described above, there may be portions of the consensus sequence that are not essential, and truncated versions of this sequence are also expected to function as a formate inducible promoter.

Methods of obtaining a consensus sequence are well-known to the skilled person.

A consensus sequence may be obtained by analysis of at least two sequences. A method of obtaining a consensus sequence may comprise the steps of aligning two or more sequences by multiple sequence alignment; analysing the frequency of each nucleotide, nucleobase or base or amino acid at each position of said alignment; and assembling a sequence wherein the nucleotide, nucleobase or base or amino acid at each given position is the most frequent nucleotide, nucleobase or base or amino acid at that position in said alignment of two or more sequences.

Accordingly, in one embodiment the isolated nucleic acid of the invention that is capable of acting as an inducible promoter in a non-methylotrophic yeast species comprises or consists of the consensus sequence defined in SEQ ID NO: 1.

The nucleic acid of the invention may be DNA, or may be RNA. Preferably the nucleic acid is DNA.

The skilled person will be familiar with the genetic code. Accordingly, at any given position within a nucleic acid sequence, ‘A’ encodes an adenine nucleotide, nucleobase or base; ‘C’ encodes a cytosine nucleotide, nucleobase or base; ‘G’ encodes a guanine nucleotide, nucleobase or base; ‘T’ encodes a Thymine nucleotide, nucleobase or base; and ‘U’ encodes a uracil nucleotide, nucleobase or base.

The skilled person will appreciate that consensus sequences such as SEQ ID NO: 1 may be degenerate sequences, comprising degenerate sites. A degenerate sequence may encode any of several different nucleotides at any given site. A degenerate site may encode any of several different nucleotides, nucleobases or bases. The skilled person will be familiar with the degenerate genetic code. Accordingly, at any given position within a nucleic acid sequence, for example within a degenerate nucleic acid sequence, ‘W’ encodes a Weak nucleotide, nucleobase or base, optionally selected from an adenine nucleotide, nucleobase or base and a thymine nucleotide, nucleobase or base; ‘K’ encodes a Keto nucleotide, nucleobase or base, optionally selected from a guanine nucleotide, nucleobase or base and a thymine nucleotide, nucleobase or base; ‘Y’ encodes a pyrimidine nucleotide, nucleobase or base, optionally selected from a cytosine nucleotide, nucleobase or base and a thymine nucleotide, nucleobase or base. In some embodiments then the isolated nucleic acid capable of acting as an inducible promoter in a non-methylotrophic yeast species comprises a sequence that comprises or consists of the consensus sequence set out in:

SEQ ID NO: 1 GTGCGGCTCGGAAATTCACAWGGKCCGT-TYGTGCGGCTCGGAAAT, where:
Y is a pyrimidine nucleotide, nucleobase or base;
W is a Weak nucleotide, nucleobase or base, optionally an A nucleotide, nucleobase or base or a T nucleotide, nucleobase or base;
K is a Keto nucleotide, nucleobase or base, optionally a G nucleotide, nucleobase or base or a T nucleotide, nucleobase or base;
   or any synthetic analogue or chemically modified nucleotide, nucleobase or base thereof.

The inventors of the present invention have identified 16 putative FDH genes in Yarrowia lipolytica, and have identified the corresponding upstream 1 kb and 1.5Kb sequence which is expected to comprise the sequences necessary for the promoters to act as formate inducible promoters. The sequences of these 16 1.5Kb regions are shown in SEQ ID Nos: 18-33. It is expected that the necessary sequences required for inducible promoter fragment will be located within a region of up to 1Kb immediately upstream of the translation start codon. The sequences of the 1Kb portion for each of the 16 Yarrowia lipopytica FDH genes are shown in SEQ ID NO: 2-17.

Accordingly, in some embodiments, the isolated nucleic acid of the invention that is capable of acting as an inducible promoter in a non-methylotrophic yeast species comprises a portion of a sequence selected from a group comprising or consisting of SEQ ID NO: 2-33; or is selected from a group comprising a sequence with at least 80%, 85%, 90%, 92%, 94%, 95%, 96%, 97%, 98% or 99% sequence identity to SEQ ID NO: 2-33.

In preferred embodiments, the invention provides an isolated nucleic acid that is capable of acting as an inducible promoter in a non-methylotrophic yeast species, wherein the sequence comprises or consists of a portion of a sequence selected from a group comprising or consisting of SEQ ID NO: 2-11 and 18-27; or is selected from a group comprising or consisting of a sequence with at least 80%, 85%, 90%, 92%, 94%, 95%, 96%, 97%, 98% or 99% sequence identity to SEQ ID NO: 2-11 and 18-27.

In one embodiment, the invention provides an isolated nucleic acid that is capable of acting as an inducible promoter in a non-methylotrophic yeast species, wherein the sequence comprises or consists of a portion of a sequence selected from a group comprising or consisting of SEQ ID NO: 18-27; or is selected from a group comprising or consisting of a sequence with at least 80%, 85%, 90%, 92%, 94%, 95%, 96%, 97%, 98% or 99% sequence identity to SEQ ID NO: 18-27.

Preferences for what is meant by a portion of a sequence, for example length and position of the portion within the recited sequences, are as described above.

The isolated nucleic acids of the invention are set out below.

1.5KB upstream fragments
>SEQ_ID_NO_18_1.5Kb promoter region YALI0A12353g
GTCGGATGCTTCTTCCACTGACGCCGAGCTGGACGACGTTCCCGATCAGGGTGCTCTGGGTGCCATTCACGAGTCCC
GGTCCGGCACTGGAAGTCCTCATCTTAACATGGAGGAGTTTCTGAAGCAGACATAGATACTAATTATTAATACATCGC
ATGATTGACCAACTGCAAGTACATACTGTATGTACTCACTTGTAGAGATTCTCGTTGCGACAAAATTGTGGACAAGACA
CGAAACGACCATGTCTGTAAAGTCACGTGACCAAAATCATCACAGCAGAAAGTCGAGTTCTTAGATATCTTCCACAAG
AGTTGCATGTTTTATATTGGACAGAGAAAAAGGGATGTGTATATGAAAAACTTTAATTTCGGGCAGATTCCAAAAAATA
ATGACATCCGAGAGATTCGAACTCTCGCCTCCGGAGAGACCAGGATATTGATTCAGTCACCTGAACCTGGCGCCTTA
GACCACTCGGCCAGAATGCCAATTGTTGAAAATTATCTGAAATTTATCCTTGTCAAGGTTACAACTGCCTAATCATCGT
CCCCAGACTCCAAGTCCGATTTGCAGGAATGACATTGTTTATAGGTCTAAGTAACTGAAAATGTATTCCTCTAGAACGA
ATAGAATATCCCCCGATTAATCTTGGATTTTTTAGACGTTTTTAACATACATACATTCATCAACATTATTCAGTAAATATT
CAGTATTTTTAGCCTAAGACGATTACAACCGTCTCACCAAATGTGTGATACATACGTCTATCAAATGTGTATGATTTTAC
TGGAAACGTCGCAAAACTGTCGTGTGGTTGTCCTTGACCGATTCAACTAATAGTGCTCTACATAGACCAAATTTTTGAG
AGGAAGACCAAGGAGGTTTCGTATACTAAGGAAAACCAAGGTGGTTTCGTATACTAAGGAAAACCAAGGTGGTTTCG
TATACTAAAAAATCAAGGCGGTTTCGTATACAAAAGAAGACCAAGAAGTCTTCGTATCAGTAAAAAAAACCAACCAGA
GTTTTAGTATACTTGCGGCCTACGATTGATATAATCGGACTAGTCTTCCACCCCGGTCATAACTGTGCGGCTCGAAAA
TGCAATATAGCCGAGAGTGCGGCTCGGAAATGTCGATCCAGGTGTTCGGAAGGTGTTCATATCTGCGAATATTGCTA
GCGAATGTGGAATAAAAACGACAGAATCAGTTAGCAAATGATTAATATATCGATTTATTGTTTTATCTGCCCAGGTGGC
AGTACAATATCTTCCACTAATGGTACATGTTCTGATCCCGGGCTGTTAAACGGGTTCCAAATTATAATTAAGCGAGCCG
CACTTGACATCCGTCTAATCACCGTGTCTGGACATGATTCGTGCAAGTCGCACAGATCGTCAGTCTCAAGGCACCACG
ATGTTTGGGGTATAAAAGGAGGCCGGGGGACGTCTCAATTCCCCATCCACATACCACCTCCACCACTACCACCACCAC
TACAACCAAT
>SEQ_ID_NO_19_1.5Kb promoter region YALI0F15983g
GTACCGTCGTGAGATACTGCTATTGTTAGCTACTCTTCTTTCTCTATATAATGCGTTGTGAGATCGGAGTGTCCCTGCC
ACGACTCAACCATGATCGACAAACTATTAGACCAGTACATTGGAGATGTGAGTTTTTCGAACGTTTATTCCGCCGAGG
AGCCGTGGAGTATCTACGACGAGTATCCTGAGATTCCGCCGCGTGTTCAACCAGTGGAGAATTCTCTGATTGGAGAC
TTGGACAATTTCAGTCTCAGTGTACCTTCTGAGATTTCTTTGGACGCGGGCGGTTTGGAAATCACGTCATCGGTCACC
GTTGTCACCAAGAGAGCGGACAGAGTTGCAGGCGGTCAGACTCTCTATGGAGACATTCTGGGGAGTTTGAGTGGTTT
GGATGGGTATGGGCAGGCTGGTTCAGAGAGAACTCATCAAGTGACACTGGAGAAGTCGACTGAAGCAGATGGGCTG
GGTAAGAGCGTGTTGGAGGAGCTGTCTGAGGAAGAGAAGTTTGAGGCCACGAAGGTCAAGGGCGAGGAAGAGTTA
GTACAGGACACGGACAAGGTGTTTGGAGCTCCCGATCTCGCCATTTACAAGAAGCGGTTTGCCTACGGCGAGAAACT
TGTGCTGGCCGAACTCAGAGCTAGGAAGAAGAGGTTGAAGCAACTTATTGAGCGCATCTCGGCAACCAAATCCAAGC
GGGAGAGAGAAAGAACCAATATCCAGCTTTCCCTGTCGCCTTTTTATGCCTGGGCCGACGAAGATGAGTTCGATGAG
GAGCTTGCCCGCTGGGATGGAAGTTATGATGATATTAACGCTAGTAGTGGTATAAGAAGAGGTATTTAATATTGCAAT
TGTTGTTATTCCACTGATTAAAAAAATAGCTTGCTACGAGTACAGTACCCGTACTTTCACTTGCACTCCTACTGTATATA
CCGCATTTGCTTGTAGAGTTAGACTCCTACGGACAGTCCAACTCTCTCAAGTCACCAAGTACAAGTACTTGTAAATGG
GAGCCATATACTGCTACGGAACTCGTCTCTAAAGTCTCGGCTGTCTTCACATGTGGCCTGCGGGCCGTTGTGCTGCG
CTCGTGCTTCCGTTGTGCGGCGTTGTAAGAATGTGGAGACGATGCCGTGGTCGAGAGAAGGCAGCGTTCTTCATGAG
CAGACACGAAATGATACTCGTCGTCAGTGATGCACAAACACATTGAACGAGTTGTTTACATTGATACCAAGGCATTGA
TACCCCCTACTGCCAAACTCGACATCCCTTACTGAAACCCGTCCGTCGAGCACAACATCACGCACATCATGCACATTAT
GCGGAGAGCCGCTCAGATCGGAGTATACTCGATCCTATCCACGGATAGTGCTGGTGTTCTTGTGCGGCGGGAGCTTC
TGGATTATGTAAGACCATTGGACTTTGGTACTTTCGTACCATATATAAGACGATCAATCACCCTGTTCAGTCTCCATCA
AACAACACCCATTTCAACATTACATC
>SEQ_ID_NO_20_1.5Kb promoter region YALI0B22506g
TCCTGAATCAAGAGCAACCCGGTCTTGAATCATCTTTTCACACCTTCAAACACCAACAGATGTAACAGTATCCCCTCTT
CGATAAAGTTGGTGTGGGGGTCAAGTGTCTCTGAACCTGTATGAGGATGTATCACATTGTATCGTACGCACGTACCG
GTGGCTATACGGGTTCGCTCATTATTGTGTATTATTAGACATGGGAAAATGAACCGGCTTGATGAACAGGAAATTAAC
ACGTCATTTGCGTCAGCCATGAACTTCCACTTCGCCGGTGATAAACTAACACACCAGTATGTAGTTGCCGTAATCTGA
AAGCCCACGTGACGCATCTCTTTTTAGCAGCAAAGTGGTCACCAAGACTACTTATGATGCAGAAGTATCCAACCATGC
ACGTACTGTACGCACATGAATTGCCGAGCAGTTCCTGGAGCTATTGTATTTGTAGTCGTACTATATTCGGAGGAGGGG
TTCCCGGCTATTGTCTTTCCATAGCTATGATATGTGTAAGAATGCATCGTTTTATTCACCACGCGTTTAATTTCACAGTT
AGGTTCCGGGGTACAGCATTTGAAAGAATCTTGATCACGTTATCATTATCATCACATACGTTCGTCCTCCGTTAATACT
TCATACAATCGTGCCAACTGGCGGTACGGAGCATGTCGGTGTTCGGTCTCATAAGCTCCTAAGCCCAGCCGGAACCC
GTCTGCCCGAGAGCCATTATCTGCAGCTCTTAACCCCTCAAAGTCCACACCTACGACTGTACACACCAAGGATGCATG
ATCTCTTTACACACTGCGGCCAAAATATTTGAGACGACGTTCTACACGAGACCAAGAAGGTTAAAAGTTGCGATTTGG
GATCCTCGAAGTCGCAGCAGAATGAGGTCTTGTACAAGGTCACCAGGGTATCACTGTCTGTATACGAGTAAAGGAGG
ACCTGCTTGTAGCAGACAATGATGGGTATCGTGTGTGATAGATGTCCAGACATAGCTGATTGCTCTGCATTAGTCTCC
TCAGCTGCAGACTAGTGTAAGCGGGCCTGGATACAATTCCAAGCCGGTTCGTGCGGCTCGGAAATACGCAATAGTAG
TATGGTTTGTATGGGGAAACACTAGTGCGGGGAGGGACTGATCTCATTGCTTTCAGGAGAATGAGGTTGAAATCAGA
AGGTGAATATGGAACGATTCTCGGTCAAAAAACATCGAGTTAGCTATGTCTTTGCAATTGTCCTGTATCCACGCATCAC
GTGAGCCATTGCTCTGGGCTTGAGAGTGCTCGCTACTTGTCTCCAGCATCGCGATAATGTGCCATATGCATGCATTGA
TGCAAGAGAATGGCTAGTGGCCTCAACAAGCACTAAGGACTGGGGCTTGTGGGGGAGACTGACGGAATAGTCGAAA
CCCATGGTTATTTTAACAGCTCCGATGTCAATATAAATACCACTGGATACCCCCTCCAGTTACAATCATCACACACAAA
CACAAACACAACCACTACA
>SEQ_ID_NO_21_1.5Kb promoter region YALI0C08074g
GTGCCTCCCAAGGACTCTCCCTTTGACAGATCGACCTATTTCGACCGGTCCATCTCGCCCACTAAGAACAGAGCAGGC
TCCCCCTCTGGCCGCGTCAGCAGTCTGTCTGCTGTCGAGATTTCAAACAAACTGAAGAAGTTTGCCGGCAAGATTGTA
CCTTCCTCACCTCCCAAACGAGGTGCCGAGTTTGAAAGCCCGTCCAGAAGCATGTCACCAGTCAAGGACCGAGTGGC
GTCCTCACCTAACCCTGGACGGCCCATGTCGGCTCTATCTGGCCATTCGGACACCTCCCGATCGTCTTCTCCTGTCGA
TTCCATCATGTCACATTCTCGTTCGTCCACTATGATGACCTCTTCCTCGGGATCTTCAGGTAACAGTATGCTCTCGGGC
TTCATTCCCAACATGAGCATGCCCAGTATGCCAAACGTGAAACTGCCCAGGGCCAGCAGCCCCCTCAAAAACCCGCT
GTCGGCTAAAGATTTGCAACACACTCCGGTCGAGCCTGCTCCCCCTAGAATTCTGCATGGAACAGGCAAAGGACCAA
GGACGTTTGAAGAGATGGGCATTAATCCGGTGACCAAGGAGGCCGACGATTGTGTTATTATGTAATGTATGATATCAG
TATTTAATCATAGTGCGGCGAGTAGGAGCTACTTGCAGGTGCGTAGAGGGCTGTTTGTAGTCATCGCACTGAGCCAG
AAGATGAGATGAAGTGTCTGGACGAAGAGCCAGCTCCTACGAATCCAGATGTTCAGATGGACCCAGCTTCTTTTCTAC
TTACTTGCTGAACTCAGGTATCACTTCCGGCTGCTTTCATACGTGTTACGGTATTCACTGTTGACATCATTTGACGCAT
GTTACCATTACTTGAAGAGTACTTGAAGATAAGTCAGCTGGGGGTGACTTCGTCATTCCTGAACCATTAAAAAGGAGT
GGGGCAGACCAAGTGATGGAGAGATAGCTATTTATTAAAAATGAAGTAAAATCAGCAACACAGCATCTACAAATGAGC
CGGGTTAAGGGTTCTGTCGTTCCAATTGAGTTGCTGCATTAGTTAGGACTTGCGTTCCGAAGACAGTGCGGCTCGGA
AATTTCCAAGGACCGTCTGTGCGGCTCAGATATTGTTTGCCAGGCTTTCGGGAGTGTCAATAAATGGCATATTTGCTA
GAAAAAATGTCGACTTTCCAGGTTCCCAAGTTGATTGTAGGGGAGTTATTGTATCATGACCAGCTTTTTTAGTGCTATT
TTCTTCTTTGAGATGCTCTTTTTAGCATCCCGAATTTCCACACTAACCGATGTTCGGTACCTTAGCGAGCACGAATAAA
TAAGCCGCACAGTAGATCCGACTAGACACCGCGACTTGACATGATTGACGCAAACCATGTGGAGACTCAACCTCAAG
GCATCATGGTGTTTGGGGTATAAAAGGGGGCGCGAAGACCTCTCTCAAGTCCCTATCAACACTCAACTCAACCCACTA
CAACCACAACTACTACAACT
>SEQ_ID_NO_22_1.5Kb promoter region YALI0F13937g
ATACAACCCATATTTATCTGTGGTATACAGTTATACAGTGGGGTCTATCCCTTATCGCTGTTCCAGTGCAAGGTAGCTT
CATTGTAATCTTTGAAGCGCTCGTATTATTCCAATGTCGAGTCGCGTTTTCTACGATTGCGGGAGGTCGTGGGGGTCA
ACTACGCCACGGTTCAGGCGGAACACATTTCTCCGCAGTTATTTTGACAGAATGAGGTAACGATATCAATCCAAATAC
ATTTTCCGCGTGGATATTCATTGATATCTCCGTTTATTTTACCTAAAGCCCTGATTTCTAGAGATAAGCGAAAGTGCCA
GCCCTGTAATTTTGGATATATAATTAGTAGCTTTTTAAACAACGAGCTTTACAAAGCTTACCACACAAGTATGAGCCAG
GGACTCGCCCAGGCTGCTTGTCTTGTTAGCCCGAACTGTCTTTACCTGGACTACTGACGTCTCCATACTATCGACCTT
ACGACTGTGTATCCCGCGTAATAGGTCAGGTGCAGTAAGATAAGTCTAGGGTGTTTTCTGTTGATATGGAAACAGGGA
ACCATGAGTTAGATAACCGACTCCGCGAAATCTCTCCGAACTCACCAATTAGAGCCAGTTCCGTGCTATTGGTATATCT
GGGCTGAGAGGTGCGCTACCCCTCCCCGTGTATGGTGGTAATACGGGAGAGAAAAGTGCAAGTACAGGAAGATACA
GAGAGCGTAAATCTTAATCTATTTTTGAGAGACAGGATATGAATAAATTGTACTTTAGAGGAGTTTTGTGGTGACTTCA
GCTTGGCTGAGGAAGGATTGTATACGATGTACTATGATTATCGAGAAAAGCAATGGTTTTCTGATTCATTGTTTTATGT
TTCCATCATACCGATTCCGCAATATAATTGTAATTGCACAAATACTAACCATTTACTTTTGCGGCCATTTTCTGGAGGTT
TCGTGTCTATGTACATCATTAACAGAGACGGTACTGTGGCGGATGAATCATGTGCGGCTCGAAAATTCAGTCGGTGC
GGCTCGAAAATTCAGGCGGTCCGTCTGTGCGGCTCAGAAATTGTCAGACGGGATGCTTGGAATAATGGCGGGATCCG
TTACCAAATTAAAATGTGTGATTAATGTTACATTAGATTGTAATTGTTGCAATCTATCGGAATCACCTGTTTGAAGTCAT
ATTTTTTCAGCAAAAATGGCAATTTTTCAGACGTGTTTAGTTAAATACAAAATTGCTTCAAGCGGCGACAAGGAATTAA
TGAGCCGCACGCTTACCCGTTGAAACACCGCGTCTCGACATGATACATGCAAGTTGGTCAGATCAAGGCGGGGGCAA
GATGGCGGTATTTGGAATATAAAAGGGCTCAAAACTCCAGTCACTTCATCATCAACACCCACACAATCCCCCACAACA
ACTACTACAGAT
>SEQ_ID_NO_23 1.5Kb promoter region_YALI0C14344g
GTCCAAACATGGTCTGTCCAGAAGGGAGACAATCAAACGAGGTCGGGGGATGTGAACGGCTCCCCGAAAGATTTCCA
TCAGTAGAAACTGGTTTCTTGCCTTTGAACTGTTTTGCTACAAGTTTCGCGATGAAGGGTCCAGTTGCACCAGTCTGA
AAAAAGTGTTCCGATTAATTTTGTATCGCAGGAATGTTGAACTTGGGAAATACAAACTGAATCAGACAGCTTGAGATT
GCTTTGATTGCAGTTGAAAACCCATCTCATTACCTCGCATCATGTCCACAATCGGAAATCTGTTCGTCCCGCAGGTCTC
ACTAGCCTCCATCCGTAGACTTCTCACCCTAGTCAGTGATGCAAGAAAACAAGTACAGTAGTGAATAGATAGAGAAAT
TGAATGAATTGTAACTAGGAAATCAACTCCACCAGCTTCTCTTCGAGTCTCAGTTTCTGTTACGCCATGTTTTGGATCG
TGCCACATCCAGGGTCGATGTTAATCCTGTATAAAACGCGAGATATACAGGAAAGGCACACACTAAAGGCAAGAATAA
CAATAATAAGTGTAACAAGAGTTATCGTGTTGCTCTGGAATTCAACCAAACCCCAAGCCACCATGTGTCAATGGTTTCA
AAAACAAGAGCTCCAAATTTGAAGTTTTCAATAACGTAGAAATCATTCAAAATAACTTGAAACTCTACAAAAAAGGAGC
AGAGGAAATTAATGACCCATAGAAGACTTTATCTTTTATTGAAATTATCACATCTAGTAAAACGGCCGATCTGGCTCAT
TATTGGATAGTTATAGAAACTTCAATTTTTGACATCAAGAAAAAAAGCGCCGATGGTTTAGTGGTAAAATCCATCGTTG
CCATCGATGGGCCCCCGGTTCGATTCCGGGTCGGCGCAATCCGTTCAGAGATCTAACTAGTAAATACTTTTTGTAGTG
CGAGATTCGTGGTTGGTGTAATCTTTTTGATATTGAATTATTGTACTTTTTATTCCTTCCCCCTAGGCTTATACACCTCT
GCGAGCTGGTGCTTTTCAGATGTGTGCATTGTTTGTTTGCATAATCCGGCCCAAGTTGGTGCGGCTCGGAAATTCACA
TGGACCGTTGATGCGGCTCGGAAATAGTTTAGCGGATGTTTCGAGACGGTCAAAAAAGCGGTATATTTGTAGAAAAA
CATGGCTTCAATAGTACCGGGTGAATTAAGATGACCTAATGATTTGATTTCCTTACTTTCACTACCAGGTAACAATCTA
ATTCGTCGAAAATGGAGCTGCAAAGTTCTTCGCTTAATTAGCAAGATATTGAAATAAATGAGCCGCACAGTCCATCCG
TCTAAACGCCGCGATTCAGCAGGATTAATGCAACCTATACAAACCAGGCGGGGACGAAATGGCGAAAAAACGATATA
AAAGGGGCTCCAAGTCCTCTCTAATCACCACCAGCACAAAAACGCACACCACCACCACCACAGCACCACCATACCACT
ACAACCACCAAG
>SEQ_ID_NO_24_1.5Kb promoter region YALI0E14256g
TTCCAGCAGAGGCAACGCCGTTGATCAAACTCTGCAGGTTGTGGGAATCCAATGAGACATCCATGGCCTTCCATAACC
AGGCTTTGGAGTCAAGGCACCACCTATGAGTTGGCCGAGACGATAACCTTCTAACTTCCTCACAGAAGATGGAACTTT
GGGGGGGATAGAGTAGCTGCCTTGGAGGTTGGTGATTGTTGGTAACACAGATGAAATAGCTATAAGCGCCTGATTAG
GGCGAGGAAAGTTGCAGGAGCCGAATCCGAAGGGTCAATTGTACATCCGAAGAAGTCGATATTCGCTCAAACTTCCG
GCTTGTGCTCTTTTGCGTCCCCGAGGATGTGTGTGCAAGGTTTGCCAAGTGCAGCAGCGGTTACATTGCTGGAAGAT
GTACACTATGGGTGTTGATTGGGGGGTGCTATGGAAGGGTGTATGGAGCAGGGGTGCTAGGTGGTCCACGGACAAA
TCATAAATAACACCTATTGCTGGTAGTTGGATACGAGACTTGTTTGACAAGAGCCAAACGATACACCAATGTGTATGA
AACCTACGCACTCTTGGCATATCTACTGGTACTGTAGCGACAGATTCACTTGTTGAGAGCTGTGCTTCCGAGCATCGG
ATGTACCTCTTTCTCATATAATTATCGTCAATAATACCGCGCATAACCCAGGCACTCATCAGGGCTGTACACCCTCCTC
TCCAATGGCAGGCGCTCGTAGCAGCAACTAAACCTTGGGGAGGGGGCGTGATCGAGGAAAGGGCTTCCAGTGCGTA
CCACACACGTATATCGACGTAATCGTGCCATGCAGACGGCGTGAGATAGTGTAGTTTGAGCTGTATTCTGAAGCCGG
TCTGCCACCGTATGTATAGGATCCACGTCCAAGAAGCCGCCTCGCTGGAGCCACCGGATCATACCCCATGTTCCAATA
CCCCGCTGAAAGGACAAACAGAAGCCGGACCGTGCGGTGCGGCGAGATATTCGGATTTGGCTCCATTATCTTTGTGT
ATCCGGTGCAAGTCGGCTTTTGCGGCTCGGAAATGGCTACTTGTAGCTCTGGGTTTGTGTTTGAGGGGAGAGTTGGA
TATGGAAAAACGTGGATGGTGAAGCCTTCGGGGAATTGGTGTGGTTCCCAATCAACTACTAGGTCAATTGATGCCGT
CTTTTTGGAGATTTCTGGACGCCATTGAATTGCTGTCCATGAGACACCCCATATTCGCTTAAGCAGCTTCCTTACCTTA
GCGAGGCACAGAACATTCCGCCTGTCAGCCCCAACCCAATCTCTGAGGGCCACAACTCTCCCCCAATAGCCAGCTGC
CCCAGTTGCTCGATCAGCCACCGAAGCTTCAGACAAGGCAGTTACACACTGAGCCTCAAGGTTGTGCGGGCGGATGG
GGTATAAGGGTTGAGGTGGTAACCGTGTGAGCTCAGAAGATATATAAAGGGGTGGCCATGTCCCCCTATCGCTCCTT
ACCAAACAACAAACAACAAACAACTACAAT
>SEQ_ID_NO_25_1.5Kb promoter region YALI0B19976g
TCTCAAGCTCAAATAGATGGATCGTGAGGAGGATGTGAAGGATTTTGTTTATTATTATTAATTGATTGTATCTTATTATG
TTATTGAGGTTGTACAGTTTGAACTAAGTACTGTAACTGGATATATACGATACTGTAAACTGTGCTGTAGTTGTGTTTG
TTACTGTAGTCAGATTCGTACGAGAGGGTCATGACGTCTTCTAGGAAAATCACGTGACCAATATGTGGTGTTTCTTTAA
TTCAATGGGTTACTAAGCACACAATACCTTATGAGTCAATTTGGGATCATACATTATAGTTGCGGCCATATCCTGGTGA
AAATACGGCTTCCCGTCCGATCAGCCATAGTCAAGCACCAGAGAGCCTAGTTAGTATTGTAGTGGGAGACCATACGA
GAATCCTAGGTGCTGCAATTCTTTTGCCACAGCGGCGGGCTTTTCTTTTTTTCTCTGTTAAGAGGCGACCTCATATCTG
TGAGAGTTGCTGATGTCTTACCACTTGAGAGAGCCAGAATGGAGTATCTACTGTATGGGATTATACTGTAGACTTTTTA
AGAGAGAACATCTCATCATTGAAAGAATTTATGTAATAACACCATCAACGTATTTCATGTAAGCTTCACCTGTTGGTGA
GCCACAATACATTGCTATTCGACTGAAGTATACAAGTAACTCTTGCCAATGAAAATCGAGAATATTATGGACGTTTGGG
ATGAATGGTGAACCAGTCAACCAATTTAGTTGAAAAGTCATCCAGCTTTACCGAAGCAACGCCGTTTAAATATACTGG
GGATCCACAGTTTTAGTACTTTCTGAGTGCCACATCTGTACACCACTACAGATTATAGGGTACCAAAAGACAAAAAAAT
GTCGGTGAAGATGATGAGATGGATTTATGAGACTCAGATGGTGACTCCAAGTGACCCAGGAAGTACATAGACAGGGG
AGGTAGCGGCGCAAATAATTCTGTTGAGTTGGATGGCGTCACGATATTTTTCTATTCGCTTAAATCTCCACATTGAGGC
GTCTTTTCAAGGCATCATCATTATTGACAGCCTTTGATCCGTCATGTCATGCGGCTTGGAAATGAAATTCGGCTCCGTC
GTGCGGCTCGAAAATGATTGTCTCGGATAAAGCGGGGGAGTAAAGAGGTGTTTGAAGTTGTGGTAGTTTGGATGGG
GAGGAATGAAGTAAAATATAAACATTTTCGTGCGGAAAAATGAAGGGTTTTTATATAAAAACCCTACTTATCTTGTCAT
TTAAAGCCGCTAAATGCGCTGTTACTGTAGCATACAGATAGTTAGTGAGAACATGTTGACAAACTCAATCGGATCCTC
CAACCTTGTATTTATGCGCCGCACAATGAGCAAACCGCGGATCAATGCCCAGGACATGGTGTGGAGACTGTGAAAAG
ATATAAAAAGAGGTCCTCACCATCCATTATTTCTTCATCGACTCACAGTCTTTAGCAACCACTACATCTAACTACAACAA
CAACTAAA
>SEQ_ID_NO_26_1.5Kb promoter region YALI0E15840g
CATTTGTTGGTGGCAATGTGAACTGGATGATTTCTGGGTCCAACAGTGCAATTATCATTACAGCAGTAGATAGTCCGG
TACCACCCATCACGAGGTAAACGCCGACGCGGGGGTGAGTGAGCTCTTTTTGACTTTTGTTAACTCCGACGAAATTTG
GGGGGGGGAGAGGGATTTCGTGCGTCCTTGCATGGTCTAGTCCGAAACTAACAACAGCTCAACTGGCGGATCAGGA
CACTTTCAGACAAGTTTCAAACAGCAAATTCGGAGTGAAGAACCCTCTTGTTGCTCTGCTTCTCTTCTGTGGGTCGCTT
CTTTGTCTTCTCCGTGTTTTTGCCGAACTGTACATCAGCCCACATTACAAAACTGCACGGTGCAGGACACGTTTAGCC
GCCCAACTATGGCGAGGTGGGGGTCGTCGGAACGAGGTTCAGAAAAGAAAAAGACCGGCTCGAGGGCTTCCGGAG
GATAAAAGGACCGTTCAAATGAGTATCTCCACCCGCCTTTGCTCCAATTGTTGTTTTTGGAACGTGTGTTGCTGGAGG
TGTGGATCTTTTGGCAGATACAGGTATGGACGTTCAGTTGATAGACACCAAGTAAACTACTTGTAGTTGGTCATATGC
ATCGTCCATCACTCGTGTTAATTTCGAAGGAAGCTGTTTATTTGATGAGTAGCTCATTTTCCAACTTACTCGGTGAACG
TTGCCAGCGGCTGGCCGGTATTGGACAAGATCTTACTGTTGGACAAGCCATTGTGAGTCCTCTTGTGGGTGTGTTTG
GATGAAAAACAGAACCAATCGCGAGTCTCGGCCCGACTCCACTCAGACCTTTTGTATCTGGATAAATGGAGTGTCGCT
TGGCTCAGGGGTACGAGACTTCGCCCAAGTTGGCTCAATTGACTAAAAGTCCATTTTCAACCCGTGTCATGGTCGTTA
AACACACCAATTTCACTCTGATACACCAATGAGTCTCATTGGACCTTCCAAAGCCCCCATTTAGTACTTTTTTAGCCCTC
TCGGGACACTGGCTACTAACATTGAACACCGGCCCCGCATCGTGCGGCTTGAAAATTCAGGTGGTCCGTTCGTGCGG
CTCGAAAATTGTAGAGCGCGGGGGCAGTTCAGGGGAGGAGCCCGAATAACTCATATTTTGGTAGCCACACCGACCTC
AAAGTAATTTCAGAGGGCCATTAATTGATTCTTAATTGATTATTAATTGATCTTTGTTGTTTCAACGTGCCGGAATGGTC
TTTTTATAGGTCCATTGTCTAGTTCACTCAGACCACATCAACAAGAGTTCTATGCAGCACGAGTGCTTTAGCGACATGC
AGGAAATAATAATGAGCCGCATAGCTCTCCGGTCTGAAAACCGTCGAACAACACCATTCCTGCAAGTCCCACAGATCG
AGGCGGACATGATATGGTGTAGGAAACGATATATAAGGGACCCAAAAGTCCTGAATTTGGTCTTCATCAACACACCG
GTCTACCACCCACTACTACC
>SEQ_ID_NO_27_1.5Kb promoter region YALI0F28765g
TAACCGTGTTAAATCCACATATCTGACCCGGATTCGGGGGTTCAGAAGAGCTTCTGATAATTGTGTTTGTTGATTCCCT
TCACTTCCTCCTCTGGACCAGATAAATACAAAATATCAACCGTCAAACATTGCAAATTGTAGGGAATTATAAAAAAAAT
TATAAAATATTTTTTTTATTCTAATGATTCATTACAGCACAGCAAAAAAAATTGGGAAAAATTGTTTTAAGCTGCTGTAG
ACAAAATCATTGTAAATCTGCATCTCTAGGCAACCTGGCCGAGCGGTCTAAGGCGCCAGGTTAAGGCTAACACCTTAA
CGAATATCCTGGTCTCTTCGGAGGCGAGAGTTCGAATCTCTCGGTTGTCAACTTTTTTGGCTGACTCTTCTATTGACGT
CGGCTTTTATCATGCATTTGTCTTAAGTACTTCTTTTTCATTGATTTGAGGTGTGTTCTGTCGATGTCATCATATAGTAC
AAGTAGTGTCTCACTACCGAGCTTCAAATGCTTGGAAGTTACGCTACTGTATATCTGGCCAACCATCGATTTACTTGTA
TAGTGTTATGTTGAAGAACAAGACGCTATAGACACACAGTATATCTACAGGCACTTGTACGCAATGAATGGACGCCGT
ATACTGTTCAAATGTTTGATATTCAGTATCACCAGCTCACATCTACAAATACTCCACTCCCACATGCCGGAGCCGCACG
AACTTATGAGATCAACTTGTGCTCCTTGTACAAGTACCGGTACAAGTACCTGTTCCTATAGATCTGCGTGATCAAATGC
AGGTTGCACACAGAACAACAACAAGAGAGTACAACACCATCAAGTGAAATCTAATATGCTGCCACACACTGATGAGAG
ACACCGCTTCTATGACATGATTGAAATGAGTTACAGTAGGATGGAAAGACTGGTCGTAGCTAGGTACAAGGATGTGG
GGAGGTATGCGTTTATTAGTTCATAGACCACATGGTCTTTCCATGGGTTAATATCTCGTATAAATGTCCAATACCAGCC
ACTCTCATTGTATACCACTGCTGCCTGCCTCACGTCCCGTTTCACGGGTCATACGTGCGGCTGAACTCTACAACTTCC
GTGCTGTGCGGCTCATCATCAAGTAGCCTTTCATTCTGTTGAGCATCCCGTTGTGATACAGAATATGATCCACATGTCC
ATCTCTTCACCACGACATCAAGTGACACGAAAACGACACATAGCAGCGCAAGATACGACGAAAAATACCCAGTGATTT
CAGCAAGAAATGTACACTCAATCATGATACCATAACGAGGTCACTAGCCTTTCAAACCACCACCTCTAGATGACATCAA
GAACGCATCACAGTGCATGTCATGCACCTCTCGTCACCCACAACCGGGTCAAGGACTACGGAAATCCACTTGAACGAT
ACCCGAAGCGTCTCGGGGTATCTATATAAGGCACCCATGGAACCCTGTTGATTCCTCATTCAAACACGCACACGAACA
AACACT
>SEQ_ID_NO_28_1.5Kb promoter region YALI0E19657g
GGAGGGAAGAGGGGTATTTAAGGCTGCGGGAGTGGTTTTGTGTCGTTTTGCGGTGGAACGATGTTTCTGTGTCACTC
AGCAATGTCTGGTGTCATATGGCTCGGAGTCCGCATGTAGTGTATCCTCTTCAATATGATGGTGACATTAAAGATGTTT
AGAGGCGGATTGAGACGGTTGTGGAGAATGCTGTGGTTTGTTATGGGCTGTATTTATATCTTTGGGGACCTTGTGTAG
TTTTTTTTTCAGGATATTCGTCGTTTGAAGTGACTTTTTTTTTCTGTATTATTCGACTACTGTACTTGATCCAAACGTTTT
TACTTGGATTTTGTTAACCATGCATTTGATAAAATAAAAAAATTAACCCAGTTTTATTATAAAAAAATAACTTCAATAATT
ACTTGGGATATATTTTTATTACTTGGGGAAGTAGTTGGGAGGAAGAGGAGCAAAAATAAATAAAAGGAACATGTGGG
AAAATGGAAGTTACAACCAAGTATGTACTGTGCATGGGAAAAATAGAAAAATGAAGAAGAAAAAAATGAAGAAAAAAA
ACATACCAACAATCAACTCCTCCTTCTTTCGTCGACTCTTACAAATCATCACGTGACCACACTTCTCCACAACATACTCC
CCCAAAATAGAATGCACTATACTGTAGTATATCTTATACCATACCACAACCGAACACACCGACTAAGAGAAAAAATCGG
GCATTTCCACACCTGGAGACACAAAATCTCCTCCCTCCAAAACAAGACATATATAAACACTCAAAAATCGCCCTTATCA
TATCCAAAAAGTCGAAAAAAATGGCACTTTTGCCAACCCTCATTTTCTTCAACCCCGGGTCATTAAACTTCCGTCAGAC
GCACGTCGCATCAACAATAGTCTTTAATATAATTACTGGAGAAGCGGAGATCACGGGGTTTGGAGGAGAGACAGGAC
ACGGCGAGGACGGTGGTCACGTGACCAGAACAAACCCCACATGACCGGGGCCAGTGTATTCACGTGACCCACCCTCT
CTCAACCGAAATGGCGACTGTTGCTGCTGTTGTATTTTGGTCTGTTATCCCAGTCTAACTTCAATGTTGGAGTTGTCGG
AAAAAAAATGTAAAAATGGATGATAAAATGTAAAAATGGAATTAAAAAAAATGGGTTAAATGTTAAAATGGATTAAATG
TAAAAAAGGAATTAATGTATAAAGTGAAAATAAGTCGGGTTTATCCGGGGTCCACCAGGGAGCTGAAAAGTTGTCAGA
TTTGAAAAAGCAGAGAACCAATGGATTCTCCGAGTTCCTGCGATTCCAGTTCTCTTTTCTCCACCCAGATCAGACCTCC
GCAACGTCCAATATTTGCATTCCACCCCGGATCCACACAAGGTTATATTCTGCAGAAATAGTACAACCAAGGGTGTCC
ACCTCAACACATTTATCCAACCACCCACCCACTCCTACTGTTACGTGTACCGACACAGAACGTCACTCTTTTAACACCC
TCATTG
>SEQ_ID_NO_29_1.5Kb promoter region YALI0B21670g
GGCCACGAGACCGGGGTGAGCGTGAATGAAGGACAGATCAGGATGCTCTCGAGCAAACTTCATGACTGCTGCAGAG
CTGAACTGCACAATATGACGGTTCTGGTTCATCATGGAGAACTTAGTTCGCAGCTGCATGTCGTCCTCCACGTAGGGA
CCCTCGTTTCCGGGTGCCAGCACAGTCATGACACGTGCATTGTCCTTGTCTCCTTCGTCGTAGGCCGTCTGAACCAGA
GGCAGCAGTTCCTTGACGGCGAGCCATCGACCGTAGTAGTTGAGCGACATTCGCAGCTCGATTCCCTCCGAGGTCTC
AGTTCTTCCAGCGGTGCTCAGAATGCCCTGGGACAGAAACAGCAGGTTGATCTTGGTCACGTCCCGCTTGACAATGT
CCGACACTCGCACCACCTCCTTCATCTGCGACAGATCGCTCTTAATGAAGGTTGCCTTTGCATTGAGCTCCTTGAGAT
CATTGACAATCTTGTTGCCTGCCTCTTCACTTCGTCCCACAATGATCACCGTGGGGCTATCGAACTTCTTAGCAAACAT
GTAGGCGGTCTCTTTTCCGATTCCGGACGTTCCGCCGAAGAAGACGGCAACGGCATCCTTAACGGTGGGCTTATAGG
CCGCATTGACCTGTTTGATATCGGCTAGAGAGGGCATGGTGTGTAGTTGCGAAAGGTGGCGAAATCCGGGGCTTAAG
TATTAGAGTCCTGCTGTGCAGGGCTGATGGCTTTAGGTATACAACGCTATTTGGACGCAAGGTACATTTGAGACAGG
GCAGCAGGAGGGAGGGGAGGAGCTAGAGGGACGGAGAATAGGCATTTCCAGCCGGAGTTTTCGGAAATCAATTGAA
TAAATAATTTTAAACAGATATATATCTTTAGATTTTTACAGTTCTCATGGGAGCTCTCCAGATAAGTCCGCCTTGGAACA
CATCTCATCTGCATGAATTACTAAGGTGGCGGAAAAAAAACAACGTCTGAGGCAAGTTCAAGCAGGCTGGCGGTGGG
ATCTAATTATTCAGCGCCAAACAGATATGACCCTTATATGATGAAGGGCTATTATAGCAGCTACAGCAGATGCAAAGG
AGGCTAGATGATAGCCTGGCATTGGTACAAGTAGCTCACCTCGTGGCGAACCTTTGCTCCCATACATGTCTCTTTCAG
ACTGCACTATTGCGCATTTATACGTGGTTATACGTCTTCTTTCTTCGAGTTACATGCCCTGTGTGTAACTGTAAGGAGT
GGTACCCCGTCTCGTACAAAACCTGCAACTGTCAATTGTAACCGCACAAGTGCGTGTGTGGGCGTGCTATTGAGTCA
GACTTTGACTCATGTGGGCCACCAATAAAAGCCTGAGGATAGAGCCCTCAAAAGTCCCAATGACCTTGTGGGGGTGC
TCCGCTTAGCGTGGGGGTCAACCTAAAGGAGGAAGTACCCAAGGAGCTAACCCCAGACTAATAGTACAGTACGCATC
CAACAACACACTCAATTGCAACACACA
>SEQ_ID_NO_30_1.5Kb promoter region YALI0F29315g
AAGGGTGTACTACAAATACGTAGCAACTGTGGGTGTGACTGACATGTGTATGTATGTATGTATAGTATGTACTTGTAC
GGGATGTACTGGTACGATACATACACGTGTTGCTACCAGTGCATGCCCATACCCTCGTTCCAGGTGACGTCACAGATT
GCTCATCACGGCTTAAACTGGTCAGGCGACATGGTTCAAGTTGTAACCATACACTCTGTGAGTCTTCAACTGCAATAG
GAGATGAGCTCGACGGAGCTGCCAGTCACGATAATTATTCAACAACCCACCATTGAACAGTACAAGTAATATTAATTT
AACCGAAGAAGTCTTGGCAACGTGCTGTGCACAGAGACTTACGTGGGGGGTCATGGGGGGGGGTGATAATGGGCCA
TAATGGCGGCGCTTGTACAATGTAGATTACTTGGTGGGATATAAACATGATGTACAAGAACAAGTATTTTTACTGAGCT
GCTGAATGGCTGTCACTCATTTGCGCTTAAAAATGTCCTCAGGTGCAGAATAACTGTCGCTATGGCTGTTAATTTGGG
GCCATATACAAGTAGCTCAAATCCACGCTCCACCGCTGCTGGGGTTGAGACAAAACCTGTGAGCCTCCACATTGCACA
AAAGAGGGAAATAATCGGCACCAATCGGTATCAATCAGAACCAACCGATTTATCTGTTTAACACCAAAACCCCTTTCTA
CAAGTAGCCACATCTAACCACTCACCAGGCCCATTACCCCGCCTGTTGATCAACGGCTATTTCTACCTCATCCATATTG
GGGGTGCATAACCCATCAGACTGGGCGTCTAATAGGAAGAGCAGGAGGAGGATATTAGACGACGTCCTCACCCAGC
TGGGTAAGAGCAGAGTGACAACCAGCTCTCTTTCCGAAAGCCGTATATACTCTCAAAACACTCCACCATCTACGATAA
GCATATCGGCGCGAGTCTTATTTCATATATCTGGTGTTTTACCGTAAGATCCACCTTCCATCCGAAGTATGAGGCTTCG
GGTTCAACCCCCGCCCTCATACACTACTTGTAGAAGGCGGAGGTGTCCACACACTTTTTTTTCTTTCTGCAAACAATAC
TCTCCGTTGCACTGCGGCTCAATAATGCTACATACGACTTATCCGGAATTAGGTTGGGACCGTTGAGTACGACCGCTG
ATCGACGACTCTGAAGATGGTGATATCGCTGAAAGAGATACCATTACACTACATTTGAAATACAGAACATTATTTCCAG
GAGTAATGTACCACTTGAAGTCTGTGATTTTAAATCTCTCATCAACTGTGTATACCTGGCTCTTGCAAACCTCCACATG
CATGCAGCACTACCTCCATGAACGGATCTCGGATCCCCTACTGCGGCGATTCAGTGGGTATATACCTCAATTGGACCT
ATAAACTAATTATTCTTCTCCTCGACTTTTCTATTTCATCACACACACTTACACGCACACGAACAAACACTACGATGGTC
CTTCTTATTCTCC
>SEQ_ID_NO_31_1.5Kb promoter region YALI0D25256g
AAACGAGTCTCAGAAGTAACTGAAACTAGACACAGTCGGAATCGGTCCACCTTTGTCATTCCCAACTGTACTGTACTT
GTAGATGAACACCTGTCTTCAGAGCTCCATGTACTATCCCCCAACCGAGCAATCTTGGTGTTTGGTACCCAACGCCTC
GCAAAAGCTCAAGAGGGGAATGGCCCGCGGCGTTGGTAAACCGACAAGACTGAGTCGATTGGTAGTTGCATGTTGG
GAATTTGGAGGGTCAATCACGAGAGATGGTACTGTTGCAGAAGTCATGGTGTATTCATAATTCAAGGTGATCAGCAG
GAACTGGAGTCCTTCAATCATCCGAAAGAATGATCATGGTATATGTGAGACTCAGTCGGTTCATTATTCCTGTTTGTCT
AATTCCCTTTCTCTACAATGATTCGATATCCGTCAATATTGGGCGAGTAAGAGAGGTTTGTAGTTCACTAGAAAGTTCC
CCAGAAAGTTTCCCAGAAAGTTCCCCAGCAAGTTCCCCAACTTCAACTAGTGCAAATCTCGGTAGTATTTTGTCATTGT
TTTATAAAAGAAAGCTCATCATTATTATGAAAGATTGCAGCATTTCACCGAGAGGTAGAGCGGCTTTTCAAAGAGTTCA
AATCGGCTCAAATCTGTTCAAATCAGCTCAATCTGTTCAAATCAGCTCAATCTGTTCAAATCAGCTCAAATCAACTCAAT
TCAGTTCAAATTCGACAAGACACTCTCATCGCTCATTCGATGAATCCCTTTTATAATCCAGCAGTATTCATATGCTCTCC
CGTCCACGTGGGCAGTTTCCTTCAGCTACAATACATCTGCAAAGACACTCTGTTCTATACCTCCCGGGTATCCGCTAT
GTTCAATTGCCTTGCTAAAACCTCACAAGCCTTGCTATGCAGTCCAGTCGAATTATCCGGGATTTGGTACGCTTCAACA
CGAAAGAGGATGTACAGTACAAGTAGACCGACAGACCTGCACCCCGCCTACTACAAGTAGCCGCTTTGACCCCCCAC
TAGACAACACACACACAGTGAGATGTAAACGGCCAGTTTCTAGCAGCCACGTACGTACTTGAGCAGTATAGAACCATG
CAACTGCATCTCTCCCTCTGTGACCCCCAATTGCAAACCGTGTTTGAAAATACCTTTGCCAATCAGCGCAGGTTGAAT
GTGGGTGAACAAGGTCAAAACGGGTGAACAAAGGTCAAAACGGGTGAACAAGGTCAAAACGGGTGAACAAATGTCA
AGCGCAAGACGGAGACAGAAGTTTCGGGGTCACGTGACTATCACGAGTTGCGATTCTACGACGCTCATCAGCGACGC
CCTTGGATTTGCTCCATTCTCGCTTAATTTCGCTTATCGCCAATTGCGTGGGGGGTGTGGGGGGTCCCAACTTTAAAA
CACTTACCCAACATGTCCCCCACGCTCTAGATATATACCACCTCGAAAATCCCACTCCCCCACACATCGCTCAATTCCT
CCACGCGTGCGCCAA
>SEQ_ID_NO_32_1.5Kb promoter region YALI0C11099g
GGTACCATCACTCAACTGGTTGCGGCACCTCTCGACATGTGTCAATGGGACACTTCCTGCTGTCCGCTTTGCGGTTGG
GGAGTTTTCGGTCAGTAAGGGACGAAGCGAAACGACCGAGGTTGGTTCGTACCCTGAACATGGTGGTATCGAAAGGT
AGCTCTGGAGTGAAGACCATGACTTCATTCTCATCTTCAATCACACCCTCCTCGTAGAGATCCTGGTTACCACCAGAAT
CAACATCTACAAAATAGTCTGACTCGTCTGAGTCGTAACAAGAGACATTATCCTCCTTATTCCTTTGCTTAGGTCCCAT
CGTCTGCTTTTCGCAGTGGCAGCAGTCCGTTGACGAGCTCTCCTCTTCTTCCACCACGCCAGGCTTTTGAGAGTCAAC
TTGCGCAGAACAGAAAACTCCAGTTCGTTGTTGAAGACGTCCTCGTGTTCTCTCGATCCAACAACCTGGGAGATCTAT
GCTAGGTGCCACCTCCTTGATAGATTCAGCAATCCTGGGCAAGGCGCTGGCTGAAAATTATCAGGCCACCTGTAGAG
CATTCTTTCCGTCCACGTGGGGACACCTTGTACTCGGTGTGGTTGACAAAAGCAGACCATCTCGGATCCCTTGAGCAT
CTTAGCAAGTGGCGTCACCTACTCGAGCTTGTCTCCCACCAGTGTCCTTGAGCTTCAAATCCAGAGACGGCCCCGATA
CATTCTCTTATGATTGTTGAATACCGTTTCCGCTCCTTGAAGAGCAGGGAATCTTGACGGACTGGTTGTGGTAGAAGA
GCCAGCCGGCAGAAACACGGCCACTGAATCAAGGAGAGATTGTAGTAGAGCGTCTTTTTGTCGCCCTTGCCTCCGAT
TCCATACTTTTTCGTCAGGGTGTGTTTGATATCCATCAGTTTGAGCTGCTTTACAGGGGCATGGGAGCCCCTTCTCCCT
CTTTACCCGTCAGACTCCATCCTTCTTCTTCGTTCTCTTCTCCGTCTTCTCTCATCACCATAGTGGTAGAGGTGTGCAG
ACCTTGATATCGTCGGAGCAGAAGTTGTCGTCGGAACTAGCCAGGCTCATTTTGTCAATGTATTGAGCTGCATTGCGT
ATCTCGCGTCTTATTCTAAGACACCGTCTCAGCACATTAGTGCGGCTTGAAATTTCATTCACGACTTGGAAATTATGCC
GCCCGTACCGTCGAAAGATGTTAGTAAGCAATAGCTGAAAATACCGGGAATCGATTGGTGCAGTCTCAATGTGTCTAT
TTAAACCAACTTGGTCTGTTTTTGCTATATTGGAGCCCCGCTAGAACTCACGATCGAGCCTCTGGTTCTTAGCGAGAA
AATAAGCGAGTCGCACCACAGCCGTCGAAACACCCGCTGGCTTGCAAGTTGAACACCTCAAGGAGGGCTCCAGATGG
TGATCATGACAATAAAAAGGACCCCAAATCCTCTCTATTCCTATCTACTCACAAACACCACCAATATGAAAGTCCTTGT
CCTCCCACGCCAGCTC
>SEQ_ID_NO_33_1.5Kb promoter region YALI0F09966g
TCGGACGCCTTTATCAATCCCCATGTCTTCTTCAGCATTTAGTAGCAGCGACAATGGTGCCTTGGAGCAATGTTTTGTT
GCGAGATTTGTGGCAGGGTCCAGAGGTAAATCACTTGGTCTCGACAAAGTGCGACGAGGACCCACTTGGGCCGCCTT
CCAAGAGGAGTGTGGAGACAAGACCAAGACCAGTGGATTCGGAGCCGTCTCGATGATTGATTCGCCGACTCTTCCCT
CCCGTAACCGTGTCATTCTCCTTCCACAGCCGGGTTCCGCCCCTTCTGGCGAAAGTTGCACTCTTTTTCAGACCCCGA
AAAAAAGACTTTGGCTTTAACTGTCGTACATGATGTCGATGTCGATAGTGGTTGAAGAGAGTTGCCAGAAAGGTCAGA
AGATCTTTCCATTGCCCGGACCTCCCTCCGGACCTCCCCTTCCGTACCTCTCTGCCCCTTCTGGACAGGTCAATGATA
GACTCAGAGCGACACACATGTCTGACGTACCATGTTAGACCTTGTATTGACCTGGACGAATGTGTGTGAGGAGTGAG
GCAGGCCAAGACGAACCACGGTCTTTATATATGCCCACGGAGTGACACGGTCTGTGTCGTCACCGCAGCTCCACTCA
CCACCCGCATCATGATCGTCCAACCAGAACCCACTCCCCAGTTTCGACCCAACACCATTCTCAACTGTAAGTATGAGT
ACCACAGTGATACTCGCCCAGTGCCGCACTCGTACTGTAGCCACTCCACTGCAACATCCGTATCGTATTGCACCGCCC
CGATTCACCTGCTTCCTTCAAGCCTTCAACCACGTACTGCTCCACCTCCTACCGTTGAGCCCACTCGGATCGGCCAGA
GTCATGTCTTAGGGTTTGGCTGCAGTTGTGGCGTAAACTATGGAGAAGGCGACGGAAACGAGAGCGCTACCGGTAG
CGACTTGGCGACACGTCTGGCTCGGGAAGGGGGCCGTTGCAGAGACCAAGACTTCCGTCACGTGACCGCTGTTTGG
TCAATTCTAACGCAGTTATTTTCCGTCTGATTCGCTGATACGAGTACTCGCTTGCTGTAGATGACTCAGACCAAGACAA
GAGAAGGGGAAATAAAAAAAACTTCCAAAAAAAACTTCCAAAAAAAAAAAATCAAAATTTGACAAACCTTTTCTGCCTG
GGACCAGGAACTTTGTGAGTCCATTGAGGGAGTTAGCCACCCATCAGCCACAGCCACAGTTTGGACAAGAAGTAAAA
GTGGATATATTTATGTTATGGAGACCATGTAGTGTTGTGGGAGGGAGGGGTTTTTTTGTTTGTTTTGGCTGAGTAATC
AACAGCAAGTGGCGTATATCGTATATCTATCGTGACTCAGACTATTCACCGCTTGTATGGTGCTATCTCGACTTGTGCT
TAGTCTCAGGTACACGTGATTGC
1Kb upstream fragments
>SEQ_ID_NO_2_1.0 Kb promoter region YALI0A12353g
TTATCTGAAATTTATCCTTGTCAAGGTTACAACTGCCTAATCATCGTCCCCAGACTCCAAGTCCGATTTGCAGGAATGA
CATTGTTTATAGGTCTAAGTAACTGAAAATGTATTCCTCTAGAACGAATAGAATATCCCCCGATTAATCTTGGATTTTTT
AGACGTTTTTAACATACATACATTCATCAACATTATTCAGTAAATATTCAGTATTTTTAGCCTAAGACGATTACAACCGT
CTCACCAAATGTGTGATACATACGTCTATCAAATGTGTATGATTTTACTGGAAACGTCGCAAAACTGTCGTGTGGTTGT
CCTTGACCGATTCAACTAATAGTGCTCTACATAGACCAAATTTTTGAGAGGAAGACCAAGGAGGTTTCGTATACTAAG
GAAAACCAAGGTGGTTTCGTATACTAAGGAAAACCAAGGTGGTTTCGTATACTAAAAAATCAAGGCGGTTTCGTATAC
AAAAGAAGACCAAGAAGTCTTCGTATCAGTAAAAAAAACCAACCAGAGTTTTAGTATACTTGCGGCCTACGATTGATAT
AATCGGACTAGTCTTCCACCCCGGTCATAACTGTGCGGCTCGAAAATGCAATATAGCCGAGAGTGCGGCTCGGAAAT
GTCGATCCAGGTGTTCGGAAGGTGTTCATATCTGCGAATATTGCTAGCGAATGTGGAATAAAAACGACAGAATCAGTT
AGCAAATGATTAATATATCGATTTATTGTTTTATCTGCCCAGGTGGCAGTACAATATCTTCCACTAATGGTACATGTTCT
GATCCCGGGCTGTTAAACGGGTTCCAAATTATAATTAAGCGAGCCGCACTTGACATCCGTCTAATCACCGTGTCTGGA
CATGATTCGTGCAAGTCGCACAGATCGTCAGTCTCAAGGCACCACGATGTTTGGGGTATAAAAGGAGGCCGGGGGAC
GTCTCAATTCCCCATCCACATACCACCTCCACCACTACCACCACCACTACAACCAAt
>SEQ_ID_NO_3_1.0 Kb promoter region YALI0F15983g
GAAGTTTGAGGCCACGAAGGTCAAGGGCGAGGAAGAGTTAGTACAGGACACGGACAAGGTGTTTGGAGCTCCCGAT
CTCGCCATTTACAAGAAGCGGTTTGCCTACGGCGAGAAACTTGTGCTGGCCGAACTCAGAGCTAGGAAGAAGAGGTT
GAAGCAACTTATTGAGCGCATCTCGGCAACCAAATCCAAGCGGGAGAGAGAAAGAACCAATATCCAGCTTTCCCTGT
CGCCTTTTTATGCCTGGGCCGACGAAGATGAGTTCGATGAGGAGCTTGCCCGCTGGGATGGAAGTTATGATGATATT
AACGCTAGTAGTGGTATAAGAAGAGGTATTTAATATTGCAATTGTTGTTATTCCACTGATTAAAAAAATAGCTTGCTAC
GAGTACAGTACCCGTACTTTCACTTGCACTCCTACTGTATATACCGCATTTGCTTGTAGAGTTAGACTCCTACGGACAG
TCCAACTCTCTCAAGTCACCAAGTACAAGTACTTGTAAATGGGAGCCATATACTGCTACGGAACTCGTCTCTAAAGTCT
CGGCTGTCTTCACATGTGGCCTGCGGGCCGTTGTGCTGCGCTCGTGCTTCCGTTGTGCGGCGTTGTAAGAATGTGGA
GACGATGCCGTGGTCGAGAGAAGGCAGCGTTCTTCATGAGCAGACACGAAATGATACTCGTCGTCAGTGATGCACAA
ACACATTGAACGAGTTGTTTACATTGATACCAAGGCATTGATACCCCCTACTGCCAAACTCGACATCCCTTACTGAAAC
CCGTCCGTCGAGCACAACATCACGCACATCATGCACATTATGCGGAGAGCCGCTCAGATCGGAGTATACTCGATCCT
ATCCACGGATAGTGCTGGTGTTCTTGTGCGGCGGGAGCTTCTGGATTATGTAAGACCATTGGACTTTGGTACTTTCGT
ACCATATATAAGACGATCAATCACCCTGTTCAGTCTCCATCAAACAACACCCATTTCAACATTACATC
>SEQ_ID_NO_4_1.0 Kb promoter region YALI0B22506g
TGTGTAAGAATGCATCGTTTTATTCACCACGCGTTTAATTTCACAGTTAGGTTCCGGGGTACAGCATTTGAAAGAATCT
TGATCACGTTATCATTATCATCACATACGTTCGTCCTCCGTTAATACTTCATACAATCGTGCCAACTGGCGGTACGGAG
CATGTCGGTGTTCGGTCTCATAAGCTCCTAAGCCCAGCCGGAACCCGTCTGCCCGAGAGCCATTATCTGCAGCTCTTA
ACCCCTCAAAGTCCACACCTACGACTGTACACACCAAGGATGCATGATCTCTTTACACACTGCGGCCAAAATATTTGA
GACGACGTTCTACACGAGACCAAGAAGGTTAAAAGTTGCGATTTGGGATCCTCGAAGTCGCAGCAGAATGAGGTCTT
GTACAAGGTCACCAGGGTATCACTGTCTGTATACGAGTAAAGGAGGACCTGCTTGTAGCAGACAATGATGGGTATCG
TGTGTGATAGATGTCCAGACATAGCTGATTGCTCTGCATTAGTCTCCTCAGCTGCAGACTAGTGTAAGCGGGCCTGGA
TACAATTCCAAGCCGGTTCGTGCGGCTCGGAAATACGCAATAGTAGTATGGTTTGTATGGGGAAACACTAGTGCGGG
GAGGGACTGATCTCATTGCTTTCAGGAGAATGAGGTTGAAATCAGAAGGTGAATATGGAACGATTCTCGGTCAAAAAA
CATCGAGTTAGCTATGTCTTTGCAATTGTCCTGTATCCACGCATCACGTGAGCCATTGCTCTGGGCTTGAGAGTGCTC
GCTACTTGTCTCCAGCATCGCGATAATGTGCCATATGCATGCATTGATGCAAGAGAATGGCTAGTGGCCTCAACAAGC
ACTAAGGACTGGGGCTTGTGGGGGAGACTGACGGAATAGTCGAAACCCATGGTTATTTTAACAGCTCCGATGTCAAT
ATAAATACCACTGGATACCCCCTCCAGTTACAATCATCACACACAAACACAAACACAACCACTACA
>SEQ_ID_NO_5_1.0 Kb promoter region YALI0C08074g
GCCTGCTCCCCCTAGAATTCTGCATGGAACAGGCAAAGGACCAAGGACGTTTGAAGAGATGGGCATTAATCCGGTGA
CCAAGGAGGCCGACGATTGTGTTATTATGTAATGTATGATATCAGTATTTAATCATAGTGCGGCGAGTAGGAGCTACT
TGCAGGTGCGTAGAGGGCTGTTTGTAGTCATCGCACTGAGCCAGAAGATGAGATGAAGTGTCTGGACGAAGAGCCA
GCTCCTACGAATCCAGATGTTCAGATGGACCCAGCTTCTTTTCTACTTACTTGCTGAACTCAGGTATCACTTCCGGCTG
CTTTCATACGTGTTACGGTATTCACTGTTGACATCATTTGACGCATGTTACCATTACTTGAAGAGTACTTGAAGATAAG
TCAGCTGGGGGTGACTTCGTCATTCCTGAACCATTAAAAAGGAGTGGGGCAGACCAAGTGATGGAGAGATAGCTATT
TATTAAAAATGAAGTAAAATCAGCAACACAGCATCTACAAATGAGCCGGGTTAAGGGTTCTGTCGTTCCAATTGAGTT
GCTGCATTAGTTAGGACTTGCGTTCCGAAGACAGTGCGGCTCGGAAATTTCCAAGGACCGTCTGTGCGGCTCAGATA
TTGTTTGCCAGGCTTTCGGGAGTGTCAATAAATGGCATATTTGCTAGAAAAAATGTCGACTTTCCAGGTTCCCAAGTTG
ATTGTAGGGGAGTTATTGTATCATGACCAGCTTTTTTAGTGCTATTTTCTTCTTTGAGATGCTCTTTTTAGCATCCCGAA
TTTCCACACTAACCGATGTTCGGTACCTTAGCGAGCACGAATAAATAAGCCGCACAGTAGATCCGACTAGACACCGCG
ACTTGACATGATTGACGCAAACCATGTGGAGACTCAACCTCAAGGCATCATGGTGTTTGGGGTATAAAAGGGGGCGC
GAAGACCTCTCTCAAGTCCCTATCAACACTCAACTCAACCCACTACAACCACAACTACTACAACT
>SEQ_ID_NO_6_1.0 Kb promoter region YALI0F13937g
ATTTCAAAAATAAAGGACCTAAATACCACTGCACCTGTTTGGAGAAATAACGACTGTGTATCCCGCGTAATAGGTCAG
GTGCAGTAAGATAAGTCTAGGGTGTTTTCTGTTGATATGGAAACAGGGAACCATGAGTTAGATAACCGACTCCGCGAA
ATCTCTCCGAACTCACCAATTAGAGCCAGTTCCGTGCTATTGGTATATCTGGGCTGAGAGGTGCGCTACCCCTCCCCG
TGTATGGTGGTAATACGGGAGAGAAAAGTGCAAGTACAGGAAGATACAGAGAGCGTAAATCTTAATCTATTTTTGAGA
GACAGGATATGAATAAATTGTACTTTAGAGGAGTTTTGTGGTGACTTCAGCTTGGCTGAGGAAGGATTGTATACGATG
TACTATGATTATCGAGAAAAGCAATGGTTTTCTGATTCATTGTTTTATGTTTCCATCATACCGATTCCGCAATATAATTG
TAATTGCACAAATACTAACCATTTACTTTTGCGGCCATTTTCTGGAGGTTTCGTGTCTATGTACATCATTAACAGAGAC
GGTACTGTGGCGGATGAATCATGTGCGGCTCGAAAATTCAGTCGGTGCGGCTCGAAAATTCAGGCGGTCCGTCTGTG
CGGCTCAGAAATTGTCAGACGGGATGCTTGGAATAATGGCGGGATCCGTTACCAAATTAAAATGTGTGATTAATGTTA
CATTAGATTGTAATTGTTGCAATCTATCGGAATCACCTGTTTGAAGTCATATTTTTTCAGCAAAAATGGCAATTTTTCAG
ACGTGTTTAGTTAAATACAAAATTGCTTCAAGCGGCGACAAGGAATTAATGAGCCGCACGCTTACCCGTTGAAACACC
GCGTCTCGACATGATACATGCAAGTTGGTCAGATCAAGGCGGGGGCAAGATGGCGGTATTTGGAATATAAAAGGGCT
CAAAACTCCAGTCACTTCATCATCAACACCCACACAATCCCCCACAACAACTACTACAGAT
>SEQ_ID_NO_7 1.0 Kb promoter region_YALI0C14344g
TAAAACGCGAGATATACAGGAAAGGCACACACTAAAGGCAAGAATAACAATAATAAGTGTAACAAGAGTTATCGTGTT
GCTCTGGAATTCAACCAAACCCCAAGCCACCATGTGTCAATGGTTTCAAAAACAAGAGCTCCAAATTTGAAGTTTTCAA
TAACGTAGAAATCATTCAAAATAACTTGAAACTCTACAAAAAAGGAGCAGAGGAAATTAATGACCCATAGAAGACTTTA
TCTTTTATTGAAATTATCACATCTAGTAAAACGGCCGATCTGGCTCATTATTGGATAGTTATAGAAACTTCAATTTTTGA
CATCAAGAAAAAAAGCGCCGATGGTTTAGTGGTAAAATCCATCGTTGCCATCGATGGGCCCCCGGTTCGATTCCGGG
TCGGCGCAATCCGTTCAGAGATCTAACTAGTAAATACTTTTTGTAGTGCGAGATTCGTGGTTGGTGTAATCTTTTTGAT
ATTGAATTATTGTACTTTTTATTCCTTCCCCCTAGGCTTATACACCTCTGCGAGCTGGTGCTTTTCAGATGTGTGCATTG
TTTGTTTGCATAATCCGGCCCAAGTTGGTGCGGCTCGGAAATTCACATGGACCGTTGATGCGGCTCGGAAATAGTTTA
GCGGATGTTTCGAGACGGTCAAAAAAGCGGTATATTTGTAGAAAAACATGGCTTCAATAGTACCGGGTGAATTAAGAT
GACCTAATGATTTGATTTCCTTACTTTCACTACCAGGTAACAATCTAATTCGTCGAAAATGGAGCTGCAAAGTTCTTCG
CTTAATTAGCAAGATATTGAAATAAATGAGCCGCACAGTCCATCCGTCTAAACGCCGCGATTCAGCAGGATTAATGCA
ACCTATACAAACCAGGCGGGGACGAAATGGCGAAAAAACGATATAAAAGGGGCTCCAAGTCCTCTCTAATCACCACC
AGCACAAAAACGCACACCACCACCACCACAGCACCACCATACCACTACAACCACCAAG
>SEQ_ID_NO_8_1.0 Kb promoter region YALI0E14256g
ACTTGTTTGACAAGAGCCAAACGATACACCAATGTGTATGAAACCTACGCACTCTTGGCATATCTACTGGTACTGTAG
CGACAGATTCACTTGTTGAGAGCTGTGCTTCCGAGCATCGGATGTACCTCTTTCTCATATAATTATCGTCAATAATACC
GCGCATAACCCAGGCACTCATCAGGGCTGTACACCCTCCTCTCCAATGGCAGGCGCTCGTAGCAGCAACTAAACCTT
GGGGAGGGGGCGTGATCGAGGAAAGGGCTTCCAGTGCGTACCACACACGTATATCGACGTAATCGTGCCATGCAGA
CGGCGTGAGATAGTGTAGTTTGAGCTGTATTCTGAAGCCGGTCTGCCACCGTATGTATAGGATCCACGTCCAAGAAG
CCGCCTCGCTGGAGCCACCGGATCATACCCCATGTTCCAATACCCCGCTGAAAGGACAAACAGAAGCCGGACCGTGC
GGTGCGGCGAGATATTCGGATTTGGCTCCATTATCTTTGTGTATCCGGTGCAAGTCGGCTTTTGCGGCTCGGAAATG
GCTACTTGTAGCTCTGGGTTTGTGTTTGAGGGGAGAGTTGGATATGGAAAAACGTGGATGGTGAAGCCTTCGGGGAA
TTGGTGTGGTTCCCAATCAACTACTAGGTCAATTGATGCCGTCTTTTTGGAGATTTCTGGACGCCATTGAATTGCTGTC
CATGAGACACCCCATATTCGCTTAAGCAGCTTCCTTACCTTAGCGAGGCACAGAACATTCCGCCTGTCAGCCCCAACC
CAATCTCTGAGGGCCACAACTCTCCCCCAATAGCCAGCTGCCCCAGTTGCTCGATCAGCCACCGAAGCTTCAGACAA
GGCAGTTACACACTGAGCCTCAAGGTTGTGCGGGCGGATGGGGTATAAGGGTTGAGGTGGTAACCGTGTGAGCTCA
GAAGATATATAAAGGGGTGGCCATGTCCCCCTATCGCTCCTTACCAAACAACAAACAACAAACAACTACAAT
>SEQ_ID_NO_9_1.0 Kb promoter region YALI0B19976g
AGAGAGCCAGAATGGAGTATCTACTGTATGGGATTATACTGTAGACTTTTTAAGAGAGAACATCTCATCATTGAAAGA
ATTTATGTAATAACACCATCAACGTATTTCATGTAAGCTTCACCTGTTGGTGAGCCACAATACATTGCTATTCGACTGA
AGTATACAAGTAACTCTTGCCAATGAAAATCGAGAATATTATGGACGTTTGGGATGAATGGTGAACCAGTCAACCAAT
TTAGTTGAAAAGTCATCCAGCTTTACCGAAGCAACGCCGTTTAAATATACTGGGGATCCACAGTTTTAGTACTTTCTGA
GTGCCACATCTGTACACCACTACAGATTATAGGGTACCAAAAGACAAAAAAATGTCGGTGAAGATGATGAGATGGATT
TATGAGACTCAGATGGTGACTCCAAGTGACCCAGGAAGTACATAGACAGGGGAGGTAGCGGCGCAAATAATTCTGTT
GAGTTGGATGGCGTCACGATATTTTTCTATTCGCTTAAATCTCCACATTGAGGCGTCTTTTCAAGGCATCATCATTATT
GACAGCCTTTGATCCGTCATGTCATGCGGCTTGGAAATGAAATTCGGCTCCGTCGTGCGGCTCGAAAATGATTGTCTC
GGATAAAGCGGGGGAGTAAAGAGGTGTTTGAAGTTGTGGTAGTTTGGATGGGGAGGAATGAAGTAAAATATAAACAT
TTTCGTGCGGAAAAATGAAGGGTTTTTATATAAAAACCCTACTTATCTTGTCATTTAAAGCCGCTAAATGCGCTGTTAC
TGTAGCATACAGATAGTTAGTGAGAACATGTTGACAAACTCAATCGGATCCTCCAACCTTGTATTTATGCGCCGCACA
ATGAGCAAACCGCGGATCAATGCCCAGGACATGGTGTGGAGACTGTGAAAAGATATAAAAAGAGGTCCTCACCATCC
ATTATTTCTTCATCGACTCACAGTCTTTAGCAACCACTACATCTAACTACAACAACAACTAAA
>SEQ_ID_NO_10_1.0 Kb promoter region YALI0E15840g
CTTTGCTCCAATTGTTGTTTTTGGAACGTGTGTTGCTGGAGGTGTGGATCTTTTGGCAGATACAGGTATGGACGTTCA
GTTGATAGACACCAAGTAAACTACTTGTAGTTGGTCATATGCATCGTCCATCACTCGTGTTAATTTCGAAGGAAGCTGT
TTATTTGATGAGTAGCTCATTTTCCAACTTACTCGGTGAACGTTGCCAGCGGCTGGCCGGTATTGGACAAGATCTTAC
TGTTGGACAAGCCATTGTGAGTCCTCTTGTGGGTGTGTTTGGATGAAAAACAGAACCAATCGCGAGTCTCGGCCCGA
CTCCACTCAGACCTTTTGTATCTGGATAAATGGAGTGTCGCTTGGCTCAGGGGTACGAGACTTCGCCCAAGTTGGCTC
AATTGACTAAAAGTCCATTTTCAACCCGTGTCATGGTCGTTAAACACACCAATTTCACTCTGATACACCAATGAGTCTC
ATTGGACCTTCCAAAGCCCCCATTTAGTACTTTTTTAGCCCTCTCGGGACACTGGCTACTAACATTGAACACCGGCCCC
GCATCGTGCGGCTTGAAAATTCAGGTGGTCCGTTCGTGCGGCTCGAAAATTGTAGAGCGCGGGGGCAGTTCAGGGG
AGGAGCCCGAATAACTCATATTTTGGTAGCCACACCGACCTCAAAGTAATTTCAGAGGGCCATTAATTGATTCTTAATT
GATTATTAATTGATCTTTGTTGTTTCAACGTGCCGGAATGGTCTTTTTATAGGTCCATTGTCTAGTTCACTCAGACCACA
TCAACAAGAGTTCTATGCAGCACGAGTGCTTTAGCGACATGCAGGAAATAATAATGAGCCGCATAGCTCTCCGGTCTG
AAAACCGTCGAACAACACCATTCCTGCAAGTCCCACAGATCGAGGCGGACATGATATGGTGTAGGAAACGATATATAA
GGGACCCAAAAGTCCTGAATTTGGTCTTCATCAACACACCGGTCTACCACCCACTACTACC
>SEQ_ID_NO_11_1.0 Kb promoter region YALI0F28765g
AAATGCTTGGAAGTTACGCTACTGTATATCTGGCCAACCATCGATTTACTTGTATAGTGTTATGTTGAAGAACAAGACG
CTATAGACACACAGTATATCTACAGGCACTTGTACGCAATGAATGGACGCCGTATACTGTTCAAATGTTTGATATTCAG
TATCACCAGCTCACATCTACAAATACTCCACTCCCACATGCCGGAGCCGCACGAACTTATGAGATCAACTTGTGCTCCT
TGTACAAGTACCGGTACAAGTACCTGTTCCTATAGATCTGCGTGATCAAATGCAGGTTGCACACAGAACAACAACAAG
AGAGTACAACACCATCAAGTGAAATCTAATATGCTGCCACACACTGATGAGAGACACCGCTTCTATGACATGATTGAA
ATGAGTTACAGTAGGATGGAAAGACTGGTCGTAGCTAGGTACAAGGATGTGGGGAGGTATGCGTTTATTAGTTCATA
GACCACATGGTCTTTCCATGGGTTAATATCTCGTATAAATGTCCAATACCAGCCACTCTCATTGTATACCACTGCTGCC
TGCCTCACGTCCCGTTTCACGGGTCATACGTGCGGCTGAACTCTACAACTTCCGTGCTGTGCGGCTCATCATCAAGTA
GCCTTTCATTCTGTTGAGCATCCCGTTGTGATACAGAATATGATCCACATGTCCATCTCTTCACCACGACATCAAGTGA
CACGAAAACGACACATAGCAGCGCAAGATACGACGAAAAATACCCAGTGATTTCAGCAAGAAATGTACACTCAATCAT
GATACCATAACGAGGTCACTAGCCTTTCAAACCACCACCTCTAGATGACATCAAGAACGCATCACAGTGCATGTCATG
CACCTCTCGTCACCCACAACCGGGTCAAGGACTACGGAAATCCACTTGAACGATACCCGAAGCGTCTCGGGGTATCT
ATATAAGGCACCCATGGAACCCTGTTGATTCCTCATTCAAACACGCACACGAACAAACACT
>SEQ_ID_NO_12_1.0 Kb promoter region YALI0E19657g
CTGTGCATGGGAAAAATAGAAAAATGAAGAAGAAAAAAATGAAGAAAAAAAACATACCAACAATCAACTCCTCCTTCT
TTCGTCGACTCTTACAAATCATCACGTGACCACACTTCTCCACAACATACTCCCCCAAAATAGAATGCACTATACTGTA
GTATATCTTATACCATACCACAACCGAACACACCGACTAAGAGAAAAAATCGGGCATTTCCACACCTGGAGACACAAA
ATCTCCTCCCTCCAAAACAAGACATATATAAACACTCAAAAATCGCCCTTATCATATCCAAAAAGTCGAAAAAAATGGC
ACTTTTGCCAACCCTCATTTTCTTCAACCCCGGGTCATTAAACTTCCGTCAGACGCACGTCGCATCAACAATAGTCTTT
AATATAATTACTGGAGAAGCGGAGATCACGGGGTTTGGAGGAGAGACAGGACACGGCGAGGACGGTGGTCACGTGA
CCAGAACAAACCCCACATGACCGGGGCCAGTGTATTCACGTGACCCACCCTCTCTCAACCGAAATGGCGACTGTTGC
TGCTGTTGTATTTTGGTCTGTTATCCCAGTCTAACTTCAATGTTGGAGTTGTCGGAAAAAAAATGTAAAAATGGATGAT
AAAATGTAAAAATGGAATTAAAAAAAATGGGTTAAATGTTAAAATGGATTAAATGTAAAAAAGGAATTAATGTATAAAG
TGAAAATAAGTCGGGTTTATCCGGGGTCCACCAGGGAGCTGAAAAGTTGTCAGATTTGAAAAAGCAGAGAACCAATG
GATTCTCCGAGTTCCTGCGATTCCAGTTCTCTTTTCTCCACCCAGATCAGACCTCCGCAACGTCCAATATTTGCATTCC
ACCCCGGATCCACACAAGGTTATATTCTGCAGAAATAGTACAACCAAGGGTGTCCACCTCAACACATTTATCCAACCA
CCCACCCACTCCTACTGTTACGTGTACCGACACAGAACGTCACTCTTTTAACACCCTCATTG
>SEQ_ID_NO_13_1.0 Kb promoter region YALI0B21670g
CCACAATGATCACCGTGGGGCTATCGAACTTCTTAGCAAACATGTAGGCGGTCTCTTTTCCGATTCCGGACGTTCCGC
CGAAGAAGACGGCAACGGCATCCTTAACGGTGGGCTTATAGGCCGCATTGACCTGTTTGATATCGGCTAGAGAGGGC
ATGGTGTGTAGTTGCGAAAGGTGGCGAAATCCGGGGCTTAAGTATTAGAGTCCTGCTGTGCAGGGCTGATGGCTTTA
GGTATACAACGCTATTTGGACGCAAGGTACATTTGAGACAGGGCAGCAGGAGGGAGGGGAGGAGCTAGAGGGACG
GAGAATAGGCATTTCCAGCCGGAGTTTTCGGAAATCAATTGAATAAATAATTTTAAACAGATATATATCTTTAGATTTTT
ACAGTTCTCATGGGAGCTCTCCAGATAAGTCCGCCTTGGAACACATCTCATCTGCATGAATTACTAAGGTGGCGGAAA
AAAAACAACGTCTGAGGCAAGTTCAAGCAGGCTGGCGGTGGGATCTAATTATTCAGCGCCAAACAGATATGACCCTT
ATATGATGAAGGGCTATTATAGCAGCTACAGCAGATGCAAAGGAGGCTAGATGATAGCCTGGCATTGGTACAAGTAG
CTCACCTCGTGGCGAACCTTTGCTCCCATACATGTCTCTTTCAGACTGCACTATTGCGCATTTATACGTGGTTATACGT
CTTCTTTCTTCGAGTTACATGCCCTGTGTGTAACTGTAAGGAGTGGTACCCCGTCTCGTACAAAACCTGCAACTGTCAA
TTGTAACCGCACAAGTGCGTGTGTGGGCGTGCTATTGAGTCAGACTTTGACTCATGTGGGCCACCAATAAAAGCCTG
AGGATAGAGCCCTCAAAAGTCCCAATGACCTTGTGGGGGTGCTCCGCTTAGCGTGGGGGTCAACCTAAAGGAGGAA
GTACCCAAGGAGCTAACCCCAGACTAATAGTACAGTACGCATCCAACAACACACTCAATTGCAACACACA
>SEQ_ID_NO_14_1.0 Kb promoter region YALI0F29315g
TGTCCTCAGGTGCAGAATAACTGTCGCTATGGCTGTTAATTTGGGGCCATATACAAGTAGCTCAAATCCACGCTCCAC
CGCTGCTGGGGTTGAGACAAAACCTGTGAGCCTCCACATTGCACAAAAGAGGGAAATAATCGGCACCAATCGGTATC
AATCAGAACCAACCGATTTATCTGTTTAACACCAAAACCCCTTTCTACAAGTAGCCACATCTAACCACTCACCAGGCCC
ATTACCCCGCCTGTTGATCAACGGCTATTTCTACCTCATCCATATTGGGGGTGCATAACCCATCAGACTGGGCGTCTA
ATAGGAAGAGCAGGAGGAGGATATTAGACGACGTCCTCACCCAGCTGGGTAAGAGCAGAGTGACAACCAGCTCTCTT
TCCGAAAGCCGTATATACTCTCAAAACACTCCACCATCTACGATAAGCATATCGGCGCGAGTCTTATTTCATATATCTG
GTGTTTTACCGTAAGATCCACCTTCCATCCGAAGTATGAGGCTTCGGGTTCAACCCCCGCCCTCATACACTACTTGTA
GAAGGCGGAGGTGTCCACACACTTTTTTTTCTTTCTGCAAACAATACTCTCCGTTGCACTGCGGCTCAATAATGCTACA
TACGACTTATCCGGAATTAGGTTGGGACCGTTGAGTACGACCGCTGATCGACGACTCTGAAGATGGTGATATCGCTG
AAAGAGATACCATTACACTACATTTGAAATACAGAACATTATTTCCAGGAGTAATGTACCACTTGAAGTCTGTGATTTT
AAATCTCTCATCAACTGTGTATACCTGGCTCTTGCAAACCTCCACATGCATGCAGCACTACCTCCATGAACGGATCTCG
GATCCCCTACTGCGGCGATTCAGTGGGTATATACCTCAATTGGACCTATAAACTAATTATTCTTCTCCTCGACTTTTCTA
TTTCATCACACACACTTACACGCACACGAACAAACACTACGATGGTCCTTCTTATTCTCC
>SEQ_ID_NO_15_1.0 Kb promoter region YALI0D25256g
TCCCCAACTTCAACTAGTGCAAATCTCGGTAGTATTTTGTCATTGTTTTATAAAAGAAAGCTCATCATTATTATGAAAGA
TTGCAGCATTTCACCGAGAGGTAGAGCGGCTTTTCAAAGAGTTCAAATCGGCTCAAATCTGTTCAAATCAGCTCAATC
TGTTCAAATCAGCTCAATCTGTTCAAATCAGCTCAAATCAACTCAATTCAGTTCAAATTCGACAAGACACTCTCATCGCT
CATTCGATGAATCCCTTTTATAATCCAGCAGTATTCATATGCTCTCCCGTCCACGTGGGCAGTTTCCTTCAGCTACAAT
ACATCTGCAAAGACACTCTGTTCTATACCTCCCGGGTATCCGCTATGTTCAATTGCCTTGCTAAAACCTCACAAGCCTT
GCTATGCAGTCCAGTCGAATTATCCGGGATTTGGTACGCTTCAACACGAAAGAGGATGTACAGTACAAGTAGACCGA
CAGACCTGCACCCCGCCTACTACAAGTAGCCGCTTTGACCCCCCACTAGACAACACACACACAGTGAGATGTAAACG
GCCAGTTTCTAGCAGCCACGTACGTACTTGAGCAGTATAGAACCATGCAACTGCATCTCTCCCTCTGTGACCCCCAAT
TGCAAACCGTGTTTGAAAATACCTTTGCCAATCAGCGCAGGTTGAATGTGGGTGAACAAGGTCAAAACGGGTGAACA
AAGGTCAAAACGGGTGAACAAGGTCAAAACGGGTGAACAAATGTCAAGCGCAAGACGGAGACAGAAGTTTCGGGGT
CACGTGACTATCACGAGTTGCGATTCTACGACGCTCATCAGCGACGCCCTTGGATTTGCTCCATTCTCGCTTAATTTCG
CTTATCGCCAATTGCGTGGGGGGTGTGGGGGGTCCCAACTTTAAAACACTTACCCAACATGTCCCCCACGCTCTAGAT
ATATACCACCTCGAAAATCCCACTCCCCCACACATCGCTCAATTCCTCCACGCGTGCGCCAA
>SEQ_ID_NO_16_1.0 Kb promoter region YALI0C11099g
ATCCTGGGCAAGGCGCTGGCTGAAAATTATCAGGCCACCTGTAGAGCATTCTTTCCGTCCACGTGGGGACACCTTGT
ACTCGGTGTGGTTGACAAAAGCAGACCATCTCGGATCCCTTGAGCATCTTAGCAAGTGGCGTCACCTACTCGAGCTTG
TCTCCCACCAGTGTCCTTGAGCTTCAAATCCAGAGACGGCCCCGATACATTCTCTTATGATTGTTGAATACCGTTTCCG
CTCCTTGAAGAGCAGGGAATCTTGACGGACTGGTTGTGGTAGAAGAGCCAGCCGGCAGAAACACGGCCACTGAATC
AAGGAGAGATTGTAGTAGAGCGTCTTTTTGTCGCCCTTGCCTCCGATTCCATACTTTTTCGTCAGGGTGTGTTTGATAT
CCATCAGTTTGAGCTGCTTTACAGGGGCATGGGAGCCCCTTCTCCCTCTTTACCCGTCAGACTCCATCCTTCTTCTTCG
TTCTCTTCTCCGTCTTCTCTCATCACCATAGTGGTAGAGGTGTGCAGACCTTGATATCGTCGGAGCAGAAGTTGTCGT
CGGAACTAGCCAGGCTCATTTTGTCAATGTATTGAGCTGCATTGCGTATCTCGCGTCTTATTCTAAGACACCGTCTCAG
CACATTAGTGCGGCTTGAAATTTCATTCACGACTTGGAAATTATGCCGCCCGTACCGTCGAAAGATGTTAGTAAGCAA
TAGCTGAAAATACCGGGAATCGATTGGTGCAGTCTCAATGTGTCTATTTAAACCAACTTGGTCTGTTTTTGCTATATTG
GAGCCCCGCTAGAACTCACGATCGAGCCTCTGGTTCTTAGCGAGAAAATAAGCGAGTCGCACCACAGCCGTCGAAAC
ACCCGCTGGCTTGCAAGTTGAACACCTCAAGGAGGGCTCCAGATGGTGATCATGACAATAAAAAGGACCCCAAATCC
TCTCTATTCCTATCTACTCACAAACACCACCAATATGAAAGTCCTTGTCCTCCCACGCCAGCTC
>SEQ_ID_NO_17_1.0 Kb promoter region YALI0F09966g
CTCCCCTTCCGTACCTCTCTGCCCCTTCTGGACAGGTCAATGATAGACTCAGAGCGACACACATGTCTGACGTACCAT
GTTAGACCTTGTATTGACCTGGACGAATGTGTGTGAGGAGTGAGGCAGGCCAAGACGAACCACGGTCTTTATATATG
CCCACGGAGTGACACGGTCTGTGTCGTCACCGCAGCTCCACTCACCACCCGCATCATGATCGTCCAACCAGAACCCA
CTCCCCAGTTTCGACCCAACACCATTCTCAACTGTAAGTATGAGTACCACAGTGATACTCGCCCAGTGCCGCACTCGT
ACTGTAGCCACTCCACTGCAACATCCGTATCGTATTGCACCGCCCCGATTCACCTGCTTCCTTCAAGCCTTCAACCACG
TACTGCTCCACCTCCTACCGTTGAGCCCACTCGGATCGGCCAGAGTCATGTCTTAGGGTTTGGCTGCAGTTGTGGCGT
AAACTATGGAGAAGGCGACGGAAACGAGAGCGCTACCGGTAGCGACTTGGCGACACGTCTGGCTCGGGAAGGGGG
CCGTTGCAGAGACCAAGACTTCCGTCACGTGACCGCTGTTTGGTCAATTCTAACGCAGTTATTTTCCGTCTGATTCGCT
GATACGAGTACTCGCTTGCTGTAGATGACTCAGACCAAGACAAGAGAAGGGGAAATAAAAAAAACTTCCAAAAAAAAC
TTCCAAAAAAAAAAAATCAAAATTTGACAAACCTTTTCTGCCTGGGACCAGGAACTTTGTGAGTCCATTGAGGGAGTTA
GCCACCCATCAGCCACAGCCACAGTTTGGACAAGAAGTAAAAGTGGATATATTTATGTTATGGAGACCATGTAGTGTT
GTGGGAGGGAGGGGTTTTTTTGTTTGTTTTGGCTGAGTAATCAACAGCAAGTGGCGTATATCGTATATCTATCGTGAC
TCAGACTATTCACCGCTTGTATGGTGCTATCTCGACTTGTGCTTAGTCTCAGGTACACGTGATTGC

As described above, mutated or truncated versions of the sequences of SEQ ID NO:2-33, preferably SEQ ID NO: 2-11 and 18-27, preferably SEQ ID NO: 18-27, are also likely to function as a formate inducible promoter in a non-methylotrophic yeast. that make use of the inventive concept are also provided by the present invention. Accordingly, sequences with at least 80%, 85%, 90%, 92%, 94%, 95%, 96%, 97%, 98% or 99% sequence identity to these sequences are considered to be useful and are considered to be nucleic acids of the invention.

The invention also provides nucleic acids comprising or consisting of mutated or truncated versions of the 1Kb and 1.5Kb sequences recited herein.

The invention also provides nucleic acids comprising or consisting of a nucleic acid that comprises or consists of a portion of the 1Kb or the 1.5Kb sequences recited herein.

It will be understood that a portion of the 1Kb or 1.5Kb sequences recited herein may consist or comprise a portion of any length. Accordingly, in some embodiments, the nucleic acid of the invention comprises a portion of one or more of the 1 kb or 1.5Kb sequences recited herein where the portion is between about 46 and 1500 bp in length, 50 and 1500 bp in length, 100 and 1500 bp in length, for example between 150 and 1400, 200 and 1300, 200 and 1200, 250 and 1100, 250 and 1000, 300 and 950, 350 and 900, 400 and 850, 450 and 800, 500 and 750, 550 and 700, 600 and 650 bp in length.

In the same or different embodiments the nucleic acid of the invention comprises a portion of one or more of the 1 kb or 1.5Kb sequences recited herein where the portion is about 46, 50, 75, 100, 150, 200, 250, 300, 350, 400, 450, 500, 550, 600, 650, 700, 750, 800, 850, 900, 950, 1000, 1100, 1200, 1300, 1400 or about 1500 bp in length. In the same or different embodiments the nucleic acid of the invention comprises a portion of one or more of the 1 kb or 1.5Kb sequences recited herein where the portion is at least 46, 50, 75, 100, 150, 200, 250, 300, 350, 400, 450, 500, 550, 600, 650, 700, 750, 800, 850, 900, 950, 1000, 1100, 1200, 1300, 1400 or at least 1500 bp in length.

It will be understood that a portion of one or more of the 1 kb or 1.5Kb sequences recited herein consist or comprise a portion spanning any range within the 1 kb or 1.5Kb sequences. Accordingly, in some embodiments, a portion one or more of the 1 kb or 1.5Kb sequences recited herein spans between about position 1 and 1500 bp, or between about position 50 and 1500 bp, 75 and 1500 bp 100 and 1400 bp, 200 and 1300, 200 and 1200, 250 and 1100, 250 and 1000, 300 and 950, 350 and 900, 400 and 850, 450 and 800, 500 and 750, 550 and 700, 600 and 650 bp.

The skilled person will appreciate that the portion could span any region of the 1Kb or 1.5Kb sequences recited herein, for instance may span from position 25 to position 254; or from position 500 to position 725. Naming convention is that the sequence is orientated 5′ to 3′.

To exemplify how the above features may be combined, a nucleic acid of the invention may comprise a 150 bp region that spans the position 200 to 350 in SEQ ID NO: 2; or may comprise a 345 bp portion of SEQ ID N: 5 starting from position 679 of SEQ ID NO: 5.

In one aspect, the invention provides an isolated nucleic acid which comprises or consists of a sequence selected from a group comprising or consisting of SEQ ID NO: 2-33; or is selected from a group comprising a sequence with at least 80%, 85%, 90%, 92%, 94%, 95%, 96%, 97%, 98% or 99% sequence identity to SEQ ID NO: 2-33. In preferred embodiments, the invention provides an isolated nucleic acid which comprises or consists of a sequence selected from a group comprising or consisting of SEQ ID NO: 2-11 and 18-27; or is selected from a group comprising a sequence with at least 80%, 85%, 90%, 92%, 94%, 95%, 96%, 97%, 98% or 99% sequence identity to SEQ ID NO: 2-11 and 18-27. Such nucleic acids are expected to act as inducible promoters according to the invention.

In one embodiment, the invention provides an isolated nucleic acid which comprises or consists of a sequence selected from the group comprising or consisting of SEQ ID NO: 18-27; or is selected from a group comprising a sequence with at least 80%, 85%, 90%, 92%, 94%, 95%, 96%, 97%, 98% or 99% sequence identity to SEQ ID NO: 18-27. Such nucleic acids are expected to act as inducible promoters according to the invention.

In some embodiments, the invention provides an isolated nucleic acid that consists of a sequence selected from SEQ ID NO: 2-33, optionally selected from SEQ ID NO: 2-11 and 18-27, optionally from SEQ ID NO: 18-27. Such nucleic acids are expected to act as inducible promoters according to the invention.

Table 1 sets out the fold induced expression from each of the promoters in Yarrowia lipolytica when cultured in YNB. In some embodiments a promoter with a high fold induction is preferred. Accordingly in some embodiments the promoter comprises or consists of a portion of a sequence selected from a group comprising or consisting of:
i) SEQ ID NO: 8, SEQ ID NO: 24, SEQ ID NO: 6, SEQ ID NO: 22, SEQ ID NO: 9, SEQ ID NO: 25, SEQ ID NO: 11, SEQ ID NO: 27, SEQ ID NO: 2, SEQ ID NO: 18, SEQ ID NO: 10, SEQ ID NO: 26, SEQ ID NO: 7, SEQ ID NO: 23, SEQ ID NO: 3, SEQ ID NO: 19, SEQ ID NO: 5, SEQ ID NO: 21, SEQ ID NO: 4, SEQ ID NO: 20, SEQ ID NO: 12, SEQ ID NO: 13, SEQ ID NO: 14, SEQ ID NO: 15, SEQ ID NO: 16, SEQ ID NO: 17, SEQ ID NO: 28, SEQ ID NO: 29, SEQ ID NO: 30, SEQ ID NO: 31, SEQ ID NO: 32, or SEQ ID NO: 33; or
comprises a portion of a sequence selected from a group comprising a sequence with at least 80%, 85%, 90%, 92%, 94%, 95%, 96%, 97%, 98% or 99% sequence identity to SEQ ID NO: 6, SEQ ID NO: 22, SEQ ID NO: 9, SEQ ID NO: 25, SEQ ID NO: 11, SEQ ID NO: 27, SEQ ID NO: 2, SEQ ID NO: 18, SEQ ID NO: 10, SEQ ID NO: 26, SEQ ID NO: 7, SEQ ID NO: 23, SEQ ID NO: 3, SEQ ID NO: 19, SEQ ID NO: 5, SEQ ID NO: 21, SEQ ID NO: 4, SEQ ID NO: 20, SEQ ID NO: 12, SEQ ID NO: 13, SEQ ID NO: 14, SEQ ID NO: 15, SEQ ID NO: 16, SEQ ID NO: 17 SEQ ID NO: 28, SEQ ID NO: 29, SEQ ID NO: 30, SEQ ID NO: 31, SEQ ID NO: 32, or SEQ ID NO: 33;
ii) SEQ ID NO: 8, SEQ ID NO: 24, SEQ ID NO: 6, SEQ ID NO: 22, SEQ ID NO: 9, SEQ ID NO: 25, SEQ ID NO: 11, SEQ ID NO: 27, SEQ ID NO: 2, SEQ ID NO: 18, SEQ ID NO: 10, SEQ ID NO: 26, SEQ ID NO: 7, SEQ ID NO: 23, SEQ ID NO: 3, SEQ ID NO: 19, SEQ ID NO: 5, SEQ ID NO: 21, SEQ ID NO: 4, SEQ ID NO: 20; or
comprises a portion of a sequence selected from a group comprising a sequence with at least 80%, 85%, 90%, 92%, 94%, 95%, 96%, 97%, 98% or 99% sequence identity to SEQ ID NO: 8, SEQ ID NO: 24, SEQ ID NO: 6, SEQ ID NO: 22, SEQ ID NO: 9, SEQ ID NO: 25, SEQ ID NO: 11, SEQ ID NO: 27, SEQ ID NO: 2, SEQ ID NO: 18, SEQ ID NO: 10, SEQ ID NO: 26, SEQ ID NO: 7, SEQ ID NO: 23, SEQ ID NO: 3, SEQ ID NO: 19, SEQ ID NO: 5, SEQ ID NO: 21, SEQ ID NO: 4, SEQ ID NO: 20;
iii) SEQ ID NO: 8, SEQ ID NO: 24, SEQ ID NO: 6, SEQ ID NO: 22, SEQ ID NO: 9, SEQ ID NO: 25, or comprises a portion of a sequence selected from a group comprising a sequence with at least 80%, 85%, 90%, 92%, 94%, 95%, 96%, 97%, 98% or 99% sequence identity to SEQ ID NO: 8, SEQ ID NO: 24, SEQ ID NO: 6, SEQ ID NO: 22, SEQ ID NO: 9, SEQ ID NO: 25; or
iv) SEQ ID NO: 8, SEQ ID NO: 24, SEQ ID NO: 6, SEQ ID NO: 22, or comprises a portion of a sequence selected from a group comprising a sequence with at least 80%, 85%, 90%, 92%, 94%, 95%, 96%, 97%, 98% or 99% sequence identity to SEQ ID NO: 8, SEQ ID NO: 24, SEQ ID NO: 6, SEQ ID NO: 22.

In some embodiments the promoter comprises or consists of a portion of a sequence selected from a group comprising or consisting of: SEQ ID NO: 8, SEQ ID NO: 24, SEQ ID NO: 6, SEQ ID NO: 22, or comprises a portion of a sequence selected from a group comprising a sequence with at least 80%, 85%, 90%, 92%, 94%, 95%, 96%, 97%, 98% or 99% sequence identity to SEQ ID NO: 8, SEQ ID NO: 24, SEQ ID NO: 6, SEQ ID NO: 22.

As stated above, it is not considered essential that the isolated nucleic acid comprises a sequence with 100% sequence identity to the claimed sequences, or to the 1Kb or 1.5 kb region directly upstream from the start codon of an FDH gene identified in a non-methylotrophic yeast, and so the isolated nucleic acid may comprise mutations relative to the sequences of any of SEQ ID NO: 2-17 or relative to the 1Kb or 1.5 kb region directly upstream from the start codon of an FDH gene identified in a non-methylotrophic yeast. Nucleic acid mutations are well known to the skilled person, and may comprise or consist a nucleotide, nucleobase or base substitution, a nucleotide, nucleobase or base deletion, a nucleotide, nucleobase or base insertion, a polynucleotide substitution, a polynucleotide insertion, or a polynucleotide deletion.

The terms “polynucleotide”, “nucleotide”, “nucleobase” and “base” are known to those skilled in the art.

A nucleotide, nucleobase or base may be a purine nucleotide, nucleobase or base or a pyrimidine nucleotide, nucleobase or base.

A purine nucleotide, nucleobase or base may be a canonical purine nucleotide, nucleobase or base or a purine nucleotide, nucleobase or base analogue.

A pyrimidine nucleotide, nucleobase or base may be a canonical pyrimidine nucleotide, nucleobase or base or a pyrimidine nucleotide, nucleobase or base analogue.

A nucleotide, nucleobase or base deletion may be defined as the deletion of one or more nucleotides, nucleobases or bases from a nucleic acid sequence at any position on said sequence.

A nucleotide, nucleobase or base insertion may be defined as the insertion of one or more nucleotides, nucleobases or bases into a nucleic acid sequence between two nucleotides, nucleobases or bases of said sequence at any position in said sequence.

A nucleotide, nucleobase or base substitution may be defined as the substitution of a first nucleotide, nucleobase or base with a second nucleotide, nucleobase or base within a nucleic acid sequence. The first nucleotide, nucleobase or base and second nucleotide, nucleobase or base may be different bases. A nucleotide, nucleobase or base substitution may comprise or consist a transition mutation or a transversion mutation.

The nucleic acids of the invention may comprise one or more mutations relative to any of the sequences of the invention. Accordingly, a mutation may be present in any of the sequences defined by SEQ ID NO: 2-33; or in a sequence with at least 80%, 85%, 90%, 92%, 94%, 95%, 96%, 97%, 98% or 99% sequence identity to SEQ ID NO: 2-33.

A mutation may be introduced at any position in the isolated nucleic acid or sequences of the invention relative to the stated sequence, or relative to the sequence upstream of the FDH or putative FDH gene identified in a non-methylotrophic yeast species.

A sequence may comprise or consist one or more mutations. Accordingly, in some embodiments, the isolated nucleic acid or sequence may comprise or consist at least one, at least two, at least three, at least four, at least five, at least six, at least seven, at least eight, or at least nine mutations relative to the claimed sequences. In some embodiments, the isolated nucleic acid or sequence may comprise or consist at least 10, at least 20, at least 30, at least 40, at least 50, at least 60, at least 70, at least 80, or at least 90 mutations relative to the claimed sequences. In some embodiments, the isolated nucleic acid or sequence may comprise or consist at least 100, at least 200, at least 300, at least 400, at least 500, at least 600, at least 700, at least 800, or at least 900 mutations relative to the claimed sequences.

It will be appreciated that the isolated nucleic acid of the invention may comprise a portion of the sequences described or claimed herein, and that portion may comprise one or more mutations relative to the claimed or described sequences. For example, in one embodiment, the isolated nucleic acid of the invention may comprise:
a portion of a sequence selected from a group comprising or consisting of SEQ ID NO: 2-33; or is selected from a group comprising a sequence with at least 80%, 85%, 90%, 92%, 94%, 95%, 96%, 97%, 98% or 99% sequence identity to SEQ ID NO: 2-33; or a portion of a region of up to 1Kb or up to 1.5Kb directly upstream of the translation start codon of a FDH gene, or of a putative FDH gene identified in a non-methylotrophic organism; and
wherein the portion comprises one or more mutations relative to the claimed sequence, for example may comprise or consist at least one, at least two, at least three, at least four, at least five, at least six, at least seven, at least eight, at least nine, at least 10, at least 20, at least 30, at least 40, at least 50, at least 60, at least 70, at least 80, at least 90, at least 100, at least 200, at least 300, at least 400, at least 500, at least 600, at least 700, at least 800, or at least 900 mutations relative to the claimed sequences.

It will also be clear to the skilled person that the nucleic acid of the invention can consist of a portion of the claimed sequences as described herein, and can also comprise a portion of the claimed sequences as described herein, i.e. the portion can be part of a longer nucleic acid.

For example, to demonstrate how the above features can be combined, the present invention provides a 500 bp portion of SEQ ID NO: 8 wherein the portion comprises 10 mutations relative to the same portion of SEQ ID NO: 8; and the invention also provides an isolated nucleic acid that is 800 bp in length that comprises a 200 bp portion from SEQ ID NO: 2, wherein the portion comprises 10 mutations relative to the said portion of SEQ ID NO: 2.

Preferences for the portion and the mutations are as described herein.

It will be appreciated that the isolated nucleic acid and nucleic acid sequences described herein are capable of driving transcription from a downstream nucleic acid, when operably positioned. Accordingly, in one embodiment the isolated nucleic acid of the invention is a promoter.

The term “promoter” is well known in the field and the skilled person will readily understand what is meant by “a promoter”. In one embodiment, a promoter is a nucleic acid sequence that is capable of initiating transcription from a downstream nucleic acid sequence, when the promoter and downstream sequence are operably linked.

The invention therefore also provides a promoter, wherein the promoter is an isolated nucleic acid or nucleic acid sequence of the invention as described herein, for example the promoter is a portion of a 1Kb or 1.5Kb region upstream of an FDH gene in a non-methylotrophic yeast, or for example the promoter consists of SEQ ID NO 7. Preference for features of the nucleic acid are as described herein.

Promoters are typically either constitutive, i.e., are active all of the time with no readable means of controlling expression; are inducible, i.e., are typically inactive but can be made active or more active by one or more particular inducing agents; or are repressible, i.e., are active but can be made less active by one or more particular repressors.

In one embodiment, the isolated nucleic acid or promoter of the invention is a constitutive promoter. However, one advantage of the present invention is the identification of promoter regions that act as inducible promoters. Accordingly, it will be appreciated that in one embodiment the promoter is an inducible promoter.

An inducible promoter is a promoter which initiates transcription from a downstream nucleic acid sequence, when the promoter and downstream sequence are operably linked, only, or to an increased level, when the inducible promoter is contacted with an inducing agent or condition.

An inducing agent condition may be a compound, a chemical, a protein, a nucleic acid, a temperature, a pH, or any combination of these. An inducing agent condition may be endogenous or exogenous.

The skilled person will understand that the process of transcription from a nucleic acid sequence is often termed “expression”, i.e., expression of the RNA transcript, regardless of whether that transcript is a protein encoding mRNA or is, for example, a gRNA. As such, the initiation of transcription from a downstream nucleic acid sequence by an upstream inducible promoter, wherein the downstream nucleic acid sequence and upstream inducible promoter are operably linked, may be termed “inducible expression”.

Accordingly, in some embodiments, expression from the inducible promoter is induced by a compound selected from the group consisting or comprising of: formate, formic acid, formaldehyde, methanol, ethanol, propanol, butanol, glycerol or any combination thereof. In some embodiments, expression from the inducible promoter is induced by a compound selected from the group consisting or comprising of: formate and methanol. In a preferred embodiment, expression from the inducible promoter is induced by formate. In another preferred embodiment, expression from the inducible promoter is induced by methanol. It is considered that the promoters and nucleic acids of the invention are induced by formate. However, it is expected that the above agents such as methanol and formaldehyde are degraded by the cell to formate, and so may also be used as an inducing agent. Accordingly, in one embodiment the inducing agent is an agent that is degraded or otherwise metabolised inside the cell, or in the external culture media, to formate.

Typically, the induction of a promoter is carried out in vivo, i.e., wherein the promoter is located within a cell, for example within a Yarrowia cell. However it is also possible that the nucleic acid or promoter of the invention may be used in a cell-free, or in vitro expression system. The skilled person is able to determine the appropriate concentration of the inducing agent, such as formate, that the cell should be exposed to, or that should be added to the in vitro expression system. The skilled person will also realise that the type of media that the cell, for example the Yarrowia cell, is grown in, will affect the concentration of inducing agent, such as formate, that is required for a given level of induction. For example, YNB is a minimal yeast media, and yeast grown in YNB are often more sensitive to particular agents than yeast grown in rich media. This is all basic and routine and the skilled person would have no problem identifying the necessary suitable concentration of inducing agent.

In one embodiment, expression from the promoter is induced in YNB media or in ACH +caa media.

In some embodiments, where the inducing agent is a solid, the concentration of inducing agent that the cell or the promoter is exposed to is between 0.0001% (w/v) and 10% (w/v). In some embodiments, the concentration of inducing agent that the cell or the promoter is exposed to is between 0.001% (w/v) and 9% (w/v), 0.01% (w/v) and 8% (w/v), 0.1% (w/v) and 7% (w/v), 1% (w/v) and 6% (w/v), 2% (w/v) and 5% (w/v), 3% (w/v) and 4% (w/v). Accordingly, in some embodiments the concentration of inducing agent that the cell or the promoter is exposed to is at least about 0.0001% (w/v), at least about 0.001% (w/v), at least about 0.01% (w/v), at least about 0.1% (w/v), at least about 1% (w/v), at least about 2% (w/v), at least about 2% (w/v), at least about 3% (w/v), at least about 4% (w/v), at least about 5% (w/v), at least about 6% (w/v), at least about 7% (w/v), at least about 8% (w/v), or at least about 9% (w/v). In some embodiments, the concentration of inducing agent that the cell or the promoter is exposed to is about 0.0001% (w/v), about 0.001% (w/v), about 0.01% (w/v), about 0.1% (w/v), about 1% (w/v), about 2% (w/v), about 2.5% (w/v), about 3% (w/v), about 4% (w/v), about 5% (w/v), about 6% (w/v), about 7% (w/v), about 8% (w/v), about 9% (w/v), or about 10% (w/v).

In some embodiments, where the inducing agent is a liquid, the concentration of the inducing agent that the cell or the promoter is exposed to is between 0.0001% (v/v) and 10% (v/v). In some embodiments, the concentration of inducing agent that the cell or the promoter is exposed to is between 0.001% (v/v) and 9% (v/v), 0.01% (v/v) and 8% (v/v), 0.1% (v/v) and 7% (v/v), 1% (v/v) and 6% (v/v), 2% (v/v) and 5% (v/v), 3% (v/v) and 4% (v/v). Accordingly, in some embodiments the concentration of inducing agent that the cell or the promoter is exposed to is at least about 0.0001% (v/v), at least about 0.001% (v/v), at least about 0.01% (v/v), at least about 0.1% (v/v), at least about 1% (v/v), at least about 2% (v/v), at least about 2% (v/v), at least about 3% (v/v), at least about 4% (v/v), at least about 5% (v/v), at least about 6% (v/v), at least about 7% (v/v), at least about 8% (v/v), or at least about 9% (v/v). In some embodiments, the concentration of inducing agent that the cell or the promoter is exposed to is about 0.0001% (v/v), about 0.001% (v/v), about 0.01% (v/v), about 0.1% (v/v), about 1% (v/v), about 2% (v/v), about 2% (v/v), about 3% (v/v), about 4% (v/v), about 5% (v/v), about 6% (v/v), about 7% (v/v), about 8% (v/v), about 9% (v/v), or about 10% (v/v).

The chemical structure of formate will be known by those skilled in the art. It will be appreciated that a formate is a salt or ester of formic acid. In some embodiments, the formate of the present invention is hydrogen formate, or formic acid. In some embodiments, the formate of the present invention is a formate salt selected from but not limited to the group comprising or consisting: ammonium formate, calcium formate, iron(II) formate dihydrate, sodium formate, iron(II) formate, potassium formate, magnesium formate, iron(III) formate, gold(III) formate, beryllium formate, manganese(II) formate dihydrate, barium formate, cobalt(II) formate, thallium(II) formate, aluminium formate, nickel(II) formate, bismuth(V) formate, zinc formate, lithium formate, titanium(IV) formate, scandium(III) formate, copper(II) formate, silver formate, chromium(III) formate. In some embodiments, the formate of the present invention is a formate ester. In some embodiments, the formate ester is selected from but not limited to the group comprising or consisting: ethyl formate and methyl formate. In a preferred embodiment, the formate is formic acid. In another preferred embodiment, the formate is sodium formate. In another preferred embodiment, the formate is potassium formate. In another preferred embodiment, the formate is ammonium formate.

The skilled person will understand that the formate may be dissolved or mixed in a variety of solvents. Accordingly, in one embodiment, the formate is dissolved or mixed in water. In one embodiment, the formate is dissolved or mixed in an organic solvent. In one embodiment, the solvent is dissolved or mixed in a mixture of an organic solvent and water. In some embodiments, the formate is dissolved or mixed in an organic solvent selected from the group comprising or consisting of: ether, acetone, ethyl acetate, glycerol, methanol, ethanol, benzene, toluene, or xylene. In one embodiment, the formate is dissolved or mixed in a mixture of ethanol and water. In some embodiments, the formate is dissolved or mixed in an appropriate culture medium.

In some embodiments, the concentration of formate that the cell or the promoter is exposed to is between 0.0001% (w/v) and 10% (w/v). In some embodiments, the concentration of formate that the cell or the promoter is exposed to is between 0.001% (w/v) and 9% (w/v), 0.01% (w/v) and 8% (w/v), 0.1% (w/v) and 7% (w/v), 1% (w/v) and 6% (w/v), 2% (w/v) and 5% (w/v), 2.5% (w/v) and 4% (w/v). Accordingly, in some embodiments the concentration of formate that the cell or the promoter is exposed to is at least about 0.0001% (w/v), at least about 0.001% (w/v), at least about 0.01% (w/v), at least about 0.1% (w/v), at least about 1% (w/v), at least about 2% (w/v), at least about 2.5% (w/v), at least about 3% (w/v), at least about 4% (w/v), at least about 5% (w/v), at least about 6% (w/v), at least about 7% (w/v), at least about 8% (w/v), or at least about 9% (w/v). In some embodiments, the concentration of formate that the cell or the promoter is exposed to is about 0.0001% (w/v), about 0.001% (w/v), about 0.01% (w/v), about 0.1% (w/v), about 1% (w/v), about 2% (w/v), about 2.5% (w/v), about 3% (w/v), about 4% (w/v), about 5% (w/v), about 6% (w/v), about 7% (w/v), about 8% (w/v), about 9% (w/v), or about 10% (w/v).

In some embodiments, the concentration of the formic acid that the cell or the promoter is exposed to is between 0.0001% (v/v) and 10% (v/v). In some embodiments, the concentration of formic acid that the cell or the promoter is exposed to is between 0.001% (v/v) and 9% (v/v), 0.01% (v/v) and 8% (v/v), 0.1% (v/v) and 7% (v/v), 1% (v/v) and 6% (v/v), 2% (v/v) and 5% (v/v), 3% (v/v) and 4% (v/v). Accordingly, in some embodiments the concentration of formic acid that the cell or the promoter is exposed to is at least about 0.0001% (v/v), at least about 0.001% (v/v), at least about 0.01% (v/v), at least about 0.1% (v/v), at least about 1% (v/v), at least about 2% (v/v), at least about 2% (v/v), at least about 3% (v/v), at least about 4% (v/v), at least about 5% (v/v), at least about 6% (v/v), at least about 7% (v/v), at least about 8% (v/v), or at least about 9% (v/v). In some embodiments, the concentration of formic acid that the cell or the promoter is exposed to is about 0.0001% (v/v), about 0.001% (v/v), about 0.01% (v/v), about 0.1% (v/v), about 1% (v/v), about 2% (v/v), about 2% (v/v), about 3% (v/v), about 4% (v/v), about 5% (v/v), about 6% (v/v), about 7% (v/v), about 8% (v/v), about 9% (v/v), or about 10% (v/v).

In some embodiments, the concentration of the formate salt that the cell or the promoter is exposed to is between 0.0001% (w/v) and 10% (w/v). In some embodiments, the concentration of the formate salt that the cell or the promoter is exposed to is between 0.001% (w/v) and 9% (w/v), 0.01% (w/v) and 8% (w/v), 0.1% (w/v) and 7% (w/v), 1% (w/v) and 6% (w/v), 2% (w/v) and 5% (w/v), 3% (w/v) and 4% (w/v). Accordingly, in some embodiments the concentration of the formate salt that the cell or the promoter is exposed to is at least about 0.0001% (w/v), at least about 0.001% (w/v), at least about 0.01% (w/v), at least about 0.1% (w/v), at least about 1% (w/v), at least about 2% (w/v), at least about 2% (w/v), at least about 3% (w/v), at least about 4% (w/v), at least about 5% (w/v), at least about 6% (w/v), at least about 7% (w/v), at least about 8% (w/v), or at least about 9% (w/v). In some embodiments, the concentration of the formate salt that the cell or the promoter is exposed to is about 0.0001% (w/v), about 0.001% (w/v), about 0.01% (w/v), about 0.1% (w/v), about 1% (w/v), about 2% (w/v), about 2% (w/v), about 3% (w/v), about 4% (w/v), about 5% (w/v), about 6% (w/v), about 7% (w/v), about 8% (w/v), about 9% (w/v), or about 10% (w/v).

In some embodiments, the concentration of the formate ester that the cell or the promoter is exposed to is between 0.0001% (w/v) and 10% (w/v). In some embodiments, the concentration of the formate ester that the cell or the promoter is exposed to is between 0.001% (w/v) and 9% (w/v), 0.01% (w/v) and 8% (w/v), 0.1% (w/v) and 7% (w/v), 1% (w/v) and 6% (w/v), 2% (w/v) and 5% (w/v), 3% (w/v) and 4% (w/v). Accordingly, in some embodiments the concentration of the formate ester that the cell or the promoter is exposed to is at least about 0.0001% (w/v), at least about 0.001% (w/v), at least about 0.01% (w/v), at least about 0.1% (w/v), at least about 1% (w/v), at least about 2% (w/v), at least about 2% (w/v), at least about 3% (w/v), at least about 4% (w/v), at least about 5% (w/v), at least about 6% (w/v), at least about 7% (w/v), at least about 8% (w/v), or at least about 9% (w/v). In some embodiments, the concentration of the formate ester that the cell or the promoter is exposed to is about 0.0001% (w/v), about 0.001% (w/v), about 0.01% (w/v), about 0.1% (w/v), about 1% (w/v), about 2% (w/v), about 2% (w/v), about 3% (w/v), about 4% (w/v), about 5% (w/v), about 6% (w/v), about 7% (w/v), about 8% (w/v), about 9% (w/v), or about 10% (w/v).

The chemical structure of methanol is known to those skilled in the art.

The skilled person will understand that methanol is miscible in a variety of solvents. Accordingly, in one embodiment, the methanol is mixed in water. In one embodiment, the methanol is mixed in an organic solvent. In one embodiment, the solvent is dissolved or mixed in a mixture of an organic solvent and water. In some embodiments, the methanol is mixed in an organic solvent selected from the group comprising or consisting of: ether, acetone, ethyl acetate, glycerol, methanol, ethanol, benzene, toluene, or xylene. In one embodiment, the methanol is mixed in a mixture of ethanol and water. In some embodiments, the methanol is mixed in an appropriate culture medium.

In some embodiments, the concentration of the methanol that the cell or the promoter is exposed to is between 0.0001% (v/v) and 10% (v/v). In some embodiments, the concentration of methanol that the cell or the promoter is exposed to is between 0.001% (v/v) and 9% (v/v), 0.01% (v/v) and 8% (v/v), 0.1% (v/v) and 7% (v/v), 1% (v/v) and 6% (v/v), 2% (v/v) and 5% (v/v), 3% (v/v) and 4% (v/v). Accordingly, in some embodiments the concentration of methanol that the cell or the promoter is exposed to is at least about 0.0001% (v/v), at least about 0.001% (v/v), at least about 0.01% (v/v), at least about 0.1% (v/v), at least about 1% (v/v), at least about 2% (v/v), at least about 2% (v/v), at least about 3% (v/v), at least about 4% (v/v), at least about 5% (v/v), at least about 6% (v/v), at least about 7% (v/v), at least about 8% (v/v), or at least about 9% (v/v). In some embodiments, the concentration of methanol that the cell or the promoter is exposed to is about 0.0001% (v/v), about 0.001% (v/v), about 0.01% (v/v), about 0.1% (v/v), about 1% (v/v), about 2% (v/v), about 2% (v/v), about 3% (v/v), about 4% (v/v), about 5% (v/v), about 6% (v/v), about 7% (v/v), about 8% (v/v), about 9% (v/v), or about 10% (v/v).

The chemical structure of formaldehyde is known by those skilled in the art.

The skilled person will understand that formaldehyde is soluble in a variety of solvents. In some embodiments, the formaldehyde is dissolved in a solvent selected from the group comprising or consisting: water and acetone.

In some embodiments, the concentration of the formaldehyde that the cell or the promoter is exposed to is between 0.0001% (v/v) and 10% (v/v). In some embodiments, the concentration of formaldehyde that the cell or the promoter is exposed to is between 0.001% (v/v) and 9% (v/v), 0.01% (v/v) and 8% (v/v), 0.1% (v/v) and 7% (v/v), 1% (v/v) and 6% (v/v), 2% (v/v) and 5% (v/v), 3% (v/v) and 4% (v/v). Accordingly, in some embodiments the concentration of formaldehyde that the cell or the promoter is exposed to is at least about 0.0001% (v/v), at least about 0.001% (v/v), at least about 0.01% (v/v), at least about 0.1% (v/v), at least about 1% (v/v), at least about 2% (v/v), at least about 2% (v/v), at least about 3% (v/v), at least about 4% (v/v), at least about 5% (v/v), at least about 6% (v/v), at least about 7% (v/v), at least about 8% (v/v), or at least about 9% (v/v). In some embodiments, the concentration of formaldehyde that the cell or the promoter is exposed to is about 0.0001% (v/v), about 0.001% (v/v), about 0.01% (v/v), about 0.1% (v/v), about 1% (v/v), about 2% (v/v), about 2% (v/v), about 3% (v/v), about 4% (v/v), about 5% (v/v), about 6% (v/v), about 7% (v/v), about 8% (v/v), about 9% (v/v), or about 10% (v/v).

The chemical structures of ethanol, propanol, butanol and glycerol will be known by those skilled in the art.

The skilled person will understand that ethanol, propanol, butanol and glycerol are miscible in a variety of solvents. Accordingly, in one embodiment, the ethanol, propanol, butanol or glycerol is mixed in water. In one embodiment, the ethanol, propanol, butanol or glycerol is mixed in an organic solvent. In one embodiment, the solvent is dissolved or mixed in a mixture of an organic solvent and water. In some embodiments, the ethanol, propanol, butanol or glycerol is mixed in an organic solvent selected from the group comprising or consisting of: ether, acetone, ethyl acetate, glycerol, ethanol, propanol, butanol or glycerol, ethanol, benzene, toluene, or xylene. In one embodiment, the ethanol, propanol, butanol or glycerol is mixed in a mixture of ethanol and water. In some embodiments, the ethanol, propanol, butanol or glycerol is mixed in an appropriate culture medium.

In some embodiments, the concentration of the ethanol, propanol, butanol or glycerol that the cell or the promoter is exposed to is between 0.0001% (v/v) and 10% (v/v). In some embodiments, the concentration of ethanol, propanol, butanol or glycerol that the cell or the promoter is exposed to is between 0.001% (v/v) and 9% (v/v), 0.01% (v/v) and 8% (v/v), 0.1% (v/v) and 7% (v/v), 1% (v/v) and 6% (v/v), 2% (v/v) and 5% (v/v), 3% (v/v) and 4% (v/v). Accordingly, in some embodiments the concentration of ethanol, propanol, butanol or glycerol that the cell or the promoter is exposed to is at least about 0.0001% (v/v), at least about 0.001% (v/v), at least about 0.01% (v/v), at least about 0.1% (v/v), at least about 1% (v/v), at least about 2% (v/v), at least about 2% (v/v), at least about 3% (v/v), at least about 4% (v/v), at least about 5% (v/v), at least about 6% (v/v), at least about 7% (v/v), at least about 8% (v/v), or at least about 9% (v/v). In some embodiments, the concentration of ethanol, propanol, butanol or glycerol that the cell or the promoter is exposed to is about 0.0001% (v/v), about 0.001% (v/v), about 0.01% (v/v), about 0.1% (v/v), about 1% (v/v), about 2% (v/v), about 2% (v/v), about 3% (v/v), about 4% (v/v), about 5% (v/v), about 6% (v/v), about 7% (v/v), about 8% (v/v), about 9% (v/v), or about 10% (v/v).

In some embodiments, the propanol is selected from the group comprising or consisting: propan-1-ol and isopropanol. In some embodiments, the butanol is selected from the group comprising or consisting: butan-1-ol and butan-2-ol.

In the absence of an inducing agent, an inducible promoter is preferably incapable of driving transcription of a downstream nucleic acid sequence that is operably linked to the inducible promoter. It will be appreciated by those skilled in the art, however, that the inducible promoter of the invention may be “leaky”. If an inducible promoter is leaky, the inducible promoter is capable of driving transcription of a downstream nucleic acid sequence that is operably linked to the inducible promoter to at least some extent, even in the absence of an inducing agent. Transcription of a downstream nucleic acid sequence that is operably linked to the leaky inducible promoter is lower in the absence of an inducing agent than in the presence of an inducing agent.

Accordingly, in some embodiments, the inducible promoter may be capable of driving transcription of a downstream nucleic acid sequence that is operably linked to the inducible promoter in the absence of an inducing agent. In some embodiments, an inducible promoter comprising or consisting any of the isolated nucleic acids or nucleic acid sequences of the invention may be leaky. In some embodiments, the inducible promoter drives transcription of a downstream nucleic acid sequence that is operably linked to the inducible promoter in the absence of an inducing agent at a lower level than in the presence of an inducing agent.

That a particular promoter drives some degree of basal transcription in the absence of an inducing agent does not mean that the promoter is not useful. The utility of an inducible promoter typically resides in the degree of induction observed upon exposure to an inducing agent. It is also not necessarily the case that only promoters that are capable of very high levels of induction are useful. There are instances where the product of transcription may be toxic to the cell, and so only a low level of induction is required, for example. The inducible promoters provided by the present invention present a wide range of options to the skilled person for inducible expression, allowing the appropriate promoter sequence to be selected for each different circumstance.

In some embodiments, the level of induction in expression from the nucleic acid or promoter of the invention upon exposure to one or more inducing agents is:
between 1.25 and 1000 fold increase in expression, for example between 1.5 and 900, 1.75 and 800, 2.0 and 700, 2.5 and 600, 3 and 500, 4 and 450, 5 and 400, 6 and 350, 7 and 300, 8 and 250, 9 and 200, 10 and 150, 15 and 100, 20 and 90, 30 and 80, 40 and 70, 50 and 60 fold expression; and/or
at least 1.25, 1.5, 1.75, 2.0, 2.5, 3, 4, 5, 7, 7, 8, 9, 10, 15, 20, 30, 40, 50, 60, 70, 80, 90, 100, 150, 200, 250, 300, 350, 400, 450, 500, 600, 700, 800, 900 and 1000 fold expression.

The above increase in expression upon exposure to an inducing agent can be dependent on the concentration of inducing agent that the cell or promoter is exposed to. For example, in one embodiment the level of induction in expression from the nucleic acid or promoter of the invention upon exposure to one or more inducing agents is:
between 1.25 and 1000 fold increase in expression, for example between 1.5 and 900, 1.75 and 800, 2.0 and 700, 2.5 and 600, 3 and 500, 4 and 450, 5 and 400, 6 and 350, 7 and 300, 8 and 250, 9 and 200, 10 and 150, 15 and 100, 20 and 90, 30 and 80, 40 and 70, 50 and 60 fold expression; and/or
at least 1.25, 1.5, 1.75, 2.0, 2.5, 3, 4, 5, 7, 7, 8, 9, 10, 15, 20, 30, 40, 50, 60, 70, 80, 90, 100, 150, 200, 250, 300, 350, 400, 450, 500, 600, 700, 800, 900 and 1000 fold expression

wherein
where the inducing agent is a solid, the concentration of inducing agent that the cell or the promoter is exposed to is between 0.0001% (w/v) and 10% (w/v). In some embodiments, the concentration of inducing agent that the cell or the promoter is exposed to is between 0.001% (w/v) and 9% (w/v), 0.01% (w/v) and 8% (w/v), 0.1% (w/v) and 7% (w/v), 1% (w/v) and 6% (w/v), 2% (w/v) and 5% (w/v), 3% (w/v) and 4% (w/v). Accordingly, in some embodiments the concentration of inducing agent that the cell or the promoter is exposed to is at least about 0.0001% (w/v), at least about 0.001% (w/v), at least about 0.01% (w/v), at least about 0.1% (w/v), at least about 1% (w/v), at least about 2% (w/v), at least about 2% (w/v), at least about 3% (w/v), at least about 4% (w/v), at least about 5% (w/v), at least about 6% (w/v), at least about 7% (w/v), at least about 8% (w/v), or at least about 9% (w/v). In some embodiments, the concentration of inducing agent that the cell or the promoter is exposed to is about 0.0001% (w/v), about 0.001% (w/v), about 0.01% (w/v), about 0.1% (w/v), about 1% (w/v), about 2% (w/v), about 2% (w/v), about 3% (w/v), about 4% (w/v), about 5% (w/v), about 6% (w/v), about 7% (w/v), about 8% (w/v), about 9% (w/v), or about 10% (w/v); or
where the inducing agent is a liquid, the concentration of the inducing agent that the cell or the promoter is exposed to is between 0.0001% (v/v) and 10% (v/v). In some embodiments, the concentration of inducing agent that the cell or the promoter is exposed to is between 0.001% (v/v) and 9% (v/v), 0.01% (v/v) and 8% (v/v), 0.1% (v/v) and 7% (v/v), 1% (v/v) and 6% (v/v), 2% (v/v) and 5% (v/v), 3% (v/v) and 4% (v/v). Accordingly, in some embodiments the concentration of inducing agent that the cell or the promoter is exposed to is at least about 0.0001% (v/v), at least about 0.001% (v/v), at least about 0.01% (v/v), at least about 0.1% (v/v), at least about 1% (v/v), at least about 2% (v/v), at least about 2% (v/v), at least about 3% (v/v), at least about 4% (v/v), at least about 5% (v/v), at least about 6% (v/v), at least about 7% (v/v), at least about 8% (v/v), or at least about 9% (v/v). In some embodiments, the concentration of inducing agent that the cell or the promoter is exposed to is about 0.0001% (v/v), about 0.001% (v/v), about 0.01% (v/v), about 0.1% (v/v), about 1% (v/v), about 2% (v/v), about 2% (v/v), about 3% (v/v), about 4% (v/v), about 5% (v/v), about 6% (v/v), about 7% (v/v), about 8% (v/v), about 9% (v/v), or about 10% (v/v);
for example where the inducing agent is formate or formic acid.

It will be appreciated that a leaky inducible promoter comprising or consisting mutations may be more or less leaky than said leaky inducible promoter that does not comprise or consist mutations. An inducible promoter comprising any isolated nucleic acid or nucleic acid sequence of the invention may comprise a mutation as described herein that increases or decreases the level the inducible promoter drives transcription of a downstream nucleic acid sequence that is operably linked to the inducible promoter in the absence of an inducing agent.

In some embodiments, the inducible promoter comprising a mutation increases or decreases the level that the inducible promoter drives transcription of a downstream nucleic acid sequence that is operably linked to the inducible promoter in the absence of an inducing agent. In one embodiment, the inducible promoter comprising a mutation increases the level that the inducible promoter drives transcription of a downstream nucleic acid sequence that is operably linked to the inducible promoter in the absence of an inducing agent. In a preferred embodiment, the inducible promoter comprising a mutation decreases the level that the inducible promoter drives transcription of a downstream nucleic acid sequence that is operably linked to the inducible promoter in the absence of an inducing agent.

The present invention also provides methods of detecting the level of expression driven by a promoter of the invention. It will be appreciated that methods of detecting the level of expression driven by a promoter generally detect the presence or quantity of an expression product produced by a downstream nucleic acid operably linked to the promoter. Expression products may include but are not limited to RNA and protein. Accordingly, methods of detecting the level of expression driven by a promoter may detect the presence or quantity of RNA or protein.

In some embodiments, the RNA is selected from the group comprising or consisting: mRNA, rRNA, miRNA, siRNA, piRNA, snRNA, snoRNA, exRNA, scaRNA, lncRNA, gRNA, sgRNA, crRNA, and tracrRNA. In some embodiments, the method of detecting the presence or quantity of RNA is selected from the group comprising or consisting: RT-PCR, qRT-PCT, Northern blot, nuclease protection assays, and in-situ hybridisation, or any combination thereof.

In some embodiments, the method of detecting the level of expression driven by a nucleic acid or promoter of the invention detects the presence or quantity of RNA produced by a downstream nucleic acid operably linked to the promoter. In some embodiments, the level of expression driven by a nucleic acid or inducible promoter in the presence of an inducing agent may be determined by detecting the presence or quantity of RNA produced by a downstream nucleic acid operably linked to the promoter. In some embodiments, the level of expression driven by a nucleic acid or inducible promoter in the absence of an inducing agent may be determined by detecting the presence or quantity of RNA produced by a downstream nucleic acid operably linked to the promoter. In some embodiments, the difference in expression driven by an inducible promoter in the presence of an inducing agent compared to expression driven by an inducible promoter in the absence of an inducing agent may be determined by a method comprising the steps of i) detecting the presence or quantity of RNA produced by a downstream nucleic acid operably linked to the promoter in the presence and absence of an inducing agent and ii) correlating the presence or quantity of RNA produced by a downstream nucleic acid operably linked to the promoter in the presence and absence of an inducing agent with the level of expression driven by the promoter.

In some embodiments, the method of detecting the level of expression driven by a promoter detects the presence of quantity of protein. Appropriate means of detecting the expression level of a protein will be apparent to the skilled person, and can include the detection of fluorescence where the protein has fluorescent properties, such as GFP; other functional assays in the cases of enzymes; and immunodetection for example on a western blot.

In one embodiment, the nucleic acid and promoter of the invention is an isolated nucleic acid or promoter, meaning that the nucleic acid has been extracted and removed from its native locus, or has been produced synthetically. In one embodiment, when the sequence of the nucleic acid and promoter of the invention is the native sequence, it is not located at the native locus. For example, a nucleic acid or promoter of the invention can be introduced, e.g. by transformation and homologous recombination, into a cell, but where the sequence of the nucleic acid or promoter is the wild-type sequence, it is not introduced into the same cell type at the same locus as the wild-type sequence. This does not mean that the nucleic acid and promoter of the invention cannot be used in a cell, or even the same host cell species, for example through introduction on a plasmid or insertion into the genome at a non-native locus. Since the nucleic acids and promoters of the invention include mutated or truncated versions of the native nucleic acids and promoters, it is possible to re-introduce these sequences into the native host species, at the native locus, yet still result in a non-naturally occurring, or engineered cell, as described further below.

The isolation process itself results in a non-naturally occurring nucleic acid, since histone modifications tend to not be preserved during the isolation process.

It will also be apparent to the skilled person that the nucleic acid and promoters of the invention can be modified, for example modified relative to the naturally occurring promoter. For example, amplification of a sequence through PCR results in a nucleic acid fragment that is distinct to that which occurs in the native genomic locus, even if the sequence is identical, since an artificially amplified fragment will not be subject to the same epigenetic modifications that the naturally occurring sequence is exposed to. For example, histone and DNA methylation status is not preserved during PCR.

Accordingly, in some embodiments, the nucleic acids and promoters of the invention are not naturally occurring products, for at least this reason. In some embodiments the nucleic acids and promoters of the invention are produced by PCR based amplification methods, or are otherwise produced synthetically.

In some embodiments the nucleic acids and promoters of the invention comprise one or more restriction enzyme digestion sites that have been engineered into the nucleic acid or prompter, for example one or more type II restriction enzyme digestion sites. These sites can be readily incorporated into the nucleic acid or promoter of the invention through the use of tailed primers and a PCR amplification reaction. In one embodiment the restriction sites flank the nucleic acid or promoter of the invention. In one embodiment, restriction sites flanking the nucleic acid or promoter of the invention aid in cloning.

It will be apparent that the isolated nucleic acid or promoters of the invention can be incorporated into a larger nucleic acid construct that comprises additional sequence portions. For example, the invention provides a nucleic acid construct comprising at least a first and a second nucleic acid sequence, wherein the first nucleic acid sequence comprises or consists of the isolated nucleic acid sequence of the invention and described above.

Preferences for the isolated nucleic acid sequence of the invention are as described herein, for example the nucleic acid sequence of the invention in some embodiments is an inducible promoter, inducible by formate. Preferences for the length, sequence, sequence identity for example are as described above.

In the nucleic acid construct of the invention, preferably the first nucleic acid sequence is an inducible promoter, as described herein.

In one embodiment, expression from the inducible promoter is performed in YNB or ACH +caa media, or other minimal media.

It will be clear that the second nucleic acid sequence can be any sequence. In one embodiment the second nucleic acid sequence is a sequence capable of being transcribed into RNA, and the first nucleic acid sequence is operably linked to the second nucleic acid sequence. In some embodiments, the 3′ end of the first nucleic acid sequence is linked to the 5′ end of the second nucleic acid sequence by a sequence comprising or consisting the sequence CACA. The CACA has been shown to increase protein expression levels (Gasmi et al 2011 Appl Microbiol Biotechnol 89: 109-119).

It will be clear that the second sequence can be an RNA encoding sequence, or can be a protein encoding sequence.

In one embodiment the second nucleic acid sequence is transcribed into mRNA. In some embodiments the second nucleic acid sequence encodes a peptide or a polypeptide.

In other embodiments, the second nucleic acid sequence is capable of being transcribed into an RNA sequence selected from the group consisting of or comprising: mRNA, rRNA, miRNA, siRNA, piRNA, snRNA, snoRNA, exRNA, scaRNA, lncRNA, gRNA, sgRNA, crRNA, and tracrRNA.

In some embodiments of the nucleic acid construct of the invention, the first sequence is operably linked to one or more sequences selected from the group consisting or comprising: an enhancer sequence, an operator sequence, a silencer sequence, a kozak sequence, a Shine-Dalgarno sequence, a TATA box, a Pribnow box, a terminator sequence, a 5′ untranslated region sequence, a 3′ untranslated region sequence, a polyadenylation signal sequence, a 5′ upstream activator sequence, or any combination thereof.

In some embodiments of the nucleic acid construct of the invention, the second sequence is operably linked to one or more sequences selected from the group consisting or comprising: an enhancer sequence, an operator sequence, a silencer sequence, a kozak sequence, a Shine-Dalgarno sequence, a TATA box, a Pribnow box, a terminator sequence, a 5′ untranslated region sequence, a 3′ untranslated region sequence, a polyadenylation signal sequence, a 5′ upstream activator sequence, or any combination thereof.

In some embodiments the second nucleic acid sequence is a nucleic acid sequence which comprises or consists a natural occurring nucleic acid sequence. For example, in some embodiments the second nucleic acid sequence may be a sequence that is isolated from an organism. The skilled person will be aware that exemplary methods of isolating such sequences includes amplification from a template nucleic acid sequence. Amplification methods include but are not limited to PCR and ligase chain reaction.

In some embodiments, the second nucleic acid sequence is a nucleic acid sequence from Yarrowia lipolytica.

In some embodiments, the second nucleic acid sequence does not encode a formate dehydrogenase (FDH) gene, for example does not encode an FDH gene from Yarrowia, or from Yarrowia lipolytica.

For example, in some embodiments, the second nucleic acid is not a gene selected from the group consisting of YALI0E14256, YALI0F28765, YALI0F15983, YALI0F13937, YALI0E15840, YALI0C14344, YALI0C08074, YALI0B22506, YALI0B19976, YALI0A21353, YALI0E19657g, YALI0B21670g, YALI0F29315g, YALI0D25256g, YALI0C11099g, YALI0F09966g;
optionally from the group consisting of YALI0E14256, YALI0F28765, YALI0F15983, YALI0F13937, YALI0E15840, YALI0C14344, YALI0C08074, YALI0B22506, YALI0B19976, YALI0A21353.

In some embodiments, where:
the first nucleic acid sequence comprises or consists of a portion of SEQ ID NO: 8 or 24, or comprises or consists of SEQ ID NO: 8 or 24, the second nucleic acid does not encode YALI0E14256 (SEQ ID NO:40);
the first nucleic acid sequence comprises or consists of a portion of SEQ ID NO: 2 or 18, or comprises or consists of SEQ ID NO: 2 or 18, the second nucleic acid does not encode YALI0A21353 (SEQ ID NO:34);
the first nucleic acid sequence comprises or consists of a portion of SEQ ID NO: 3 or 19, or comprises or consists of SEQ ID NO: 3 or 19, the second nucleic acid does not encode YALI0F15983 (SEQ ID NO:35);
the first nucleic acid sequence comprises or consists of a portion of SEQ ID NO: 4 or 20, or comprises or consists of SEQ ID NO: 4 or 20, the second nucleic acid does not encode YALI0B22506 (SEQ ID NO:36);
the first nucleic acid sequence comprises or consists of a portion of SEQ ID NO: 5 or 21, or comprises or consists of SEQ ID NO: 5 or 21, the second nucleic acid does not encode YALI0C08074 (SEQ ID NO:37);
the first nucleic acid sequence comprises or consists of a portion of SEQ ID NO: 6 or 22, or comprises or consists of SEQ ID NO: 6 or 22, the second nucleic acid does not encode YALI0F13937 (SEQ ID NO:38);
the first nucleic acid sequence comprises or consists of a portion of SEQ ID NO: 7 or 23, or comprises or consists of SEQ ID NO: 7 or 23, the second nucleic acid does not encode YALI0C14344 (SEQ ID NO:39);
the first nucleic acid sequence comprises or consists of a portion of SEQ ID NO: 9 or 25, or comprises or consists of SEQ ID NO: 9 or 25, the second nucleic acid does not encode YALI0B19976 (SEQ ID NO:41);
the first nucleic acid sequence comprises or consists of a portion of SEQ ID NO: 10 or 26, or comprises or consists of SEQ ID NO: 10 or 26, the second nucleic acid does not encode YALI0E15840 (SEQ ID NO:42);
the first nucleic acid sequence comprises or consists of a portion of SEQ ID NO: 11 or 27, or comprises or consists of SEQ ID NO: 11 or 27, the second nucleic acid does not encode YALI0F28765 (SEQ ID NO:43);
the first nucleic acid sequence comprises or consists of a portion of SEQ ID NO: 12 or 28, or comprises or consists of SEQ ID NO: 12 or 28, the second nucleic acid does not encode YALI0E19657g (SEQ ID NO:44);
the first nucleic acid sequence comprises or consists of a portion of SEQ ID NO: 13 or 29, or comprises or consists of SEQ ID NO: 13 or 29, the second nucleic acid does not encode YALI0B21670g (SEQ ID NO:45);
the first nucleic acid sequence comprises or consists of a portion of SEQ ID NO: 14 or 30, or comprises or consists of SEQ ID NO: 14 or 30, the second nucleic acid does not encode YALI0F29315g (SEQ ID NO:46);
the first nucleic acid sequence comprises or consists of a portion of SEQ ID NO: 15 or 31, or comprises or consists of SEQ ID NO: 15 or 31, the second nucleic acid does not encode YALI0D25256g (SEQ ID NO:47);
the first nucleic acid sequence comprises or consists of a portion of SEQ ID NO: 16 or 32, or comprises or consists of SEQ ID NO: 16 or 32, the second nucleic acid does not encode YALI0C11099g (SEQ ID NO:48);
the first nucleic acid sequence comprises or consists of a portion of SEQ ID NO: 17 or 33, or comprises or consists of SEQ ID NO: 17 or 33, the second nucleic acid does not encode YALI0F09966g (SEQ ID NO:49);
optionally with a sequence with at least 80%, 85%, 90%, 92%, 94%, 95%, 96%, 97%, 98% or 99% sequence identity to the stated sequences.

The sequences of Yarrowia lipolytica FDH genes described herein are set out below.

>YALI0A12353g
SEQ ID NO: 34
ATGAAGGTCCTTCTTATTCTCTACGATGCCGGCTCCCACGCCAAGGACGAGCCCAAGCTGCTTGGATGCACCGAGAA
CGAGCTTGGTATTCGAGACTGGCTCGAGTCGCAGGGCCACACTCTCGTCACCACCTCTTCCAAGGACGGTGCCGACT
CTGTGCTTGACAAGGAGATTGTTGACGCTGATGTAGTCATCACCACCCCGTTCCACCCCGGCTACATCAACAAGGAGC
GAATTGACAAGGCCAAGAAGCTCAAGATCTGCATCACCGCCGGTGTCGGCTCCGACCACGTGGACCTCGATGCTGCC
AACGCCCGAAACATTGCCGTTCTCGAGGTCACCGGCTCGAACGTCCAGTCCGTCGCTGAGCATGTTGTCATGACCAT
GCTGGTGCTGGTCCGAAACTTTGTCCCTGCCCACGAGCAGATTATTTCTGGCGGCTGGGACGTTGCTGCGGTTGCCA
AGGACTCTTACGATCTCGAGGGCAAGGTCATCGGTACCGTTGGAGGCGGTCGAATCGGCCAGCGAGTGCTCAAGCG
ATGCAAGCCCTTTGACCCCATGGAGATGCTCTACTACGACTACCAGGCTATGCCAGCTGATGTGGAGAATGAGATTG
GATGCCGACGAGTCGAGTCTCTGGAGGAGATGCTCTCTCTCTGTGACGTTGTAACCATCAACTGTCCTCTCCATGCCT
CCACCAAGGGTCTCTTCAACAAGGAGCTCATTTCCCACATGAAGGACGGCGCTTGGCTCGTCAACACTGCTCGAGGA
GCCATCTGCGTCACCGAGGACATTGTCGACGCTCTCGAGTCCGGCAAGATCCGAGGCTACGGTGGTGACGTCTGGTT
CCCCCAGCCTGCCCCCAAGGACCACCCCTGGAGAACCATGCGAAACAAGTACGGCGGTGGAAACGCCATGACTCCC
CATATCTCCGGTACTTCCATCGACGCCCAGGGCCGATACGCTGAGGGTACCAAGAAGATTCTTGAGGTCTTTTTCTCT
GGCAAGCAGGATTACCGCCCCCAGGACATTATCTGCATCAACGGTCACTACGGTACCAAGGCTTACGGTGACGACAA
GGAGCACAAGGCCCACGTTTCCAAGTAGCTGTTTAACTTATATAAGCAATTGACTGGATTTGATAACGA
>YALI0F15983g
SEQ ID NO: 35
ATGAAGGTCCTTCTTATTCTCTACTCTGCCGGTTCTCACGCCGTTGACGAGCCCAAGCTACTCGGTTGCACTGAGAAC
GAGCTCGGTCTCCGAAAGTGGCTCGAGTCCCGGGGCCACACGCTTGTGACCACCTCTTCAAAGGAGGGAGCCGACT
CTGTGCTCGACAAGGAGATTGTCGATGCCGACATTGTCATCACTACTCCCTTCCACCCCGGTTACATCACCCGAGAGC
GAATCGCCAAGGCCAAGAACCTCAAAATTTGCATCACTGCCGGTGTTGGTTCGGACCACGTCGATCTTGATGCTGCCA
ACGAGCGAGATATTGCCGTTCTCGAGGTCACTGGCTCCAACGTGCAGTCTGTGGCTGAGCACGTTGTCATGACCATG
CTGGTCCTGGTCCGAAACTTTGTGCCTGCCCACGAGCAGGTCATGGCTGGTGGCTGGGACGTTGCTGCTGTCGCCAA
GGACTCCTACGACATTGAGGGAAAGGTCATTGGTACTGTGGGAGGTGGCCGAATTGGTCAGCGAGTCCTAAAGCGA
GTTGCTCCCTTCAACCCTAAGGAGATGCTCTACTACGACTACCAGGGTCTGTCTGCTGAGACCGAGAAGGAGCTGAA
CTGCCGACGAGTCGAGAAGCTCGAGGATATGCTTGCCCAGTGTGATATCGTCACCATCAACTGTCCCCTCCACGAGT
CCACCAAGGGTCTCTTCAACAAGGAGATGCTCTCTCACATGAAGAAAGGTGCCTGGCTTGTCAACACCGCTCGAGGA
GCCATCTGTGTCAAGGAAGACGTTGCTGAGGCTCTCAAGAACGGCCAGCTGCGAGGCTACGGAGGTGACGTCTGGT
TCCCCCAGCCTGCTCCCGCTGACCACCCCTGGAGATCCATGCGAAACAAGTACGGAGCTGGAAACGCCATGACCCCC
CATATCTCCGGTACTTGCATCGATGCCCAGGTTCGGTACGCCCAGGGAACCAAGAACATTCTCGACATGTTCTTCTCC
GGTAAGCAGGACTACCGACCCCAGGACATCATCTGCATCAACGGTCACTACGGAACCAAGGCCTACGGTGATGACAA
GGAGCATGCTAAAAAGTAGGCTGGACGTCGAAATAAACACATTGTTCATCTCTGGGTAGCCCAATAGTAAATAGGTAA
GCTGTGATCATTA
>YALI0B22506g
SEQ ID NO: 36
ATGAAGATTCTCCTTATCCTTTACGACGCCGGCTCCCACGCTGCTGACGAACCCAAGCTGCTAGGTTGCACCGAGAAC
GAGCTCGGCATCCGATCCTGGCTCGAGTCCCAGGGCCATACCCTGGTCACCACCTCTTCCAAGGAGGGTGCTGACTC
TGTTCTCGACAAGGAGATTGTTGATGCCGATGTTGTCATCACCACTCCCTTCCACCCCGGCTACATCACCCGAGAGCG
AATTGCCAAGGCCAAGAACCTCAAGATCTGTGTCACTGCCGGTGTTGGCTCTGACCACGTTGATCTTGCTGCCGCCAA
CGAGAGAAACATTGCCGTGCTCGAGGTCACTGGTTCCAACGTGACTTCTGTTGCTGAGCACGTTGTCATGACCATGCT
TGTTCTTGTCCGAAACTTCGTGCCTGCCAACGAGCAGGTTCGAGGCGGCGGCTGGGACGTTGCCGGTGTTGCCAAG
GATTCCTACGATATTGAGGGTAAGGTCATTGGTACCGTCGGAGTCGGCCGAATCGGCAAGCGAGTGCTCCAGCGACT
CAAGCCCTTCGACCCCAAGGAGCTGCTCTACTACGACTATCAGCCTCTGTCTGCTGCCGACGAGAAGGAGATTGGCG
CCCGACGAGTTGAGAAGCTCGAGGACATGCTTGCCCAGTGTGATGTTGTCACCATCAACTGCCCCCTGCACGAATCC
ACCAAGGGTCTCTTTAACAAGGAGCTGCTGTCCCACATGAAGAAGGGCGCCTGGCTCGTCAACACCGCTCGAGGAGC
CATCTGTGTAAAGGAGGACGTTGCCGCCGCTCTCAAGTCTGGTCAGCTCCGAGGCTACGGCGGAGATGTCTGGTTCC
CCCAGCCTGCCCCTGCTGACCACCCCTGGAGAAAGATGGTCAACAAGTACGGTGCCGGTAACGCCATGACCCCCCAC
ATGTCTGGAACCTCTCTGGACGCCCAGGCCCGATACGCTGCTGGTGTCAAGCAGATTCTCGACGAGTTCTTCTCCGG
CCGAGAACAGTACCGACCCCAGGACATTATCTGCTACGGTGGTAACTACGGCACCAAGGCCTACGGAGACGACAAGA
AGGTCGTCGACAAGAAGTAATCCGTCCAACGTTCTTCTCAGCGCACAGGTACCTATACTACTGGTAGTATCAGGTTAA
ACCTCTTTTAGTTTAATGATAAGATAAGATAACATACCGT
>YALI0C08074g
SEQ ID NO: 37
ATGAAGGTCCTTCTTGTTCTTTACGATGCTGGCTCCCATGCCAAGGATGAGCCCAAGCTGCTTGGATGCACCGAGAAC
GAGCTCGGTATCAGAGACTGGCTCGAGTCTCAGGGACATACCCTTGTAACTACGTCTTCCAAGGACGGTGCCGACTC
TGTCCTCGACAAGGAGATTGTCGATGCCGATGTAGTCATCACCACCCCCTTTCATCCTGGATACATCAACAAGGAGCG
AATCGACAAAGCCAAGAAGCTCAAGATTTGCATCACTGCTGGTGTCGGCTCTGACCACGTCGATCTCGACGCTGCCA
ACGCCCGAGATATTGCTGTTCTCGAAGTCACCGGCTCCAATGTCCAGTCTGTTGCCGAGCACGTTATCATGACCATGT
TGGTCCTGGTGCGAAACTTTGTCCCTGCTCACGAGCAGATCATCTCTGGCGGTTGGGACGTTGCTGCTGTTGCCAAG
GACTCATACGATCTCGAGGGTAAGGTCATCGGCACCGTTGGAGGAGGCCGAATTGGCCAGCGAGTGCTCAAGCGAT
GCAAGCCCTTCGACCCCATGGAGATGCTCTACTACGACTATCAGGCCATGCCAGCTGACGTTGAGAAGGAGATTGGA
TGTCGTCGAGTCGAGTCTCTCGAGGAGATGCTCTCTCTTTGTGACGTGGTCACCATCAACTGCCCTCTGCACGCTTCC
ACCAAGGGCCTGTTCAACAAAGAGCTCATCTCGCACATGAAGGATGGTGCCTGGCTCGTTAACACCGCTCGAGGAGC
CATTTGTGTCACCGAGGACATTGTCGAGGCTCTCGAGTCCGGTAAGATCCGAGGTTACGGCGGAGACGTTTGGTTCC
CCCAGCCTGCCCCTAAGGACCACCCCTGGAGAACCATGCGAAACAAGTACGGGGGAGGAAACGCCATGACTCCTCAT
ATCTCCGGAACTTCCATCGACGCCCAGGGCCGATATGCCGAGGGCACCAAGAAGATCCTTGAGGTTTTCTTTTCTGGA
AAGCAGGATTATCGACCCCAGGACATTATCTGCATCAATGGTCACTACGGCACAAAAGCTTACGGAGATGACAAGGA
GCACAAGGAGCACCAGCTCAAGTAAATATATATAGCTAGAAACATATCTCGTTGTTGCTT
>YALI0F13937g
SEQ ID NO: 38
ATGAAGGTCCTTCTTATTCTCTATGATGCCGGCTCTCACGCTAAGGATGAGCCCAAGCTCCTTGGATGCACCGAGAAC
GAGCTCGGTATCCGAGACTGGCTCGAGTCTCAGGGCCATACTCTCGTTACCACCTCTTCCAAGGATGGCGCCGACTC
TGTGCTTGACAAGGAGATTGTTGACGCCGATGTAGTCATCACTACCCCCTTCCACCCTGGCTACATCAACAAGGAGCG
AATCGACAAGGCCAAGAAGCTCAAGATCTGCATCACTGCCGGTGTGGGCTCGGACCACGTTGATCTCGATGCTGCCA
ACGCTCGAGATATCGCTGTTCTGGAAGTCACTGGTTCTAATGTCCAGTCCGTCGCTGAGCATGTTGTCATGACTATGC
TCGTTCTGGTCCGAAACTTTGTCCCCGCCCACGAGCAAATCATCTCTGGCGGCTGGGATGTTGCTGCTGTCGCCAAG
GACTCCTACGATCTCGAGGGTAAGGTCATCGGTACCGTTGGTGGCGGCCGAATCGGCCAGCGTGTCCTCAAGCGAT
GCAAGCCCTTCGACCCCATGGAAATGCTCTACTACGACTACCAGCCCATGCCTGCCGACGTCGAAAAGGAGATTGGT
TGCCGACGAGTTGAGTCTCTGGAGGAGATGCTCTCTCTGTGTGATGTCGTGACCATCAATTGCCCTCTGCACGCCTCC
ACCAAGGGCCTCTTCAACAAGGAGCTCATCTCGCACATGAAGAATGGTGCTTGGCTCGTCAACACTGCTCGAGGAGC
CATCTGCGTTACCGAGGACATTGTCGAGGCACTCGAGTCCGGCAAGATGCGAGGATACGGTGGTGATGTCTGGTTCC
CCCAGCCTGCCCCCAAGGACCATCCCTGGAGAACCATGCGAAACAAGTACGGTGGTGGAAACGCCATGACTCCTCAT
ATCTCTGGCACTTCCATCGATGCCCAGGGCCGATACGCCGAGGGAACAAAGAAGATCCTCGAGGTCTTCTTCTCTGG
CAAGCAGGATTACCGACCCCAGGACATTATCTGCATCAACGGTCACTACGGTACCAAGGCTTACGGTGACGACAAGG
AGCACAAGGAGCACCAGCTTAAGTAAACATTTTTATAGTAACATCTCATTTTGT
>YALI0C14344g
SEQ ID NO: 39
ATGAAGGTCCTTCTTGTTCTATACGATGCCGGCTCCCACGCCAAGGATGAGCCAAAGCTACTTGGATGCACCGAGAA
CGAGCTCGGAATCAGAGACTGGCTCGAGTCACAGGGCCACACTCTTGTCACCACCTCTTCCAAGGACGGTGCCCACT
CTGTGCTTGACAAGGAGATTGTCGATGCCGATGTAGTCATCACCACCCCTTTCCACCCCGGCTACATCAACAAGGAGC
GAATTGACAAGGCCAAAAAGCTCAAGATCTGCATCACCGCTGGTGTTGGCTCCGACCACGTCGATCTCGACGCAGCC
AACGCCCGAGATATCTCTGTTCTTGAGGTTACTGGTTCTAATGTCCAGTCTGTCGCAGAGCACGTCGTTATGACCATG
CTGGTGCTGGTCCGAAACTTTGTGCCCGCCCACGAGCAGATCATCGAGGGCGGCTGGAATGTGGCTGCTGTCGCCA
AGGACTCGTACGATCTCGAGGGAAAGGTCATTGGCACCGTTGGAGGCGGTCGAATCGGCCAGCGAGTGCTCAAGCG
ATGCAAGCCCTTTGACCCCATGGAGATGCTCTACTACGACTACCAGGCTATGCCAGCTGATGTGGAGAAGGAGATTG
GATGCCGACGAGTCGAGTCTCTGGAGGAGAAGCTCTCTCTCTGTGACGTTGTCACCATCAACTGTCCCCTTCACGCCT
CCACCAAGGGTCTCTTCAACAAGGAGCTCATTTCCCACATGAAGGACGGCGCTTGGCTCGTCAACACTGCTCGAGGA
GCCATCTGCGTCACCGAGGACATTGTCGACGCTCTCGAGTTGGGCAAGATCCGAGGATATGGCGGTGACGTCTGGTT
CCCCCAGCCTGCCTCCAAGGACCACCCCTGGAGAACCATGCGAAATAAGTACGGCGGTGGAAACGCCATGACTCCCC
ACATCTCCGGCACTTCCATCGACGCCCAGGGCCGATACGCTGAGGGTACCAAGAAGATCCTCGAGGTCTTTTTCTCG
GGAAAGCAGAATTACAGACCCCAGGATATCATCTGTATCAACGGCCATTATGGTACAAAGGCCTACGGTGACGACAA
GGAGCACAAGGGGCACCAGCAGAAGTGA
>YALI0E14256g
SEQ ID NO: 40
ATGAAGATCCTTCTTGTTCTCTACGACGCCGGTTCCCACGCCAAGGATGAGCCTCGACTTCTCGGATGTACCGAGAAC
GAGCTTGGTCTCCGTGACTGGATCGAGTCCCAGGGACACACCTTGGTGACCACCTCCGACAAGGACGGTGAGAACTC
AACCGTCGACAAGGAGATTGTGGATGCTGAGATTGTTATCACCACCCCCTTCCACCCCGCTTACATCACCAAGGAGCG
AATTGACAAGGCCAAGAAGCTCAAGATCTGTATCACTGCCGGTGTTGGTTCCGACCATGTCGACCTTGATGCCGCCAA
CGCCCGAGACATTGCCGTCCTCGAGGTTACCGGATCTAACGTCCAGTCCGTCGCTGAGCACGTTGTCATGACCATGC
TGGTTCTGGTCCGAAACTTCGTCCCCGCCCACGAGCAGATCATCGAGGGTGGCTGGAATGTCGCTGCTGTCGCCAAG
GACTCTTACGACATTGAGGGTAAGGTCATTGGTACTGTCGGCGGTGGCCGAATTGGTCAGCGAGTCCTGAAGCGACT
TGCACCCTTCAACCCCATGGAGCTCCTCTACTACGACTACCAGCCCATGCCCAAGGACGTGGAGAAGGAGATTGGCT
GCCGACACGTCCCTGATCTTAAGGAGATGCTCTCTGTCTGTGACATTGTTACCATCAACTGTCCTCTCCACGACTCTAC
CAAGGGTATGTTCAACAAGGAACTCATCTCCCACATGAAGGATGGTGCTTGGCTCGTCAACACCGCCCGAGGTGCCA
TCTGTGTCACTGACGACATTGTTGAGGCCCTCAAGTCCGGTAAGATCCGAGGCTACGGTGGTGATGTCTGGAACCCC
CAGCCTGCCCCCAAGGACCACCCCTGGAGATACATGCGAAACAAGTGGGGCGGTGGAAACGCCATGACCCCCCATA
TCTCCGGTACCTCCATCGATGCCCAGGGCCGATACTCCGAGGGTACCAAGAACATTCTCGAGGTCTACTTCTCCGGAA
AGCAGAACTACCGACCTCAGGATGTCATCTGTATCAACGGCCACTACGGCACTAAGGCTTACGGTGACGACAAGGAG
CACAAGGCCCACGTTTCCAAATAAGCACTTTAGCTGTTTAACTTATAAGCAATTGACTACTAGAATTTGC
>YALI0B19976g
SEQ ID NO: 41
ATGAAGGTCCTTCTTGTTCTCTACGACGCCGGCTCCCACGCCAAGGATGAGCCCAAGCTACTTGGATGCACTGAAAAC
GAGCTCGGTATCCGAGACTGGCTCGAGTCCCAGGGCCATACCCTGGTAACTACCTCTTCTAAGGATGGCGCCGACTC
CGTTCTCGACAAGGAGATTGTTGATGCCGATGTTGTCATCACTACCCCCTTCCATCCCGGTTACATCAACAAGGAGAG
AATTGACAAGGCCAAGAAGCTCAAGATCTGTATCACTGCCGGTGTTGGTTCTGACCATGTCGATCTAGATGCCGCCAA
CGCCCGAGACATTGCTGTCCTTGAAGTCACTGGCTCCAACGTTCAGTCGGTCGCTGAGCATGTTGTCATGACCATGCT
TGTTCTGGTCAGAAACTTCGTCCCTGCTCATGAGCAGATCATTTCTGGTGGCTGGGACGTTGCCGCTGTCGCCAAGG
ACTCTTACGACCTAGAGGGTAAGGTCATTGGTACAGTTGGAGGTGGGCGAATCGGTCAGCGAGTCCTCAAGCGATGC
AAGCCCTTCGACCCCATGGAGATGCTCTACTACGACTACCAGCCCATGCCCGCTGATGTCGAGAAGGAGATTGGCTG
TCGACGAGTGGAGTCTCTCGAGGAGATGCTCTCTCTCTGTGACGTTGTCACCATCAACTGTCCTCTGCACGCCTCCAC
CAAGGGCCTCTTCAACAAGAAGCTCATCTCCCACATGAAGGATGGCGCCTGGCTCGTCAACACCGCTCGAGGAGCTA
TTTGTGTCACTGAGGACATTGTTGAGGCTCTCGAGTCCGGAAAGATTCGAGGCTACGGTGGTGATGTCTGGTTCCCT
CAGCCCGCCCCAAAGGACCACCCCTGGAGAACAATGCGAAACAAGTACGGTGGAGGAAACGCCATGACCCCTCATAT
CTCTGGTACTTCTATTGATGCCCAAGGCCGATACGCTGAGGGCACCAAGAAAATCCTTGAGGTCTTCTTCTCAGGAAA
GCAAGACTACCGACCCCAGGATATCATCTGTATCAACGGTCACTACGGTACCAAGGCTTACGGTGACGACAAGGAGC
ACAAGGAGCACAAGGAGCACGAGGTCAAATAAGCTATCAGCTCTATAGTACTGTACAGTAGGTACAATAGGATCTTGA
TATTACAGT
>YALI0E15840g
SEQ ID NO: 42
ATGAAGGTCCTTCTTGTTCTTTACGATGCTGGCTCTCACGCTGCTGACGAGCCCAAGCTCCTTGGATGCACCGAAAAC
GAGCTCGGTATCCGAGACTGGCTCGAGTCCCAGGGCCACACGCTTGTCACCACCTCTTCCAAGGATGGCGCCGACTC
TGTGCTCGACAAGGAAATTGTTGACGCCGACGTTGTCATCACCACCCCCTTCCACCCCGGCTACATCAACAAGGAGC
GAATCGACAAGGCCAAGAAGCTCAAGATCTGCATCACCGCCGGAGTCGGCTCGGACCACGTTGATCTCGATGCTGCC
AACGCTCGAGATATCGCTGTTCTGGAGGTCACTGGTTCTAATGTCCAATCCGTCGCTGAGCATGTTGTCATGACTATG
CTCGTTCTGGTCCGAAACTTTGTCCCCGCCCACGAGCAGATCATTTCCGGCGGGTGGGACGTTGCGGCGGTCGCCAA
GGACTCCTACGATCTAGAGGGTAAGGTCATCGGTACCGTGGGAGGCGGCCGAATCGGTCAGCGTGTCCTCAAGCGA
TGCAAGCCTTTCGACCCCATGGAAATGCTCTACTACGATTACCAGCCCATGCCTGCCGACGTCGAAGAGGAGATTGG
CTGCCGACGAGTCGAGTCGCTCGAGCAAATGCTTTCTCTGTGTGATGTTGTCACTATCAACTGCCCTCTGCACGCCTC
CACCAAGGGTCTCTTCAACAAGGAGCTCATCTCCCACATGAAGGATGGAGCCTGGCTCGTCAACACTGCTCGAGGAG
CCATCTGCGTCACCGAGGACATTGTCGAGGCCCTAGAGTCCGGCAAAATCCGAGGATACGGAGGAGACGTCTGGTTC
CCCCAGCCTGCCCCCAAGGACCACCCCTGGAGAACCATGCGAAACAATTACGGTGGTGGAAACGCCATGACTCCCCA
CATCTCCGGAACCTCCATTGATGCCCAGGGCCGATACGCTGAGGGTACCAAGAAGATCCTCGAGGTCTTTTTCTCGG
GCAAGCAGGATTATCGACCCCAGGATATCATCTGTATCAACGGCCATTATGGTACCAAGGCCTACGGTGACGACAAA
GAGCATAAGGAGCATCAGCTCAAGTAA
>YALI0F28765g
SEQ ID NO: 43
ATGAAGGTCCTTCTCATTCTCTACGACGCCGGCTCCCACGCTGTTGATGAGCCCAAACTACTCGGATGCACCGAGAAC
GAGCTCGGTATCCGATCATGGCTCGAGTCCCAGGGCCATACTCTGGTAACCACCTCTTCCAAGGATGGCGATGACTC
TGTCCTCGACAAGGAAATTGTCGACGCCGATGTCGTCATCACCACTCCCTTCCATCCCGGTTACATTACCCGAGAGCG
AATTGCCAAGGCCAAGAACCTCAAAATATGTGTTACTGCAGGTGTTGGTTCCGACCATGTCGACCTTGATGCCGCCAA
CGAGCGAGACATTGCCGTTCTCGAGGTCACTGGTTCCAACGTCCAATCCGTCGCTGAGCACGTCGTCATGACCATGC
TCGTCCTCGTCCGAAACTTTGTCCCCGCTCACGAGCAGGTCATGGCTGGTGGTTGGGATGTTGCTGCTGTCGCCAAG
GACTCTTATGACATCGAGGGTAAGGTCATTGGTACCGTTGGTGGTGGCCGAATTGGTCAGCGAGTCCTCAAGCGAGT
GGCTCCCTTCAACCCTAAGGAGATGCTCTACTACGACTACCAAGGACTTTCTGCTGAGACCGAGCAGGAGCTCAACT
GTCGACGAGTCGAGAAGCTTGAAGATATGCTTGCCCAATGTGACATTGTCACCATTAACTGTCCTCTCCACGAGTCTA
CCAAGGGCCTCTTCAACAAGGAGATGCTCTCTCACATGAAGAAGGGTGCTTGGCTTGTCAACACCGCTCGAGGAGCT
ATCTGTGTCAAGGAGGATGTCGCCGAGGCTCTTGCCAACGGCCAGCTTCGAGGCTACGGTGGTGATGTGTGGTTCCC
TCAGCCAGCCCCTGCCGACCACCCTTGGAGATCGATGCGAAACAAGTACGGCGCCGGTAACGCTATGACCCCACACA
TATCTGGTACTTCCATTGACGCCCAGGCCCGATACGCCGAGGGCACCAAGAACATTCTCGAGGTCTTCTTCTCCGGAA
AGCAGGACTACCGACCCCAGGATATCATCTGTATCAACGGTCACTACGGTACCAAGGCTTACGGCGACGACAAGGAG
CACGCCAAGAAATAGATAATATTCATTAACATACAAATACAGTCTACAGTCCTATATCGATTCGGTCGCTTCGAG
>YALI0E19657g
SEQ ID NO: 44
ATGTCTACGAAACCAACCATTGCGCTAGCGGGAGGGACGTCGCGGTTCGACCAGGTTCTGAGCCGGCTGGAACACTT
CAACACCATCTTCTACCCCGTGCCTGCCACCAAGGAGGAGCTGTTCAAAGACTGTCTACCCGGCGGTCCCCTGGCCA
ACGTGGAGGGTCTGTTTTGCTCCTGGCCGGCATTCTACGCCATGGGAGGCCTCAAGACCGAGGAGGAGATTGCCCA
GTTGCCGGCGAGTCTCAAGGTGGTGGCTCTCTGTGCCACGGGGTACGACCAGTTCAACGTTGCTGCGTTCCGGAAAC
GTGGCATCATTGTGACCAACACTCCGTCGATGGAGCCCTCTCACAGCGGCGAGCAGGTCGCGGACATTGCGCTCTAT
CTGTCGATTGGATGCATGCGCAAAATCCCCCTGTTTGAGGGCTCGTTCCAGAGAGAGAAGAACTCGATCGACGCGCG
GCGTGTGGTGGCGCAGGGCGAGTTTGACCCCGCTTCGGGCCAGGTTCTCGGTGGTCCGCCCTCAGGATTCGCCTTT
GGAGATCTCACCGCGAAAGGACCCGCCAGAATGTGCCGAAACCGGGCGGTGGGCGTGGCGGGACTCGGGAACATT
GGAAAGGCCATTGTGCGACGCTTGGCGCCTCTGGGGGTCGAAATTCACTACTACAAGCGATCCGAGCTGAGCGCTGA
GGAGCTGGCGGCCTCAAATCTCCCCTCAGACCTCATCTTCCACTCCTCCTTTGACGATCTGTGCGCCGCTTCGGACCA
GCTCATTCTGGCGCTCCCCGGAGGAGCAGACACCCTCAACATTGTCAACAAACGGACCCTGAACCTCATGCCCCGAG
GAGCGTCCATCGTCAACATTGGTCGAGGCACTCTCATCAACGAAGACGATCTGCTCGAGGCGCTGGGCTCCAAACAG
ATTGCCACCGCAGGCCTCGACGTCCAGGTGGGTGAGCCGTTTGTCAATCCCAAGCTATTTGGGCGGTGGGATATCCA
ACTGCTGCCCCATCTGGGCAGTGGAACCGAGGACAATGCCCTGGCGGCCGAGCTCAATGTGATTGACAACATTGAAA
ATGTCCTGAACGGCGGTCCTGGTCTTCATCCTGTCAATTGAGCTTTGACAATGACAACCATGACAACCATGACAACTA
TGGCAACCATGACGAACAGCGACCAACAACGACCAACAATGACAAACAGCGACGAACAACGACGACAGCGACGAAC
AGCGACGACAGCGACGAAACATGTTCATGGATAGTACATGCCCTACAAAAGGGACATTCGTTCCCTAGACTAGCTAAT
ACGAGCCCCTTTCTCGTCGCCAAAGACATCTATAAGCTATATTGAACTAAACTGTACGAAACTTGTGGTAACATGAACC
ACTAGAGTCACTG
>YALI0B21670g
SEQ ID NO: 45
ATGCTCCGTATCCGACCACAGTTTGCGCGACAGTCGCTCGCCCGATTGTACTCGACCCAAAGACAGAAGATCCTGTTC
CTTGACAACGTGGTGGACGCCAAAGACGAGTTTGAAAAGCTCAAGAAGAAGGCCGACGTGGTGACTCTCAAGGATG
GAACTGACCGAGAATCGTTTCTCAAGGACCTCAAAAACACCTACAAGGACATCAATGCCATCTTCCGAACCTTCATCA
GTGTCAAAAAGACCGGCCGGTTCGACGAGGAGCTGGCCAAGGCTCTTCCTGAGTCCTGCAAAGCTGTCTGCCATTAC
GGAGCAGGCTATGACCAGATCGATGTGCCCTTCTTCTCCGAAAGAGGCATTCAGGTCAGCAACGTGCAGTCCATGGC
CGACGAGTCGACTGCTCTCACGAACCTGTATCTAATGATCGGCACCCTGCGAAACTTCGGAGACGGAGCTCTCAACTT
GCAAAAGGGCCAATGGCTCAAGGGAGTGGCCCTGGGCAACGACATCTCCGGCAAGACTCTCGGAATTCTCGGTATG
GGCGGAATCGGCCGAGAAATCAGAGACTACGTGGCTCCTCTGGGTTTCAGCAAGGTGCTGTACTACAACCGAAATCG
CCTGGCCCCCGAGCTGGAAAAGGACTCTGTCTACTGCCAGTCGCCTGAAGATCTATTTTCCAAGGCCGATGTCATTTC
CATCAACGTGCCTCTTAATGCTGCTACCAAGCACCTCGTCAACGCAGAGTCCATCTCCAAAATGAAGGACGGAGTCAT
CATTGTCAACACCGCTCGAGGCCCTGTTTGCGACGAGAAGGCCCTAGTGGACGGTCTAAACTCTGGAAAGATTGGAG
GAGTCGGTCTCGATGTATTCGAGCGAGAGCCTGCCATTGAAGAAGGTCTTTTGAAACACCCTAGAACCCTGCTTCTGC
CTCATATGGGAACCTGGTCCCACGAGACCCATTTCAAGATGGAGAAGGCGGTTCTGGACAACCTCGAGAGCTTTGTT
GATACTGGAAAGGTTATCTCCATTGTTCCTGAGCAGAAGGGCAAGTTTTAAAATGGTAACAACTAACATAGATAGGGG
GAGGGGGAGCTACAGTTTCCAGTAGCTAAGTAAGTACAAGTAGAGCTAAACGCATAAACATGCATTGTGTAGAAAGT
>YALI0F29315g
SEQ ID NO: 46
CACGCGGCTGATGAGCCCAAACTCCTCGGATGCACCGAGAACGAGCTCGGTATCCGATCATTGCTCGAGTCCCAGGG
CCATACTCTGGTAACCACCTCTTCCAAGGATGGCGATGATTCTGTCCTCGACATGGAAATTGTCGACGCCGATGTCGT
CATCACCACTCCCTTCCACCCCGGTTACATTACTCGAGAGCGAATTGACAAGGCCAAGAAGCTCAAGATCTGTATCAC
TGCCGGTGTTGGTTCCGACCATGTCGATCTAGATGCCGCCAACGCCCGAGACATTGCTGTCCTTGAAGTCACTGGCT
CCAACGTTCAGTCGGTCGCTGAGCATGTTGTCATGACCATGCTTGTTCTGGTCAGAAACTTCGTCCCTGCTCATGAGC
AGATCATTTCTGGTGGCTGGGACGTTGCCGCTGTCGCCAAGGACTCTTACGACCTTGAGGGTAAGGTCATTGGTACA
GTTGGAGGGACGAATTGGTCAGCGAGTACTCAAGCGATGCAAGCCCTTCGACCCCATGGAGATGCTCTACTACGACT
ACCAGCCCATGCCCGCTGATGTCGAGAAGGAGATTGGCTGTCGACGAGTGGAGTCTCTCGAGAAGATGCTCTCTCCC
TGTGACGTTGTCACCATCAACTGTCCTCTGCACGCCTCCACCAAGGGCCTCTCCAACAAGAAGCTCATTTCCCACATG
AAGGATGGTGCCTGGCTCGTCAACACCGCTCGAGGAGCTATCTGTGTCACCGAGGACATTGTTGAGGCTCTCGAGTC
CGGAAAGATTCGAGGCTACGTGGGCGATGTCTGGTTCCCTCAGCCCGAGAATAATGCGAAACAAGTACGGTGGAGG
AAACGCCATGACCCCTCATATCTCTGGTACTTCTATTGATGCCCAGGCCCGATACGCCAAAGGTACCAAGAACATTTT
CGAGGTCTTCTTCTCCGGAAAGCAGGGCTATCGACCCCAGGACATCATCTGTATCAACGGTAACTACGGTACCAAGG
CTTACGGTGATGACAAGGAACATTCTAAGAAATAAGTTAGTTACGTGAATCCACTAGATTATTT
>YALI0D25256g
SEQ ID NO: 47
ATGACACAAAAAGTACTTTTCCTGGACGAGATCCACGACGCCACCAAGGACTACGCGGCTCTGGCGCAGAAATGCGA
TATTCAGCATGTCGGAACAACGTCGCGAGAGCAGTTTCTCAAGGAGTGCAAGGAGGGCAAGTACGACGGGTTTGTG
GCCATCTACCGAACGTTCACCACGCTCGGCAAGGTCGGAAGGTTCGACAAGGAACTATGTGACGCTCTGCCGGCCTC
CATCAAGGCTGTGTGCCACTACGGAGCCGGCTACGACCAGGTGGATGTGGCGCCGTTCACCGAGCGGGGAATCCAG
GTCAGCAACGTCCAAGGAGCCGCTGACGCCGCCACGGCGCTGACCAACGTATATTTGATGCTGGGCTGTCTGCGAAA
CTTTGGTCATGCGGCCATTTCGCTCCGACAGGGCAATTGGATTGGAGACGTGCCGTTGGGCCATGATCCCGACGGCA
AAGTGCTCGGAATCATGGGCATGGGCGGCATTGGCCGTCAGGTGAGAGATTACGTCAAGCCGTTCGGGTTCGAGAA
AATCATCTACTACAATCGAAGCCGACTGTCTCCGGAGCTCGAGGGCGGATGCCATTACGTGACTCTGGACGAGCTGT
ATGCCCAGGCCGACGTCATCTCCGTCAACGTCCCCCTCAACGCCGCCACCCGTCACATGATCAACTCCGAGTCCATTT
CCAAGATGAAGGACGGTGTTATCATCGTCAACACCGCCCGAGGCCCGGTTATTGACGAGCAGGCTCTTGTTGATGGT
CTCAACTCCGGCAAGATCAGCTCCGCCGGTCTGGACGTGTATGAGCACGAGCCCAAGATTAACCCCGGTCTGCTGAA
GAACCCCCAGGCCCTGCTGCTGCCCCACTTTGGCACTTTTACCATTGAGACCCATCGAAAAATGGAGGAGGCGGTAC
TCAACAACATTGAGACGTTCCTGAAGACCGGTAAGGTGGCCACGATTGTACCCGAGCAGAACGGCAAGTTCTAGCTC
GGTTGCGCGGCGAGATTGTAATGGAAGTGCCCGGCCAAAGGTCAGGCACGGACCCGATAACCAGTTGTGGGAGAAG
GACAGTTTGATTGTTCAACCAAATTCGGTCAATCTGCTAACCCTTTGATCCTGGTGAGACTACAAGTAGCCAGAAATA
GAGAATGAAATGTTGACATAGAGCCACTAGGTGGCTGCTTGGATGA
>YALI0C11099g
SEQ ID NO: 48
AAGCTCCTTGGATGCACCGAGAACGAGCTCGGTATTTGAGACTGGCTAGAGTAACAGGACCACACTCTCGTTACCAA
CTCTTTGAAGGACAGCCGACTCCGTTTTCGACAAGGAGATTGTCGACGCCGACGTAGTTATCACCACCCCCTTCCACC
CCGGCTACATCAACAAGGAGTAAATTAACAAGGCCAAGAGGCTCAAGATCTACATCACCGCCGGTGTCGGCTCGGAC
CACGCCAATATTGACGCAGCCAACGCCCGAGACATTGCCGTACTCGAGGTCACCGGCTTCAACGTCCAGTCCGTTGC
TGAGAACGTTGTTATGACCATGCTGGTGCTGGTCCGAAACTTGTCGCTGCCCACGAGCAGATCATTTCTGGTGGCAG
CGAGTCCTGAAGAGACTTGCACCCTTCAACCCCTTGGAGCTCCTCTACTACGACTACCAGCCCATGCCTGCCAACGTC
GAGAAGGAAATTGGATGCCGACAAGTCGAGTCTCTGGAGAAGATGCTTTCTCTCTGTGACGCTGTTACCATCAATTGT
CCCCTTCACACCTCCACCAAGGGTCTCTTCAACAAAGTGCTCTTCTCCCACATGAAGGACGGAGTCTGGCTCGTCAAC
ACCGCCCGAGGAGCCATCTGCGTCACCGAGGACATTGCCAAAGCCCTCAAGTCTGGCAAGATCCGAGCATACGGCG
GAGACGTCTGGTTTCTCCAGCCTGCCCCCAAATACCACCCCTGGAGAACCATGCGAAACAAGTACAGCGGGGGAAAC
GCCATGACTCCCCGTATCTCCGGCACTTCCATTGGTGCCCAGGGCCGGTCCGCCGAGGGTAACAAAAAGATCCTCAA
GGTCTTTTTGTCGGGCAAGCAGGAATACCGACCTCAGGATATCATCTGTATCAACGGCCATTATGGTACCAAGGCTTA
CGGTCACGACAAAGAGCACAAGGAGCACCAGACCAAGTAG
>YALI0F09966g
SEQ ID NO: 49
ATGAAATGGATGCGTCATAGTTCAGACAAAATTTCGAACCTAAAACGGCCCACAGATAAAACGGTGCATTGTCGCACA
TACGAACAGACCAGTCGTCGTTACATCGCCAACAACCCCCGCCACACCATGTCCGCCGTCAACATTCCCGTGAACGAA
AACGAGGTTTCGGTCTCGTCTTCGCCCATCACGTCGTACGGATCGCCCGTCTCGGGCTCGTTCCAGGGCAAGCCCCG
ACAGCGACGATACTCTTACACCGCTGCGCGAACCAACAAGCTGAAGCCGTTTTCCACCGGCGACATCAAGATCCTGC
TGCTGGAAAACGTCAACCAGACAGCCATTGACATTCTCGAGGGCCAGGGCTACCAGGTGGAGACCCACAAGTCGTCG
CTGGACGAGGAGGAGCTGATTGAGAAGATCCGAGATGTGCAGGTGGTTGGTATCCGATCCAAGACCAAGCTCAACTC
ACGGGTGCTCAAGGAGGCCAAGAACCTCATTGCCATTGGTTGTTTCTGCATCGGTACCAACCAGGTCGATCTCGAGT
ACGCCGCCAACAACGGTATTGCCGTGTTCAACTCGCCTTTCTCCAACTCGCGGTCTGTGGCCGAGCTGGTCATCTGC
GAGATCATCATGCTGTCGCGACAGCTGGGCGACCGAAACATCGAGATGCACGCCGGCACCTGGAACAAGGTCTCCG
CCAAGTGCTGGGAGATCCGAGGCAAGACTCTCGGAATTGTGGGCTACGGCCACATTGGCTCCCAGCTGTCCGTTCTC
GCCGAGTCCATGGGCATGAACGTCATTTACTACGACGTGATCATGATCATGGGTCTCGGTACCGCCAAGCAGGTGCC
CACCCTTGCCGAGCTGCTGGCCCAGGCCGACTACGTCTCTCTGCACGTGCCCGAGCTGCCCGAGACCATGAACCTCA
TGTCCAAGGCCCAGTTCGACGGCATGAAGAACGGCTCGTACCTCATCAACAACGCCCGAGGCAAGGTCATTGACATT
CCCGAGCTCATCTCTGCCATGAAGTCCGGAAAGCTTGCTGGAGCCGCCGTCGACGTCTTCCCCAAGGAGCCTGCCAA
GAACGGCTCAAACGAGTTTGGCTCCCACCTCAACGAGTGGACCAATGAGCTCCTCACCCTGCCCAACCTCATCATGTC
TCCCCATATTGGAGGCTCCACCGAGGAGGCCCAGTCCGCCATTGGTATCGAGGTCGGCACCGCTCTCACCAAGTACA
TCAACGAGGGTTCCTCTGTTGGTGCCGTCAACTTCCCCGAGGTCAACCTCAACGTGGTCAACCACGCCGAGGAGCAC
GACCACGTGCGAATCCTGTACGTGCATAAGAACGTGCCTGGTGTGCTTCTGTCCGTCAACGAGATTTTCGCCTCCCAT
AACATTGAGAAGCAGTACTCTGAGTCGCGTGGTGATATTGCCTACCTGATGGCCGACATTGCTGACGTCGATCAGGC
CGACATCAAGACTCTGTACGAGAAGCTCGAGCAGACCCCCTTCAAGATCTCCACCCGACTGTTGTATTAAGTGGTGAG
GAGAGACCAATATGCTAAGACAAGACAAACAAGAGATTCAACTAATTTTATAGATGTAAATACATTCAACCGTGGTTAA
CGACAATGCTG

In other embodiments, the second nucleic acid sequence is a non naturally-occurring nucleic acid sequence, for example is generated by amplification from a template or is generated synthetically. Such a nucleic acid could have a naturally occurring sequence, but the structure is such that it is different to that found in nature, for example, PCR amplification results in a nucleic acid structure devoid of certain modifications found on the naturally occurring sequence. In other embodiments, the nucleic acid sequence itself may be a non naturally-occurring sequence.

In some embodiments the second nucleic acid sequence is designed in silico, for example through rational sequence design.

It will be clear to the skilled person that the nucleic acid construct of the invention may be linear, or may be circular.

It will be clear to the skilled person that the nucleic acid construct of the invention can be part of a nucleic acid expression cassette. Accordingly, the invention also provides an expression cassette that comprises the isolated nucleic acid or the nucleic acid construct of the invention.

The expression vector of the invention may be linear or may be circular.

The invention also provides a vector comprising the isolated nucleic acid of the invention, or the nucleic acid construct of the invention.

The vector may be selected from a group comprising a plasmid or an artificial chromosome. The artificial chromosome may be selected from a bacterial artificial chromosome (BAC), a yeast artificial chromosome (YAC), and a Human artificial chromosome (HAC).

In some instances, the isolated nucleic acid of the invention, the nucleic acid construct of the invention, the expression vector of the invention or the vector of the invention may be loaded into a viral vector. In some embodiments the viral vector is selected from a group comprising a retroviral vector, a lentiviral vector, an adenoviral vector, an adeno-associated viral vector, a bacteriophage vector, and a hybrid viral vector.

It will be clear to the skilled person that the isolated nucleic acid of the invention, the nucleic acid construct of the invention, the expression vector of the invention or the vector of the invention have particular uses when located with a cell.

The invention therefore also provides a cell comprising the isolated nucleic acid of the invention, the nucleic acid construct of the invention, the expression vector of the invention or the vector of the invention.

In one embodiment the cell is not a naturally occurring cell, for example because the cell comprises the isolated nucleic acid of the invention, the nucleic acid construct of the invention, the expression vector of the invention or the vector of the invention, and comprises any of these at a non-naturally location. This maybe in addition to the cell comprises a copy of the isolated nucleic acid of the invention, the nucleic acid construct of the invention, the expression vector of the invention or the vector of the invention at a natural location.

In one embodiment the cell is an engineered cell, since it has been engineered to comprise the isolated nucleic acid of the invention, the nucleic acid construct of the invention, the expression vector of the invention or the vector of the invention, and comprises any of these at a non-naturally location.

In some embodiments the cell is not a Yarrowia lipolytica cell that has not been engineered to introduce at least one isolated nucleic acid of the invention, the nucleic acid construct of the invention, the expression vector of the invention or the vector of the invention.

The skilled person will understand that the isolated nucleic acid, nucleic acid construct, expression vector or vector of the invention may be applied usefully in a variety of cell types. Accordingly, in some embodiments, the cell is selected from the group comprising or consisting: a prokaryotic cell and a eukaryotic cell.

As the skilled person will be aware, prokaryotic cells are generally highly genetically tractable and readily cultured in conditions known to the skilled person. Bacterial cells are useful for the production of several of the products of the invention described herein. Therefore, in some embodiments, the cell is a prokaryotic cell. In some embodiments the cell is selected from a group comprising or consisting: a bacterial cell and an archaeal cell. In one embodiment, the cell is a bacterial cell. In one embodiment, the cell is an archaeal cell.

It will be appreciated that in some embodiments, the bacterial cell is a gram-negative bacterial cell. In some embodiments, the gram-negative bacterial cell belongs to a genus selected from the group consisting or comprising of: Escherichia, Pseudomonas and Vibrio. In one embodiment, the gram-negative bacterial cell is an Escherichia coli cell. In one embodiment, the cell is a Vibrio natriegens cell.

In some embodiments, the bacterial cell is a gram-positive bacterial cell. In some embodiments, the gram-positive bacterial cell belongs to a genus selected from the group consisting or comprising of: Bacillus, Clostridium, Lactobacillus, Lactococcus, Paenibacillus, and Streptomyces.

For the production of eukaryotic proteins, and in particular those which require extensive post-translational modifications, expression in a eukaryotic cell is typically preferred to prokaryotic expression. may not be readily conducted in a prokaryotic cell.

In some embodiments, the cell is a eukaryotic cell. In some embodiments, the cell is a cell selected from a group comprising a fungal cell, a plant cell, and an animal cell. In one embodiment, the cell is a fungal cell. In one embodiment, the cell is a plant cell. In one embodiment, the cell is an animal cell.

In a preferred embodiment, the cell is a fungal cell. The skilled person will be aware of the diversity of fungal phylogeny and cell types. As such, in some embodiments, the fungal cell is a cell selected from a list comprising or consisting, but not limited to: a yeast cell and a hyphal cell. In a preferred embodiment, the fungal cell is a yeast cell.

Yeast cells may be classified according to their metabolism. For example, a yeast cell may be classified according to classifications selected from but not limited to the group comprising or consisting: a methylotrophic yeast cell, a non-methylotrophic yeast cell, and an oleaginous yeast cell. In some embodiments, the cell is a methylotrophic yeast cell. In some embodiments, the methylotrophic yeast cell belongs to a genus selected from a group consisting or comprising: Candida, Hansenula, Komagatella, Pichia. In some embodiments, the yeast cell is a non-methylotrophic yeast cell. In some embodiments, the yeast cell belongs to a genus selected from a group consisting or comprising: Ashbya, Blastobotrys, Cryptococcus, Cutaneotrichosporon, Dekkera, Kluveromyces, Rhodosporidium, Rhodotorula, Lipomyces, Saccharomyces, and Yarrowia. In a preferred embodiment, the yeast cell is a cell belonging to the species Yarrowia lipolytica.

In some preferred embodiments, the cell in which the isolated nucleic acid, nucleic acid, expression cassette, or vector provided herein is employed is of the same species as that which the isolated nucleic acid sequences was originally derived, i.e. a autologous species. For example, where the isolated nucleic acid of the invention comprises or consists of a portion of the upstream 1Kb or 1.5Kb region of a Yarrowia lipolyitca FDH gene, for example such as those promoter regions specified in SEQ ID NO: 2-33, the cell is a Yarrowia lipolytica cell. For example, generally, where the isolated nucleic acid of the invention comprises or consists of a portion of the upstream 1Kb or 1.5Kb region of a species X FDH gene, the cell is a cell of species X. In such embodiments, since the nucleic acid sequence/promoter sequence is largely native to that species (potentially with one or more mutations, as described herein or truncations) it is expected that that species will comprise the necessary transcription factors and other agents to allow the nucleic acid to result in inducible expression.

In other embodiments, where the isolated nucleic acid of the invention comprises or consists of a portion of the upstream 1Kb or 1.5Kb region of a Yarrowia lipolyitca FDH gene, for example such as those promoter regions specified in SEQ ID NO: 2-33, the cell is a cell other than a Yarrowia lipolytica cell. It is expected that there will be some degeneracy between species that allows an inducible promoter from one species to also act as an inducible promoter in a different species. For example, in some embodiments, where the isolated nucleic acid of the invention comprises a portion of the upstream 1Kb or 1.5Kb region of a species X FDH gene, the cell is not a cell of species X.

Preferably the nucleic acid sequence is employed in a cell of the same species.

It will be appreciated that the isolated nucleic acid, nucleic acid, expression cassette, or vector provided herein may be maintained by the cell of the invention. By “maintained” it is meant that the isolated nucleic acid, nucleic acid, expression cassette, or vector of the invention is replicated by the cell of the invention and is segregated into at least or both of the cells that result from cell division, e.g. into the mother and daughter yeast cell.

It will be appreciated that the isolated nucleic acid, nucleic acid, expression cassette, or vector provided herein may be maintained by the cell of the invention in several ways. In one embodiment, the isolated nucleic acid, nucleic acid, expression cassette, or vector is episomally maintained by the cell. In one embodiment, the isolated nucleic acid, expression cassette, or vector is integrated into the genome of said cell.

The cell may comprise any number of copies of the isolated nucleic acid, expression cassette, or vector of the invention. Accordingly, in some embodiments, the cell comprises at least about one, at least about two, at least about three, at least about four, at least about five, at least about six, at least about seven, at least about eight, at least about nine, at least about 10, at least about 20, at least about 30, at least about 40, at least about 50, at least about 60, at least about 70, at least about 80, at least about 90, at least about 100 or more copies of the isolated nucleic acid, expression cassette, or vector of the invention.

It will be understood that integration of the isolated nucleic acid, expression cassette, or vector of the invention into the genome of a cell of the invention may drive expression of a second sequence located in the genome. In one embodiment, the isolated nucleic acid, nucleic acid, expression cassette, or vector is integrated upstream of a second sequence located in the genome, and following integration the isolated nucleic acid, inducible promoter, nucleic acid, expression cassette, or vector is capable of driving transcription of the second sequence.

In some embodiments, the isolated nucleic acid, inducible promoter, nucleic acid, expression cassette, or vector is integrated into the genome of said cell at a different locus to the locus of the native promoter. For example, in some embodiments, where:
the isolated nucleic acid has a sequence of 80%, 85%, 90%, 92%, 94%, 95%, 96%, 97%, 98%, 99% or 100% identity to SEQ ID NO: 2 or 18 the isolated nucleic acid, inducible promoter, nucleic acid, expression cassette, or vector is integrated at a genomic locus that is different to the locus of native SEQ ID NO: 2 or 18, i.e., is not operably inserted upstream of the gene encoding SEQ ID NO: 34;
the isolated nucleic acid has a sequence of 80%, 85%, 90%, 92%, 94%, 95%, 96%, 97%, 98%, 99% or 100% identity to SEQ ID NO: 3 or 19 the isolated nucleic acid, inducible promoter, nucleic acid, expression cassette, or vector is integrated at a genomic locus that is different to the locus of native SEQ ID NO: 3 or 19, i.e., is not operably inserted upstream of the gene encoding SEQ ID NO: 35;
the isolated nucleic acid has a sequence of 80%, 85%, 90%, 92%, 94%, 95%, 96%, 97%, 98%, 99% or 100% identity to SEQ ID NO: 4 or 20 the isolated nucleic acid, inducible promoter, nucleic acid, expression cassette, or vector is integrated at a genomic locus that is different to the locus of native SEQ ID NO: 4 or 20, i.e., is not operably inserted upstream of the gene encoding SEQ ID NO: 36;
the isolated nucleic acid has a sequence of 80%, 85%, 90%, 92%, 94%, 95%, 96%, 97%, 98%, 99% or 100% identity to SEQ ID NO: 5 or 21 the isolated nucleic acid, inducible promoter, nucleic acid, expression cassette, or vector is integrated at a genomic locus that is different to the locus of native SEQ ID NO: 5 or 21, i.e., is not operably inserted upstream of the gene encoding SEQ ID NO: 37;
the isolated nucleic acid has a sequence of 80%, 85%, 90%, 92%, 94%, 95%, 96%, 97%, 98%, 99% or 100% identity to SEQ ID NO: 6 or 22 the isolated nucleic acid, inducible promoter, nucleic acid, expression cassette, or vector is integrated at a genomic locus that is different to the locus of native SEQ ID NO: 6 or 22, i.e., is not operably inserted upstream of the gene encoding SEQ ID NO: 38;
the isolated nucleic acid has a sequence of 80%, 85%, 90%, 92%, 94%, 95%, 96%, 97%, 98%, 99% or 100% identity to SEQ ID NO: 7 or 23 the isolated nucleic acid, inducible promoter, nucleic acid, expression cassette, or vector is integrated at a genomic locus that is different to the locus of native SEQ ID NO: 7 or 23, i.e., is not operably inserted upstream of the gene encoding SEQ ID NO: 39;
the isolated nucleic acid has a sequence of 80%, 85%, 90%, 92%, 94%, 95%, 96%, 97%, 98%, 99% or 100% identity to SEQ ID NO: 8 or 24 the isolated nucleic acid, inducible promoter, nucleic acid, expression cassette, or vector is integrated at a genomic locus that is different to the locus of native SEQ ID NO: 8 or 24, i.e., is not operably inserted upstream of the gene encoding SEQ ID NO: 40;
the isolated nucleic acid has a sequence of 80%, 85%, 90%, 92%, 94%, 95%, 96%, 97%, 98%, 99% or 100% identity to SEQ ID NO: 9 or 25 the isolated nucleic acid, inducible promoter, nucleic acid, expression cassette, or vector is integrated at a genomic locus that is different to the locus of native SEQ ID NO: 9 or 25, i.e., is not operably inserted upstream of the gene encoding SEQ ID NO: 41;
the isolated nucleic acid has a sequence of 80%, 85%, 90%, 92%, 94%, 95%, 96%, 97%, 98%, 99% or 100% identity to SEQ ID NO: 10 or 26 the isolated nucleic acid, inducible promoter, nucleic acid, expression cassette, or vector is integrated at a genomic locus that is different to the locus of native SEQ ID NO: 10 or 26, i.e., is not operably inserted upstream of the gene encoding SEQ ID NO: 42;
the isolated nucleic acid has a sequence of 80%, 85%, 90%, 92%, 94%, 95%, 96%, 97%, 98%, 99% or 100% identity to SEQ ID NO: 11 or 27 the isolated nucleic acid, inducible promoter, nucleic acid, expression cassette, or vector is integrated at a genomic locus that is different to the locus of native SEQ ID NO: 11 or 27, i.e., is not operably inserted upstream of the gene encoding SEQ ID NO: 43;
the isolated nucleic acid has a sequence of 80%, 85%, 90%, 92%, 94%, 95%, 96%, 97%, 98%, 99% or 100% identity to SEQ ID NO: 12 or 28 the isolated nucleic acid, inducible promoter, nucleic acid, expression cassette, or vector is integrated at a genomic locus that is different to the locus of native SEQ ID NO: 12 or 28, i.e., is not operably inserted upstream of the gene encoding SEQ ID NO: 44;
the isolated nucleic acid has a sequence of 80%, 85%, 90%, 92%, 94%, 95%, 96%, 97%, 98%, 99% or 100% identity to SEQ ID NO: 13 or 29 the isolated nucleic acid, inducible promoter, nucleic acid, expression cassette, or vector is integrated at a genomic locus that is different to the locus of native SEQ ID NO: 13 or 29, i.e., is not operably inserted upstream of the gene encoding SEQ ID NO: 45;
the isolated nucleic acid has a sequence of 80%, 85%, 90%, 92%, 94%, 95%, 96%, 97%, 98%, 99% or 100% identity to SEQ ID NO: 14 or 30 the isolated nucleic acid, inducible promoter, nucleic acid, expression cassette, or vector is integrated at a genomic locus that is different to the locus of native SEQ ID NO: 14 or 30, i.e., is not operably inserted upstream of the gene encoding SEQ ID NO: 46;
the isolated nucleic acid has a sequence of 80%, 85%, 90%, 92%, 94%, 95%, 96%, 97%, 98%, 99% or 100% identity to SEQ ID NO: 15 or 31 the isolated nucleic acid, inducible promoter, nucleic acid, expression cassette, or vector is integrated at a genomic locus that is different to the locus of native SEQ ID NO: 15 or 31, i.e., is not operably inserted upstream of the gene encoding SEQ ID NO: 47;
the isolated nucleic acid has a sequence of 80%, 85%, 90%, 92%, 94%, 95%, 96%, 97%, 98%, 99% or 100% identity to SEQ ID NO: 16 or 32 the isolated nucleic acid, inducible promoter, nucleic acid, expression cassette, or vector is integrated at a genomic locus that is different to the locus of native SEQ ID NO: 16 or 32, i.e., is not operably inserted upstream of the gene encoding SEQ ID NO: 48; or
the isolated nucleic acid has a sequence of 80%, 85%, 90%, 92%, 94%, 95%, 96%, 97%, 98%, 99% or 100% identity to SEQ ID NO: 17 or 33 the isolated nucleic acid, inducible promoter, nucleic acid, expression cassette, or vector is integrated at a genomic locus that is different to the locus of native SEQ ID NO: 17 or 33, i.e., is not operably inserted upstream of the gene encoding SEQ ID NO: 49.

For example, the above nucleic acids are not inserted into a Yarrowia lipolytica cell at the above cited genomic loci.

The present invention also provides methods of preparing a cell of the invention that comprises an isolated nucleic acid, nucleic acid, expression cassette, or vector of the invention. In one embodiment, the method comprises introducing the isolated nucleic acid, nucleic acid, expression cassette, or vector of the invention into the cell. The skilled person will be aware of appropriate methods of introducing the isolated nucleic acid, nucleic acid, expression cassette, or vector of the invention into any of the cells described herein. As such, the isolated nucleic acid, nucleic acid, expression cassette, or vector of the invention may be introduced into the cells described herein by a method selected from but not limited to the group comprising or consisting: electroporation, heat-shock, alkaline transformation, spheroplast-mediated transformation methods, conjugation, transfection, lipofection, viral transduction, microinjection, macroinjection, fibre-mediated DNA delivery, laser-mediated gene transfer or delivery, pollen transformation, direct DNA uptake, ballistic transformation, Yoshida effect, Aminclay-induced transformation, or any combination thereof.

Also provided herein are methods of producing products using the isolated nucleic acids, nucleic acids, expression cassettes, vectors, or cells of the invention. In one embodiment, the product is an expression product of a gene, wherein the method comprises the use of the isolated nucleic acid, nucleic acid, expression cassette, vector, or cell of the invention.

In one embodiment, the method of producing a product comprises the step of culturing any of the cells provided herein in an appropriate growth medium. The skilled person is capable of determining appropriate culture media for use with the cells provided herein. However, for illustrative purposes, in some embodiments the culture media is selected from but not limited to the group comprising or consisting: Abiotrophia medium, acetamide medium, Acetobacter medium, ACH medium, Actinoplanes medium, Agrobacterium medium, Alicyclobacillus medium, allantoin mineral medium, α-MEM, Ashbya full medum, Azotobacter medium, Bacillus medium, Bennett's medium, Bifidobacterium medium, blue green algae medium, BME, brain heart infusion (BHI) medium, Caulobacter medium, Cantharellus medium, CASO medium, Clostridium medium, CMRL1066, Corynebacterium medium, creatinine medium, Czapek medium, Desulfovibrio medium, DMEM, DMEM/F-12, Eagle media, EMB medium, Fisher's medium, fruit fly medium, Gluconobacter medium, glucose peptone yeast extract (GYP) medium, glucose yeast extract medium, Halobacterium medium, Ham's F-10, Ham's F-12, Ham's F-12K, IMEM, Leibovitz's L-15, lysogeny broth (LB), luminous medium, M17 medium, M9 minimal medium, mannitol medium, marine medium, MCDB202, MCDB301, MCDB153, MCDB110, MCDB402, MCDB170, MCDB131, medium 199, MEM, methylamine salts medium, mixed media, modified chopped meat medium, MRS medium, MS medium, Mueller-Hinton medium, MY medium, N4 mineral medium, N-Z amine medium, NCTC109, Nitrosomonas europaea medium, nutrient medium, NZCYM medium, NZM medium, NZYM medium, oatmeal medium, Oenococcus medium, osmophilic medium, potato-carrot medium, Propionibacterium medium, PYS medium, R medium, rolled oats mineral medium, RPMI1640, RPMI1640/DMEM/F-12, saccharose medium, super optimal broth, super optimal broth with catabolite repression, 5% sorbitol medium, sour dough medium, starch-mineral salt medium, styrene mineral salts medium, synthetic sea water, terrific broth, Thermus medium, Thiobacillus medium, tomato juice medium, tomato juice yeast extract medium, Trowell's T-8, TSY medium, TYG medium, TYX medium, urea medium, uric acid medium, Waymouth's MB752/1, whey medium, Wickerham salt medium, yeast extract glucose medium, YEL medium, YMF medium, YMG medium, YNB medium, YPD medium, YPG medium, YPM medium, YT medium, YT (2×medium), or any combination thereof.

In one embodiment the media is YNB or ACH +caa media.

It will be appreciated that the media provided herein may be modified. For example, the media may be buffered, may comprise additional selective agents such as antibiotics and salts, or may contain indicator compounds.

In one embodiment, the method of producing products comprises the step of contacting the cell with an appropriate inducer agent provided and described herein. In some embodiments, the inducer agent is selected from a group comprising or consisting of: ethanol, methanol, propanol, butanol, glycerol, formaldehyde, formate, or any combination thereof. In one embodiment, the inducer agent is methanol. In a preferred embodiment, the inducer agent is formate.

In one embodiment, the expression product is a nucleic acid. In one embodiment, the expression product is RNA. In some embodiments, the RNA is selected from a group consisting or comprising of: mRNA, rRNA, miRNA, siRNA, piRNA, snRNA, snoRNA, exRNA, scaRNA, lncRNA, gRNA, sgRNA, crRNA, and tracrRNA. In one preferred embodiment, the RNA is mRNA. In one preferred embodiment, the RNA is sgRNA.

In one embodiment, the expression product is a protein comprising an amino acid sequence. It will be appreciated that the protein may be a natural protein selected from any organism. In some embodiments, the protein is a protein that is not selected from Yarrowia lipolytica. In some embodiments, the protein is a protein selected from Yarrowia lipolytica.

In some embodiments, the protein is not a natural protein. In one embodiment, the 30 protein is an artificial protein. In one embodiment, the protein is designed by rational protein design.

A protein may also be a variant of a protein that is a natural protein or a protein that is not a natural protein. Variants of protein may or may not comprise at least one or more amino acid substitution(s), deletion(s), insertion(s), covalent alteration(s) to amino acid residue(s), covalent linkage(s) between amino acid residue(s), or any combination thereof. Variant proteins may have altered secondary, tertiary, quaternary, or quinary structure relative to the natural protein that does not comprise the at least one or more amino acid substitution.

It will be appreciated that the proteins of the invention may be trafficked by a cell in different ways. Accordingly, the protein of the invention may have different localisations. In one embodiment, a protein of the invention is exported by a cell from within said cell into the extracellular milieu. In one embodiment, a protein of the invention is retained by the cell on the cell membrane of a cell. In one embodiment, a protein of the invention is retained within a cell.

Proteins of the invention may be purified. Methods of protein purification include but are not limited to methods selected from the group comprising or consisting: size exclusion chromatography, gel permeation chromatography, hydrophobic interaction chromatography, ion exchange chromatography, free-flow electrophoresis, affinity chromatography, immunoaffinity chromatography, HPLC, or any combination thereof.

Purified proteins of the invention may be concentrated. Methods of protein purification include but are not limited to methods selected from the group comprising or consisting: dialysis, lyophilisation, precipitation, and ultrafiltration.

Protein purification methods may require the target protein to be tagged. Accordingly, any protein of the invention may comprise a first protein optionally linked by an amino acid linker to a short protein tag, a full-length protein tag, or any combination thereof. Short protein tags may be selected from a group comprising or consisting: an ALFA-tag, an AviTag, a C-tag, a Calmodulin-tag, a DogTag a polyglutamine tag, an E-tag, a FLAG-tag, and HA-tag, a His-tag, an Isopeptag, a Myc-tag, an NE-tag, a Rho1D4-tag, an S-tag, an SBP-tag, an SdyTag, a SnoopTag, a Softag 1, a Softag 2, a Spot-tag, a SpyTag, a Strep-tag, a T7-tag, a TC-tag, a Ty-tag, a V5-tag, a VSV-tag, and an Xpress-tag, or any combination thereof. Full-length protein tags may be selected from the group comprising or consisting: a BCCP tag, a glutathione-S-transferase tag, a GFP tag, a HaloTag, a SNAP-tag, a CLIP-tag, a HUH-tag, a maltose binding protein tag, a Nus-tag, a Thioredoxin tag, an Fc tag, and a CRDSAT tag, or any combination thereof. Proteins of the invention may comprise a short protein tag or a full-length protein tag at the N-terminus of the protein, the C-terminus of the protein, or at any position in the amino acid sequence of a protein of the invention.

Also provided herein is a method of producing a secondary metabolite, wherein the method comprises the use of the isolated nucleic acid sequence, expression cassette, vector, or cell of any of the preceding claims. The skilled person will understand which chemical species are encompassed by the term “secondary metabolite”. A secondary metabolite may be selected from but not limited to the group comprising or consisting: terpenes, steroids, phenolic compounds, glycoside compounds, alkaloids, polyketides, flavonoids, fatty acid derivatives, non-ribosomal peptides, and enzyme co-factors.

Secondary metabolites may be exported by a cell, retained on the cell membrane of a cell, or retained within a cell. In one embodiment, the secondary metabolite is exported by the cell into the extracellular milieu. In one embodiment, the secondary metabolite is retained by the cell on the cell membrane of the cell. In one embodiment, the secondary metabolite is retained within said the cell.

It will be appreciated that multiple biosynthetic steps may be required to generate the secondary metabolites produced by the method of the invention. As such, multiple proteins, and hence multiple genes, may be required for the biosynthesis of the secondary metabolites produced by the method provided herein. The method of producing a secondary metabolite provided herein may therefore comprise the use of a cell comprising at least one isolated nucleic acid, nucleic acid construct, expression vector or vector provided herein. In some embodiments, the cell of the invention comprises multiple copies of the isolated nucleic acid, nucleic acid construct, expression vector or vector, as described hereinabove.

In some embodiments, the cell comprises multiple isolated nucleic acids, nucleic acid constructs, expression vectors or vectors of the invention. In some embodiments, the cell comprises several isolated nucleic acids, nucleic acid constructs, expression vectors or vectors, wherein each isolated nucleic acid sequence is operably linked to a different and distinct second nucleic acid sequence, or wherein each nucleic acid construct, expression vector or vector comprises a first nucleic acid sequence operably linked to a different and distinct second nucleic acid sequence. In some embodiments, the cell comprises at least about one, at least about two, at least about three, at least about four, at least about five, at least about six, at least about seven, at least about eight, at least about nine, at least about 10, or more isolated nucleic acids, nucleic acid constructs, expression vectors or vectors comprising a first nucleic acid sequence operably linked to a second nucleic acid sequence, wherein the second nucleic acid sequence of each isolated nucleic acid, nucleic acid construct, expression vector or vector is different from each other second sequence of each isolated nucleic acid, nucleic acid construct, expression vector or vector. In some embodiments, the cell comprises fewer than about two, fewer than about three, fewer than about four, fewer than about five, fewer than about six, fewer than about seven, fewer than about eight, fewer than about nine, fewer than about 10 isolated nucleic acids, nucleic acid constructs, expression vectors or vectors comprising a first nucleic acid sequence operably linked to a second nucleic acid sequence, wherein the second nucleic acid sequence of each isolated nucleic acid, nucleic acid construct, expression vector or vector is different from each other second sequence of each isolated nucleic acid, nucleic acid construct, expression vector or vector. In some embodiments, the cell comprises about one, about two, about three, about four, about five, about six, about seven, about eight, about nine, about 10 or more isolated nucleic acids, nucleic acid constructs, expression vectors or vectors comprising a first nucleic acid sequence operably linked to a second nucleic acid sequence, wherein the second nucleic acid sequence of each isolated nucleic acid, nucleic acid construct, expression vector or vector is different from each other second sequence of each isolated nucleic acid, nucleic acid construct, expression vector or vector.

In some embodiments, the cell comprises several isolated nucleic acids, nucleic acid constructs, expression vectors or vectors, wherein each isolated nucleic acid sequence is operably linked to the same second nucleic acid sequence, or wherein each nucleic acid construct, expression vector or vector comprises a first nucleic acid sequence operably linked to the same second nucleic acid sequence. In some embodiments, the cell comprises at least about one, at least about two, at least about three, at least about four, at least about five, at least about six, at least about seven, at least about eight, at least about nine, at least about 10 isolated nucleic acids, nucleic acid constructs, expression vectors or vectors comprising a first nucleic acid sequence operably linked to a second nucleic acid sequence, wherein the second nucleic acid sequence of each isolated nucleic acid, nucleic acid construct, expression vector or vector is the same as each other second sequence of each isolated nucleic acid, nucleic acid construct, expression vector or vector. In some embodiments, the cell comprises fewer than about two, fewer than about three, fewer than about four, fewer than about five, fewer than about six, fewer than about seven, fewer than about eight, fewer than about nine, fewer than about 10 isolated nucleic acids, nucleic acid constructs, expression vectors or vectors comprising a first nucleic acid sequence operably linked to a second nucleic acid sequence, wherein the second nucleic acid sequence of each isolated nucleic acid, nucleic acid construct, expression vector or vector is the same as each other second sequence of each isolated nucleic acid, nucleic acid construct, expression vector or vector. In some embodiments, the cell comprises about one, about two, about three, about four, about five, about six, about seven, about eight, about nine, about 10 isolated nucleic acids, nucleic acid constructs, expression vectors or vectors comprising a first nucleic acid sequence operably linked to a second nucleic acid sequence, wherein the second nucleic acid sequence of each isolated nucleic acid, nucleic acid construct, expression vector or vector is the same as each other second sequence of each isolated nucleic acid, nucleic acid construct, expression vector or vector.

Also provided herein is a method of detecting the induction state of an isolated nucleic acid, nucleic acid, expression cassette, and/or vector of the invention in any of the cells provided herein.

In one embodiment, the induction state of an isolated nucleic acid, nucleic acid, expression cassette, and/or vector is measured by the detection of a product of the second nucleic acid sequence.

In one embodiment, the product of the second nucleic acid sequence is RNA. Methods of detecting RNA are well-known to those skilled in the art, and may be selected from the group consisting or comprising: RT-PCR, qRT-PCT, Northern blot, nuclease protection assays, and in-situ hybridisation, RNAseq, RNA microarray, Nanopore sequencing, or any combination thereof.

In one embodiment, the product of the second nucleic acid sequence is a protein comprising an amino acid sequence. In one embodiment, the protein is a fluorescent protein or a luminescent protein which emits light. Illustrative examples of appropriate fluorescent or luminescent proteins that emit light for the purposes of detecting the induction state of an isolated nucleic acid, nucleic acid, expression cassette, and/or vector of the invention are given below. In one embodiment, the protein is detected by detection of an emitted light signal. Illustrative methods of detecting bioluminescence and/or fluorescence are given below.

In some embodiments, it may be advantageous to detect expression of a protein of interest conjugated to a detectible protein. For example, it may be advantageous to ensure read-through of the protein of interest using a C-terminal tag. Accordingly, in some embodiments, the product of the second nucleic acid sequence comprises a first protein comprising an amino acid sequence linked by an amino acid linker to a second protein comprising an amino acid sequence. In some embodiments, the second protein is a fluorescent protein or a luminescent protein which emits light. Illustrative examples of appropriate fluorescent or luminescent proteins that emit light for the purposes of detecting the induction state of an isolated nucleic acid, nucleic acid, expression cassette, and/or vector of the invention are given below. In one embodiment, the protein is detected by detection of an emitted light signal. Illustrative methods of detecting bioluminescence and/or fluorescence are given below.

Illustrative examples of a fluorescent protein or luminescent protein which emits light may be selected from the group comprising or consisting: aequorin, Allophycocyanin, AmCyan1, AsRed2, Azami Green, Azurite, B-phycoerythrin, CyPet, DsRed, DsRed2, GFP, GFPuv, EBFP, EBFP2, ECFP, EGFP, Emerald, EYFP, HcRed1, horseradish peroxidase, J-Red, Katusha, Kusabira Orange, luciferase, mCardinal, mCFP, mCherry, mCitrine, mEmerald, Midoriishi Cyan, mKate, mKeima-Red, mKO, mNeonGreen, mOrange, mPlum, mRaspberry, mRFP1, mStrawberry, mTFP1, mTurqoise2, P3, PerCP, R-phcoerythrin, RFP, T-Sapphire, TagCFP, TagGFP, TagRFP, TagYFP, tdTomato, Topaz, TurboFP602, TurboFP635, TurboGFP, TurboRFP, TurboYFP, Venus, YFP, YPet, ZsGreen1, and any functional variant thereof.

Illustrative examples of methods used to detect light emitted by a fluorescent protein or a luminescent protein include but are not limited to methods selected from the group comprising or consisting: spectrophotometry, FACS, flow cytometry, and fluorescence microscopy, or any combination thereof.

In one embodiment, the second protein is a protein tag. In one embodiment, the protein tag is a short peptide tag. Illustrative examples of tags are described above.

In one embodiment, the protein is not a fluorescent or luminescent protein which emits light.

In one embodiment, the first or second protein is detected by an antibody method. In one embodiment, the antibody method is selected from a group comprising or consisting: ELISA, western blot, immunoprecipitation, immunoelectrophoresis, and protein immunostaining.

In one embodiment, the protein is detected by a spectrometric method. In one embodiment, the spectrometric method is selected from a group comprising or consisting: HPLC, MS, and LC/MS.

Also provided herein are kits comprising or consisting the materials of the invention. In one embodiment, the kit comprises the isolated nucleic acid, inducible promoter, nucleic acid, expression cassette, vector, or cell of the invention, or any combination of these. In one embodiment, the kit comprises primers, nucleotides, buffers, and/or enzymes for use in the production or amplification of the isolated nucleic acid, inducible promoter, nucleic acid, expression cassette, vector of the invention.

In some embodiments, the kit comprises any one or more of the isolated nucleic acid, inducible promoter, nucleic acid, expression cassette, vector, or cell of the invention, and any one or more inducing agents, for example the inducing agents described herein, for example formate.

Also provided herein are kits comprising or consisting of the materials of the invention for use in the methods of the invention. In one embodiment, the kit comprises the isolated nucleic acid, inducible promoter, nucleic acid, expression cassette, vector, or cell of the invention, or any combination of these for use in the methods of the invention. In one embodiment, the method comprises the production of an expression product as defined herein. In one embodiment, the method comprises the production of a secondary metabolite as defined herein. In one embodiment, the kit provides media for use in culturing the cell of the invention, as described herein. In one embodiment, the kit provides the inducer agent as provided herein. In one embodiment, the kit provides reagents for purifying the expression product or secondary metabolite as described herein.

In one embodiment, the kit provides reagents for use in purifying, detecting, and quantifying the expression state of the isolated nucleic acid, inducible promoter, nucleic acid, expression cassette, or vector, as described herein.

It will be appreciated that the isolated nucleic acid, inducible promoter, nucleic acid, expression cassette, vector, expression products, and secondary metabolites of the invention have diverse applications.

In one embodiment, the invention provides a method of producing animal feed using the isolated nucleic acid, inducible promoter, nucleic acid, expression cassette, vector, cell, method, or kit of the invention, or any combination thereof. In one embodiment, the method comprises producing feed for an animal belonging to a phylum selected from but not limited to the group comprising or consisting: annelids, arthropods, bryozoan, chordates, cnidaria, echinoderms, molluscs, nematodes, platyhelminths, rotifers, sponges, or any combination thereof. In one embodiment, the method comprises producing feed for an arthropod animal selected from but not limited to the group consisting or comprising: insects, arachnids, myriapods, and crustaceans. In one embodiment, the method comprises producing feed for a chordate animal selected from but not limited to the group consisting or comprising: amphibians, birds, crocodiles, fish, lizards, mammals, reptiles, and snakes, or any combination thereof. The invention also provides the animal feed produced using the method.

In one embodiment, the invention provides a composition comprising the isolated nucleic acid, inducible promoter, nucleic acid, expression cassette, vector, cell, method, or kit of the invention, or any combination thereof for use as or in an animal feed. In one embodiment, the composition comprises the isolated nucleic acid, inducible promoter, nucleic acid, expression cassette, vector, cell, method, or kit of the invention, or any combination thereof an animal feed for an animal belonging to a phylum selected from but not limited to the group comprising or consisting: annelids, arthropods, bryozoan, chordates, cnidaria, echinoderms, molluscs, nematodes, platyhelminths, rotifers, sponges, or any combination thereof. In one embodiment, the chordate animal is selected from but not limited to the group consisting or comprising: amphibians, birds, crocodiles, fish, lizards, mammals, reptiles, and snakes, or any combination thereof.

In one embodiment, the invention provides an isolated nucleic acid, inducible promoter, nucleic acid, expression cassette, vector, cell, method or kit, or any combination thereof, for use in a method of producing a vaccine. In one embodiment, the vaccine comprises a protein. In one embodiment, the vaccine comprises a natural protein selected from a pathogen. In one embodiment, the pathogen is selected from the group comprising or consisting: a bacterial pathogen, a viral pathogen, a fungal pathogen, and a parasitic pathogen, or any combination thereof. In one embodiment, the vaccine is a nucleic acid vaccine. In one embodiment, the vaccine is a DNA vaccine. In one embodiment, the vaccine is an RNA vaccine. In one embodiment the vaccine comprise the cell of the invention.

In one embodiment, the invention provides a method of producing a vaccine for use in an animal belonging the group consisting or comprising: amphibians, birds, crocodiles, fish, lizards, mammals, reptiles, and snakes, or any combination thereof.

In one embodiment, the invention provides an isolated nucleic acid, inducible promoter, nucleic acid, expression cassette, vector, cell, method or kit, or any combination thereof, for use in a method of producing an antibody or antigen binding fragment thereof.

Also provided herein is a food product, wherein the food product comprises a cell according to any of the previous claims, or an expression product of a gene produced according to any of the methods of the preceding claims. In some embodiments, the food product is used to feed an animal belonging to a phylum selected from but not limited to the group comprising or consisting: annelids, arthropods, bryozoan, chordates, cnidaria, echinoderms, molluscs, nematodes, platyhelminths, rotifers, sponges, or any combination thereof. In one preferred embodiment, the chordate animal is selected from but not limited to the group consisting or comprising: amphibians, birds, crocodiles, fish, lizards, mammals, reptiles, and snakes, or any combination thereof.

The listing or discussion of an apparently prior-published document in this specification should not necessarily be taken as an acknowledgement that the document is part of the state of the art or is common general knowledge.

Preferences and options for a given aspect, feature or parameter of the invention should, unless the context indicates otherwise, be regarded as having been disclosed in combination with any and all preferences and options for all other aspects, features and parameters of the invention. For example, the invention provides a Yarrowia lipolytic cell comprising a vector wherein the vector comprises a nucleic acid sequence that comprises a 500 bp region of SEQ ID NO: 5 operably linked to a second nucleic acid sequence that encodes a protein. The invention also provides a 900 bp fragment of any of SEQ ID NO: 1-33 wherein the fragment comprises at least 80% sequence identity to the relevant 900 bp sequence of SEQ ID NO: 1-33.

The invention also provides the following numbered embodiment paragraphs:

1. An isolated nucleic acid capable of acting as an inducible promoter in a non-methylotrophic yeast species, wherein expression from the promoter is induced by an inducing agent where the inducing agent is any one or more compound selected from the group consisting or comprising of: formate, formic acid, formaldehyde, methanol, ethanol, propanol, butanol and glycerol, and:
wherein the nucleic acid:
   a) comprises a sequence that comprises or consists of the consensus sequence set out in
   SEQ ID NO: 1 GTGCGGCTCGGAAATTCACAWGGKCCGT-TYGTGCGGCTCGGAAAT, where:
      Y is a pyrimidine nucleotide, nucleobase or base;
      W is a Weak nucleotide, nucleobase or base, optionally an A nucleotide, nucleobase or base or a T nucleotide, nucleobase or base;
      K is a Keto nucleotide, nucleobase or base, optionally a G nucleotide, nucleobase or base or a T nucleotide, nucleobase or base;
   or any synthetic analogue or chemically modified nucleotide, nucleobase or base thereof; and/or
   b) comprises or consists of a portion of a sequence selected from a group comprising or consisting of:
      i) SEQ ID NO: 8, SEQ ID NO: 24, SEQ ID NO: 6, SEQ ID NO: 22, SEQ ID NO: 9, SEQ ID NO: 25, SEQ ID NO: 11, SEQ ID NO: 27, SEQ ID NO: 2, SEQ ID NO: 18, SEQ ID NO: 10, SEQ ID NO: 26, SEQ ID NO: 7, SEQ ID NO: 23, SEQ ID NO: 3, SEQ ID NO: 19, SEQ ID NO: 5, SEQ ID NO: 21, SEQ ID NO: 4, SEQ ID NO: 20, SEQ ID NO: 12, SEQ ID NO: 13, SEQ ID NO: 14, SEQ ID NO: 15, SEQ ID NO: 16, SEQ ID NO: 17, SEQ ID NO: 28, SEQ ID NO: 29, SEQ ID NO: 30, SEQ ID NO: 31, SEQ ID NO: 32, or SEQ ID NO: 33; or
      comprises a portion of a sequence selected from a group comprising a sequence with at least 80%, 85%, 90%, 92%, 94%, 95%, 96%, 97%, 98% or 99% sequence identity to SEQ ID NO: 6, SEQ ID NO: 22, SEQ ID NO: 9, SEQ ID NO: 25, SEQ ID NO: 11, SEQ ID NO: 27, SEQ ID NO: 2, SEQ ID NO: 18, SEQ ID NO: 10, SEQ ID NO: 26, SEQ ID NO: 7, SEQ ID NO: 23, SEQ ID NO: 3, SEQ ID NO: 19, SEQ ID NO: 5, SEQ ID NO: 21, SEQ ID NO: 4, SEQ ID NO: 20, SEQ ID NO: 12, SEQ ID NO: 13, SEQ ID NO: 14, SEQ ID NO: 15, SEQ ID NO: 16, SEQ ID NO: 17 SEQ ID NO: 28, SEQ ID NO: 29, SEQ ID NO: 30, SEQ ID NO: 31, SEQ ID NO: 32, or SEQ ID NO: 33;
      ii) SEQ ID NO: 8, SEQ ID NO: 24, SEQ ID NO: 6, SEQ ID NO: 22, SEQ ID NO: 9, SEQ ID NO: 25, SEQ ID NO: 11, SEQ ID NO: 27, SEQ ID NO: 2, SEQ ID NO: 18, SEQ ID NO: 10, SEQ ID NO: 26, SEQ ID NO: 7, SEQ ID NO: 23, SEQ ID NO: 3, SEQ ID NO: 19, SEQ ID NO: 5, SEQ ID NO: 21, SEQ ID NO: 4, SEQ ID NO: 20; or
      comprises a portion of a sequence selected from a group comprising a sequence with at least 80%, 85%, 90%, 92%, 94%, 95%, 96%, 97%, 98% or 99% sequence identity to SEQ ID NO: 8, SEQ ID NO: 24, SEQ ID NO: 6, SEQ ID NO: 22, SEQ ID NO: 9, SEQ ID NO: 25, SEQ ID NO: 11, SEQ ID NO: 27, SEQ ID NO: 2, SEQ ID NO: 18, SEQ ID NO: 10, SEQ ID NO: 26, SEQ ID NO: 7, SEQ ID NO: 23, SEQ ID NO: 3, SEQ ID NO: 19, SEQ ID NO: 5, SEQ ID NO: 21, SEQ ID NO: 4, SEQ ID NO: 20;
      iii) SEQ ID NO: 8, SEQ ID NO: 24, SEQ ID NO: 6, SEQ ID NO: 22, SEQ ID NO: 9, SEQ ID NO: 25, or comprises a portion of a sequence selected from a group comprising a sequence with at least 80%, 85%, 90%, 92%, 94%, 95%, 96%, 97%, 98% or 99% sequence identity toSEQ ID NO: 8, SEQ ID NO: 24, SEQ ID NO: 6, SEQ ID NO: 22, SEQ ID NO: 9, SEQ ID NO: 25; or
      iv) SEQ ID NO: 8, SEQ ID NO: 24, SEQ ID NO: 6, SEQ ID NO: 22, or comprises a portion of a sequence selected from a group comprising a sequence with at least 80%, 85%, 90%, 92%, 94%, 95%, 96%, 97%, 98% or 99% sequence identity to SEQ ID NO: 8, SEQ ID NO: 24, SEQ ID NO: 6, SEQ ID NO: 22.

2. The isolated nucleic acid according to embodiment 1 wherein expression from the promoter in the absence of the inducing agent is low or absent.

3. The isolated nucleic acid according to any of embodiments 1 or 2 wherein expression from the promoter is increased by at least 2-fold or at least 3, 4, 5, 6, 7, 8, 9, 10, 11, 12, 14, 15, 20, 22, 24, 26, 28, 30, 32, 34, 36, 38, 40, 45 or at least 50-fold when the non-methylotrophic yeast species is cultured in YNB with 0.5% sodium format.

4. The isolated nucleic acid of any of embodiments 1-3 wherein the portion of the sequence is
between about 46 and 1500 bp in length, for example between 50 and 1500 bp in length, for example between 75 and 1500 bp in length, for example between 100 and 1500 bp in length, for example between 150 and 1400, 200 and 1300, 200 and 1200, 250 and 1100, 250 and 1000, 300 and 950, 350 and 900, 400 and 850, 450 and 800, 500 and 750, 550 and 700, 600 and 650 bp in length; and/or is at least 50, 46, 50, 75, 100, 150, 200, 250, 300, 350, 400, 450, 500, 550, 600, 650, 700, 750, 800, 850, 900, 950, 1000, 1100, 1200, 1300, 1400 or at least 1500 bp in length.

5. The isolated nucleic acid according to any of embodiments 1-4 wherein the nucleic acid is less than 1500 bp in length, optionally is about 46, 50, 75, 100, 150, 200, 250, 300, 350, 400, 450, 500, 550, 600, 650, 700, 750, 800, 850, 900, 950, 1000, 1100, 1200, 1300, 1400 or about 1500 bp in length.

6. The isolated nucleic acid according to any of embodiments 1-5 wherein the nucleic acid comprises or consists of a sequence of a portion of a region of up to 1Kb or up to 1.5Kb directly upstream of the translation start codon of a FDH gene, or of a putative FDH gene identified in a non-methylotrophic organism, optionally wherein said portion is:
about 46, 50, 75, 100, 150, 200, 250, 300, 350, 400, 450, 500, 550, 600, 650, 700, 750, 800, 850, 900, 950, 1000, 1100, 1200, 1300, 1400 or about 1500 bp in length; and/or is
between about 100 and 1500 bp in length, for example between 150 and 1400, 200 and 1300, 200 and 1200, 250 and 1100, 250 and 1000, 300 and 950, 350 and 900, 400 and 850, 450 and 800, 500 and 750, 550 and 700, 600 and 650 bp in length.

7. The isolated nucleic acid of any of embodiments 1-6, wherein expression from the inducible promoter is induced by formate.

8. The isolated nucleic acid of any of embodiments 1 to 7 wherein the nucleic acid is flanked by one or more restriction enzyme digestion sites, optionally by one or more type II restriction enzyme digestion sites.

9. A nucleic acid construct comprising at least a first and a second nucleic acid sequence, wherein the first nucleic acid sequence comprises or consists of the isolated nucleic acid sequence of any of embodiments 1 to 8.

10. The nucleic acid construct of embodiment 9, wherein the second nucleic acid sequence is a sequence capable of being transcribed into RNA, and wherein the first nucleic acid sequence is operably linked to the second nucleic acid sequence, optionally wherein the 3′ end of the first nucleic acid sequence is linked to the 5′ end of the second nucleic acid sequence by a sequence comprising or consisting the sequence CACA.

11. The nucleic acid construct of any of embodiments 9 or 10 wherein the second nucleic acid sequence is transcribed into mRNA, optionally wherein the second nucleic acid sequence encodes a peptide or polypeptide.

12. The nucleic acid construct of any of embodiments 9-11 wherein the second nucleic acid sequence is capable of being transcribed into an RNA sequence selected from the group consisting of or comprising: mRNA, rRNA, miRNA, siRNA, piRNA, snRNA, snoRNA, exRNA, scaRNA, lncRNA, gRNA, sgRNA, crRNA, and tracrRNA.

13. The nucleic acid construct of any of embodiments 9-12, wherein the second nucleic acid sequence does not encode a formate dehydrogenase (FDH) gene,
optionally
   does not encode a formate dehydrogenase gene from Yarrowia, optionally from Yarrowia lipolytica,
   wherein where:
   the first nucleic acid sequence comprises or consists of a portion of SEQ ID NO: 8 or 24, or comprises or consists of SEQ ID NO: 8 or 24, the second nucleic acid does not encode YALI0E14256 (SEQ ID NO:40);
   the first nucleic acid sequence comprises or consists of a portion of SEQ ID NO: 2 or 18, or comprises or consists of SEQ ID NO: 2 or 18, the second nucleic acid does not encode YALI0A21353 (SEQ ID NO:34);
   the first nucleic acid sequence comprises or consists of a portion of SEQ ID NO: 3 or 19, or comprises or consists of SEQ ID NO: 3 or 19, the second nucleic acid does not encode YALI0F15983 (SEQ ID NO:35);
   the first nucleic acid sequence comprises or consists of a portion of SEQ ID NO: 4 or 20, or comprises or consists of SEQ ID NO: 4 or 20, the second nucleic acid does not encode YALI0B22506 (SEQ ID NO:36);
   the first nucleic acid sequence comprises or consists of a portion of SEQ ID NO: 5 or 21, or comprises or consists of SEQ ID NO: 5 or 21, the second nucleic acid does not encode YALI0C08074 (SEQ ID NO:37);
   the first nucleic acid sequence comprises or consists of a portion of SEQ ID NO: 6 or 22, or comprises or consists of SEQ ID NO: 6 or 22, the second nucleic acid does not encode YALI0F13937 (SEQ ID NO:38);
   the first nucleic acid sequence comprises or consists of a portion of SEQ ID NO: 7 or 23, or comprises or consists of SEQ ID NO: 7 or 23, the second nucleic acid does not encode YALI0C14344 (SEQ ID NO:39);
   the first nucleic acid sequence comprises or consists of a portion of SEQ ID NO: 9 or 25, or comprises or consists of SEQ ID NO: 9 or 25, the second nucleic acid does not encode YALI0B19976 (SEQ ID NO:41);
   the first nucleic acid sequence comprises or consists of a portion of SEQ ID NO: 10 or 26, or comprises or consists of SEQ ID NO: 10 or 26, the second nucleic acid does not encode YALI0E15840 (SEQ ID NO:42);
   the first nucleic acid sequence comprises or consists of a portion of SEQ ID NO: 11 or 27, or comprises or consists of SEQ ID NO: 11 or 27, the second nucleic acid does not encode YALI0F28765 (SEQ ID NO:43);
   the first nucleic acid sequence comprises or consists of a portion of SEQ ID NO: 12 or 28, or comprises or consists of SEQ ID NO: 12 or 28, the second nucleic acid does not encode YALI0E19657g (SEQ ID NO:44);
   the first nucleic acid sequence comprises or consists of a portion of SEQ ID NO: 13 or 29, or comprises or consists of SEQ ID NO: 13 or 29, the second nucleic acid does not encode YALI0B21670g (SEQ ID NO:45);
   the first nucleic acid sequence comprises or consists of a portion of SEQ ID NO: 14 or 30, or comprises or consists of SEQ ID NO: 14 or 30, the second nucleic acid does not encode YALI0F29315g (SEQ ID NO:46);
   the first nucleic acid sequence comprises or consists of a portion of SEQ ID NO: 15 or 31, or comprises or consists of SEQ ID NO: 15 or 31, the second nucleic acid does not encode YALI0D25256g (SEQ ID NO:47);
   the first nucleic acid sequence comprises or consists of a portion of SEQ ID NO: 16 or 32, or comprises or consists of SEQ ID NO: 16 or 32, the second nucleic acid does not encode YALI0C11099g (SEQ ID NO:48);
   the first nucleic acid sequence comprises or consists of a portion of SEQ ID NO: 17 or 33, or comprises or consists of SEQ ID NO: 17 or 33, the second nucleic acid does not encode YALI0F09966g (SEQ ID NO:49).

14. An expression cassette comprising the isolated nucleic acid or nucleic acid construct of any of the preceding embodiments.

15. A vector comprising the isolated nucleic acid, or nucleic acid construct of any of the preceding embodiments, optionally wherein the vector is selected from a group comprising a plasmid or an artificial chromosome, optionally wherein the artificial chromosome is selected from a bacterial artificial chromosome (BAC), a yeast artificial chromosome (YAC), and a Human artificial chromosome (HAC).

16. A cell comprising:
i) the isolated nucleic acid of any of embodiments 1-8;
ii) the nucleic acid construct of any of embodiments 9-13;
iii) the expression cassette of embodiment 14; and/or
iv) the vector of embodiment 15;
   optionally wherein the cell is an engineered cell.

17. The cell of embodiment 16, wherein the cell is a eukaryotic cell, optionally wherein the cell is a cell selected from a group comprising:
a fungal cell;
a plant cell; and
an animal cell;
   optionally wherein the fungal cell is a yeast cell, optionally wherein the yeast cell is:
not a methylotrophic yeast cell, optionally from a genus selected from a group consisting or comprising: Ashbya, Blastobotrys, Crytococcus, Cutaneotrichosporon, Dekkera, Kluveromyces, Rhodosporidium, Rhodotorula, Lipomyces, Saccharomyces, and Yarrowia; or
a methylotrophic yeast cell, optionally from a genus selected from a group consisting or comprising: Candida, Hansenula, Komagataella, and Pichia.

18. The cell of embodiment 17, wherein the yeast cell is a cell belonging to the species Yarrowia lipolytica.

19. The cell of any of embodiments 16-18, wherein the isolated nucleic acid, nucleic acid construct, expression cassette, or vector is episomally maintained by said cell.

20. The cell of any of embodiments 16-18, wherein the isolated nucleic acid, inducible promoter, nucleic acid construct, expression cassette, or vector is integrated into the genome of said cell.

21. The cell of embodiment 20 wherein the isolated nucleic acid, inducible promoter, nucleic acid construct, expression cassette, or vector is integrated upstream of a second sequence located in the genome, and wherein following integration the isolated nucleic acid, inducible promoter, nucleic acid construct, expression cassette, or vector is capable of driving transcription of the second sequence.

22. The cell of either of embodiments 20 or 21, wherein the isolated nucleic acid, inducible promoter, nucleic acid, expression cassette, or vector is integrated into the genome of said cell at a different locus to the locus of the native promoter, optionally wherein where:
the isolated nucleic acid has a sequence of 80%, 85%, 90%, 92%, 94%, 95%, 96%, 97%, 98%, 99% or 100% identity to SEQ ID NO: 8 or 24 the isolated nucleic acid, inducible promoter, nucleic acid, expression cassette, or vector is integrated at a genomic locus that is different to the locus of native SEQ ID NO: 8 or 24, i.e., is not operably inserted upstream of the gene encoding SEQ ID NO: 40;
   the isolated nucleic acid has a sequence of 80%, 85%, 90%, 92%, 94%, 95%, 96%, 97%, 98%, 99% or 100% identity to SEQ ID NO: 2 or 18 the isolated nucleic acid, inducible promoter, nucleic acid, expression cassette, or vector is integrated at a genomic locus that is different to the locus of native SEQ ID NO: 2 or 18, i.e., is not operably inserted upstream of the gene encoding SEQ ID NO: 34;
the isolated nucleic acid has a sequence of 80%, 85%, 90%, 92%, 94%, 95%, 96%, 97%, 98%, 99% or 100% identity to SEQ ID NO: 3 or 19 the isolated nucleic acid, inducible promoter, nucleic acid, expression cassette, or vector is integrated at a genomic locus that is different to the locus of native SEQ ID NO: 3 or 19, i.e., is not operably inserted upstream of the gene encoding SEQ ID NO: 35;
the isolated nucleic acid has a sequence of 80%, 85%, 90%, 92%, 94%, 95%, 96%, 97%, 98%, 99% or 100% identity to SEQ ID NO: 4 or 20 the isolated nucleic acid, inducible promoter, nucleic acid, expression cassette, or vector is integrated at a genomic locus that is different to the locus of native SEQ ID NO: 4 or 20, i.e., is not operably inserted upstream of the gene encoding SEQ ID NO: 36;
the isolated nucleic acid has a sequence of 80%, 85%, 90%, 92%, 94%, 95%, 96%, 97%, 98%, 99% or 100% identity to SEQ ID NO: 5 or 21 the isolated nucleic acid, inducible promoter, nucleic acid, expression cassette, or vector is integrated at a genomic locus that is different to the locus of native SEQ ID NO: 5 or 21, i.e., is not operably inserted upstream of the gene encoding SEQ ID NO: 37;
the isolated nucleic acid has a sequence of 80%, 85%, 90%, 92%, 94%, 95%, 96%, 97%, 98%, 99% or 100% identity to SEQ ID NO: 6 or 22 the isolated nucleic acid, inducible promoter, nucleic acid, expression cassette, or vector is integrated at a genomic locus that is different to the locus of native SEQ ID NO: 6 or 22, i.e., is not operably inserted upstream of the gene encoding SEQ ID NO: 38;
the isolated nucleic acid has a sequence of 80%, 85%, 90%, 92%, 94%, 95%, 96%, 97%, 98%, 99% or 100% identity to SEQ ID NO: 7 or 23 the isolated nucleic acid, inducible promoter, nucleic acid, expression cassette, or vector is integrated at a genomic locus that is different to the locus of native SEQ ID NO: 7 or 23, i.e., is not operably inserted upstream of the gene encoding SEQ ID NO: 39;
the isolated nucleic acid has a sequence of 80%, 85%, 90%, 92%, 94%, 95%, 96%, 97%, 98%, 99% or 100% identity to SEQ ID NO: 9 or 25 the isolated nucleic acid, inducible promoter, nucleic acid, expression cassette, or vector is integrated at a genomic locus that is different to the locus of native SEQ ID NO: 9 or 25, i.e., is not operably inserted upstream of the gene encoding SEQ ID NO: 41;
the isolated nucleic acid has a sequence of 80%, 85%, 90%, 92%, 94%, 95%, 96%, 97%, 98%, 99% or 100% identity to SEQ ID NO: 10 or 26 the isolated nucleic acid, inducible promoter, nucleic acid, expression cassette, or vector is integrated at a genomic locus that is different to the locus of native SEQ ID NO: 10 or 26, i.e., is not operably inserted upstream of the gene encoding SEQ ID NO: 42;
the isolated nucleic acid has a sequence of 80%, 85%, 90%, 92%, 94%, 95%, 96%, 97%, 98%, 99% or 100% identity to SEQ ID NO: 11 or 27 the isolated nucleic acid, inducible promoter, nucleic acid, expression cassette, or vector is integrated at a genomic locus that is different to the locus of native SEQ ID NO: 11 or 27, i.e., is not operably inserted upstream of the gene encoding SEQ ID NO: 43;
the isolated nucleic acid has a sequence of 80%, 85%, 90%, 92%, 94%, 95%, 96%, 97%, 98%, 99% or 100% identity to SEQ ID NO: 12 or 28 the isolated nucleic acid, inducible promoter, nucleic acid, expression cassette, or vector is integrated at a genomic locus that is different to the locus of native SEQ ID NO: 12 or 28, i.e., is not operably inserted upstream of the gene encoding SEQ ID NO: 44;
the isolated nucleic acid has a sequence of 80%, 85%, 90%, 92%, 94%, 95%, 96%, 97%, 98%, 99% or 100% identity to SEQ ID NO: 13 or 29 the isolated nucleic acid, inducible promoter, nucleic acid, expression cassette, or vector is integrated at a genomic locus that is different to the locus of native SEQ ID NO: 13 or 29, i.e., is not operably inserted upstream of the gene encoding SEQ ID NO: 45;
the isolated nucleic acid has a sequence of 80%, 85%, 90%, 92%, 94%, 95%, 96%, 97%, 98%, 99% or 100% identity to SEQ ID NO: 14 or 30 the isolated nucleic acid, inducible promoter, nucleic acid, expression cassette, or vector is integrated at a genomic locus that is different to the locus of native SEQ ID NO: 14 or 30, i.e., is not operably inserted upstream of the gene encoding SEQ ID NO: 46;
the isolated nucleic acid has a sequence of 80%, 85%, 90%, 92%, 94%, 95%, 96%, 97%, 98%, 99% or 100% identity to SEQ ID NO: 15 or 31 the isolated nucleic acid, inducible promoter, nucleic acid, expression cassette, or vector is integrated at a genomic locus that is different to the locus of native SEQ ID NO: 15 or 31, i.e., is not operably inserted upstream of the gene encoding SEQ ID NO: 47;
the isolated nucleic acid has a sequence of 80%, 85%, 90%, 92%, 94%, 95%, 96%, 97%, 98%, 99% or 100% identity to SEQ ID NO: 16 or 32 the isolated nucleic acid, inducible promoter, nucleic acid, expression cassette, or vector is integrated at a genomic locus that is different to the locus of native SEQ ID NO: 16 or 32, i.e., is not operably inserted upstream of the gene encoding SEQ ID NO: 48; or
the isolated nucleic acid has a sequence of 80%, 85%, 90%, 92%, 94%, 95%, 96%, 97%, 98%, 99% or 100% identity to SEQ ID NO: 17 or 33 the isolated nucleic acid, inducible promoter, nucleic acid, expression cassette, or vector is integrated at a genomic locus that is different to the locus of native SEQ ID NO: 17 or 33, i.e., is not operably inserted upstream of the gene encoding SEQ ID NO: 49.

23. A method of producing an expression product of a gene, wherein the method comprises the use of the isolated nucleic acid, inducible promoter, nucleic acid construct, expression cassette, vector, or cell of any of the preceding embodiments.

24. The method according to embodiment 23, wherein the method further comprises the step of contacting the cell with an appropriate inducer agent, optionally wherein the inducer agent is formate.

25. A method of producing a secondary metabolite, wherein the method comprises the use of the isolated nucleic acid sequence, nucleic acid construct, inducible promoter, expression cassette, vector, or cell of any of the preceding embodiments.

26. A kit comprising at least two of any of:
the isolated nucleic acid of any of the preceding embodiments;
the nucleic acid construct of any of the preceding embodiments;
the inducible promoter of any of the preceding embodiments;
the expression cassette of any of the preceding embodiments;
the vector of any of the preceding embodiments;
the cell of any of the preceding embodiments;
an inducing agent, optionally wherein the inducing agent is formate.

27. The isolated nucleic acid, inducible promoter, nucleic acid construct, expression cassette, vector, cell, method or kit of any of the preceding embodiments for use in a method of producing animal feed.

28. The isolated nucleic acid, inducible promoter, nucleic acid construct, expression cassette, vector, cell, method or kit of any of the preceding embodiments wherein the expression from the promoter is induced in YNB media or in ACH +caa media.

29. The isolated nucleic acid, inducible promoter, nucleic acid construct, expression cassette, vector, cell, method or kit of any of the preceding embodiments for use in a method of producing a vaccine.

30. A food product wherein the food product comprises a cell according to any of the previous embodiments, or an expression product of a gene produced according to any of the methods of the preceding embodiments.

### FIGURE LEGENDS

FIG. 1: Consensus motif of ten formate induced promoters of Yarrowia lipolytica. The consensus motif sequence was derived by aligning 1500 bp upstream of the 10 fdh genes identified herein using MUSCLE to identify conserved regions.

FIG. 2: Multiple sequence alignment of all putative Yarrowia lipolytica FDH protein sequences with YALI0E14256g1_1.

FIG. 3: Transcription levels of the FDH genes of Yarrowia lipolytica W29 measured by qPCR, normalised to actin (ACT1) expression. Yarrowia lipolytica was grown in YNB supplemented with 1% glucose and 0.5% yeast extract at 15 h at 28° C., before being washed and transferred into YNB with 0.5% of sodium formate for induction and grown for 6 h.

FIG. 4: OD-normalized fluorescence of YL DSM 17984 transformed with empty vector (nc, p15021), pFDH-sfGFP (p15032) or pTEF sfGFP (p28003) grown in YNB (100 mg/L lysine, 260 mg/L leucine), with and without 1% of sodium formate. Cultivation in 30 mL of medium in shake flasks with n=3.

FIG. 5a: Measured fluorescence values for three different strains of Y. lipolytica grown in YNB supplemented with or without 0.05%, 1%, or 2.5% sodium formate. Formate was added at the start of incubation (early), after 24 hours (late), or not at all (no). Strain S29008 carries a genomically integrated sfGFP controlled by a putative formate induced promoter. Strain S11082 acts as a negative control carrying no sfGFP. Strain S06003 (positive control) expresses sfGFP constitutively under a TEF promoter (genomically integrated).

FIG. 5b: Measured fluorescence values for three different strains of Y. lipolytica grown in YPD supplemented with or without 0.05%, 1%, or 2.5% sodium formate. Formate was added at the start of incubation (early), after 24 hours (late), or not at all (no). Strain S29008 carries a genomically integrated sfGFP controlled by a putative formate induced promoter. Strain S11082 acts as a negative control carrying no sfGFP. Strain S06003 (positive control) expresses sfGFP constitutively under a TEF promoter (genomically integrated).

FIG. 5c: Measured fluorescence values for strains of Y. lipolytica carrying different plasmids, grown in ACH media supplemented with 14 g/L casamino acids (CAA) and 0.05%, 1%, or 2.5% sodium formate. Formate was added at the start of incubation (early), after 24 hours (late), or not at all (no). Plasmid p28003 carries a sfGFP sequence controlled by a constitutive pTEF promoter. Plasmid p15032 uses p28003 as a backbone but has the pTEF promoter replaced by the putative formate induced promoter. Plasmid p15021 acts as a negative control carrying no sfGFP.

FIG. 5d: Measured fluorescence values for strains of Y. lipolytica carrying different plasmids, grown in ACH media supplemented with 3.5 g/L casamino acids (CAA) and 0.05%, 1%, or 2.5% sodium formate. Formate was added at the start of incubation (early), after 24 hours (late), or not at all (no). Plasmid p28003 carries a sfGFP sequence controlled by a constitutive pTEF promoter. Plasmid p15032 uses p28003 as a backbone but has the pTEF promoter replaced by the putative formate induced promoter. Plasmid p15021 acts as a negative control carrying no sfGFP.

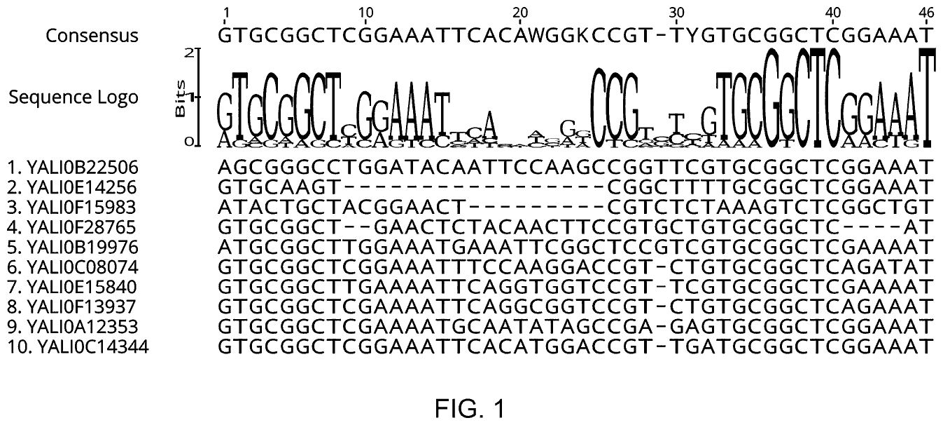

The listing or discussion of an apparently prior-published document in this specification should not necessarily be taken as an acknowledgement that the document is part of the state of the art or is common general knowledge.

Preferences and options for a given aspect, feature or parameter of the invention should, unless the context indicates otherwise, be regarded as having been disclosed in combination with any and all preferences and options for all other aspects, features and parameters of the invention. For example, the invention provides an isolated nucleic acid capable of acting as an inducible promoter in a non-methylotrophic yeast species wherein the nucleic acid comprises a 150 bp region of SEQ ID NO: 3 and which is inducible by formate in YNB media. The invention also provides a nucleic acid construct comprising a nucleic acid capable of acting as an inducible promoter, wherein the nucleic acid comprises a sequence according to SEQ ID NO: 1 and a second nucleic acid, wherein the second nucleic acid encodes a protein involved in the production of phenolic compounds.

### EXAMPLES

The invention will now be exemplified with the following non-limiting Examples.

### Example 1

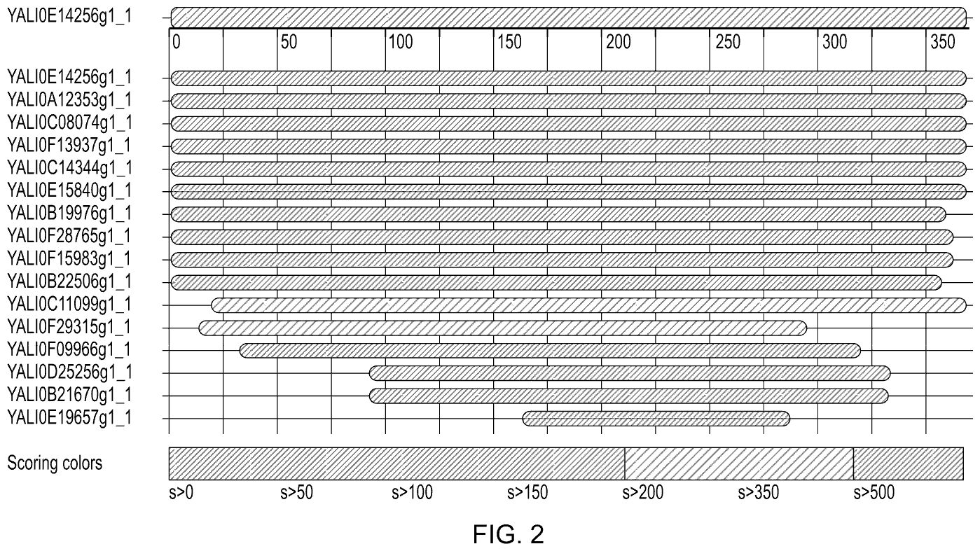

In order to identify formate-inducible promoters in the non-methylotrophic yeast Yarrowia lipolytica, formate dehydrogenase (FDH) genes encoded in the Yarrowia lipolytica E150 were first identified by homology to the Y. lipolytica FDH protein, YALI0E14256g1_1. 16 putative FDH proteins were identified in total (FIG. 2), corresponding to the 16 putative FDH genes described herein (SEQ ID NO: 18-33).

The inducibility of the putative FDH genes with formate was then tested by qPCR. YALI0E14256g1_1 and the nine putative FDH with the highest % identity to YALI0E14256g1_1 were selected for testing. Y. lipolytica JMY2900 was cultured in YNB media supplemented with 1% glucose and 0.5% yeast extract for 15 h at 28° C. Cells were washed with distilled water and cultured in fresh liquid YNB media supplemented with 0.5% sodium formate at 28° C. with agitation at 160 rpm, before being harvested at 6 h post-inoculated. Cells were frozen in liquid nitrogen and stored at −80° C. RNA was subsequently extracted using the RNeasy Mini Kit (Qiagen), and 2 μg was treated with DNAse (Ambion; Life Science Technologies, Saint-Aubain, France). cDNA was synthesised using the Maxima First Strand cDNA synthesis kit (Thermo Fischer Scientific, Villebon sur Yvette, France). qPCR was then performed using the SYBRgreen mastermix (Thermo Fischer Scientific) with gene-specific primers designed using Primer3 software. Relative expression levels were calculated using ΔC_{T} and ΔΔC_{T} methods. Expression levels were normalised to actin.

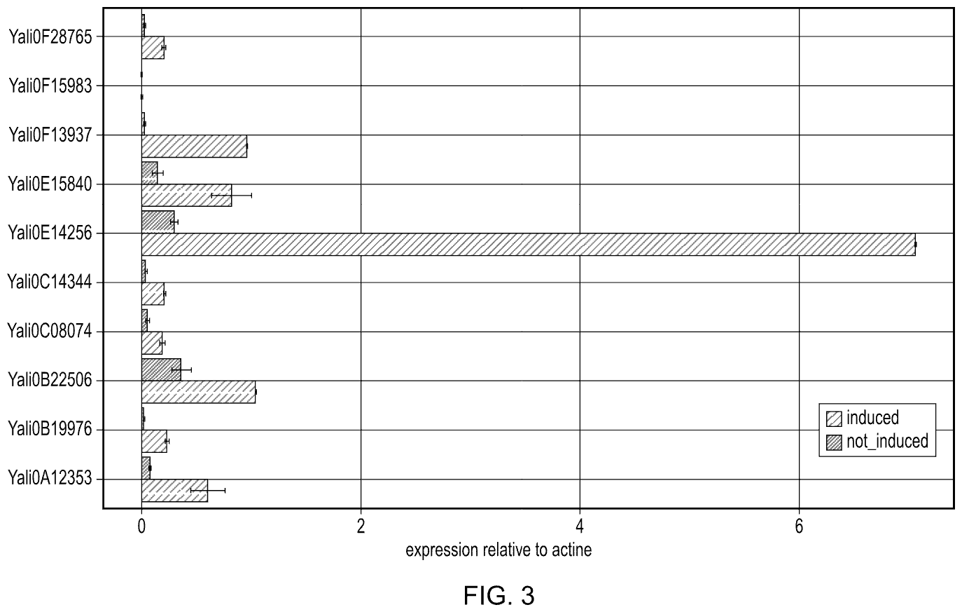

qPCR analysis of 10 putative FDH genes demonstrated increased expression in all ten genes tested when Y. lipolytica is cultured with 0.5% sodium formate, compared to culturing in the absence of formate. See FIG. 3 (induced=+0.5% sodium formate; not_induced=−0.5% sodium formate) and Table 1.

TABLE 1
fold change expression of putative FDH genes
upon induction with sodium formate

| Putative FDH gene | Fold increase on induction |
|---|---|
| Yali0F28765 | 8.1 |
| Yali0F15983 | 4.2 |
| Yali0C08074 | 3.7 |
| Yali0A21353 | 7.9 |
| Yali0E15840 | 5.7 |
| Yali0F13937 | 38.6 |
| Yali0B22506 | 2.9 |
| Yali0B19976 | 11.4 |
| Yali0E14256 | 23.9 |
| Yali0C14344 | 4.9 |

### Example 2

Expression from the putative FDH promoters was characterised by a promoter-GFP assay. A 1500 bp fragment directly upstream of the YALI0E14256 gene was amplified from the Y. lipolytica H222 genome and cloned into the p28003 backbone plasmid with sfGFP by SLiCE cloning (Messerschmidt et al., 2016), resulting in the plasmid pFDH-sfGFP, containing a fusion of the 1500 bp FDH promoter operably linked to the sfGFP gene. pFDH-sfGFP was transformed into Y. lipolytica s15028 for testing. A positive control plasmid, p28803 or pTEF-sfGFP, comprising the constitutive TEF promoter was also transformed into Y. lipolytica s15028 as a fluorescence positive control.

Fluorescence signal produced by untransformed cells and cells transformed with pFDH-sfGFP or pTEF-sfGFP was tested by culturing the cells in a 24-well plate in YNB supplemented with 20 gL⁻¹ glucose, 100 mgL⁻¹ lysine and 260 mgL⁻¹ leucine at 30° C. with agitation at 150 rpm. GFP expression was induced by the addition of sodium formate to a final concentration of 1% (w/v). Fluorescence was detected and normalised to the OD₆₀₀ of the cultures.

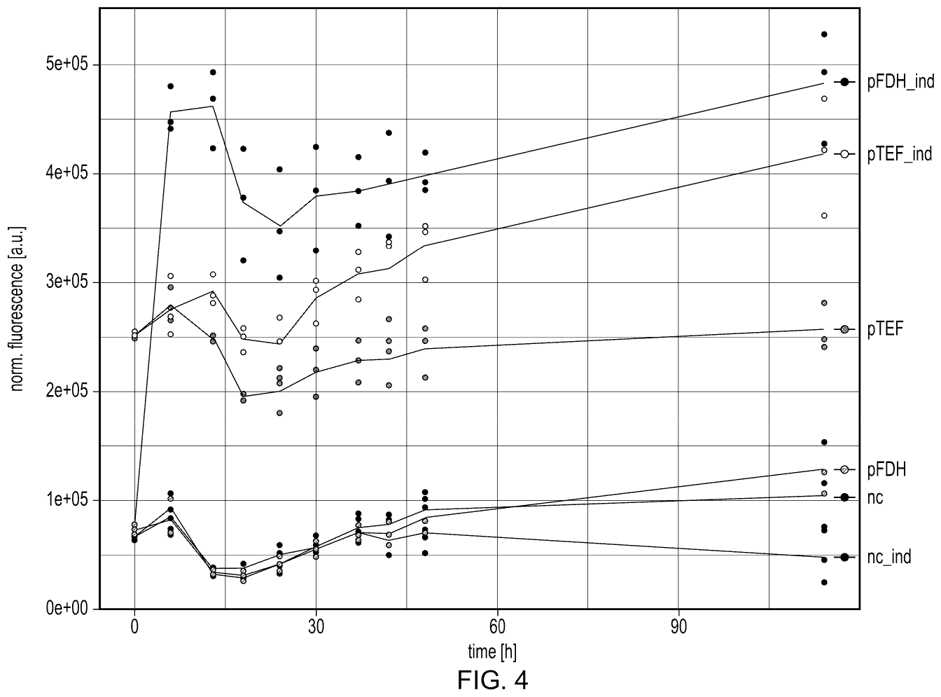

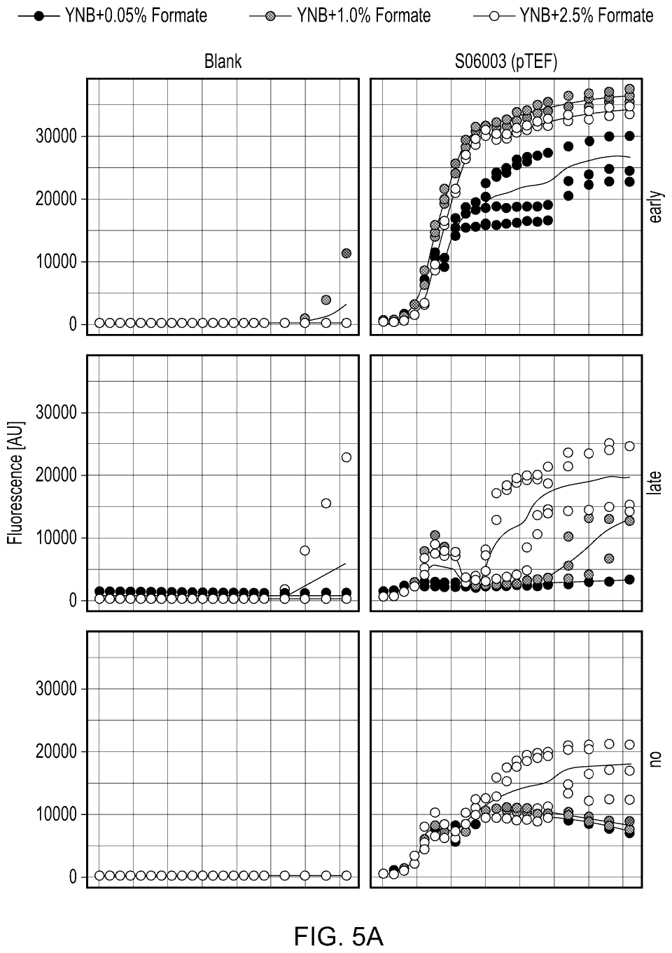

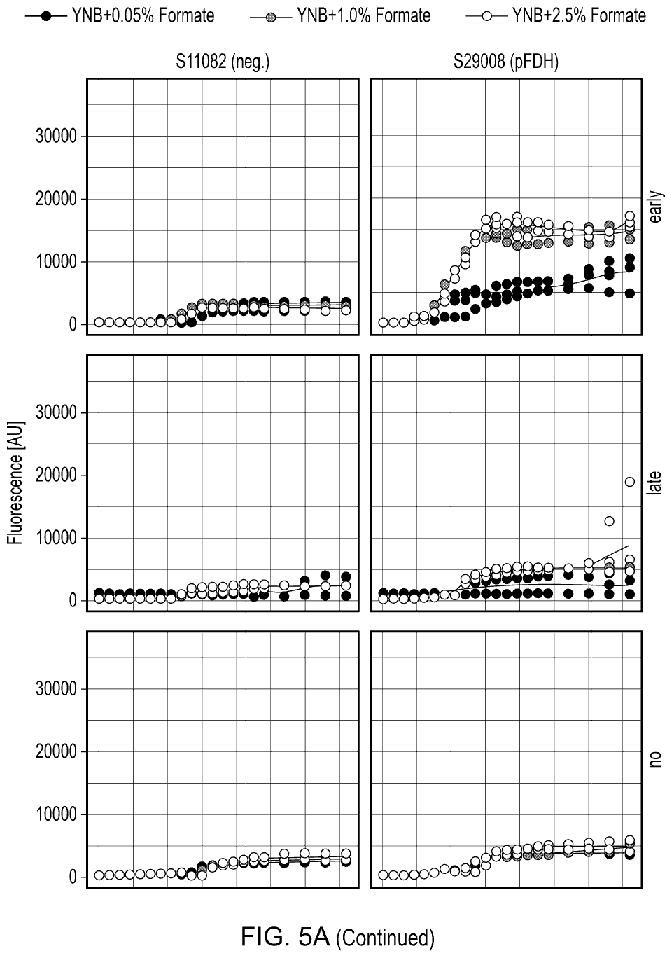

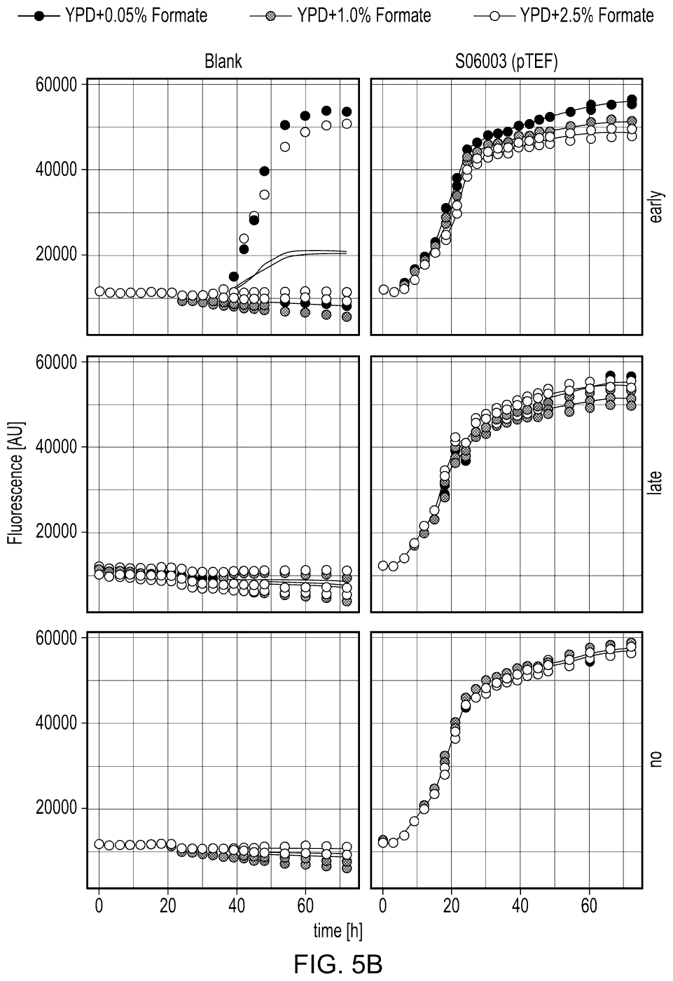

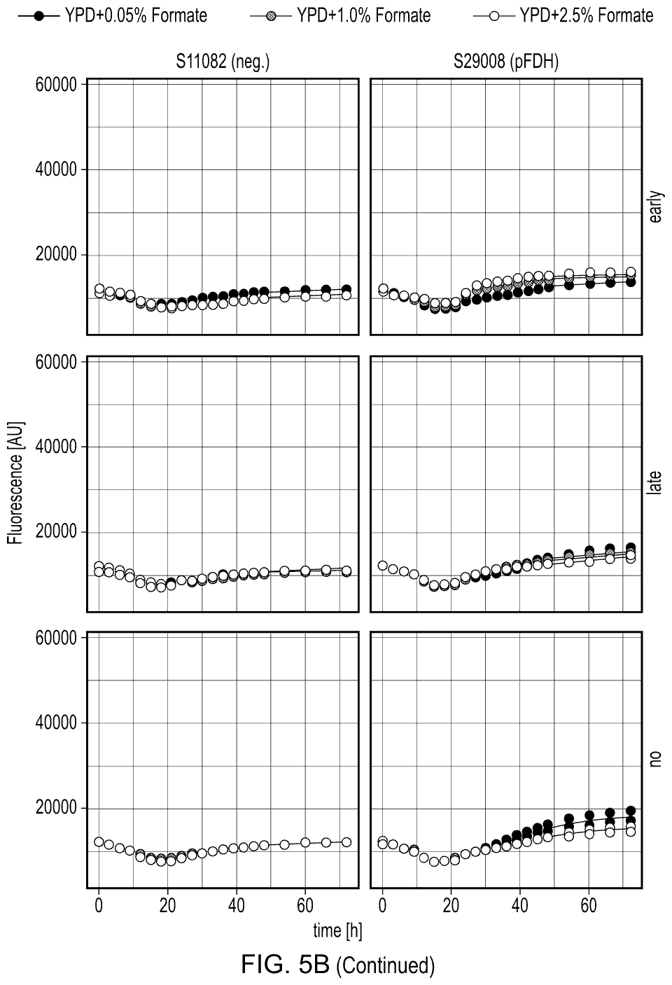

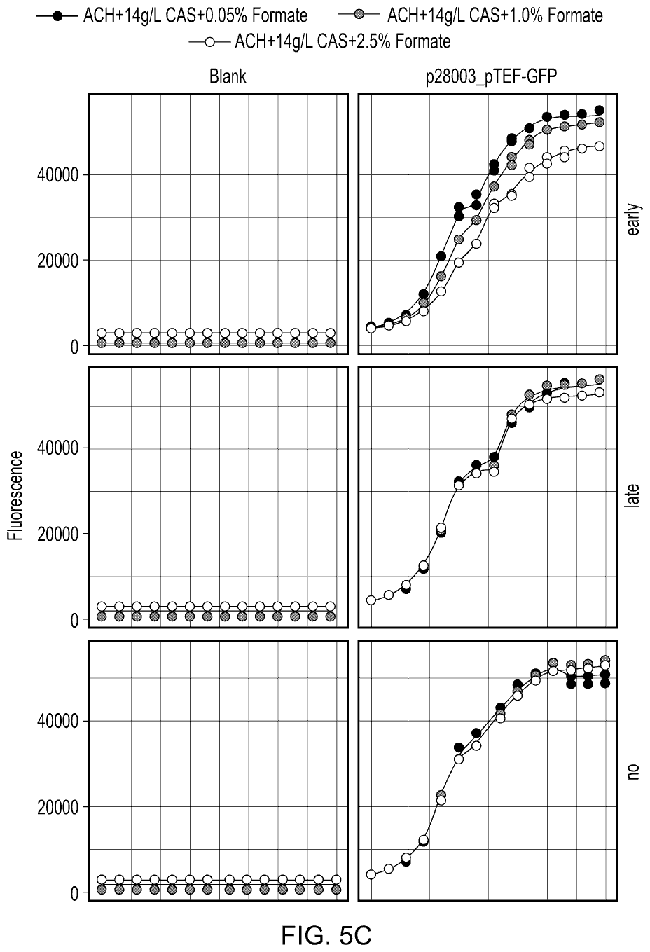

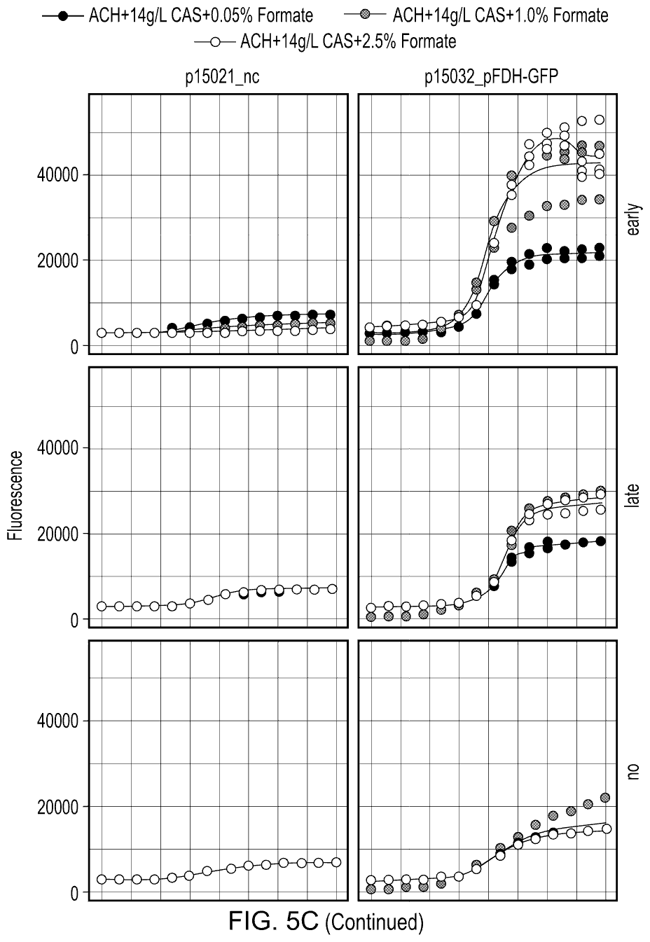

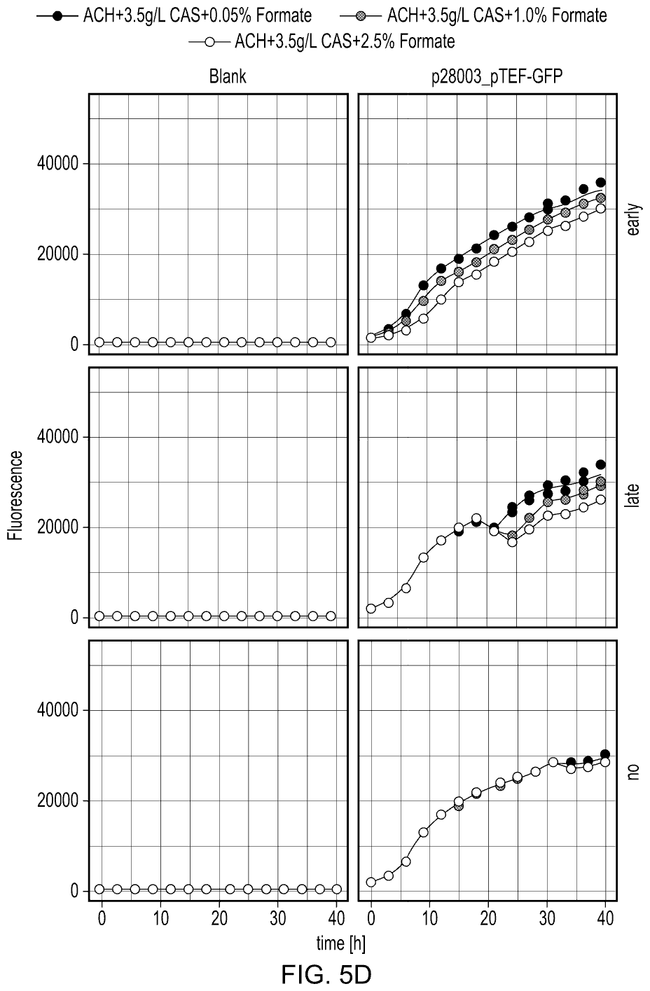

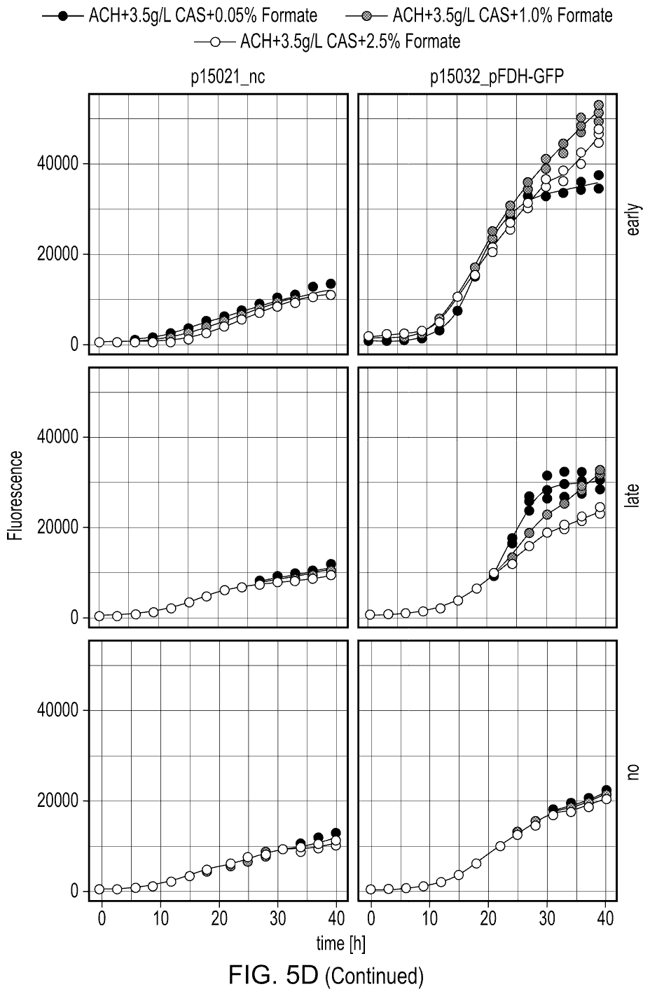

FIG. 4 shows that the addition of 1% (w/v) sodium formate induces expression of sfGFP from pFDH-sfGFP. No difference was observed in the fluorescence of untransfected cells. A small increase was observed in fluorescence produced by cells transformed with pTEF-sfGFP. These data show that FDH promoters are inducible to a high level by formate (FIG. 3, nc=untransformed cells; pFDH=pFDH-sfGFP; pTEF =pTEF-sfGFP; _ind=+sodium formate).

### Example 3

To measure the inducible expression of GFP under the control of Y. lipolytica FDH promoters under different conditions, three different media for the cultivation of Y. lipolytica were compared: yeast extract peptone dextrose media (YPD; Carl Roth—Karlsruhe, Germany; 50 g/L); yeast nitrogen base without amino acids media (YNB; Carl Roth—Karlsruhe, Germany; 6.8 g/L); and ACH media (6.7 g/L YNB, 14 g/L CAS amino acids, 10 g/L glucose, all obtained from Carl Roth—Karlsruhe, Germany).

Precultures were grown overnight in TPD, YNB, or ACH. Cells were pelleted by centrifugation before being resuspended in the corresponding media. The OD of each suspension was measured. The suspension was then diluted before 10 μl diluted cell suspension was inoculated into 180 μl of the corresponding media in the wells of a 96-well plate, to a final OD₆₀₀=0.1. Inoculated plates were incubated in a Cytomat 2 tower shaker at 28° C. for 72 h with agitation at 1000 rpm. Expression was induced by addition of sodium formate to a final concentration of 0.05%, 1%, or 2.5% (w/v) at the start of plate incubation (early) or after 24 hours (late). A third group remained without formate addition (no). Four repeats were conducted per condition.

Every 3 hours during incubation, each plate was transferred from the incubator to a PHERAstar FSX plate reader, where OD, fluorescence and fluorescence polarization at two different gain levels were measured. Plates were transferred by a four-axis Precise Flex 760 (Precise Automation) laboratory robot. An excitation wavelength of 485 nm was used, while emission was measured at 520 nm. “Late” addition of sodium formate was conducted at 24 h post-inoculation by pipetting 10 μL of sodium formate solution from a storage plate into each well using a CyBio Felix with a 96-channel head. Wells filled with media, were used as blanks. Into the respective blanks, formate was added along the corresponding conditions for each induction timepoint.

As a result, we observed that no induction was observed in YPD while a clear induction was found in both YNB and ACH+caa when formate was added in the culture media. FDH allowed the transition from close to zero expression (with no addition of formate) to strong expression (when formate was added). As a control pTEF was used, which showed expression in any media regardless of the addition of formate. Some variations was observed in TEF expression when formate was added, which could be explained by general metabolic changes caused by the metabolism of this compound by FDHs genes, which affect redox state of the cell.

1. An isolated nucleic acid capable of acting as an inducible promoter in a non-methylotrophic yeast species, wherein expression from the promoter is induced by an inducing agent where the inducing agent is any one or more compound selected from the group consisting or comprising of: formate, formic acid, formaldehyde, methanol, ethanol, propanol, butanol and glycerol, and:
wherein the nucleic acid:
a) comprises a sequence that comprises or consists of the consensus sequence set out in SEQ ID NO: 1 GTG CG G CTCG G AA ATT C AC A W G G KCCGT-TY GTG CG G CTCG G AA AT, where:
Y is a pyrimidine nucleotide, nucleobase or base;
W is a Weak nucleotide, nucleobase or base, optionally an A nucleotide,
nucleobase or base or a T nucleotide, nucleobase or base;
K is a Keto nucleotide, nucleobase or base, optionally a G nucleotide, nucleobase or base or a T nucleotide, nucleobase or base; or any synthetic analogue or chemically modified nucleotide, nucleobase or base thereof; and/or
b) comprises or consists of a portion of a sequence selected from a group comprising or consisting of:
i) SEQ ID NO: 8, SEQ ID NO: 24, SEQ ID NO: 6, SEQ ID NO: 22, SEQ ID NO: 9, SEQ ID NO: 25, SEQ ID NO: 11, SEQ ID NO: 27, SEQ ID NO: 2, SEQ ID NO: 18, SEQ ID NO: 10, SEQ ID NO: 26, SEQ ID NO: 7, SEQ ID NO: 23, SEQ ID NO: 3, SEQ ID NO: 19, SEQ ID NO: 5, SEQ ID NO: 21, SEQ ID NO: 4, SEQ ID NO: 20, SEQ ID NO: 12, SEQ ID NO: 13, SEQ ID NO: 14, SEQ ID NO: 15, SEQ ID NO: 16, SEQ ID NO: 17, SEQ ID NO: 28, SEQ ID NO: 29, SEQ ID NO: 30, SEQ ID NO: 31, SEQ ID NO: 32, or SEQ ID NO: 33; or
comprises a portion of a sequence selected from a group comprising a sequence with at least 80%, 85%, 90%, 92%, 94%, 95%, 96%, 97%, 98% or 99% sequence identity to SEQ ID NO: 6, SEQ ID NO: 22, SEQ ID NO: 9, SEQ ID NO: 25, SEQ ID NO: 11, SEQ ID NO: 27, SEQ ID NO: 2, SEQ ID NO: 18, SEQ ID NO: 10, SEQ ID NO: 26, SEQ ID NO: 7, SEQ ID NO: 23, SEQ ID NO: 3, SEQ ID NO: 19, SEQ ID NO: 5, SEQ ID NO: 21, SEQ ID NO: 4, SEQ ID NO: 20, SEQ ID NO: 12, SEQ ID NO: 13, SEQ ID NO: 14, SEQ ID NO: 15, SEQ ID NO: 16, SEQ ID NO: 17 SEQ ID NO: 28, SEQ ID NO: 29, SEQ ID NO: 30, SEQ ID NO: 31, SEQ ID NO: 32, or SEQ ID NO: 33;
ii) SEQ ID NO: 8, SEQ ID NO: 24, SEQ ID NO: 6, SEQ ID NO: 22, SEQ ID NO: 9, SEQ ID NO: 25, SEQ ID NO: 11, SEQ ID NO: 27, SEQ ID NO: 2, SEQ ID NO: 18, SEQ ID NO: 10, SEQ ID NO: 26, SEQ ID NO: 7, SEQ ID NO: 23, SEQ ID NO: 3, SEQ ID NO: 19, SEQ ID NO: 5, SEQ ID NO: 21, SEQ ID NO: 4, SEQ ID NO: 20: or
comprises a portion of a sequence selected from a group comprising a sequence with at least 80%, 85%, 90%, 92%, 94%, 95%, 96%, 97%, 98% or 99% sequence identity to SEQ ID NO: 8, SEQ ID NO: 24, SEQ ID NO: 6, SEQ ID NO: 22, SEQ ID NO: 9, SEQ ID NO: 25, SEQ ID NO: 11, SEQ ID NO: 27, SEQ ID NO: 2, SEQ ID NO: 18, SEQ ID NO: 10, SEQ ID NO: 26, SEQ ID NO: 7, SEQ ID NO: 23, SEQ ID NO: 3, SEQ ID NO: 19, SEQ ID NO: 5, SEQ ID NO: 21, SEQ ID NO: 4, SEQ ID NO: 20;
iii) SEQ ID NO: 8, SEQ ID NO: 24, SEQ ID NO: 6, SEQ ID NO: 22, SEQ ID NO: 9, SEQ ID NO: 25, or
comprises a portion of a sequence selected from a group comprising a sequence with at least 80%, 85%, 90%, 92%, 94%, 95%, 96%, 97%, 98% or 99% sequence identity toSEQ ID NO: 8, SEQ ID NO: 24, SEQ ID NO: 6, SEQ ID NO: 22, SEQ ID NO: 9, SEQ ID NO: 25; or
iv) SEQ ID NO: 8, SEQ ID NO: 24, SEQ ID NO: 6, SEQ ID NO: 22, or
comprises a portion of a sequence selected from a group comprising a sequence with at least 80%, 85%, 90%, 92%, 94%, 95%, 96%, 97%, 98% or 99% sequence identity to SEQ ID NO: 8, SEQ ID NO: 24, SEQ ID NO: 6, SEQ ID NO: 22.

2. The isolated nucleic acid according to claim 1 wherein expression from the promoter in the absence of the inducing agent is low or absent or expression from the promoter is induced in YNB media or in ACH +caa media.

3. The isolated nucleic acid according to claim 1 wherein expression from the promoter is increased by at least 2-fold or at least 3, 4, 5, 6, 7, 8, 9, 10, 11, 12, 14, 15, 20, 22, 24, 26, 28, 30, 32, 34, 36, 38, 40, 45 or at least 50-fold when the non-methylotrophic yeast species is cultured in YNB with 0.5% sodium format.

4. The isolated nucleic acid of claim 1 wherein the portion of the sequence is
between about 46 and 1500 bp in length, for example between 50 and 1500 bp in length, for example between 75 and 1500 bp in length, for example between 100 and 1500 bp in length, for example between 150 and 1400, 200 and 1300, 200 and 1200, 250 and 1100, 250 and 1000, 300 and 950, 350 and 900, 400 and 850, 450 and 800, 500 and 750, 550 and 700, 600 and 650 bp in length; and/or
is at least 50, 46, 50, 75, 100, 150, 200, 250, 300, 350, 400, 450, 500, 550, 600, 650, 700, 750, 800, 850, 900, 950, 1000, 1100, 1200, 1300, 1400 or at least 1500 bp in length.

5. The isolated nucleic acid according to claim 1 wherein:
(a) the nucleic acid is less than 1500 bp in length, optionally is about 46, 50, 75, 100, 150, 200, 250, 300, 350, 400, 450, 500, 550, 600, 650, 700, 750, 800, 850, 900, 950, 1000, 1100, 1200, 1300, 1400 or about 1500 bp in length;
(b) the nucleic acid comprises or consists of a sequence of a portion of a region of up to 1Kb or up to 1.5Kb directly upstream of the translation start codon of a FD-1 gene, or of a putative FDH gene identified in a non-methylotrophic organism, optionally wherein said portion is:
about 46, 50, 75, 100, 150, 200, 250, 300, 350, 400, 450, 500, 550, 600, 650, 700, 750, 800, 850, 900, 950, 1000, 1100, 1200, 1300, 1400 or about 1500 bp in length; and/or is
between about 100 and 1500 bp in length, for example between 150 and 1400, 200 and 1300, 200 and 1200, 250 and 1100, 250 and 1000, 300 and 950, 350 and 900, 400 and 850, 450 and 800, 500 and 750, 550 and 700,600 and 650 bp in length;
(c) expression from the inducible promoter is induced by formate; and/or
(d) wherein the nucleic acid is flanked by one or more restriction enzyme digestion sites, optionally by one or more type II restriction enzyme digestion sites.

6. (canceled)

7. (canceled)

8. (canceled)

9. A nucleic acid construct comprising at least a first and a second nucleic acid sequence, wherein the first nucleic acid sequence comprises or consists of the isolated nucleic acid sequence of claim 1, optionally, wherein expression from the promoter is induced in YNB media or in ACH +caa media.

10. The nucleic acid construct of claim 9, wherein the second nucleic acid sequence is a sequence capable of being transcribed into RNA, and wherein the first nucleic acid sequence is operably linked to the second nucleic acid sequence, optionally wherein the 3′ end of the first nucleic acid sequence is linked to the 5′ end of the second nucleic acid sequence by a sequence comprising or consisting the sequence CACA.

11. The nucleic acid construct of claim 9 wherein the second nucleic acid sequence is transcribed into mRNA, optionally wherein the second nucleic acid sequence encodes a peptide or polypeptide.

12. The nucleic acid construct of claim 9 wherein the second nucleic acid sequence is capable of being transcribed into an RNA sequence selected from the group consisting of or comprising: mRNA, rRNA, miRNA, siRNA, piRNA, snRNA, snoRNA, exRNA, scaRNA, lncRNA, gRNA, sgRNA, crRNA, and tracrRNA.

13. The nucleic acid construct of claim 9, wherein the second nucleic acid sequence does not encode a formate dehydrogenase (FDH) gene, optionally
does not encode a formate dehydrogenase gene from Yarrowia, optionally from Yarrowia lipolytica,
wherein where:
the first nucleic acid sequence comprises or consists of a portion of SEQ ID NO: 8 or 24, or comprises or consists of SEQ ID NO: 8 or 24, the second nucleic acid does not encode YALI0E14256 (SEQ ID NO:40);
the first nucleic acid sequence comprises or consists of a portion of SEQ ID NO: 2 or 18, or comprises or consists of SEQ ID NO: 2 or 18, the second nucleic acid does not encode YALI0A21353 (SEQ ID NO:34);
the first nucleic acid sequence comprises or consists of a portion of SEQ ID NO: 3 or 19, or comprises or consists of SEQ ID NO: 3 or 19, the second nucleic acid does not encode YALI0F15983 (SEQ ID NO:35);
the first nucleic acid sequence comprises or consists of a portion of SEQ ID NO: 4 or 20, or comprises or consists of SEQ ID NO: 4 or 20, the second nucleic acid does not encode YALI0B22506 (SEQ ID NO:36);
the first nucleic acid sequence comprises or consists of a portion of SEQ ID NO: 5 or 21, or comprises or consists of SEQ ID NO: 5 or 21, the second nucleic acid does not encode YALI0C08074 (SEQ ID NO:37);
the first nucleic acid sequence comprises or consists of a portion of SEQ ID NO: 6 or 22, or comprises or consists of SEQ ID NO: 6 or 22, the second nucleic acid does not encode YALI0F13937 (SEQ ID NO:38);
the first nucleic acid sequence comprises or consists of a portion of SEQ ID NO: 7 or 23, or comprises or consists of SEQ ID NO: 7 or 23, the second nucleic acid does not encode YALI0C14344 (SEQ ID NO:39);
the first nucleic acid sequence comprises or consists of a portion of SEQ ID NO: 9 or 25, or comprises or consists of SEQ ID NO: 9 or 25, the second nucleic acid does not encode YALI0B19976 (SEQ ID NO:41);
the first nucleic acid sequence comprises or consists of a portion of SEQ ID NO: 10 or 26, or comprises or consists of SEQ ID NO: 10 or 26, the second nucleic acid does not encode YALI0E15840 (SEQ ID NO:42);
the first nucleic acid sequence comprises or consists of a portion of SEQ ID NO: I1 or 27, or comprises or consists of SEQ ID NO: 11 or 27, the second nucleic acid does not encode YALI0F28765 (SEQ ID NO:43);
the first nucleic acid sequence comprises or consists of a portion of SEQ ID NO: 12 or 28, or comprises or consists of SEQ ID NO: 12 or 28, the second nucleic acid does not encode YALI0E19657g (SEQ ID NO:44);
the first nucleic acid sequence comprises or consists of a portion of SEQ ID NO: 13 or 29, or comprises or consists of SEQ ID NO: 13 or 29, the second nucleic acid does not encode YALI0B21670g (SEQ ID NO:45);
the first nucleic acid sequence comprises or consists of a portion of SEQ ID NO: 14 or 30, or comprises or consists of SEQ ID NO: 14 or 30, the second nucleic acid does not encode YALI0F29315g (SEQ ID NO:46);
the first nucleic acid sequence comprises or consists of a portion of SEQ ID NO: 15 or 31, or comprises or consists of SEQ ID NO: 15 or 31, the second nucleic acid does not encode YALI0D25256g (SEQ ID NO:47);
the first nucleic acid sequence comprises or consists of a portion of SEQ ID NO: 16 or 32, or comprises or consists of SEQ ID NO: 16 or 32, the second nucleic acid does not encode YALI0C11099g (SEQ ID NO:48);
the first nucleic acid sequence comprises or consists of a portion of SEQ ID NO: 17 or 33, or comprises or consists of SEQ ID NO: 17 or 33, the second nucleic acid does not encode YALI0F09966g (SEQ ID NO:49).

14. An expression cassette comprising the isolated nucleic acid of claim 1 or nucleic acid construct comprising at least a first and a second nucleic acid sequence, wherein the first nucleic acid sequence comprises or consists of the isolated nucleic acid sequence of claim 1, optionally, wherein expression from the promoter is induced in YNB media or in ACH +caa media.

15. A vector comprising the isolated nucleic acid of claim 1, or nucleic acid construct of comprising at least a first and a second nucleic acid sequence, wherein the first nucleic acid sequence comprises or consists of the isolated nucleic acid sequence of claim 1, optionally wherein the vector is selected from a group comprising a plasmid or an artificial chromosome, optionally wherein the artificial chromosome is selected from a bacterial artificial chromosome (BAC), a yeast artificial chromosome (YAC), and a Human artificial chromosome (HAC), optionally, wherein expression from the promoter is induced in YNB media or in ACH +caa media.

16. A cell comprising:
i) the isolated nucleic acid of claim 1;
ii) a nucleic acid construct comprising at least a first and a second nucleic acid sequence, wherein the first nucleic acid sequence comprises or consists of the isolated nucleic acid sequence of claim 1;
iii) an expression cassette of comprising the isolated nucleic acid of claim 1 or nucleic acid construct comprising at least a first and a second nucleic acid sequence, wherein the first nucleic acid sequence comprises or consists of the isolated nucleic acid sequence of claim 1; and/or
iv) a vector of comprising the isolated nucleic acid of claim 1, or nucleic acid construct of comprising at least a first and a second nucleic acid sequence, wherein the first nucleic acid sequence comprises or consists of the isolated nucleic acid sequence of claim 1, optionally wherein the vector is selected from a group comprising a plasmid or an artificial chromosome, optionally wherein the artificial chromosome is selected from a bacterial artificial chromosome (BAC), a yeast artificial chromosome (YAC), and a Human artificial chromosome (HAC);
optionally wherein the cell is an engineered cell, and optionally, wherein expression from the promoter is induced in YNB media or in ACH +caa media.

17. The cell of claim 16, wherein:
(a) the yeast cell is a cell belonging to the species Yarrowia lipolytica:
(b) the cell is a eukaryotic cell, optionally wherein the cell is a cell selected from a group comprising: a fungal cell; a plant cell; and an animal cell;
optionally wherein the fungal cell is a yeast cell, optionally wherein
the yeast cell is:
not a methylotrophic yeast cell, optionally from a genus selected from a group consisting or comprising: Ashbya, Blastobotrys, Crytococcus, Cutaneotrichosporon, Dekkera, Kluveromyces, Rhodosporidium, Rhodotorula, Lipomyces, Saccharomyces, and Yarrowia; or
a methylotrophic yeast cell, optionally from a genus selected from a group consisting or comprising: Candida, Hansenula, Komagataella, and Pichia;
(c) the isolated nucleic acid, nucleic acid construct, expression cassette, or vector is episomally maintained by said cell and/or
(d) the isolated nucleic acid, inducible promoter, nucleic acid construct, expression cassette, or vector is integrated into the genome of said cell.

18. (canceled)

19. (canceled)

20. (canceled)

21. (canceled)

22. The cell of claim 17, wherein:
(a) the isolated nucleic acid, inducible promoter, nucleic acid construct, expression cassette, or vector is integrated upstream of a second sequence located in the genome, and wherein following integration the isolated nucleic acid, inducible promoter, nucleic acid construct, epression cassete, or vector is capable of driving transcription of the second sequence; and/or
(b) wherein following integration the isolated nucleic acid, inducible promoter, nucleic acid construct, expression cassette, or vector is capable of driving transcription of the second sequence the isolated nucleic acid, inducible promoter, nucleic acid, expression cassette, or vector is integrated into the genome of said cell at a different locus to the locus of the native promoter, optionally wherein where:
the isolated nucleic acid has a sequence of 80%, 85%, 90%, 92%, 94%, 95%, 96%, 97%, 98%, 99% or 100% identity to SEQ ID NO: 8 or 24 the isolated nucleic acid, inducible promoter, nucleic acid, expression cassette, or vector is integrated at a genomic locus that is different to the locus of native SEQ ID NO: 8 or 24, i.e., is not operably inserted upstream of the gene encoding SEQ ID NO: 40;
the isolated nucleic acid has a sequence of 80%, 85%, 90%, 92%, 94%, 95%, 96%, 97%, 98%, 99% or 100% identity to SEQ II) NO: 2 or 18 the isolated nucleic acid, inducible promoter, nucleic acid, expression cassette, or vector is integrated at a genomic locus that is different to the locus of native SEQ ID NO: 2 or 18, i.e., is not operably inserted upstream of the gene encoding SEQ ID NO: 34;
the isolated nucleic acid has a sequence of 80%, 85%, 90%, 92%, 94%, 95%, 96%, 97%, 98%, 99% or 100% identity to SEQ ID NO: 3 or 19 the isolated nucleic acid, inducible promoter, nucleic acid, expression cassette, or vector is integrated at a genomic locus that is different to the locus of native SEQ ID NO: 3 or 19, i.e., is not operably inserted upstream of the gene encoding SEQ ID NO: 35;
the isolated nucleic acid has a sequence of 80%, 85%, 90%, 92%, 94%, 95%, 96%, 97%, 98%, 99% or 100% identity to SEQ ID NO: 4 or 20 the isolated nucleic acid, inducible promoter, nucleic acid, expression cassette, or vector is integrated at a genomic locus that is different to the locus of native SEQ ID NO: 4 or 20, i.e., is not operably inserted upstream of the gene encoding SEQ ID NO: 36;
the isolated nucleic acid has a sequence of 80%, 85%, 90%, 92%, 94%, 95%, 96%, 97%, 98%, 99% or 100% identity to SEQ ID NO: 5 or 21 the isolated nucleic acid, inducible promoter, nucleic acid, expression cassette, or vector is integrated at a genomic locus that is different to the locus of native SEQ ID NO: 5 or 21, i.e., is not operably inserted upstream of the gene encoding SEQ ID NO: 37;
the isolated nucleic acid has a sequence of 80%, 85%, 90%, 92%, 94%, 95%, 96%, 97%, 98%, 99% or 100% identity to SEQ ID NO: 6 or 22 the isolated nucleic acid, inducible promoter, nucleic acid, expression cassette, or vector is integrated at a genomic locus that is different to the locus of native SEQ ID NO: 6 or 22, i.e., is not operably inserted upstream of the gene encoding SEQ ID NO: 38;
the isolated nucleic acid has a sequence of 80%, 85%, 90%, 92%, 94%, 95%, 96%, 97%, 98%, 99% or 100% identity to SEQ ID NO: 7 or 23 the isolated nucleic acid, inducible promoter, nucleic acid, expression cassette, or vector is integrated at a genomic locus that is different to the locus of native SEQ ID NO: 7 or 23, i.e., is not operably inserted upstream of the gene encoding SEQ ID NO: 39;
the isolated nucleic acid has a sequence of 80%, 85%, 90%, 92%, 94%, 95%, 96%, 97%, 98%, 99% or 100% identity to SEQ ID NO: 9 or 25 the isolated nucleic acid, inducible promoter, nucleic acid, expression cassette, or vector is integrated at a genomic locus that is different to the locus of native SEQ ID NO: 9 or 25, i.e., is not operably inserted upstream of the gene encoding SEQ ID NO: 41;
the isolated nucleic acid has a sequence of 80%, 85%, 90%, 92%, 94%, 95%, 96%, 97%, 98%, 99% or 100% identity to SEQ ID NO: 10 or 26 the isolated nucleic acid, inducible promoter, nucleic acid, expression cassette, or vector is integrated at a genomic locus that is different to the locus of native SEQ ID NO: 10 or 26, i.e., is not operably inserted upstream of the gene encoding SEQ ID NO: 42;
the isolated nucleic acid has a sequence of 80%, 85%, 90%, 92%, 94%, 95%, 96%, 97%, 98%, 99% or 100% identity to SEQ ID NO: 11 or 27 the isolated nucleic acid, inducible promoter, nucleic acid, expression cassette, or vector is integrated at a genomic locus that is different to the locus of native SEQ ID NO: 11 or 27, i.e., is not operably inserted upstream of the gene encoding SEQ ID NO: 43;
the isolated nucleic acid has a sequence of 80%, 85%, 90%, 92%, 94%, 95%, 96%, 97%, 98%, 99% or 100% identity to SEQ ID NO: 12 or 28 the isolated nucleic acid, inducible promoter, nucleic acid, expression cassette, or vector is integrated at a genomic locus that is different to the locus of native SEQ ID NO: 12 or 28, i.e., is not operably inserted upstream of the gene encoding SEQ ID NO: 44;
the isolated nucleic acid has a sequence of 80%, 85%, 90%, 92%, 94%, 95%, 96%, 97%, 98%, 99% or 100% identity to SEQ ID NO: 13 or 29 the isolated nucleic acid, inducible promoter, nucleic acid, expression cassette, or vector is integrated at a genomic locus that is different to the locus of native SEQ ID NO: 13 or 29, i.e., is not operably inserted upstream of the gene encoding SEQ ID NO: 45;
the isolated nucleic acid has a sequence of 80%, 85%, 90%, 92%, 94%, 95%, 96%, 97%, 98%, 99% or 100% identity to SEQ ID NO: 14 or 30 the isolated nucleic acid, inducible promoter, nucleic acid, expression cassette, or vector is integrated at a genomic locus that is different to the locus of native SEQ ID NO: 14 or 30, i.e., is not operably inserted upstream of the gene encoding SEQ ID NO: 46;
the isolated nucleic acid has a sequence of 80%, 85%, 90%, 92%, 94%, 95%, 96%, 97%, 98%, 99% or 100% identity to SEQ ID NO: 15 or 31 the isolated nucleic acid, inducible promoter, nucleic acid, expression cassette, or vector is integrated at a genomic locus that is different to the locus of native SEQ ID NO: 15 or 31, i.e., is not operably inserted upstream of the gene encoding SEQ ID NO: 47;
the isolated nucleic acid has a sequence of 80%, 85%, 90%, 92%, 94%, 95%, 96%, 97%, 98%, 99% or 100% identity to SEQ ID NO: 16 or 32 the isolated nucleic acid, inducible promoter, nucleic acid, expression cassette, or vector is integrated at a genomic locus that is different to the locus of native SEQ ID NO: 16 or 32, i.e., is not operably inserted upstream of the gene encoding SEQ ID NO: 48; or
the isolated nucleic acid has a sequence of 80%, 85%, 90%, 92%, 94%, 95%, 96%, 97%, 98%, 99% or 100% identity to SEQ ID NO: 17 or 33 the isolated nucleic acid, inducible promoter, nucleic acid, expression cassette, or vector is integrated at a genomic locus that is different to the locus of native SEQ ID NO: 17 or 33, i.e., is not operably inserted upstream of the gene encoding SEQ ID NO: 49.

23. A method of producing an expression product of a gene or a secondary metabolite or animal feed, wherein the method comprises the use of the isolated nucleic acid of claim 1 and optionally an, inducible promoter, optionally wherein the isolated nucleic acid is in a nucleic acid construct, optionally wherein the isolated nucleic acid is in an expression cassette, a vector, and/or a cell.

24. (canceled)

25. (canceled)

26. A kit comprising at least two of any of:
(a) the isolated nucleic acid of claim 1;
(b) a nucleic acid construct comprising at least a first and a second nuclei acid sequence, wherein the first nucleic acid sequence comprises or consists of the isolated nucleic acid sequence of claim 1, optionally, wherein expression from the promoter is induced in YNB media or in ACH +caa media;
(b) an expression cassette comprising the isolated nucleic acid of claim 1 or nucleic acid construct comprising at least a first and a second nucleic acid sequence, wherein the first nucleic acid sequence comprises or consists of the isolated nucleic acid sequence of claim 1, optionally, wherein expression from the promoter is induced in YNB media or in ACH +caa media;
(c) a vector comprising the nucleic acid of claim 1;
(d) a cell comprising the nucleic acid of claim 1;
an inducing agent, optionally wherein the inducing agent is formate, optionally, wherein the expression from the promoter is induced in YNB media or in ACH +caa media.

27. (canceled)

28. (canceled)

29. A method of producing a vaccine comprising using the isolated nucleic acid of claim 1, optionally, inducible promoter, nucleic acid construct comprising the nucleic acid of claim 1, an expression cassette comprising the nucleic acid of claim 1, a vector comprising the nucleic acid of claim 1, or a cell comprising the nucleic acid of claim 1.

30. A food product wherein the food product comprises a cell according to claim 16.
